SEQUENCE LISTING (157 sequences)

SEQ ID NO: 1 - protein, 5 aa, Artificial sequence
  Anti-CD20 ( GA101)  CDR H1 Kabat
YSWIN

SEQ ID NO: 2 - protein, 17 aa, Artificial sequence
  Anti-CD20 ( GA101)  CDR H2 Kabat
RIFPGDGDTDYNGKFKG

SEQ ID NO: 3 - protein, 10 aa, Artificial sequence
  Anti-CD20 ( GA101)  CDR H3 Kabat
NVFDGYWLVY

SEQ ID NO: 4 - protein, 16 aa, Artificial sequence
  Anti-CD20 ( GA101)  CDR L1 Kabat
RSSKSLLHSNGITYLY

SEQ ID NO: 5 - protein, 7 aa, Artificial sequence
  Anti-CD20 ( GA101)  CDR L2 Kabat
QMSNLVS

SEQ ID NO: 6 - protein, 9 aa, Artificial sequence
  Anti-CD20 ( GA101)  CDR L3 Kabat
AQNLELPYT

SEQ ID NO: 7 - protein, 407 aa, Artificial sequence
  Anti-CD20-(GA101)-Fab heavy chain-CD28ATD- CD28CSD-CD3zSSD fusion pETR 17097
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 222 aa, Artificial sequence
  Anti-CD20-(GA101)-Fab heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 219 aa, Artificial sequence
  Anti-CD20-(GA101)-Fab light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 112 aa, Artificial sequence
  Anti-CD20-(GA101) VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 11 - protein, 107 aa, Artificial sequence [sequence omitted]

SEQ ID NO: 12 - protein, 119 aa, Artificial sequence
  Anti-CD20-(GA101) VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 13 - protein, 103 aa, Artificial sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 27 aa, Artificial sequence
  CD28ATD
FWVLVVVGGVLACYSLLVTVAFIIFWV

SEQ ID NO: 15 - protein, 41 aa, Artificial sequence
  CD28CSD
RSKRSRLLHSDYMNMTPRRPGPTRKHYQPYAPPRDFAAYRS

SEQ ID NO: 16 - protein, 112 aa, Artificial sequence
  CD3zSSD
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 180 aa, Artificial sequence
  CD28ATD-CD28CSD-CD3zSSD
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 238 aa, Artificial sequence
  eGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 19 - protein, 20 aa, Artificial sequence
  (G4S)4 linker
GGGGSGGGGSGGGGSGGGGS

SEQ ID NO: 20 - protein, 5 aa, Artificial sequence
  G4S linker
GGGGS

SEQ ID NO: 21 - protein, 19 aa, Artificial sequence
  T2A linker
GEGRGSLLTCGDVEENPGP

SEQ ID NO: 22 - DNA, 2659 bp, Artificial sequence
  Anti-CD20-(GA101)-Fab-CD28ATD-CD28CSD-CD3zSSD pETR17097
atgggatggagctgtatcatcctcttcttggtagcaacagctaccggtgtgcattccgatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaaccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaatagaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactcccaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 23 - DNA, 336 bp, Artificial sequence
  Anti-CD20-(GA101)-Fab-VL
gatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaa

SEQ ID NO: 24 - DNA, 324 bp, Artificial sequence
  Fab CL
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 25 - DNA, 357 bp, Artificial sequence
  Anti-CD20-(GA101)-Fab-VH
caggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagc

SEQ ID NO: 26 - DNA, 309 bp, Artificial sequence
  Fab CH1
gctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgc

SEQ ID NO: 27 - DNA, 647 bp, Artificial sequence
  IRES EV71 internal ribosomal entry side
cccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaac

SEQ ID NO: 28 - DNA, 15 bp, Artificial sequence
  G4S linker
ggagggggcggatcc

SEQ ID NO: 29 - DNA, 81 bp, Artificial sequence
  CD28ATD
ttttgggtgctggtggtggttggtggagtcctggcttgctatagcttgctagtaacagtggcctttattattttctgggtg

SEQ ID NO: 30 - DNA, 123 bp, Artificial sequence
  CD28CSD
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 31 - DNA, 336 bp, Artificial sequence
  CD3zSSD
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - DNA, 540 bp, Artificial sequence
  CD28ATD-CD28CSD-CD3zSSD
ttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 33 - DNA, 63 bp, Artificial sequence
  T2A linker
tccggagagggcagaggaagtcttctaacatgcggtgacgtggaggagaatcccggccctagg

SEQ ID NO: 34 - DNA, 717 bp, Artificial sequence
  eGFP
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 35 - DNA, 3438 bp, Artificial sequence
  Anti-CD20-(GA101)-Fab-CD28ATD-CD28CSD-CD3zSSD- eGFP pETR17097
atgggatggagctgtatcatcctcttcttggtagcaacagctaccggtgtgcattccgatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaatagaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactcccaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 36 - protein, 411 aa, Artificial sequence
  Anti-CD20-(GA101)-crossFab VH-CL light chain-ATD-CD28ATD-CD28CSD-CD3zSSD fusion pETR17098
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 226 aa, Artificial sequence
  Anti-CD20-(GA101)-crossFab VH-CL light chain pETR17098
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 217 aa, Artificial sequence
  Anti-CD20-(GA101)-crossFab VL-CH1 heavy chain- pETR17098
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - protein, 107 aa, Artificial sequence
  crossFab CL
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 105 aa, Artificial sequence
  crossFab CH1
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 402 aa, Artificial sequence
  Anti-CD20-(GA101)-crossFab VL-CH1 heavy-chain-ATD-CD28ATD-CD28CSD-CD3zSSD fusion
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 217 aa, Artificial sequence; identical to SEQ ID NO: 38
  Anti-CD20-(GA101)-crossFab VL-CH1 heavy chain

SEQ ID NO: 43 - protein, 226 aa, Artificial sequence; identical to SEQ ID NO: 37
  Anti-CD20-(GA101)-crossFab VH-CL light chain

SEQ ID NO: 44 - protein, 107 aa, Artificial sequence; identical to SEQ ID NO: 39
  crossFab CL

SEQ ID NO: 45 - protein, 105 aa, Artificial sequence; identical to SEQ ID NO: 40
  crossFab CH1

SEQ ID NO: 46 - DNA, 2664 bp, Artificial sequence
  Anti-CD20-(GA101)-crossFabVH-CL CD28ATD- CD28CSD-CD3zSSD pETR17098
atgggatggagctgtatcatcctcttcttggtagcaacagctaccggtgtgcattcccaggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaacagccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgctgataaggaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactccgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgatcggttctccggttccgggtcaggcactgatttcacactgaaaatcagcagggtggaggctgaggatgttggagtttattactgcgctcagaatctagaacttccttacaccttcggcggagggaccaaggtggagatcaaatccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 47 - DNA, 324 bp, Artificial sequence
  crossFab CL
gctagcgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaacagccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgctga

SEQ ID NO: 48 - DNA, 315 bp, Artificial sequence
  crossFab CH1
tccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgc

SEQ ID NO: 49 - DNA, 3444 bp, Artificial sequence
  Anti-CD20-(GA101)-crossFabVH-CL CD28ATD- CD28CSD-CD3zSSD-eGFP pETR17098
atgggatggagctgtatcatcctcttcttggtagcaacagctaccggtgtgcattcccaggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaacagccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgctgataaggaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactccgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgatcggttctccggttccgggtcaggcactgatttcacactgaaaatcagcagggtggaggctgaggatgttggagtttattactgcgctcagaatctagaacttccttacaccttcggcggagggaccaaggtggagatcaaatccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 50 - protein, 404 aa, Artificial sequence
  Anti-CD20-(GA101)-Fab VL-CL light chain- CD28ATD-CD28CSD-CD3zSSD fusion
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 658 aa, Artificial sequence
  Anti-CD20-(GA101)-scFab-CD28ATD-CD28CSD-CD3zSSD fusion pETR17101
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 473 aa, Artificial sequence
  Anti-CD20-(GA101)-scFab
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 106 aa, Artificial sequence
  scFab-CL
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 54 - protein, 32 aa, Artificial sequence
  (G4S)6G2 linker
GGGGSGGGGSGGGGSGGGGSGGGGSGGGGSGG

SEQ ID NO: 55 - DNA, 2031 bp, Artificial sequence
  Anti-CD20-(GA101)-scFab-CD28ATD-CD28CSD-CD3zSSD fusion pETR170101
atgggatggagctgtatcatcctcttcttggtagcaacagctacgggtgtgcattccgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgatcggttctccggttccgggtcaggcactgatttcacactgaaaatcagcagggtggaggctgaggatgttggagtttattactgcgctcagaatctagaacttccttacaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggcggaggatctggtggcggaggtagtggtggtggtggatctggcggaggcggctccggcggaggtggaagcggaggtggtggttccggaggacaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 56 - DNA, 336 bp, Artificial sequence
  scFab-VL
gacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgatcggttctccggttccgggtcaggcactgatttcacactgaaaatcagcagggtggaggctgaggatgttggagtttattactgcgctcagaatctagaacttccttacaccttcggcggagggaccaaggtggagatcaaa

SEQ ID NO: 57 - DNA, 321 bp, Artificial sequence
  scFab-CL
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - DNA, 357 bp, Artificial sequence
  Anti-CD20-(GA101)- scFab-VH
caggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctca

SEQ ID NO: 59 - DNA, 2806 bp, Artificial sequence
  Anti-CD20-(GA101)-scFab-CD28ATD-CD28CSD- CD3zSSD-eGFP fusion pETR170101
atggagctgtatcatcctcttcttggtagcaacagctacgggtgtgcattccgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgatcggttctccggttccgggtcaggcactgatttcacactgaaaatcagcagggtggaggctgaggatgttggagtttattactgcgctcagaatctagaacttccttacaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggcggaggatctggtggcggaggtagtggtggtggtggatctggcggaggcggctccggcggaggtggaagcggaggtggtggttccggaggacaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 60 - protein, 436 aa, Artificial sequence
  Anti-CD20-(GA101)-ds-scFv-CD28ATD-CD28CSD- CD3zSSD fusion pETR17162
QVQLVQSGAEVKKPGSSVKVSCKASGYAFSYSWINWVRQAPGQCLEWMGRIFPGDGDTDYNGKFKGRVTITADKSTSTAYMELSSLRSEDTAVYYCARNVFDGYWLVYWGQGTLVTVSSGGGGSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGCGTKVEIKGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 251 aa, Artificial sequence
  Anti-CD20-(GA101)-ds-scFv
QVQLVQSGAEVKKPGSSVKVSCKASGYAFSYSWINWVRQAPGQCLEWMGRIFPGDGDTDYNGKFKGRVTITADKSTSTAYMELSSLRSEDTAVYYCARNVFDGYWLVYWGQGTLVTVSSGGGGSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGCGTKVEIK

SEQ ID NO: 62 - protein, 119 aa, Artificial sequence
  Anti-CD20-(GA101)- ds-Fab VH
QVQLVQSGAEVKKPGSSVKVSCKASGYAFSYSWINWVRQAPGQCLEWMGRIFPGDGDTDYNGKFKGRVTITADKSTSTAYMELSSLRSEDTAVYYCARNVFDGYWLVYWGQGTLVTVSS

SEQ ID NO: 63 - protein, 112 aa, Artificial sequence
  Anti-CD20-(GA101)- ds-Fab VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGCGTKVEIK

SEQ ID NO: 64 - DNA, 1364 bp, Artificial sequence
  Anti-CD20-(GA101)-ds-Fab-CD28ATD-CD28CSD- CD3zSSD fusion pETR17162
atgggatggagctgtatcatcctcttcttggtagcaacagctaccggtgtgcattcccaggtgcaattggtgcagtctggcgctgaagttaagaagcctgggagttcagtgaaggtctcctgcaaggcttccggttacgccttcagctattcttggatcaattgggtgcggcaggcgcctggacaatgtctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcaggagggggcggaagtggtggcgggggaagcggcgggggtggcagcggagggggcggatctgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccgcttctccggttccgggtcaggcactgatttcacactgaaaatcagcagggtggaggctgaggatgttggagtttattactgcgctcagaatctagaacttccttacaccttcggctgtgggaccaaggtggagatcaaggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 65 - DNA, 357 bp, Artificial sequence
  Anti-CD20-(GA101)- ds-Fab VH
caggtgcaattggtgcagtctggcgctgaagttaagaagcctgggagttcagtgaaggtctcctgcaaggcttccggttacgccttcagctattcttggatcaattgggtgcggcaggcgcctggacaatgtctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctca

SEQ ID NO: 66 - DNA, 336 bp, Artificial sequence
  Anti-CD20-(GA101)- ds-Fab VL
gacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccgcttctccggttccgggtcaggcactgatttcacactgaaaatcagcagggtggaggctgaggatgttggagtttattactgcgctcagaatctagaacttccttacaccttcggctgtgggaccaaggtggagatcaaa

SEQ ID NO: 67 - DNA, 2151 bp, Artificial sequence
  Anti-CD20-(GA101)-ds-scFv-CD28ATD-CD28CSD- CD3zSSD-eGFP fusion pETR17162
gccaccatgggatggagctgtatcatcctcttcttggtagcaacagctaccggtgtgcattcccaggtgcaattggtgcagtctggcgctgaagttaagaagcctgggagttcagtgaaggtctcctgcaaggcttccggttacgccttcagctattcttggatcaattgggtgcggcaggcgcctggacaatgtctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcaggagggggcggaagtggtggcgggggaagcggcgggggtggcagcggagggggcggatctgacatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccgcttctccggttccgggtcaggcactgatttcacactgaaaatcagcagggtggaggctgaggatgttggagtttattactgcgctcagaatctagaacttccttacaccttcggctgtgggaccaaggtggagatcaaaggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 68 - protein, 5 aa, Artificial sequence
  Anti-PDL1 CDR H1 Kabat
DSWIH

SEQ ID NO: 69 - protein, 17 aa, Artificial sequence
  Anti-PDL1 CDR H2 Kabat
WISPYGGSTYYADSVKG

SEQ ID NO: 70 - protein, 9 aa, Artificial sequence
  Anti-PDL1 CDR H3 Kabat
RHWPGGFDY

SEQ ID NO: 71 - protein, 11 aa, Artificial sequence
  Anti- PDL1 CDR 1 L1 Kabat
RASQDVSTAVA

SEQ ID NO: 72 - protein, 7 aa, Artificial sequence
  Anti- PDL1 CDR L2 Kabat
SASFLYS

SEQ ID NO: 73 - protein, 9 aa, Artificial sequence
  Anti- PDL1 CDR L3 Kabat
QQYLYHPAT

SEQ ID NO: 74 - protein, 406 aa, Artificial sequence
  Anti-PDL1 Fab heavy chain-CD28ATD-CD28CSD- CD3zSSD fusion
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 221 aa, Artificial sequence
  Anti-PDL1 Fab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - protein, 215 aa, Artificial sequence
  Anti-PDL1 Fab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 108 aa, Artificial sequence
  Anti-PDL1-VL
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 78 - protein, 118 aa, Artificial sequence [sequence omitted]

SEQ ID NO: 79 - protein, 410 aa, Artificial sequence
  Anti-PDL1-crossFab VH-CL light chain-CD28ATD- CD28CSD-CD3zSSD fusion pETR17098
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 225 aa, Artificial sequence
  Anti-PDL1-crossFab VH-CL light chain pETR17098
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 213 aa, Artificial sequence
  Anti-PDL1-crossFab VL-CH heavy chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 82 - protein, 398 aa, Artificial sequence
  Anti-PDL1-crossFab VL-CH heavy chain-ATD- CD28ATD-CD28CSD-CD3zSSD fusion
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 213 aa, Artificial sequence; identical to SEQ ID NO: 81
  Anti-PDL1-crossFab VL-CH heavy chain

SEQ ID NO: 84 - protein, 225 aa, Artificial sequence; identical to SEQ ID NO: 80
  Anti-PDL1-crossFab VH-CL light chain

SEQ ID NO: 85 - protein, 400 aa, Artificial sequence
  Anti-PDL1-Fab light chain-ATD-CD28ATD-CD28CSD- CD3zSSD fusion
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 651 aa, Artificial sequence
  Anti-PDL1-scFab-CD28ATD-CD28CSD-CD3zSSD fusion
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 466 aa, Artificial sequence
  Anti-PDL1-scFab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 430 aa, Artificial sequence
  Anti-PDL1-ds-scFv-CD28ATD-CD28CSD-CD3zSSD fusion
EVQLVESGGGLVQPGGSLRLSCAASGFTFSDSWIHWVRQAPGKCLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGCKVEIKGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 245 aa, Artificial sequence
  Anti-PDL1-ds-scFv
EVQLVESGGGLVQPGGSLRLSCAASGFTFSDSWIHWVRQAPGKCLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGCKVEIK

SEQ ID NO: 90 - protein, 118 aa, Artificial sequence
  Anti-PDL1-ds-VH
EVQLVESGGGLVQPGGSLRLSCAASGFTFSDSWIHWVRQAPGKCLEWVAWISPYGGSTYYADSVKGRFTISADTSKNTAYLQMNSLRAEDTAVYYCARRHWPGGFDYWGQGTLVTVSS

SEQ ID NO: 91 - protein, 107 aa, Artificial sequence
  Anti-PDL1-ds-VL
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGCKVEIK

SEQ ID NO: 92 - DNA, 780 bp, Homo sapiens
atggcgcgcccgcatccgtggtggctgtgcgtgctgggcaccctggtgggcctgagcgcgaccccggcgccgaaaagctgcccggaacgccattattgggcgcagggcaaactgtgctgccagatgtgcgaaccgggcacctttctggtgaaagattgcgatcagcatcgcaaagcggcgcagtgcgatccgtgcattccgggcgtgagctttagcccggatcatcatacccgcccgcattgcgaaagctgccgccattgcaacagcggcctgctggtgcgcaactgcaccattaccgcgaacgcggaatgcgcgtgccgcaacggctggcagtgccgcgataaagaatgcaccgaatgcgatccgctgccgaacccgagcctgaccgcgcgcagcagccaggcgctgagcccgcatccgcagccgacccatctgccgtatgtgagcgaaatgctggaagcgcgcaccgcgggccatatgcagaccctggcggattttcgccagctgccggcgcgcaccctgagcacccattggccgccgcagcgcagcctgtgcagcagcgattttattcgcattctggtgatttttagcggcatgtttctggtgtttaccctggcgggcgcgctgtttctgcatcagcgccgcaaatatcgcagcaacaaaggcgaaagcccggtggaaccggcggaaccgtgccattatagctgcccgcgcgaagaagaaggcagcaccattccgattcaggaagattatcgcaaaccggaaccggcgtgcagcccg

SEQ ID NO: 93 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 94 - DNA, 750 bp, Mus musculus
atggcgtggccgccgccgtattggctgtgcatgctgggcaccctggtgggcctgagcgcgaccctggcgccgaacagctgcccggataaacattattggaccggcggcggcctgtgctgccgcatgtgcgaaccgggcaccttttttgtgaaagattgcgaacaggatcgcaccgcggcgcagtgcgatccgtgcattccgggcaccagctttagcccggattatcatacccgcccgcattgcgaaagctgccgccattgcaacagcggctttctgattcgcaactgcaccgtgaccgcgaacgcggaatgcagctgcagcaaaaactggcagtgccgcgatcaggaatgcaccgaatgcgatccgccgctgaacccggcgctgacccgccagccgagcgaaaccccgagcccgcagccgccgccgacccatctgccgcatggcaccgaaaaaccgagctggccgctgcatcgccagctgccgaacagcaccgtgtatagccagcgcagcagccatcgcccgctgtgcagcagcgattgcattcgcatttttgtgacctttagcagcatgtttctgatttttgtgctgggcgcgattctgttttttcatcagcgccgcaaccatggcccgaacgaagatcgccaggcggtgccggaagaaccgtgcccgtatagctgcccgcgcgaagaagaaggcagcgcgattccgattcaggaagattatcgcaaaccggaaccggcgttttatccg

SEQ ID NO: 95 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 96 - DNA, 660 bp, Homo sapiens
atgctgcgcctgctgctggcgctgaacctgtttccgagcattcaggtgaccggcaacaaaattctggtgaaacagagcccgatgctggtggcgtatgataacgcggtgaacctgagctgcaaatatagctataacctgtttagccgcgaatttcgcgcgagcctgcataaaggcctggatagcgcggtggaagtgtgcgtggtgtatggcaactatagccagcagctgcaggtgtatagcaaaaccggctttaactgcgatggcaaactgggcaacgaaagcgtgaccttttatctgcagaacctgtatgtgaaccagaccgatatttatttttgcaaaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgcgcagcaaacgcagccgcctgctgcatagcgattatatgaacatgaccccgcgccgcccgggcccgacccgcaaacattatcagccgtatgcgccgccgcgcgattttgcggcgtatcgcagc

SEQ ID NO: 97 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 98 - DNA, 654 bp, Mus musculus
atgaccctgcgcctgctgtttctggcgctgaacttttttagcgtgcaggtgaccgaaaacaaaattctggtgaaacagagcccgctgctggtggtggatagcaacgaagtgagcctgagctgccgctatagctataacctgctggcgaaagaatttcgcgcgagcctgtataaaggcgtgaacagcgatgtggaagtgtgcgtgggcaacggcaactttacctatcagccgcagtttcgcagcaacgcggaatttaactgcgatggcgattttgataacgaaaccgtgacctttcgcctgtggaacctgcatgtgaaccataccgatatttatttttgcaaaattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccg

SEQ ID NO: 99 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 100 - DNA, 768 bp, Homo sapiens
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 101 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 102 - DNA, 768 bp, Mus musculus
atgggcaacaactgctataacgtggtggtgattgtgctgctgctggtgggctgcgaaaaagtgggcgcggtgcagaacagctgcgataactgccagccgggcaccttttgccgcaaatataacccggtgtgcaaaagctgcccgccgagcacctttagcagcattggcggccagccgaactgcaacatttgccgcgtgtgcgcgggctattttcgctttaaaaaattttgcagcagcacccataacgcggaatgcgaatgcattgaaggctttcattgcctgggcccgcagtgcacccgctgcgaaaaagattgccgcccgggccaggaactgaccaaacagggctgcaaaacctgcagcctgggcacctttaacgatcagaacggcaccggcgtgtgccgcccgtggaccaactgcagcctggatggccgcagcgtgctgaaaaccggcaccaccgaaaaagatgtggtgtgcggcccgccggtggtgagctttagcccgagcaccaccattagcgtgaccccggaaggcggcccgggcggccatagcctgcaggtgctgaccctgtttctggcgctgaccagcgcgctgctgctggcgctgatttttattaccctgctgtttagcgtgctgaaatggattcgcaaaaaatttccgcatatttttaaacagccgtttaaaaaaaccaccggcgcggcgcaggaagaagatgcgtgcagctgccgctgcccgcaggaagaagaaggcggcggcggcggctatgaactg

SEQ ID NO: 103 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 104 - DNA, 831 bp, Homo sapiens
atgtgcgtgggcgcgcgccgcctgggccgcggcccgtgcgcggcgctgctgctgctgggcctgggcctgagcaccgtgaccggcctgcattgcgtgggcgatacctatccgagcaacgatcgctgctgccatgaatgccgcccgggcaacggcatggtgagccgctgcagccgcagccagaacaccgtgtgccgcccgtgcggcccgggcttttataacgatgtggtgagcagcaaaccgtgcaaaccgtgcacctggtgcaacctgcgcagcggcagcgaacgcaaacagctgtgcaccgcgacccaggataccgtgtgccgctgccgcgcgggcacccagccgctggatagctataaaccgggcgtggattgcgcgccgtgcccgccgggccattttagcccgggcgataaccaggcgtgcaaaccgtggaccaactgcaccctggcgggcaaacataccctgcagccggcgagcaacagcagcgatgcgatttgcgaagatcgcgatccgccggcgacccagccgcaggaaacccagggcccgccggcgcgcccgattaccgtgcagccgaccgaagcgtggccgcgcaccagccagggcccgagcacccgcccggtggaagtgccgggcggccgcgcggtggcggcgattctgggcctgggcctggtgctgggcctgctgggcccgctggcgattctgctggcgctgtatctgctgcgccgcgatcagcgcctgccgccggatgcgcataaaccgccgggcggcggcagctttcgcaccccgattcaggaagaacaggcggatgcgcatagcaccctggcgaaaatt

SEQ ID NO: 105 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 106 - DNA, 816 bp, Mus musculus
atgtatgtgtgggtgcagcagccgaccgcgctgctgctgctggcgctgaccctgggcgtgaccgcgcgccgcctgaactgcgtgaaacatacctatccgagcggccataaatgctgccgcgaatgccagccgggccatggcatggtgagccgctgcgatcatacccgcgataccctgtgccatccgtgcgaaaccggcttttataacgaagcggtgaactatgatacctgcaaacagtgcacccagtgcaaccatcgcagcggcagcgaactgaaacagaactgcaccccgacccaggataccgtgtgccgctgccgcccgggcacccagccgcgccaggatagcggctataaactgggcgtggattgcgtgccgtgcccgccgggccattttagcccgggcaacaaccaggcgtgcaaaccgtggaccaactgcaccctgagcggcaaacagacccgccatccggcgagcgatagcctggatgcggtgtgcgaagatcgcagcctgctggcgaccctgctgtgggaaacccagcgcccgacctttcgcccgaccaccgtgcagagcaccaccgtgtggccgcgcaccagcgaactgccgagcccgccgaccctggtgaccccggaaggcccggcgtttgcggtgctgctgggcctgggcctgggcctgctggcgccgctgaccgtgctgctggcgctgtatctgctgcgcaaagcgtggcgcctgccgaacaccccgaaaccgtgctggggcaacagctttcgcaccccgattcaggaagaacataccgatgcgcattttaccctggcgaaaatt

SEQ ID NO: 107 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 108 - DNA, 597 bp, Homo sapiens
atgaaaagcggcctgtggtatttttttctgttttgcctgcgcattaaagtgctgaccggcgaaattaacggcagcgcgaactatgaaatgtttatttttcataacggcggcgtgcagattctgtgcaaatatccggatattgtgcagcagtttaaaatgcagctgctgaaaggcggccagattctgtgcgatctgaccaaaaccaaaggcagcggcaacaccgtgagcattaaaagcctgaaattttgccatagccagctgagcaacaacagcgtgagcttttttctgtataacctggatcatagccatgcgaactattatttttgcaacctgagcatttttgatccgccgccgtttaaagtgaccctgaccggcggctatctgcatatttatgaaagccagctgtgctgccagctgaaattttggctgccgattggctgcgcggcgtttgtggtggtgtgcattctgggctgcattctgatttgctggctgaccaaaaaaaaatatagcagcagcgtgcatgatccgaacggcgaatatatgtttatgcgcgcggtgaacaccgcgaaaaaaagccgcctgaccgatgtgaccctg

SEQ ID NO: 109 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 110 - DNA, 600 bp, Mus musculus
atgaaaccgtatttttgccgcgtgtttgtgttttgctttctgattcgcctgctgaccggcgaaattaacggcagcgcggatcatcgcatgtttagctttcataacggcggcgtgcagattagctgcaaatatccggaaaccgtgcagcagctgaaaatgcgcctgtttcgcgaacgcgaagtgctgtgcgaactgaccaaaaccaaaggcagcggcaacgcggtgagcattaaaaacccgatgctgtgcctgtatcatctgagcaacaacagcgtgagcttttttctgaacaacccggatagcagccagggcagctattatttttgcagcctgagcatttttgatccgccgccgtttcaggaacgcaacctgagcggcggctatctgcatatttatgaaagccagctgtgctgccagctgaaactgtggctgccggtgggctgcgcggcgtttgtggtggtgctgctgtttggctgcattctgattatttggtttagcaaaaaaaaatatggcagcagcgtgcatgatccgaacagcgaatatatgtttatggcggcggtgaacaccaacaaaaaaagccgcctggcgggcgtgaccagc

SEQ ID NO: 111 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 112 - DNA, 279 bp, Homo sapiens
atgattcatctgggccatattctgtttctgctgctgctgccggtggcggcggcgcagaccaccccgggcgaacgcagcagcctgccggcgttttatccgggcaccagcggcagctgcagcggctgcggcagcctgagcctgccgctgctggcgggcctggtggcggcggatgcggtggcgagcctgctgattgtgggcgcggtgtttctgtgcgcgcgcccgcgccgcagcccggcgcaggaagatggcaaagtgtatattaacatgccgggccgcggc

SEQ ID NO: 113 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 114 - DNA, 237 bp, Mus musculus
atggatccgccgggctatctgctgtttctgctgctgctgccggtggcggcgagccagaccagcgcgggcagctgcagcggctgcggcaccctgagcctgccgctgctggcgggcctggtggcggcggatgcggtgatgagcctgctgattgtgggcgtggtgtttgtgtgcatgcgcccgcatggccgcccggcgcaggaagatggccgcgtgtatattaacatgccgggccgcggc

SEQ ID NO: 115 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 116 - DNA, 342 bp, Homo sapiens
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 117 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 118 - DNA, 345 bp, Mus musculus
atgggggctctggagccctcctggtgccttctgttccttcctgtcctcctgactgtgggaggattaagtcccgtacaggcccagagtgacactttcccaagatgcgactgttcttccgtgagccctggtgtactggctgggattgttctgggtgacttggtgttgactctgctgattgccctggctgtgtactctctgggccgcctggtctcccgaggtcaagggacagcggaagggacccggaaacaacacattgctgagactgagtcgccttatcaggagcttcagggtcagagaccagaagtatacagtgacctcaacacacagaggcaatattacagatga

SEQ ID NO: 119 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 120 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 121 - DNA, 492 bp, Homo sapiens
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 122 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 123 - DNA, 495 bp, Mus musculus
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 124 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 125 - DNA, 762 bp, Homo sapiens
atgtggcagctgctgctgccgaccgcgctgctgctgctggtgagcgcgggcatgcgcaccgaagatctgccgaaagcggtggtgtttctggaaccgcagtggtatcgcgtgctggaaaaagatagcgtgaccctgaaatgccagggcgcgtatagcccggaagataacagcacccagtggtttcataacgaaagcctgattagcagccaggcgagcagctattttattgatgcggcgaccgtggatgatagcggcgaatatcgctgccagaccaacctgagcaccctgagcgatccggtgcagctggaagtgcatattggctggctgctgctgcaggcgccgcgctgggtgtttaaagaagaagatccgattcatctgcgctgccatagctggaaaaacaccgcgctgcataaagtgacctatctgcagaacggcaaaggccgcaaatattttcatcataacagcgatttttatattccgaaagcgaccctgaaagatagcggcagctatttttgccgcggcctgtttggcagcaaaaacgtgagcagcgaaaccgtgaacattaccattacccagggcctggcggtgagcaccattagcagcttttttccgccgggctatcaggtgagcttttgcctggtgatggtgctgctgtttgcggtggataccggcctgtattttagcgtgaaaaccaacattcgcagcagcacccgcgattggaaagatcataaatttaaatggcgcaaagatccgcaggataaa

SEQ ID NO: 126 - protein, 261 aa, Mus musculus
MFQNAHSGSQWLLPPLTILLLFAFADRQSAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPREYWRKSLSIRKHQAPQDK

SEQ ID NO: 127 - DNA, 786 bp, Mus musculus
atgtttcagaatgcacactctggaagccaatggctacttccaccactgacaattctgctgctgtttgcttttgcagacaggcagagtgcagctcttccgaaggctgtggtgaaactggaccccccatggatccaggtgctcaaggaagacatggtgacactgatgtgcgaagggacccacaaccctgggaactcttctacccagtggttccacaacgggaggtccatccggagccaggtccaagccagttacacgtttaaggccacagtcaatgacagtggagaatatcggtgtcaaatggagcagacccgcctcagcgaccctgtagatctgggagtgatttctgactggctgctgctccagacccctcagcgggtgtttctggaaggggaaaccatcacgctaaggtgccatagctggaggaacaaactactgaacaggatctcattcttccataatgaaaaatccgtgaggtatcatcactacaaaagtaatttctctatcccaaaagccaaccacagtcacagtggggactactactgcaaaggaagtctaggaagtacacagcaccagtccaagcctgtcaccatcactgtccaagatccagcaactacatcctccatctctctagtctggtaccacactgctttctccctagtgatgtgcctcctgtttgcagtggacacgggcctttatttctacgtacggagaaatcttcaaaccccgagggagtactggaggaagtccctgtcaatcagaaagcaccaggctcctcaagacaagtga

SEQ ID NO: 128 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 129 - DNA, 648 bp, Homo sapiens
atgggctggattcgcggccgccgcagccgccatagctgggaaatgagcgaatttcataactataacctggatctgaaaaaaagcgattttagcacccgctggcagaaacagcgctgcccggtggtgaaaagcaaatgccgcgaaaacgcgagcccgttttttttttgctgctttattgcggtggcgatgggcattcgctttattattatggtggcgatttggagcgcggtgtttctgaacagcctgtttaaccaggaagtgcagattccgctgaccgaaagctattgcggcccgtgcccgaaaaactggatttgctataaaaacaactgctatcagttttttgatgaaagcaaaaactggtatgaaagccaggcgagctgcatgagccagaacgcgagcctgctgaaagtgtatagcaaagaagatcaggatctgctgaaactggtgaaaagctatcattggatgggcctggtgcatattccgaccaacggcagctggcagtgggaagatggcagcattctgagcccgaacctgctgaccattattgaaatgcagaaaggcgattgcgcgctgtatgcgagcagctttaaaggctatattgaaaactgcagcaccccgaacacctatatttgcatgcagcgcaccgtg

SEQ ID NO: 130 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 131 - DNA, 696 bp, Mus musculus
atggcgctgattcgcgatcgcaaaagccatcatagcgaaatgagcaaatgccataactatgatctgaaaccggcgaaatgggataccagccaggaacagcagaaacagcgcctggcgctgaccaccagccagccgggcgaaaacggcattattcgcggccgctatccgattgaaaaactgaaaattagcccgatgtttgtggtgcgcgtgctggcgattgcgctggcgattcgctttaccctgaacaccctgatgtggctggcgatttttaaagaaacctttcagccggtgctgtgcaacaaagaagtgccggtgagcagccgcgaaggctattgcggcccgtgcccgaacaactggatttgccatcgcaacaactgctatcagttttttaacgaagaaaaaacctggaaccagagccaggcgagctgcctgagccagaacagcagcctgctgaaaatttatagcaaagaagaacaggattttctgaaactggtgaaaagctatcattggatgggcctggtgcagattccggcgaacggcagctggcagtgggaagatggcagcagcctgagctataaccagctgaccctggtggaaattccgaaaggcagctgcgcggtgtatggcagcagctttaaagcgtataccgaagattgcgcgaacctgaacacctatatttgcatgaaacgcgcggtg

SEQ ID NO: 132 - protein, 4 aa, Artificial sequence
  CD28 YMNM
YMNM

SEQ ID NO: 133 - protein, 4 aa, Artificial sequence
  CD28 PYAP
PYAP

SEQ ID NO: 134 - protein, 4 aa, Artificial sequence
  CD28 FMNM
FMNM

SEQ ID NO: 135 - protein, 4 aa, Artificial sequence
  CD28 AYAA
AYAA

SEQ ID NO: 136 - protein, 21 aa, Artificial sequence
  Signal peptide
ATMGWSCIILFLVATATGVHS

SEQ ID NO: 137 - DNA, 57 bp, Artificial sequence
  Signal peptide DNA sequence
atgggatggagctgtatcatcctcttcttggtagcaacagctaccggtgtgcactcc

SEQ ID NO: 138 - protein, 5 aa, Artificial sequence
  Anti-CEA (98/99) CDR H1 Kabat
EFGMN

SEQ ID NO: 139 - protein, 17 aa, Artificial sequence
  Anti-CEA (98/99) CDR H2 Kabat
WINTKTGEATYVEEFKG

SEQ ID NO: 140 - protein, 12 aa, Artificial sequence
  Anti-CEA (98/99) CDR H3 Kabat
WDFAYYVEAMDY

SEQ ID NO: 141 - protein, 11 aa, Artificial sequence
  Anti-CEA (98/99) CDR 1 L1 Kabat
KASAAVGTYVA

SEQ ID NO: 142 - protein, 7 aa, Artificial sequence
  Anti-CEA (98/99) CDR L2 Kabat
SASYRKR

SEQ ID NO: 143 - protein, 10 aa, Artificial sequence
  Anti-CEA (98/99) CDR L3 Kabat
HQYYTYPLFT

SEQ ID NO: 144 - protein, 436 aa, Artificial sequence
  Anti-CEA (98/99)-scFv-CD28ATD-CD28CSD-CD3zSSD fusion
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGGGSDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 251 aa, Artificial sequence
  Anti-CEA (98/99)-scFv
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGGGSDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRT

SEQ ID NO: 146 - protein, 121 aa, Artificial sequence
  Anti-CEA (98/99)-VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 147 - protein, 110 aa, Artificial sequence
  Anti-CEA (98/99)-VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRT

SEQ ID NO: 148 - protein, 5 aa, Artificial sequence
  Anti-CEA (T84.66) CDR H1 Kabat
DTYMH

SEQ ID NO: 149 - protein, 17 aa, Artificial sequence
  Anti-CEA (T84.66) CDR H2 Kabat
RIDPANGNSKYVPKFQG

SEQ ID NO: 150 - protein, 12 aa, Artificial sequence
  Anti-CEA (T84.66) CDR H3 Kabat
FGYYVSDYAMAY

SEQ ID NO: 151 - protein, 15 aa, Artificial sequence
  Anti-CEA (T84.66) CDR 1 L1 Kabat
RAGESVDIFGVGFLH

SEQ ID NO: 152 - protein, 7 aa, Artificial sequence
  Anti-CEA (T84.66) CDR L2 Kabat
RASNRAT

SEQ ID NO: 153 - protein, 9 aa, Artificial sequence
  Anti-CEA (T84.66) CDR L3 Kabat
QQTNEDPYT

SEQ ID NO: 154 - protein, 437 aa, Artificial sequence
  Anti-CEA (T84.66)-scFv-CD28ATD-CD28CSD-CD3zSSD fusion
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSGGGGSGGGGSGGGGSGGGGSEIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 252 aa, Artificial sequence
  Anti-CEA (T84.66)-scFv
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSGGGGSGGGGSGGGGSGGGGSEIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 156 - protein, 121 aa, Artificial sequence
  Anti-CEA (T84.66)-VH
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 157 - protein, 111 aa, Artificial sequence
  Anti-CEA (T84.66)-VL
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

(19) United States
(12) Patent Application Publication
(10) Pub. No.: US 2020/0093861 A1
(43) Pub. Date: Mar. 26, 2020

(54) ANTIGEN BINDING RECEPTOR FORMATS

(71) Applicant: Hoffmann-La Roche Inc., Little Falls, NJ (US)

(72) Inventors: Christian KLEIN, Schlieren (CH); Ekkehard MOESSNER, Schlieren (CH); Diana DAROWSKI, Schlieren (CH); Kay-Gunnar STUBENRAUCH, Penzberg (DE)

(73) Assignee: Hoffmann-La Roche Inc., Little Falls, NJ (US)

(21) Appl. No.: 16/576,586

(22) Filed: Sep. 19, 2019

Related U.S. Application Data
(63) Continuation of application No. PCT/EP2018/057567, filed on Mar. 26, 2018.

(30) Foreign Application Priority Data
Mar. 27, 2017 (EP) 17163090.8
May 18, 2017 (EP) 17171775.4

(51) Int. Cl. A61K 35/17; C07K 14/725; C07K 14/705; C07K 16/28; C07K 16/30

(52) U.S. Cl. CPC A61K 35/17; C07K 14/7051; C07K 14/70517; C07K 14/70596; C07K 16/283; C07K 2319/00; C07K 14/70578; C07K 16/2827; C07K 16/3007; C07K 2317/565; C07K 14/70521

(57) ABSTRACT

The present invention generally relates to antigen binding receptors in new formats capable of specific binding to a tumor associated antigen. More precisely, the present invention relates to an antigen binding receptor which efficiently and specifically binds to/interacts with an antigen on the surface of a tumor cell, and to a T cell transfected/transduced with the antigen binding receptor. Furthermore, the invention relates to nucleic acid molecules and vectors encoding antigen binding receptors of the present invention. The invention also provides the production and use of T cells in a method for the treatment of particular diseases as well as pharmaceutical compositions/medicaments comprising antigen binding receptors and/or T cells of the present invention.

### CROSS REFERENCE TO RELATED APPLICATIONS

This application is a continuation of International Application No. PCT/EP2018/057567, filed Mar. 26, 2018, the content of which is herein incorporated by reference in its entirety, which claims priority to EP Application No. 17171775.4 filed May 18, 2017 and EP Application No. 17163090.8 filed Mar. 27, 2017.

### SEQUENCE LISTING

This application contains a Sequence Listing which has been submitted electronically in ASCII format and is hereby incorporated by reference in its entirety. Said ASCII copy, created on Sep. 17, 2019, is named “ P34241-US_sequence_listing.txt” and is 224,004 bytes in size.

### FIELD OF THE INVENTION

The present invention generally relates to antigen binding receptors in new formats capable of specific binding to a tumor associated antigen. More precisely, the present invention relates to an antigen binding receptor which efficiently and specifically binds to/interacts with an antigen on the surface of a tumor cell, and to a T cell transfected/transduced with the antigen binding receptor. Furthermore, the invention relates to nucleic acid molecules and vectors encoding antigen binding receptors of the present invention. The invention also provides the production and use of T cells in a method for the treatment of particular diseases as well as pharmaceutical compositions/medicaments comprising antigen binding receptors and/or T cells of the present invention.

### BACKGROUND

Adoptive T cell therapy (ACT) is a powerful treatment approach using cancer-specific T cells (Rosenberg and Restifo, Science 348(6230) (2015), 62-68). ACT may use naturally occurring tumor-specific cells or T cells rendered specific by genetic engineering using chimeric antigen receptors (Rosenberg and Restifo, Science 348(6230) (2015), 62-68). ACT can successfully treat and induce remission in patients suffering even from advanced and otherwise treatment refractory diseases such as acute lymphatic leukemia, non-hodgkins lymphoma or melanoma (Dudley et al., J Clin Oncol 26(32) (2008), 5233-5239; Grupp et al., N Engl J Med 368 (16) (2013), 1509-1518; Kochenderfer et al., J Clin Oncol. (2015) 33(6):540-549, doi: 10.1200/JCO.2014.56.2025. Epub 2014 Aug. 25).

However, despite impressive clinical efficacy, ACT can also lead to life-threatening toxicities due to off-effects of introduced chimeric antigen receptors or to expression of the target antigen in healthy tissue. Indeed, most targeted antigens are tumor-associated but not completely tumor-selective. Resulting off-target effects led to severe toxicity in several trials, e.g. CAR T cells targeting ErbB2, which is highly expressed by cancer cells but also at lower level in healthy cells, caused acute toxicity toward cardiopulmonary epithelia (Morgan et al., Mol Ther 18 (2010), 843-851). One strategy to overcome toxicity currently assessed is the reduction of CAR affinity towards the target antigen. However, these approaches may also limit the efficacy of ACT at the site of intended action.

Additionally, ACT is further limited due to the fact that once accumulated at the tumor site, the T cell response is repressed by various means. The tumor microenvironment may prevent efficient infiltration by repressor cells, secreted soluble factors from the tumor or stroma cells and by nutrient deprivation. Moreover, T cells express multiple immune repressive receptors which, upon activation, repress the T cell response, including e.g. cytotoxic T lymphocyte-associated antigen-4 (CTLA-4) and programmed cell death-1 (PD-1). Future clinical models need to counteract and overcome T cell repression while retaining tumor specificity and cytotoxicity.

Accordingly, the targeted tumor therapy, particularly the adoptive T cell therapy is still in need of more differentiated tools in order to suffice the needs of the cancer patients. Thus, there is still a need to provide new means having the potential to improve safety and efficacy of ACT and overcome the above disadvantages.

### SUMMARY OF THE INVENTION

The present invention generally relates to new antigen binding receptor formats capable of specific binding to distinct targets, i.e. a tumor associated antigen (TAA) and T cells expressing these antigen binding receptors. The antigen binding receptors of the invention lead to strong and selective activation of T cells upon binding of one or more antigen binding receptors to a target cell, i.e. to a tumor cell.

In one aspect the invention relates to an antigen binding receptor comprising an anchoring transmembrane domain and an extracellular domain comprising an antigen binding moiety, wherein the antigen binding moiety is a Fab, crossFab or a scFab fragment.

In one embodiment, the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof.

In one embodiment, the anchoring transmembrane domain is the CD28 transmembrane domain or a fragment thereof, in particular wherein the anchoring transmembrane domain comprises the amino acid sequence of SEQ ID NO:14.

In one embodiment, the antigen binding receptor further comprises at least one stimulatory signaling domain and/or at least one co-stimulatory signaling domain.

In one embodiment, the at least one stimulatory signaling domain is individually selected from the group consisting of the intracellular domain of CD3z, of FCGR3A and of NKG2D, or fragments thereof.

In one embodiment, the at least one stimulatory signaling domain is the intracellular domain of CD3z or a fragment thereof, in particular wherein the at least one stimulatory signaling domain comprises the amino acid sequence of SEQ ID NO:16.

In one embodiment, the at least one co-stimulatory signaling domain is individually selected from the group consisting of the intracellular domain of CD27, of CD28, of CD137, of OX40, of ICOS, of DAP10 and of DAP12, or fragments thereof.

In one embodiment, the at least one co-stimulatory signaling domain is the CD28 intracellular domain or a fragment thereof, in particular, wherein the at least one co-stimulatory signaling domain comprises the amino acid sequence of SEQ ID NO:15.

In one embodiment, the antigen binding receptor comprises one stimulatory signaling domain comprising the intracellular domain of CD3z, or a fragment thereof, and wherein the antigen binding receptor comprises one co-stimulatory signaling domain comprising the intracellular domain of CD28, or a fragment thereof.

In one embodiment, the stimulatory signaling domain comprises the amino acid sequence of SEQ ID NO:16 and the co-stimulatory signaling domain comprises the amino acid sequence of SEQ ID NO:15.

In one embodiment, the extracellular domain is connected to the anchoring transmembrane domain, optionally through a peptide linker.

In one embodiment, the peptide linker comprises the amino acid sequence GGGGS (SEQ ID NO:20).

In one embodiment, the anchoring transmembrane domain is connected to a co-signaling domain or to a signaling domain, optionally through a peptide linker.

In one embodiment, the signaling and/or co-signaling domains are connected, optionally through at least one peptide linker.

In one embodiment, the antigen binding moiety comprises a heavy chain constant (CH) domain and a light chain constant domain (CL), wherein the CH domain or the CL domain is connected at the C-terminus to the N-terminus of the anchoring transmembrane domain, optionally through a peptide linker.

In one embodiment, the antigen binding receptor comprises one co-signaling domain, wherein the co-signaling domain is connected at the N-terminus to the C-terminus of the anchoring transmembrane domain.

In one embodiment, the antigen binding receptor additionally comprises one stimulatory signaling domain, wherein the stimulatory signaling domain is connected at the N-terminus to the C-terminus of the co-stimulatory signaling domain. In one embodiment, the antigen binding moiety is capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

In one embodiment, the antigen binding moiety is capable of specific binding to an antigen selected from the group consisting of fibroblast activation protein (FAP), carcinoembryonic antigen (CEA), mesothelin (MSLN), CD20, folate receptor 1 (FOLR1), tenascin (TNC) and programmed death-ligand 1(PDL1).

In one embodiment, the antigen binding moiety is a capable of specific binding to CD20, wherein the antigen binding moiety comprises:

(i) a heavy chain variable region (VH) comprising
(a) the heavy chain complementarity-determining region (CDR H) 1 amino acid sequence YSWIN (SEQ ID NO:1);
(b) the CDR H2 amino acid sequence RIFPGDGDTDYNGKFKG (SEQ ID NO:2); and
(c) the CDR H3 amino acid sequence NVFDGYWLVY (SEQ ID NO:3); and

(ii) a light chain variable region (VL) comprising
(d) the light chain complementary-determining region (CDR L) 1 amino acid sequence RSSKSLLHSNGITYLY (SEQ ID NO:4);
(e) the CDR L2 amino acid sequence QMSNLVS (SEQ ID NO:5); and
(f) the CDR L3 amino acid sequence AQNLELPYT (SEQ ID NO:6).

In one embodiment, the antigen binding moiety is capable of specific binding to CD20, wherein the antigen binding moiety comprises a heavy chain variable region (VH) comprising an amino acid sequence that is at least about 95%, 96%, 97%, 98%, 99% or 100% identical to the amino acid of SEQ ID NO:12, and a light chain variable region (VL) comprising an amino acid sequence that is at least about 95%, 96%, 97%, 98%, 99% or 100% identical to the amino acid sequence of SEQ ID NO:10.

In one embodiment, the antigen binding moiety comprises the heavy chain variable region (VH) of SEQ ID NO:12 and the light chain variable region (VL) of SEQ ID NO:10.

In one embodiment, the antigen binding moiety is a Fab fragment capable of specific binding to CD20, wherein the antigen binding receptor comprises

a) a first polypeptide that is at least about 95%, 96%, 97%, 98%, 99% or 100% identical to an amino acid sequence selected from the group consisting of SEQ ID NO:7 and SEQ ID NO:50; and

b) a second polypeptide that is at least about 95%, 96%, 97%, 98%, 99% or 100% identical to an amino acid sequence selected from the group consisting of SEQ ID NO:9 and SEQ ID NO:8.

In one embodiment, the antigen binding moiety is a crossFab fragment capable of specific binding to CD20, wherein the antigen binding receptor comprises

a) a first polypeptide that is at least about 95%, 96%, 97%, 98%, 99% or 100% identical to an amino acid sequence selected from the group consisting of SEQ ID NO:36 and SEQ ID NO:41; and

b) a second polypeptide that is at least about 95%, 96%, 97%, 98%, 99% or 100% identical to an amino acid sequence selected from the group consisting of SEQ ID NO:38 and SEQ ID NO:43.

In one embodiment, the antigen binding moiety is a scFab fragment capable of specific binding to CD20, wherein the antigen binding receptor comprises a polypeptide that is at least about 95%, 96%, 97%, 98%, 99% or 100% identical to the amino acid sequence of SEQ ID NO:51.

In one embodiment, the antigen binding moiety is a capable of specific binding to PDL1, wherein the antigen binding moiety comprises:

(i) a heavy chain variable region (VH) comprising
(a) the heavy chain complementarity-determining region (CDR H) 1 amino acid sequence DSWIH (SEQ ID NO:68);
(b) the CDR H2 amino acid sequence WISPYGGSTYYADSVKG (SEQ ID NO:69); and
(c) the CDR H3 amino acid sequence RHWPGGFDY (SEQ ID NO:70); and

(ii) a light chain variable region (VL) comprising
(d) the light chain complementary-determining region (CDR L) 1 amino acid sequence RASQDVSTAVA (SEQ ID NO:71);
(e) the CDR L2 amino acid sequence SASFLYS (SEQ ID NO:72); and
(f) the CDR L3 amino acid sequence QQYLYHPAT (SEQ ID NO:73).

In one embodiment, the antigen binding moiety is capable of specific binding to PDL1, wherein the antigen binding moiety comprises a heavy chain variable region (VH) comprising an amino acid sequence that is at least about 95%, 96%, 97%, 98%, 99% or 100% identical to the amino acid of SEQ ID NO:78, and a light chain variable region (VL) comprising an amino acid sequence that is at least about 95%, 96%, 97%, 98%, 99% or 100% identical to the amino acid sequence of SEQ ID NO:77.

In one embodiment, the antigen binding moiety comprises the heavy chain variable region (VH) of SEQ ID NO:78 and the light chain variable region (VL) of SEQ ID NO:77.

In one embodiment, the antigen binding moiety is a Fab fragment capable of specific binding to PDL1, wherein the antigen binding receptor comprises

a) a first polypeptide that is at least about 95%, 96%, 97%, 98%, 99% or 100% identical to an amino acid sequence selected from the group consisting of SEQ ID NO:74 and SEQ ID NO:85; and

b) a second polypeptide that is at least about 95%, 96%, 97%, 98%, 99% or 100% identical to an amino acid sequence selected from the group consisting of SEQ ID NO:76 and SEQ ID NO:75.

In one embodiment, the antigen binding moiety is a crossFab fragment capable of specific binding to PDL1, wherein the antigen binding receptor comprises

a) a first polypeptide that is at least about 95%, 96%, 97%, 98%, 99% or 100% identical to an amino acid sequence selected from the group consisting of SEQ ID NO:79 and SEQ ID NO:82; and

b) a second polypeptide that is at least about 95%, 96%, 97%, 98%, 99% or 100% identical to an amino acid sequence selected from the group consisting of SEQ ID NO:81 and SEQ ID NO:84.

In one embodiment, the antigen binding moiety is a scFab fragment capable of specific binding to PDL1, wherein the antigen binding receptor comprises a polypeptide that is at least about 95%, 96%, 97%, 98%, 99% or 100% identical to the amino acid sequence of SEQ ID NO:86. In one embodiment, the antigen binding moiety is a capable of specific binding to CEA, wherein the antigen binding moiety comprises:

(i) a heavy chain variable region (VH) comprising
(a) the heavy chain complementarity-determining region (CDR H) 1 amino acid sequence EFGMN (SEQ ID NO:138);
(b) the CDR H2 amino acid sequence WINTKTGEATYVEEFKG (SEQ ID NO:139); and
(c) the CDR H3 amino acid sequence WDFAYYVEAMDY (SEQ ID NO:140); and

(ii) a light chain variable region (VL) comprising
(d) the light chain complementary-determining region (CDR L) 1 amino acid sequence KASAAVGTYVA (SEQ ID NO:141);
(e) the CDR L2 amino acid sequence SASYRKR (SEQ ID NO:142); and
(f) the CDR L3 amino acid sequence HQYYTYPLFT (SEQ ID NO:143).

In one embodiment, the antigen binding moiety is a capable of specific binding to CEA, wherein the antigen binding moiety comprises:

(i) a heavy chain variable region (VH) comprising
(a) the heavy chain complementarity-determining region (CDR H) 1 amino acid sequence DTYMH (SEQ ID NO:148);
(b) the CDR H2 amino acid sequence RIDPANGNSKYVPKFQG (SEQ ID NO:149); and
(c) the CDR H3 amino acid sequence FGYYVSDYAMAY (SEQ ID NO:150); and

(ii) a light chain variable region (VL) comprising
(d) the light chain complementary-determining region (CDR L) 1 amino acid sequence RAGESVDIFGVGFLH (SEQ ID NO:151);
(e) the CDR L2 amino acid sequence RASNRAT (SEQ ID NO:152); and
(f) the CDR L3 amino acid sequence QQTNEDPYT (SEQ ID NO:153).

In one embodiment, provided is an isolated polynucleotide encoding the antigen binding receptor as described herein.

In one embodiment, provided is a composition encoding the antigen binding receptor as described herein, comprising a first isolated polynucleotide encoding a first polypeptide, and a second isolated polynucleotide encoding a second polypeptide.

In one embodiment, provided is a polypeptide encoded by the polynucleotide as described herein or by the composition as described herein.

In one embodiment, provided is a vector, particularly an expression vector, comprising the polynucleotide as described herein or the composition as described herein.

In one embodiment, provided is a transduced T cell comprising the polynucleotide as described herein, the composition as described herein or the vector as described herein.

In one embodiment, provided is a transduced T cell capable of expressing at least one of the antigen binding receptors as described herein.

In one embodiment, provided is the transduced T cell as described herein, wherein the cell comprises

(i) not more than one antigen binding receptor comprising a Fab (VH-CH-ATD) antigen binding domain;

(ii) not more than one antigen binding receptor comprising a Fab (VL-CL-ATD) antigen binding domain;

(iii) not more than one antigen binding receptor comprising a crossFab (VL-CH-ATD) antigen binding domain; and

(iv) not more one antigen binding receptor comprising a crossFab (VH-CL-ATD) antigen binding domain.

In one embodiment, provided is the transduced T cell as described herein, wherein the cell comprises a first antigen binding receptor as described herein, wherein a first antigen binding receptor comprises a Fab antigen binding moiety, and wherein the cell comprises a second antigen binding receptor as described herein, wherein the second antigen binding receptor comprises a crossFab antigen binding moiety.

In one embodiment, provided is the transduced T cell as described herein, wherein the cell comprises a first antigen binding receptor as described, wherein the first antigen binding receptor comprises a Fab (VH-CH-ATD) antigen binding moiety, and wherein the cell comprises a second antigen binding receptor as described herein, wherein the second antigen binding receptor comprises a Fab (VL-CL-ATD) antigen binding moiety.

In one embodiment, provided is the transduced T cell as described herein, wherein the cell comprises a first antigen binding receptor as described herein, wherein the first antigen binding receptor comprises a crossFab (VL-CH-ATD) antigen binding moiety, and wherein the cell comprises a second antigen binding receptor as described herein, wherein the second antigen binding receptor comprises a crossFab (VH-CL-ATD) antigen binding moiety.

In one embodiment, provided is the transduced T cell as described herein, wherein the cell comprises a first antigen binding receptor as described herein, wherein a first antigen binding receptor comprises a scFab antigen binding moiety, and wherein the cell comprises a second antigen binding receptor as described herein, wherein the second antigen binding receptor comprises an scFv, a Fab or crossFab antigen binding moiety.

In one embodiment, provided is the transduced T cell as described herein, wherein the cell comprises a first antigen binding receptor capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

In one embodiment, provided is the transduced T cell as described herein, wherein the cell comprises a second antigen binding receptor capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

In one embodiment, provided is the transduced T cell as described herein, wherein the cell comprises a first antigen binding receptor capable of specific binding to a first tumor associated antigen (TAA), and wherein the cell comprises a second antigen binding receptor capable of specific binding to a TAA.

In one embodiment, provided is the transduced T cell as described herein, wherein the cell comprises a first antigen binding receptor capable of specific binding to programmed death-ligand 1 (PDL1), and wherein the cell comprises a second antigen binding receptor capable of specific binding to an antigen selected from the group consisting of fibroblast activation protein (FAP), carcinoembryonic antigen (CEA), mesothelin (MSLN), CD20, folate receptor 1 (FOLR1), and tenascin (TNC).

In one embodiment, provided is the transduced T cell as described herein, wherein the cell comprises a first antigen binding receptor capable of specific binding to PDL1, and wherein the cell comprises a second antigen binding receptor capable of specific binding to CD20.

In one embodiment, provided is the transduced T cell as described herein, wherein the transduced T cell is co-transduced with a T cell receptor (TCR) capable of specific binding of a target antigen.

In one embodiment, provided is the antigen binding receptor as described herein or the transduced T cell as described herein for use as a medicament.

In one embodiment, provided is the antigen binding receptor as described herein or the transduced T cell as described herein for use in the treatment of a malignant disease, wherein the treatment comprises administration of a transduced T cell expressing the antigen binding receptor.

In one embodiment, provided is the antigen binding receptor or the transduced T cell for use as described herein, wherein said malignant disease is selected from cancer of epithelial, endothelial or mesothelial origin and cancer of the blood.

In one embodiment, provided is the transduced T cell for use as described herein, wherein the transduced T cell is derived from a cell isolated from the subject to be treated.

In one embodiment, provided is the transduced T cell for use as described herein, wherein the transduced T cell is not derived from a cell isolated from the subject to be treated.

In one embodiment, provided is a method of treating a disease in a subject, comprising administering to the subject a transduced T cell capable of expressing the antigen binding receptor as described herein. In one embodiment, the method additionally comprises isolating a T cell from the subject and generating the transduced T cell by transducing the isolated T cell with the polynucleotide as described herein, the composition as described herein or the vector as described herein. In one embodiment, the T cell is transduced with a retroviral or lentiviral vector construct or with a non-viral vector construct. In one embodiment, the non-viral vector construct is a sleeping beauty minicircle vector. In one embodiment, the transduced T cell is administered to the subject by intravenous infusion. In one embodiment, the transduced T cell is contacted with anti-CD3 and/or anti-CD28 antibodies prior to administration to the subject. In one embodiment, the transduced T cell is contacted with at least one cytokine prior to administration to the subject, preferably with interleukin-2 (IL-2), interleukin-7 (IL-7), interleukin-15 (IL-15), and/or interleukin-21, or variants thereof. In one embodiment, the disease is a malignant disease. In one embodiment, the disease is selected from cancer of epithelial, endothelial or mesothelial origin and cancer of the blood.

In one embodiment, provided is a method for inducing lysis of a target cell, comprising contacting the target cell with a transduced T cell capable of expressing the antigen binding receptor as described herein. In one embodiment, the target cell is a cancer cell. In one embodiment, the target cell expresses an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1. In one embodiment, the target cell expresses an antigen selected from the group consisting of fibroblast activation protein (FAP), carcinoembryonic antigen (CEA), mesothelin (MSLN), CD20, folate receptor 1 (FOLR1), tenascin (TNC), and programmed death-ligand 1 (PDL1).

In one embodiment provided is the use of the antigen binding receptor as described herein, the polynucleotide as described herein, the composition as described herein, or the transduced T cell as described herein for the manufacture of a medicament. In one embodiment, the medicament is for treatment of a malignant disease. In one embodiment, the malignant disease is selected from cancer of epithelial, endothelial or mesothelial origin and cancer of the blood.

### SHORT DESCRIPTION OF THE FIGURES

FIGS. 1A to 1F depict the architecture of different antigen binding receptor formats of the invention, in particular the Fab, crossFab and scFab formats. FIG. 1A shows the architecture of the Fab format. Depicted is the extracellular domain comprising an antigen binding moiety which consists of an Ig heavy chain and an Ig light chain. Attached to the heavy chain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 1B shows the architecture of the Fab format with heavy and light chain swap. Depicted is the extracellular domain comprising an antigen binding moiety which consists of an Ig heavy chain and an Ig light chain. Attached to the light chain constant domain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 1C shows the architecture of the scFab format. Depicted is the extracellular domain comprising an antigen binding moiety which consists of an Ig heavy chain and an Ig light chain, both connected by a linker. Attached to the heavy chain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 1D shows the architecture of the crossFab format with VH-VL swap. Depicted is the extracellular domain comprising an antigen binding moiety which consists of an Ig heavy chain and an Ig light chain wherein the VH and VL domains are exchanged. Attached to the heavy chain constant domain, a linker connects the antigen recognition domain with an anchoring transmembrane domain (ATD) which is fused to an intracellular co-stimulatory signaling domain (C SD) which in turn is fused to a stimulatory signaling domain (SSD). FIG. 1E shows the architecture of the crossFab format with CH-CL swap. Depicted is the extracellular domain comprising an antigen binding moiety which consists of an Ig heavy chain and an Ig light chain wherein the CH and CL domains are exchanged. Attached to the light chain constant domain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 1F shows the architecture of the classic scFv format with an extracellular antigen recognition domain, consisting of a variable heavy and variable light chain, both connected by a linker. Attached to the variable light chain, a linker connects the antigen recognition domain with an anchoring transmembrane domain (ATD) which is fused to an intracellular co-stimulatory signaling domain (CSD) which in turn is fused to a stimulatory signaling domain (SSD).

FIGS. 2A to 2F depict a schematic representation illustrating the modular composition of exemplary expression constructs encoding antigen binding receptors of the invention. FIG. 2A and FIG. 2B depict exemplary Fab formats. FIG. 2C depicts an exemplary scFab format.

FIG. 2D and FIG. 2E depict exemplary crossFab formats. FIG. 2F depicts a classic scFv format.

FIG. 3 shows a schematic representation of a Jurkat NFAT T cell reporter assay. A tumor associated antigen (TAA) can be recognized by the anti-TAA antigen binding receptor expressing Jurkat NFAT T cell. This recognition leads to the activation of the cell which can be detected by measuring luminescence (cps).

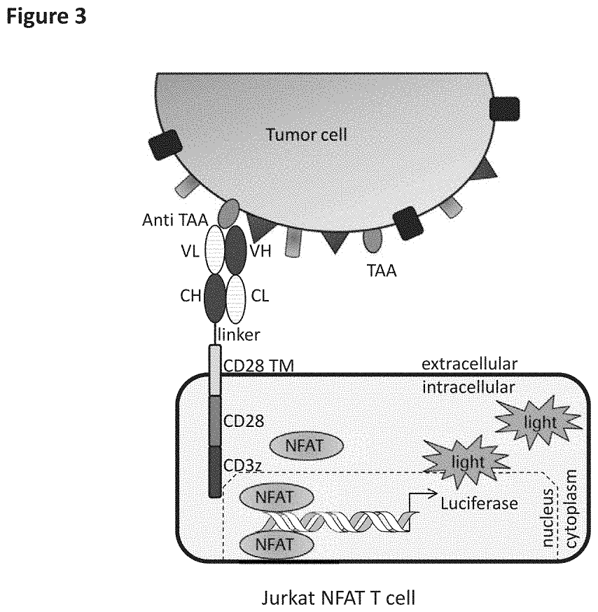

FIG. 4 depicts the Jurkat NFAT T cell reporter assay using CD20 expressing SUDHDL4 tumor cells as target cells. A single clone of anti-CD20-Fab-CD28ATD-CD28CSD-CD3zSSD expressing Jurkat NFAT T cells was used as effector cells.

FIG. 5 depicts the Jurkat NFAT T cell reporter assay using CD20 expressing SUDHDL4 tumor cells as target cells. A pool of anti-CD20-Fab-CD28ATD-CD28CSD-CD3zSSD or anti-CD20-crossFab-CD28ATD-CD28CSD-CD3zSSD expressing Jurkat NFAT T cells was used as effector cells.

FIG. 6 depicts the Jurkat NFAT T cell reporter assay using CD20 expressing SUDHDL4 tumor cells as target cells. A pool of anti-CD20-scFab-CD28ATD-CD28CSD-CD3zSSD expressing Jurkat NFAT T cells was used as effector cells.

FIG. 7 depicts the Jurkat NFAT T cell reporter assay using CD20 expressing SUDHDL4 tumor cells as target cells. A pool of anti-CD20-Fab-CD28ATD-CD28CSD-CD3zSSD or anti-CD20-scFv-CD28ATD-CD28CSD-CD3zSSD expressing Jurkat NFAT T cells was used as effector cells.

FIG. 8 depicts a killing assay using CD20 expressing SUDHDL4 tumor cells as target cells. A pool of anti-CD20-scFv-CD28ATD-CD28CSD-CD3zSSD expressing T cells was used as effector cells.

### DETAILED DESCRIPTION

Definitions

Terms are used herein as generally used in the art, unless otherwise defined in the following. An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcaRI (CD89).

Antibody-dependent cell-mediated cytotoxicity (“ADCC”) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been mutated. For example the reduction in ADCC mediated by an antibody comprising in its Fc domain an amino acid mutation that reduces ADCC, is relative to the ADCC mediated by the same antibody without this amino acid mutation in the Fc domain. Suitable assays to measure ADCC are well known in the art (see e.g., PCT publication no. WO 2006/082515 or PCT publication no. WO 2012/130831).

An “effective amount” of an agent refers to the amount that is necessary to result in a physiological change in the cell or tissue to which it is administered.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen and/or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same.

Affinity can be measured by well-established methods known in the art, including those described herein. A preferred method for measuring affinity is Surface Plasmon Resonance (SPR) and a preferred temperature for the measurement is 25° C.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics. Amino acid sequence deletions and insertions include amino- and/or carboxy-terminal deletions and insertions of amino acids. Particular amino acid mutations are amino acid substitutions. For the purpose of altering e.g., the binding characteristics of an antigen binding moiety, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

The term “antibody” herein is used in the broadest sense and encompasses various antibody structures, including but not limited to monoclonal antibodies, polyclonal antibodies, and antibody fragments so long as they exhibit the desired antigen-binding activity. Accordingly, in context of the present invention, the term antibody relates to full immunoglobulin molecules as well as to parts of such immunoglobulin molecules. Furthermore, the term relates, as discussed herein, to modified and/or altered antibody molecules, in particular to mutated antibody molecules. The term also relates to recombinantly or synthetically generated/synthesized antibodies. In the context of the present invention the term antibody is used interchangeably with the term immunoglobulin.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv or scFab), and single-domain antibodies. For a review of certain antibody fragments, see Hudson et al., Nat Med 9, 129-134 (2003). For a review of scFv fragments, see e.g., Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody (Domantis, Inc., Waltham, Mass.; see e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

As used herein, the term “antigen binding molecule” refers in its broadest sense to a molecule that specifically binds an antigenic determinant. Examples of antigen binding molecules are immunoglobulins and derivatives, e.g., fragments, thereof as well as antigen binding receptors and derivatives thereof.

As used herein, the term “antigen binding moiety” refers to a polypeptide molecule that specifically binds to an antigenic determinant. In one embodiment, an antigen binding moiety is able to direct the entity to which it is attached (e.g., an immunoglobulin or an antigen binding receptor) to a target site, for example to a specific type of tumor cell or tumor stroma bearing the antigenic determinant or to an immunoglobulin binding to the antigenic determinant on a tumor cell. In another embodiment an antigen binding moiety is able to activate signaling through its target antigen, for example signaling is activated upon binding of an antigenic determinant to an antigen binding receptor on a T cell. In the context of the present invention, antigen binding moieties may be included in antibodies and fragments thereof as well as in antigen binding receptors and fragments thereof as further defined herein. Antigen binding moieties include an antigen binding domain, comprising an immunoglobulin heavy chain variable region and an immunoglobulin light chain variable region. In certain embodiments, the antigen binding moieties may comprise immunoglobulin constant regions as further defined herein and known in the art. Useful heavy chain constant regions include any of the five isotypes: α, δ, ε, γ, or μ. Useful light chain constant regions include any of the two isotypes: κ and λ.

In the context of the present invention the term “antigen binding receptor” relates to an antigen binding molecule comprising an anchoring transmembrane domain and an extracellular domain comprising at least one antigen binding moiety. An antigen binding receptor can be made of polypeptide parts from different sources. Accordingly, it may be also understood as a “fusion protein” and/or a “chimeric protein”. Usually, fusion proteins are proteins created through the joining of two or more genes (or preferably cDNAs) that originally coded for separate proteins. Translation of this fusion gene (or fusion cDNA) results in a single polypeptide, preferably with functional properties derived from each of the original proteins. Recombinant fusion proteins are created artificially by recombinant DNA technology for use in biological research or therapeutics. Further details to the antigen binding receptors of the present invention are described herein below. In the context of the present invention a CAR (chimeric antigen receptor) is understood to be an antigen binding receptor comprising an extracellular portion comprising an antigen binding moiety fused by a spacer sequence to an anchoring transmembrane domain which is itself fused to the intracellular signaling domains of CD3z and CD28.

An “antigen binding site” refers to the site, i.e. one or more amino acid residues, of an antigen binding molecule which provides interaction with the antigen. For example, the antigen binding site of an antibody or an antigen binding receptor comprises amino acid residues from the complementarity determining regions (CDRs). A native immunoglobulin molecule typically has two antigen binding sites; a Fab, crossFab, scFab or a scFv molecule typically has a single antigen binding site.

The term “antigen binding domain” refers to the part of an antibody or an antigen binding receptor that comprises the area which specifically binds to and is complementary to part or all of an antigen. An antigen binding domain may be provided by, for example, one or more immunoglobuling variable domains (also called variable regions). Particularly, an antigen binding domain comprises an immunoglobulin light chain variable region (VL) and an immunoglobulin heavy chain variable region (VH).

The term “variable region” or “variable domain” refers to the domain of an immunoglobulin heavy or light chain that is involved in binding the antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W. H. Freeman and Co, page 91 (2007). A single VH or VL domain is usually sufficient to confer antigen-binding specificity.

The term “ATD” as used herein refers to “anchoring transmembrane domain” which defines a polypeptide stretch capable of integrating in (the) cellular membrane(s) of a cell. The ATD can be fused to further extracellular and/or intracellular polypeptide domains wherein these extracellular and/or intracellular polypeptide domains will be confined to the cell membrane as well. In the context of the antigen binding receptors of the present invention the ATD confers membrane attachment and confinement of the antigen binding receptor of the present invention. The antigen binding receptors of the present invention comprise at least one ATD and an extracellular domain comprising an antigen binding moiety. Additionally, the ATD may be fused to further intracellular signaling domains.

The term “binding to” as used in the context of the antigen binding receptors of the present invention defines a binding (interaction) of an “antigen-interaction-site” and an antigen with each other. The term “antigen-interaction-site” defines, in accordance with antigen binding receptors of the present invention, a motif of a polypeptide which shows the capacity of specific interaction with a specific antigen or a specific group of antigens. Said binding/interaction is also understood to define a “specific recognition”. The term “specifically recognizing” means in accordance with this invention that the antigen binding receptor is capable of specifically interacting with and/or binding to a tumor associated antigen (TAA) molecule as defined herein. The antigen binding moiety of an antigen binding receptor can recognize, interact and/or bind to different epitopes on the same molecule. This term relates to the specificity of the antigen binding receptor, i.e., to its ability to discriminate between the specific regions of a molecule as defined herein. The specific interaction of the antigen-interaction-site with its specific antigen may result in an initiation of a signal, e.g. due to the induction of a change of the conformation of the polypeptide comprising the antigen, an oligomerization of the polypeptide comprising the antigen, an oligomerization of the antigen binding receptor, etc. Thus, a specific motif in the amino acid sequence of the antigen-interaction-site and the antigen bind to each other as a result of their primary, secondary or tertiary structure as well as the result of secondary modifications of said structure. Accordingly, the term binding to does not only relate to a linear epitope but may also relate to a conformational epitope, a structural epitope or a discontinuous epitope consisting of two regions of the target molecules or parts thereof. In the context of this invention, a conformational epitope is defined by two or more discrete amino acid sequences separated in the primary sequence which comes together on the surface of the molecule when the polypeptide folds to the native protein (Sela, Science 166 (1969), 1365 and Laver, Cell 61 (1990), 553-536). Moreover, the term “binding to” is interchangeably used in the context of the present invention with the term “interacting with”. The ability of the antigen binding moiety (e.g. a Fab, crossFab, scFab or scFv domain) of an antigen binding receptor or an antibody to bind to a specific target antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Lilj eblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the target antigen as measured, in particular by SPR. In certain embodiments, an antigen binding moiety that binds to the target antigen, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). The term “specific binding” as used in accordance with the present invention means that the molecules of the invention do not or do not essentially cross-react with (poly-) peptides of similar structures. Cross-reactivity of a panel of constructs under investigation may be tested, for example, by assessing binding of a panel of antigen binding moieties under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the antigen of interest as well as to unrelated antigens. Only those constructs (i.e. Fab fragments, scFvs and the like) that bind to the antigen of interest but do not or do not essentially bind to unrelated antigens are considered specific for the antigen of interest and selected for further studies in accordance with the method provided herein. These methods may comprise, inter alia, binding studies, blocking and competition studies with structurally and/or functionally closely related polypeptides. The binding studies also comprise FACS analysis, surface plasmon resonance (SPR, e.g. with BIAcore®), analytical ultracentrifugation, isothermal titration calorimetry, fluorescence anisotropy, fluorescence spectroscopy or by radiolabeled ligand binding assays.

The term “CDR” as employed herein relates to “complementary determining region”, which is well known in the art. The CDRs are parts of immunoglobulins or antigen binding receptors that determine the specificity of said molecules and make contact with a specific ligand. The CDRs are the most variable part of the molecule and contribute to the antigen binding diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in “Kabat” (Sequences of Proteins of Immunological Interest”, 5th edit. NIH Publication no. 91-3242 U.S. Department of Health and Human Services (1991); Chothia J. Mol. Biol. 196 (1987), 901-917) or “Chothia” (Nature 342 (1989), 877-883).

The term “ CD3z” refers to T-cell surface glycoprotein CD3 zeta chain, also known as “T-cell receptor T3 zeta chain” and “CD247”.

The term “chimeric antigen receptor” or “chimeric receptor” or “CAR” refers to an antigen binding receptor constituted of an extracellular portion of an antigen binding moiety (e.g. a scFv domain) fused by a spacer sequence to the intracellular signaling domains of CD3z and CD28. The invention additionally provides antigen binding receptors wherein the antigen binding moiety is a Fab, a crossFab or a scFab fragment. The term “CAR” is understood in its broadest form to comprise antigen binding receptors constituted of an extracellular portion comprising an antigen binding moiety fused to CD3z and fragment thereof and to CD28 and fragments thereof, optionally through one or several peptide linkers.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

By a “crossover Fab molecule” (also termed “crossFab” or “crossover Fab fragment”) is meant a Fab molecule wherein either the variable regions or the constant regions of the Fab heavy and light chain are exchanged, i.e. the crossFab fragment comprises a peptide chain composed of the light chain variable region and the heavy chain constant region, and a peptide chain composed of the heavy chain variable region and the light chain constant region. Accordingly, a crossFab fragment comprises a polypeptide composed of the heavy chain variable and the light chain constant regions (VH-CL), and a polypeptide composed of the light chain variable and the heavy chain constant regions (VL-CH1). For clarity, the polypeptide chain comprising the heavy chain constant region is referred to herein as the heavy chain and the polypeptide chain comprising the light chain constant regions is referred to herein as the light chain of the crossFab fragment.

By a “Fab” or “conventional Fab” molecule is meant a Fab molecule in its natural format, i.e. comprising a heavy chain composed of the heavy chain variable and constant regions (VH-CH1), and a light chain composed of the light chain variable and constant regions (VL-CL). The term “CSD” as used herein refers to co-stimulatory signaling domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the terms “engineer”, “engineered”, “engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode antigen binding molecules of the invention or fragments thereof.

A “Fab molecule” refers to a protein consisting of the VH and CH1 domain of the heavy chain (the “Fab heavy chain”) and the VL and CL domain of the light chain (the “Fab light chain”) of an antigen binding molecule.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the “EU numbering” system, also called the EU index, as described in Kabat et al., Sequences of Proteins of Immunological Interest, 5th Ed. Public Health Service, National Institutes of Health, Bethesda, Md., 1991. A subunit of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG1 and IgG2), IgM, IgA, IgD, and IgE)

By “fused” is meant that the components (e.g., a Fab and a transmembrane domain) are linked by peptide bonds, either directly or via one or more peptide linkers.

The terms “host cell”, “host cell line” and “host cell culture” are used interchangeably and refer to cells into which exogenous nucleic acid has been introduced, including the progeny of such cells. Host cells include “transformants” and “transformed cells” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate an antibody used according to the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody and/or an antigen binding receptor or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of Kabat numbering to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antigen binding moiety variable region are according to the Kabat numbering system. The polypeptide sequences of the sequence listing are not numbered according to the Kabat numbering system. However, it is well within the ordinary skill of one in the art to convert the numbering of the sequences of the Sequence Listing to Kabat numbering.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

By “isolated nucleic acid” molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed below for polypeptides (e.g., ALIGN-2).

By an “isolated polypeptide” or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. The term “nucleic acid molecule” relates to the sequence of bases comprising purine- and pyrimidine bases which are comprised by polynucleotides, whereby said bases represent the primary structure of a nucleic acid molecule. Herein, the term nucleic acid molecule includes DNA, cDNA, genomic DNA, RNA, synthetic forms of DNA and mixed polymers comprising two or more of these molecules. In addition, the term nucleic acid molecule includes both, sense and antisense strands. Moreover, the herein described nucleic acid molecule may contain non-natural or derivatized nucleotide bases, as will be readily appreciated by those skilled in the art. The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical composition” refers to a preparation which is in such form as to permit the biological activity of an active ingredient contained therein to be effective, and which contains no additional components which are unacceptably toxic to a subject to which the formulation would be administered. A pharmaceutical composition usually comprises one or more pharmaceutically acceptable carrier(s).

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. As used herein, term “polypeptide” refers to a molecule composed of monomers (amino acids) linearly linked by amide bonds (also known as peptide bonds). The term polypeptide refers to any chain of two or more amino acids, and does not refer to a specific length of the product. Thus, peptides, dipeptides, tripeptides, oligopeptides, protein, amino acid chain, or any other term used to refer to a chain of two or more amino acids, are included within the definition of polypeptide, and the term polypeptide may be used instead of, or interchangeably with any of these terms. The term polypeptide is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term nucleic acid molecule refers to any one or more nucleic acid segments, e.g., DNA or RNA fragments, present in a polynucleotide.

“Reduced binding”, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

The term “regulatory sequence” refers to DNA sequences, which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

As used herein, the term “single-chain” refers to a molecule comprising amino acid monomers linearly linked by peptide bonds. In certain embodiments, one of the antigen binding moieties is a scFv fragment, i.e. a VH domain and a VL domain connected by a peptide linker. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. The term “SSD” as used herein refers to stimulatory signaling domain.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, cell expressing antigen binding receptors of the invention are used to delay development of a disease or to slow the progression of a disease.

As used herein, the term “target antigenic determinant” is synonymous with “target antigen”, “target epitope”, “tumor associated antigen” and “target cell antigen” and refers to a site (e.g., a contiguous stretch of amino acids or a conformational configuration made up of different regions of non-contiguous amino acids) on a polypeptide macromolecule to which an antibody binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g., CD20, CEA, FAP, TNC) can be any native form of the proteins from any vertebrate source, including mammals such as primates (e.g., humans) and rodents (e.g., mice and rats), unless otherwise indicated. In a particular embodiment the target antigen is a human protein. Where reference is made to a specific target protein herein, the term encompasses the “full-length”, unprocessed target protein as well as any form of the target protein that results from processing in the target cell. The term also encompasses naturally occurring variants of the target protein, e.g., splice variants or allelic variants. Exemplary human target proteins useful as antigens include, but are not limited to: CD20, CEA, FAP, TNC, MSLN, Fo1R1, HER1 and HER2. The ability of an antigen binding receptor to bind to a specific target antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of the antigen binding receptor to an unrelated protein is less than about 10% of the binding of the antibody to the target antigen as measured, e.g., by SPR. In certain embodiments, the antigen binding receptro binds to the target antigen with an affinity dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The antigen binding receptors of the invention are capable of inducing T cell activation. Suitable assays to measure T cell activation are known in the art described herein.

In accordance with this invention, the term “T cell receptor” or “TCR” is commonly known in the art. In particular, herein the term “T cell receptor” refers to any T cell receptor, provided that the following three criteria are fulfilled: (i) tumor specificity, (ii) recognition of (most) tumor cells, which means that an antigen or target should be expressed in (most) tumor cells and (iii) that the TCR matches to the HLA-type of the subjected to be treated. In this context, suitable T cell receptors which fulfill the above mentioned three criteria are known in the art such as receptors recognizing NY-ESO-1 (for sequence information(s) see, e.g., PCT/GB2005/001924) and/or HER2neu (for sequence information(s) see WO-A1 2011/0280894).

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode antigen binding receptors of the invention or fragments thereof.

Antigen Binding Receptor Formats

The present invention relates to antigen binding receptors capable of specific binding to a target antigen, i.e. a tumor associated antigen (TAA). In particular, the present invention relates to antigen binding receptors comprising an extracellular domain comprising at least one antigen binding moiety, wherein the antigen binding moiety is a Fab, crossFab or a scFab fragment. The present invention further relates to the transduction of T cells, such as CD8+ T cells, CD4+ T cells, CD3+ T cells, γδ T cells or natural killer (NK) T cells, preferably CD8+ T cells, with an antigen binding receptor as described herein and their targeted recruitment, e.g., to a tumor. As shown in the appended Examples, as a proof of the inventive concept, the antigen binding receptor comprising an anchoring transmembrane domain and an extracellular domain according to the invention pETR17097 (SEQ ID NO:7 as encoded by the DNA sequence shown in SEQ ID NO:22) was constructed which is capable of specific binding to CD20. Transduced T cells (Jurkat NFAT T cells) expressing the Anti-CD20-Fab-CD28ATD-CD28CSD-CD3zSSD protein (SEQ ID NO:7 as encoded by the DNA sequence shown in SEQ ID NO:22) could be strongly activated by CD20 positive tumor cells. The inventors further provided multiple formats of the antigen binding receptor capable of specific binding to a tumor antigen. The Fab and crossFab formats of the present invention are particularly preferred due to the differentiated activation of T cells by antigen binding receptors comprising antigen binding moiety according to one of these formats. The differentiated activating of T cells was demonstrated with Fab and crossFab formats and compared to a scFv format. Fab and crossFab formats according to the present invention ensure correct pairing of heavy and light chains of distinct antigen binding moieties and, surprisingly, lead to differentiated activation of T cells compared to the scFv format. Furthermore, more than one Fab based antigen binding receptor can be expressed according to the invention within the same cell, i.e. a T cell, wherein the antigen binding receptors of the invention are assembled correctly and the functional properties of the antigen binding receptors, e.g. activation of T cells, remain strong. This further increases the possibilities to tune the T cell response without changing binder affinities. Accordingly, the invention provides such combinations of antigen binding receptors in one cell, in particular the combination of multiple Fab and crossFab formats.

It was found that T cells, preferably CD8+ T cells that were transduced with an antigen binding receptor of the present invention comprising a Fab or crossFab antigen binding moiety were strongly activated and recruited by the tumor-associated antigen (TAA) to the tumor cell. It was surprisingly and unexpectedly shown in the present invention that integrating a Fab and/or crossFab antigen binding moiety would result in a differentiated activation of the T cells, dependent on further T cell stimulation (e.g., CD3 signalling) and subsequent lysis of the tumor cell compared to the classic scFv format. Furthermore, the antigen binding receptor formats of the invention bear significant advantages over conventional scFv based approaches, as the Fab format of the present invention is more stable. Importantly, antigen binding moieties deriving from and/or generated by the use of phage display libraries can be easily converted into the antigen binding receptors of the present invention.

Accordingly, the invention provides a versatile therapeutic platform wherein antigen binding moieties targeting cell antigens derived from known sources or newly developed binders can be easily integrated into a binding and signaling receptor for T cell guidance towards a tumor and providing T cell activation after specific binding. Importantly, more than one antigen binding receptor can be integrated into one cell providing multiple specificities for binding and activation of the T cell, e.g. a CD8+ T cell. After binding to the tumor antigen(s) on the surface of a tumor cell, the transduced T cell as described herein becomes activated and the tumor cell will subsequently be lysed. The platform is flexible and specific by allowing the use of diverse (existing or newly developed) target binders or co-application of multiple antigen binding receptors with different antigen specificity. The degree of T cell activation can further be adjusted by combination of antigen binding moiety/moieties capable of specific binding to immune checkpoint inhibitors and antigen binding moiety/moieties capable of specific binding to tumor antigens and/or by switching to different antigen binder formats. Transduced T cells according to the invention are inert without exposure to the specified antigen(s) or combinations of antigen(s) and immune checkpoint inhibitors as described herein.

In the context of the present invention, the antigen binding receptor comprises an extracellular domain that does not naturally occur in or on T cells. Thus, the antigen binding receptor is capable of providing tailored binding specificity to cells expressing the antigen binding receptor according to the invention. Cells, e.g. T cells, transduced with (an) antigen binding receptor(s) of the invention become capable of specific binding to cells expressing the target antigen (e.g., tumor cells) but not or essentially not to the non-related healthy cells. Specificity is provided by one or several antigen binding moieties of the extracellular domain of the one or more antigen binding receptor(s), such antigen binding moieties are considered to be specific for tumor associated antigens as defined herein. In the context of the present invention and as explained herein, the antigen binding moiety capable of specific binding to a tumor antigen bind to/interact with the tumor cells but not to/with healthy cells/tissue.

Accordingly, the present invention relates to an antigen binding receptor comprising an extracellular domain comprising at least one antigen binding moiety, wherein the antigen binding moiety is a Fab, crossFab or a scFab fragment. The antigen binding receptors of the present invention can be combined in a variety of combinations without affecting the potency of the individual antigen binding receptors. E.g. a first antigen binding receptor comprising a Fab fragment as described herein can be combined with a second antigen binding receptor comprising a crossFab fragments. In addition, the present invention described two individual configurations both of the Fab format as well as the crossFab format further expanding the possible combinations of different receptors. Furthermore, a scFab format is described further expanding combinatorial flexibility. Importantly, correct combination of the different antigen binding receptor format as described herein ensures correct pairing of polypeptide subunits of the antigen binding receptors, i.e., correct assembly of heavy chain and light chain of the Fab formats.

Antigen Binding Moieties

In an illustrative embodiment of the present invention, as a proof of concept, antigen binding receptors are provided comprising an anchoring transmembrane domain and an extracellular domain comprising at least one antigen binding moiety, wherein the antigen binding moiety is a Fab, crossFab or a scFab fragment.

In certain embodiment, at least one of the antigen binding moieties is a conventional Fab fragment, i.e. a Fab molecule consisting of a Fab light chain and a Fab heavy chain. In certain embodiment, at least one of the antigen binding moieties is a crossFab fragment, i.e. a Fab molecule consisting of a Fab light chain and a Fab heavy chain, wherein either the variable regions or the constant regions of the Fab heavy and light chain are exchanged. In certain embodiments, at least one of the antigen binding moieties is a scFv fragment. In a particular such embodiment, the C-terminus of the variable heavy chain (VH) is connected to the N-terminus of the variable light chain (VL) in the scFv molecule, optionally through a peptide linker. In certain embodiments, at least one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule, optionally through a peptide linker.

Antigen binding moieties capable of specific binding to tumor associated antigen may be generated by immunization of e.g. a mammalian immune system. Such methods are known in the art and e.g. are described in Burns in Methods in Molecular Biology 295:1-12 (2005). Alternatively, antigen binding moieties of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antigen binding moieties possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992) and in Marks and Bradbury in Methods in Molecular Biology 248:161-175 (Lo, ed., Human Press, Totowa, N.J., 2003). ;Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antigen binding moieties to the immunogen without the requirement of constructing hybridomas. Alternatively, the naive repertoire can be cloned (e.g., from human) to provide a single source of antigen binding moieties to a wide range of non-self and also self antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936. and 2009/0002360. Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997). A particular advantage of the antigen binding receptor formats according to the present invention is the straight-forward integration of a library derived antigen binding moiety without changing the format, e.g. a Fab antigen binder deriving from screening a phage display library can be included in the Fab and/or crossFab format as described herein. Accordingly, antigen binding moieties deriving form Fab displaying phage libraries can be included in an antigen binding receptor of the present invention without changing the format to e.g., a scFv format which might affect the binding properties of the library derived binder negatively.

In the context of the present invention, provided herein are antigen binding receptors comprising at least one antigen binding moiety capable of specific binding to target antigen, i.e. a tumor associated antigen. Accordingly, transduced cells, i.e. T cells, expressing an antigen binding receptor according to the invention are capable of specific binding to the tumor cell.

In an illustrative embodiment of the present invention, as a proof of concept, provided are antigen binding receptors capable of specific binding CD20 and effector cells expressing said antigen binding receptors. The target cell is one which expresses a CD20 polypeptide and is of a cell type which specifically expresses or overexpresses a CD20 polypeptide. The cells may be cancerous or normal cells of the particular cell type. The cell may be a normal B cell involved in autoimmunity. In one embodiment the cell is a cancer cell, preferably a malignant B cell. Other tumor associated antigens can be targeted according to the invention and as described herein.

Accordingly, in one specific embodiment the extracellular domain of the antigen binding receptor comprises an antigen binding moiety capable of specific binding to CD20, wherein the antigen binding moiety comprises:

(i) a heavy chain variable region (VH) comprising
(a) the heavy chain complementarity-determining region (CDR H) 1 amino acid sequence YSWIN (SEQ ID NO:1);
(b) the CDR H2 amino acid sequence RIFPGDGDTDYNGKFKG (SEQ ID NO:2); and
(c) the CDR H3 amino acid sequence NVFDGYWLVY (SEQ ID NO:3); and

(ii) a light chain variable region (VL) comprising
(d) the light chain complementary-determining region (CDR L) 1 amino acid sequence RSSKSLLHSNGITYLY (SEQ ID NO:4);
(e) the CDR L2 amino acid sequence QMSNLVS (SEQ ID NO:5); and
(f) the CDR L3 amino acid sequence AQNLELPYT (SEQ ID NO:6).

In one embodiment the extracellular domain of the antigen binding receptor comprises an antigen binding moiety capable of specific binding to CD20, wherein the antigen binding moiety comprises a heavy chain variable region (VH) comprising an amino acid sequence that is at least about 95%, 96%, 97%, 98%, 99% or 100% identical to the amino acid of SEQ ID NO:12, and a light chain variable region (VL) comprising an amino acid sequence that is at least about 95%, 96%, 97%, 98%, 99% or 100% identical to the amino acid sequence of SEQ ID NO:10.

In one embodiment the extracellular domain of the antigen binding receptor comprises an antigen binding moiety capable of specific binding CD20, wherein the antigen binding moiety comprises the heavy chain variable region (VH) of SEQ ID NO:12 and the light chain variable region (VL) of SEQ ID NO:10.

In one embodiment, the at least one antigen binding moiety is a Fab, a crossFab or a scFab fragment.

In one preferred embodiment the extracellular domain of the antigen binding receptor comprises an antigen binding moiety capable of specific binding to CD20, wherein the antigen binding moiety is a Fab fragment.

In one embodiment, the extracellular domain of the antigen binding receptor comprises an antigen binding moiety capable of specific binding to CD20, wherein the Fab fragment comprising a heavy chain of SEQ ID NO:8 and a light chain of SEQ ID NO:9.

In one embodiment the extracellular domain of the antigen binding receptor comprises an antigen binding moiety capable of specific binding to CD20, wherein the at least one antigen binding moiety is a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In one embodiment the extracellular domain of the antigen binding receptor comprises an antigen binding moiety capable of specific binding to CD20, wherein the scFv fragment comprises the amino acid sequence of SEQ ID NO:60.

In one preferred embodiment the extracellular domain of the antigen binding receptor comprises an antigen binding moiety capable of specific binding to CD20, wherein the antigen binding moiety is a crossFab fragment.

In one preferred embodiment, the extracellular domain of the antigen binding receptor comprises an antigen binding moiety capable of specific binding to CD20, wherein the crossFab fragment comprises a polypeptide of SEQ ID NO:37 and a polypeptide of SEQ ID NO:38.

In one embodiment the extracellular domain of the antigen binding receptor comprises an antigen binding moiety capable of specific binding to CD20, wherein the at least one antigen binding moiety is a scFab fragment which is a polypeptide consisting of a heavy chain (VH-CH1), a light chain (VL-CL) and a linker, wherein said heavy and light chains and said linker have one of the following configurations in N-terminal to C-terminal direction: a) VL-CL-linker-VH-CH1 or b) VH-CH-linker-VL-CL. In a preferred embodiment, the scFab fragment has the configuration VL-CL-linker-VH-CH1.

In one embodiment the extracellular domain of the antigen binding receptor comprises an antigen binding moiety capable of specific binding to CD20, wherein the scFab fragment comprises the amino acid sequence of SEQ ID NO:51.

In an alternative particular embodiment, the extracellular domain of the antigen binding receptor comprises an antigen binding moiety capable of specific binding to programmed death-ligand 1 (PDL1). Accordingly, in one specific embodiment the extracellular domain of the antigen binding receptor comprises an antigen binding moiety capable of specific binding to PDL1, wherein the antigen binding moiety comprises:

(i) a heavy chain variable region (VH) comprising
(a) the heavy chain complementarity-determining region (CDR H) 1 amino acid sequence DSWIH (SEQ ID NO:68);
(b) the CDR H2 amino acid sequence WISPYGGSTYYADSVKG (SEQ ID NO:69); and
(c) the CDR H3 amino acid sequence RHWPGGFDY (SEQ ID NO:70); and

(ii) a light chain variable region (VL) comprising
(d) the light chain complementary-determining region (CDR L) 1 amino acid sequence RASQDVSTAVA (SEQ ID NO:71);
(e) the CDR L2 amino acid sequence SASFLYS (SEQ ID NO:72); and
(f) the CDR L3 amino acid sequence QQYLYHPAT (SEQ ID NO:73).

In one embodiment the extracellular domain of the antigen binding receptor comprises an antigen binding moiety capable of specific binding to PDL1, wherein the antigen binding moiety comprises a heavy chain variable region (VH) comprising an amino acid sequence that is at least about 95%, 96%, 97%, 98%, 99% or 100% identical to the amino acid of SEQ ID NO:78, and a light chain variable region (VL) comprising an amino acid sequence that is at least about 95%, 96%, 97%, 98%, 99% or 100% identical to the amino acid sequence of SEQ ID NO:77.

In one embodiment the extracellular domain of the antigen binding receptor comprises an antigen binding moiety capable of specific binding PDL1, wherein the antigen binding moiety comprises the heavy chain variable region (VH) of SEQ ID NO:78 and the light chain variable region (VL) of SEQ ID NO:77.

In one embodiment, the at least one antigen binding moiety is a Fab, a crossFab or a scFab fragment.

In one preferred embodiment the extracellular domain of the antigen binding receptor comprises an antigen binding moiety capable of specific binding to PDL1, wherein the antigen binding moiety is a Fab fragment.

In one embodiment, the extracellular domain of the antigen binding receptor comprises an antigen binding moiety capable of specific binding to PDL1, wherein the Fab fragment comprising a heavy chain of SEQ ID NO:75 and a light chain of SEQ ID NO:76.

In one embodiment the extracellular domain of the antigen binding receptor comprises an antigen binding moiety capable of specific binding to PDL1, wherein the at least one antigen binding moiety is a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In one embodiment the extracellular domain of the antigen binding receptor comprises an antigen binding moiety capable of specific binding to PDL1, wherein the scFv fragment comprises the amino acid sequence of SEQ ID NO:88.

In one preferred embodiment the extracellular domain of the antigen binding receptor comprises an antigen binding moiety capable of specific binding to PDL1, wherein the antigen binding moiety is a crossFab fragment.

In one preferred embodiment, the extracellular domain of the antigen binding receptor comprises an antigen binding moiety capable of specific binding to PDL1, wherein the crossFab fragment comprising a polypeptide of SEQ ID NO:80 and a polypeptide of SEQ ID NO:81.

In one embodiment the extracellular domain of the antigen binding receptor comprises an antigen binding moiety capable of specific binding to PDL1, wherein the at least one antigen binding moiety is a scFab fragment which is a polypeptide consisting of a heavy chain (VH-CH1), a light chain (VL-CL) and a linker, wherein said heavy and light chains and said linker have one of the following configurations in N-terminal to C-terminal direction: a) VL-CL-linker-VH-CH1 or b) VH-CH-linker-VL-CL. In a preferred embodiment, the scFab fragment has the configuration VL-CL-linker-VH-CH1.

In one embodiment the extracellular domain of the antigen binding receptor comprises an antigen binding moiety capable of specific binding to PDL1, wherein the scFab fragment comprises the amino acid sequence of SEQ ID NO:86.

In an alternative particular embodiment, the extracellular domain of the antigen binding receptor comprises an antigen binding moiety capable of specific binding to carcinoembryonic antigen (CEA). Accordingly, in one specific embodiment the extracellular domain of the antigen binding receptor comprises an antigen binding moiety capable of specific binding to CEA, wherein the antigen binding moiety comprises:

(i) a heavy chain variable region (VH) comprising
(a) the heavy chain complementarity-determining region (CDR H) 1 amino acid sequence EFGMN (SEQ ID NO:138);
(b) the CDR H2 amino acid sequence WINTKTGEATYVEEFKG (SEQ ID NO:139); and
(c) the CDR H3 amino acid sequence WDFAYYVEAMDY (SEQ ID NO:140); and

(ii) a light chain variable region (VL) comprising
(d) the light chain complementary-determining region (CDR L) 1 amino acid sequence KASAAVGTYVA (SEQ ID NO:141);
(e) the CDR L2 amino acid sequence SASYRKR (SEQ ID NO:142); and
(f) the CDR L3 amino acid sequence HQYYTYPLFT (SEQ ID NO:143).

In another specific embodiment the extracellular domain of the antigen binding receptor comprises an antigen binding moiety capable of specific binding to CEA, wherein the antigen binding moiety comprises:

(i) a heavy chain variable region (VH) comprising
(a) the heavy chain complementarity-determining region (CDR H) 1 amino acid sequence DTYMH (SEQ ID NO:148);
(b) the CDR H2 amino acid sequence RIDPANGNSKYVPKFQG (SEQ ID NO:149); and
(c) the CDR H3 amino acid sequence FGYYVSDYAMAY (SEQ ID NO:150); and

(ii) a light chain variable region (VL) comprising
(d) the light chain complementary-determining region (CDR L) 1 amino acid sequence RAGESVDIFGVGFLH (SEQ ID NO:151);
(e) the CDR L2 amino acid sequence RASNRAT (SEQ ID NO:152); and
(f) the CDR L3 amino acid sequence QQTNEDPYT (SEQ ID NO:153).

In one embodiment the extracellular domain of the antigen binding receptor comprises an antigen binding moiety capable of specific binding to CEA, wherein the antigen binding moiety comprises a heavy chain variable region (VH) comprising an amino acid sequence that is at least about 95%, 96%, 97%, 98%, 99% or 100% identical to an amino acid selected from SEQ ID NO:146 and SEQ ID NO:156, and a light chain variable region (VL) comprising an amino acid sequence that is at least about 95%, 96%, 97%, 98%, 99% or 100% identical to an amino acid sequence selected from SEQ ID NO:147 and SEQ ID NO:157.

In one embodiment the extracellular domain of the antigen binding receptor comprises an antigen binding moiety capable of specific binding CEA, wherein the antigen binding moiety comprises the heavy chain variable region (VH) of SEQ ID NO:146 and the light chain variable region (VL) of SEQ ID NO:147.

In one embodiment the extracellular domain of the antigen binding receptor comprises an antigen binding moiety capable of specific binding CEA, wherein the antigen binding moiety comprises the heavy chain variable region (VH) of SEQ ID NO:156 and the light chain variable region (VL) of SEQ ID NO:157.

In one embodiment, the at least one antigen binding moiety is a Fab, a crossFab or a scFab fragment.

In one preferred embodiment the extracellular domain of the antigen binding receptor comprises an antigen binding moiety capable of specific binding to CEA, wherein the antigen binding moiety is a Fab fragment.

In one embodiment the extracellular domain of the antigen binding receptor comprises an antigen binding moiety capable of specific binding to CEA, wherein the at least one antigen binding moiety is a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In one embodiment the extracellular domain of the antigen binding receptor comprises an antigen binding moiety capable of specific binding to CEA, wherein the scFv fragment comprises an amino acid sequence selected from SEQ ID NO:145 and SEQ ID NO:155.

In one embodiment the extracellular domain of the antigen binding receptor comprises an antigen binding moiety capable of specific binding to CEA, wherein the at least one antigen binding moiety is a scFab fragment which is a polypeptide consisting of a heavy chain (VH-CH1), a light chain (VL-CL) and a linker, wherein said heavy and light chains and said linker have one of the following configurations in N-terminal to C-terminal direction: a) VL-CL-linker-VH-CH1 or b) VH-CH-linker-VL-CL. In a preferred embodiment, the scFab fragment has the configuration VL-CL-linker-VH-CH1.

In a further alternative particular embodiment of the present invention, provided are antigen binding receptors capable of specific binding CEA and effector cells expressing said antigen binding receptors. The target cell is one which expresses a CEA polypeptide and is of a cell type which specifically expresses or overexpresses a CEA polypeptide. The cells may be cancerous or normal cells of the particular cell type. In one embodiment the cell is a cancer cell. Accordingly, in one specific embodiment the extracellular domain of the antigen binding receptor comprises an antigen binding moiety capable of specific binding to CEA, wherein the antigen binding moiety is a Fab, crossFab or a scFab.

Anchoring Transmembrane Domain

In the context of the present invention, the anchoring transmembrane domain of the antigen binding receptors of the present invention may be characterized by not having a cleavage site for mammalian proteases. In the context of the present invention, proteases refer to proteolytic enzymes that are able to hydrolyze the amino acid sequence of a transmembrane domain comprising a cleavage site for the protease. The term proteases include both endopeptidases and exopeptidases. In the context of the present invention any anchoring transmembrane domain of a transmembrane protein as laid down among others by the CD-nomenclature may be used to generate the antigen binding receptors of the invention, which activate T cells, preferably CD8+ T cells, upon binding to an antigen as defined herein.

Accordingly, in the context of the present invention, the anchoring transmembrane domain may comprise part of a murine/mouse or preferably of a human transmembrane domain. An example for such an anchoring transmembrane domain is a transmembrane domain of CD28, for example, having the amino acid sequence as shown herein in SEQ ID NO:14 (as encoded by the DNA sequence shown in SEQ ID NO:29). In the context of the present invention, the transmembrane domain of the antigen binding receptor of the present invention may comprise/consist of an amino acid sequence as shown in SEQ ID NO:14 (as encoded by the DNA sequence shown in SEQ ID NO:29).

In an illustrative embodiment of the present invention, as a proof of concept, an antigen binding receptor is provided comprising an amino acid sequence of SEQ ID NO:7 (as encoded by the DNA sequence shown in SEQ ID NO:22), and comprising a fragment/polypeptide part of CD28 (the Uniprot Entry number of the human CD28 is P10747 (with the version number 173 and version 1 of the sequence)) as shown herein as SEQ ID NO:97 (as encoded by the DNA sequence shown in SEQ ID NO:96). Alternatively, any protein having a transmembrane domain, as provided among others by the CD nomenclature, may be used as an anchoring transmembrane domain of the antigen binding receptor protein of the invention. As described above, the herein provided antigen binding receptor may comprise the anchoring transmembrane domain of CD28 which is located at amino acids 153 to 179, 154 to 179, 155 to 179, 156 to 179, 157 to 179, 158 to 179, 159 to 179, 160 to 179, 161 to 179, 162 to 179, 163 to 179, 164 to 179, 165 to 179, 166 to 179, 167 to 179, 168 to 179, 169 to 179, 170 to 179, 171 to 179, 172 to 179, 173 to 179, 174 to 179, 175 to 179, 176 to 179, 177 to 179 or 178 to 179 of the human full length CD28 protein as shown in SEQ ID NO:97 (as encoded by the cDNA shown in SEQ ID NO:96). Accordingly, in context of the present invention the anchoring transmembrane domain may comprise or consist of an amino acid sequence as shown in SEQ ID NO:14 (as encoded by the DNA sequence shown in SEQ ID NO:29).

In one embodiment provided is an antigen binding receptor comprising an anchoring transmembrane domain and an extracellular domain comprising a Fab fragment capable of specific binding to CD20, wherein antigen binding receptor comprises a
(a) a heavy chain comprising the amino acid sequence of SEQ ID NO:8 fused at the C-terminus to the N-terminus of the anchoring transmembrane domain of SEQ ID NO:14, optionally through the peptide linker of SEQ ID NO:20; and
(b) a light chain comprising the amino acid sequence of SEQ ID NO:9.

In an alternative embodiment provided is an antigen binding receptor comprising an anchoring transmembrane domain and an extracellular domain comprising a Fab fragment capable of specific binding to CD20, wherein antigen binding receptor comprises a
(a) a light chain comprising the amino acid sequence of SEQ ID NO:9 fused at the C-terminus to the N-terminus of the anchoring transmembrane domain of SEQ ID NO:14, optionally through the peptide linker of SEQ ID NO:20; and
(b) a heavy chain comprising the amino acid sequence of SEQ ID NO:8.

In an alternative embodiment provided is an antigen binding receptor comprising an anchoring transmembrane domain and an extracellular domain comprising a crossFab fragment capable of specific binding to CD20, wherein antigen binding receptor comprises a
(a) a heavy chain comprising the amino acid sequence of SEQ ID NO:42 fused at the C-terminus to the N-terminus of the anchoring transmembrane domain of SEQ ID NO:14, optionally through the peptide linker of SEQ ID NO:20; and
(b) a light chain comprising the amino acid sequence of SEQ ID NO:43.

In an alternative embodiment provided is an antigen binding receptor comprising an anchoring transmembrane domain and an extracellular domain comprising a crossFab fragment capable of specific binding to CD20, wherein antigen binding receptor comprises a
(a) a light chain comprising the amino acid sequence of SEQ ID NO:37 fused at the C-terminus to the N-terminus of the anchoring transmembrane domain of SEQ ID NO:14, optionally through the peptide linker of SEQ ID NO:20; and
(b) a heavy chain comprising the amino acid sequence of SEQ ID NO:38.

In one embodiment provided is an antigen binding receptor comprising an anchoring transmembrane domain and an extracellular domain comprising a scFab fragment capable of specific binding to CD20, wherein the scFab fragment comprises the amino acid sequence of SEQ ID NO:51 fused at the C-terminus to the N-terminus of the anchoring transmembrane domain of SEQ ID NO:14, optionally through a peptide linker of SEQ ID NO:20.

In one embodiment provided is an antigen binding receptor comprising an anchoring transmembrane domain and an extracellular domain comprising a Fab fragment capable of specific binding to PDL1, wherein the antigen binding receptor comprises a
(a) a heavy chain comprising the amino acid sequence of SEQ ID NO:75 fused at the C-terminus to the N-terminus of the anchoring transmembrane domain of SEQ ID NO:14, optionally through the peptide linker of SEQ ID NO:20; and
(b) a light chain comprising an amino acid sequence of SEQ ID NO:76.

In an alternative embodiment, provided is an antigen binding receptor comprising an anchoring transmembrane domain and an extracellular domain comprising a Fab fragment capable of specific binding to PDL1, wherein the antigen binding receptor comprises a
(a) a light chain comprising the amino acid sequence of SEQ ID NO:76 fused at the C-terminus to the N-terminus of the anchoring transmembrane domain of SEQ ID NO:14, optionally through the peptide linker of SEQ ID NO:20; and
(b) a heavy chain comprising an amino acid sequence of SEQ ID NO:75.

In an alternative embodiment provided is an antigen binding receptor comprising an anchoring transmembrane domain and an extracellular domain comprising a crossFab fragment capable of specific binding to PDL1, wherein the antigen binding receptor comprises a
(a) a heavy chain comprising the amino acid sequence of SEQ ID NO:83 fused at the C-terminus to the N-terminus of the anchoring transmembrane domain of SEQ ID NO:14, optionally through the peptide linker of SEQ ID NO:20; and
(b) a light chain comprising an amino acid sequence of SEQ ID NO:84.

In an alternative embodiment, provided is an antigen binding receptor comprising an anchoring transmembrane domain and an extracellular domain comprising a crossFab fragment capable of specific binding to PDL1, wherein the antigen binding receptor comprises a
(a) a light chain comprising the amino acid sequence of SEQ ID NO:80 fused at the C-terminus to the N-terminus of the anchoring transmembrane domain of SEQ ID NO:14, optionally through the peptide linker of SEQ ID NO:20; and
(b) a heavy chain comprising an amino acid sequence of SEQ ID NO:81.

In one embodiment provided is an antigen binding receptor comprising an anchoring transmembrane domain and an extracellular domain comprising a scFab fragment capable of specific binding to PDL1, wherein the scFab fragment comprises the amino acid sequence of SEQ ID NO:86 fused at the C-terminus to the N-terminus of the anchoring transmembrane domain of SEQ ID NO:14, optionally through a peptide linker of SEQ ID NO:20.

Stimulatory Signaling Domain (SSD) and Co-Stimulatory Signaling Domain (CSD)

Preferably, the antigen binding receptor of the present invention comprises at least one stimulatory signaling domain and/or at least one co-stimulatory signaling domain. Accordingly, the herein provided antigen binding receptor preferably comprises a stimulatory signaling domain, which provides T cell activation. The herein provided antigen binding receptor may comprise a stimulatory signaling domain which is a fragment/polypeptide part of murine/mouse or human CD3z (the UniProt Entry of the human CD3z is P20963 (version number 177 with sequence number 2; the UniProt Entry of the murine/mouse CD3z is P24161 (primary citable accession number) or Q9D3G3 (secondary citable accession number) with the version number 143 and the sequence number 1)), FCGR3A (the UniProt Entry of the human FCGR3A is P08637 (version number 178 with sequence number 2)), or NKG2D (the UniProt Entry of the human NKG2D is P26718 (version number 151 with sequence number 1); the UniProt Entry of the murine/mouse NKG2D is 054709 (version number 132 with sequence number 2)).

Thus, the stimulatory signaling domain which is comprised in the herein provided antigen binding receptor may be a fragment/polypeptide part of the full length of CD3z, FCGR3A or NKG2D. The amino acid sequence of the murine/mouse full length of CD3z is shown herein as SEQ ID NO: 94 (murine/mouse as encoded by the DNA sequences shown in SEQ ID NO:95). The amino acid sequence of the human full length CD3zis shown herein as SEQ ID NO:92 (human as encoded by the DNA sequence shown in SEQ ID NO:93). The antigen binding receptor of the present invention may comprise fragments of CD3z, FCGR3A or NKG2D as stimulatory domain, provided that at least one signaling domain is comprised. In particular, any part/fragment of CD3z, FCGR3A, or NKG2D is suitable as stimulatory domain as long as at least one signaling motive is comprised. However, more preferably, the antigen binding receptor of the present invention comprises polypeptides which are derived from human origin.

Preferably, the herein provided antigen binding receptor comprises the amino acid sequence as shown herein as SEQ ID NO:92 (CD3z) (human as encoded by the DNA sequence shown in SEQ ID NO:93 (CD3z)). For example, the fragment/polypeptide part of the human CD3z which may be comprised in the antigen binding receptor of the present invention may comprise or consist of the amino acid sequence shown in SEQ ID NO:16 (as encoded by the DNA sequence shown in SEQ ID NO:31). Accordingly, in one embodiment the antigen binding receptor comprises the sequence as shown in SEQ ID NO:16 or a sequence which has up to 1, 2, 3, 4, 5, 6, 7, 8, 9, 10, 11, 12, 13, 14, 15, 16, 17, 18, 19, 20, 21, 22, 23, 23, 24, 25, 26, 27, 28, 29 or 30 substitutions, deletions or insertions in comparison to SEQ ID NO:16 and which is characterized by having a stimulatory signaling activity. Specific configurations of antigen binding receptors comprising a stimulatory signaling domain (SSD) are provided herein below and in the Examples and Figures. The stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays.

Furthermore, the herein provided antigen binding receptor preferably comprises at least one co-stimulatory signaling domain which provides additional activity to the T cell. The herein provided antigen binding receptor may comprise a co-stimulatory signaling domain which is a fragment/polypeptide part of murine/mouse or human CD28 (the UniProt Entry of the human CD28 is P10747 (version number 173 with sequence number 1); the UniProt Entry of the murine/mouse CD28 is P31041 (version number 134 with sequence number 2)), CD137 (the UniProt Entry of the human CD137 is Q07011 (version number 145 with sequence number 1); the UniProt Entry of murine/mouse CD137 is P20334 (version number 139 with sequence number 1)), OX40 (the UniProt Entry of the human OX40 is P23510 (version number 138 with sequence number 1); the UniProt Entry of murine/mouse OX40 is P43488 (version number 119 with sequence number 1)), ICOS (the UniProt Entry of the human ICOS is Q9Y6W8 (version number 126 with sequence number 1)); the UniProt Entry of the murine/mouse ICOS is Q9WV40 (primary citable accession number) or Q9JL17 (secondary citable accession number) with the version number 102 and sequence version 2)), CD27 (the UniProt Entry of the human CD27 is P26842 (version number 160 with sequence number 2); the Uniprot Entry of the murine/mouse CD27 is P41272 (version number 137 with sequence version 1)), 4-1-BB (the UniProt Entry of the murine/mouse 4-1-BB is P20334 (version number 140 with sequence version 1); the UniProt Entry of the human 4-1-BB is Q07011 (version number 146 with sequence version)), DAP10 (the UniProt Entry of the human DAP10 is Q9UBJ5 (version number 25 with sequence number 1); the UniProt entry of the murine/mouse DAP10 is Q9QUJ0 (primary citable accession number) or Q9R1E7 (secondary citable accession number) with the version number 101 and the sequence number 1)) or DAP12 (the UniProt Entry of the human DAP12 is O43914 (version number 146 and the sequence number 1); the UniProt entry of the murine/mouse DAP12 is O054885 (primary citable accession number) or Q9R1E7 (secondary citable accession number) with the version number 123 and the sequence number 1). In certain embodiments of the present invention the antigen binding receptor of the present invention may comprise one or more, i.e. 1, 2, 3, 4, 5, 6 or 7 of the herein defined co-stimulatory signaling domains. Accordingly, in the context of the present invention, the antigen binding receptor of the present invention may comprise a fragment/polypeptide part of a murine/mouse or preferably of a human CD28 as first co-stimulatory signaling domain and the second co-stimulatory signaling domain is selected from the group consisting of the murine/mouse or preferably of the human CD27, CD28, CD137, OX40, ICOS, DAP10 and DAP12, or fragments thereof. Preferably, the antigen binding receptor of the present invention comprises a co-stimulatory signaling domain which is derived from a human origin. Thus, more preferably, the co-stimulatory signaling domain(s) which is (are) comprised in the antigen binding receptor of the present invention may comprise or consist of the amino acid sequence as shown in SEQ ID NO:15 (as encoded by the DNA sequence shown in SEQ ID NO:30).

Thus, the co-stimulatory signaling domain which may be optionally comprised in the herein provided antigen binding receptor is a fragment/polypeptide part of the full length CD27, CD28, CD137, OX40, ICOS, DAP10 and DAP12. The amino acid sequence of the murine/mouse full length CD28 is shown herein as SEQ ID NO:99 (murine/mouse as encoded by the DNA sequences shown in SEQ ID NO:98). However, because human sequences are most preferred in the context of the present invention, the co-stimulatory signaling domain which may be optionally comprised in the herein provided antigen binding receptor protein is a fragment/polypeptide part of the human full length CD27, CD28, CD137, OX40, ICOS, DAP10 or DAP12. The amino acid sequences of the human full length CD28 is shown herein as SEQ ID NO:97 (human as encoded by the DNA sequences shown in SEQ ID NO:96)).

In one preferred embodiment, the antigen binding receptor comprises CD28 or a fragment thereof as co-stimulatory signaling domain. The herein provided antigen binding receptor may comprise a fragment of CD28 as co-stimulatory signaling domain, provided that at least one signaling domain of CD28 is comprised. In particular, any part/fragment of CD28 is suitable for the antigen binding receptor of the invention as long as at least one of the signaling motives of CD28 is comprised. For example, the CD28 polypeptide which is comprised in the antigen binding receptor protein of the present invention may comprise or consist of the amino acid sequence shown in SEQ ID NO:15 (as encoded by the DNA sequence shown in SEQ ID NO:30). In the present invention the intracellular domain of CD28, which functions as a co-stimulatory signaling domain, may comprise a sequence derived from the intracellular domain of the CD28 polypeptide having the sequence(s) YMNM (SEQ ID NO:132) and/or PYAP (SEQ ID NO:133). Preferably, the antigen binding receptor of the present invention comprises polypeptides which are derived from human origin. For example, the fragment/polypeptide part of the human CD28 which may be comprised in the antigen binding receptor of the present invention may comprise or consist of the amino acid sequence shown in SEQ ID NO:15 (as encoded by the DNA sequence shown in SEQ ID NO:30). Accordingly, in the context of the present invention the antigen binding receptor comprises the sequence as shown in SEQ ID NO:15 or a sequence which has up to 1, 2, 3, 4, 5, 6, 7, 8, 9 or 10 substitutions, deletions or insertions in comparison to SEQ ID NO:15 and which is characterized by having a co-stimulatory signaling activity. Specific configurations of antigen binding receptors comprising a co-stimulatory signaling domain (CSD) are provided herein below and in the Examples and Figures. The co-stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays.

As mentioned above, in an embodiment of the present invention, the co-stimulatory signaling domain of the antigen binding receptor may be derived from the human CD28 gene (Uni Prot Entry No: P10747 (accession number with the entry version: 173 and version 1 of the sequence)) and provides CD28 activity, defined as cytokine production, proliferation and lytic activity of the transduced cell described herein, like a transduced T cell. CD28 activity can be measured by release of cytokines by ELISA or flow cytometry of cytokines such as interferon-gamma (IFN-γ) or interleukin 2 (IL-2), proliferation of T cells measured e.g. by ki67-measurement, cell quantification by flow cytometry, or lytic activity as assessed by real time impedence measurement of the target cell (by using e.g. an ICELLligence instrument as described e.g. in Thakur et al., Biosens Bioelectron. 35(1) (2012), 503-506; Krutzik et al., Methods Mol Biol. 699 (2011), 179-202; Ekkens et al., Infect Immun. 75(5) (2007), 2291-2296; Ge et al., Proc Natl Acad Sci U S A. 99(5) (2002), 2983-2988; Düwell et al., Cell Death Differ. 21(12) (2014), 1825-1837, Erratum in: Cell Death Differ. 21(12) (2014), 161). The co-stimulatory signaling domains PYAP (AA 208 to 211 of SEQ ID NO:133 and YMNM (AA 191 to 194 of SEQ ID NO:132) are beneficial for the function of the CD28 polypeptide and the functional effects enumerated above. The amino acid sequence of the YMNM domain is shown in SEQ ID NO:132; the amino acid sequence of the PYAP domain is shown in SEQ ID NO:133. Accordingly, in the antigen binding receptor of the present invention, the CD28 polypeptide preferably comprises a sequence derived from intracellular domain of a CD28 polypeptide having the sequences YMNM (SEQ ID NO:132) and/or PYAP (SEQ ID NO:133). In the context of the present invention an intracellular domain of a CD28 polypeptide having the sequences YMNM (SEQ ID NO:132) and/or PYAP (SEQ ID NO:133) characterized by a CD28 activity, defined as cytokine production, proliferation and lytic activity of a transduced cell described herein, like e.g. a transduced T cell. Accordingly, in the context of the present invention the co-stimulatory signaling domain of the antigen binding receptors of the present invention has the amino acid sequence of SEQ ID NO:15 (human) (as encoded by the DNA sequence shown in SEQ ID NO:30). However, in the antigen binding receptor of the present invention, one or both of these domains may be mutated to FMNM (SEQ ID NO:134) and/or AYAA (SEQ ID NO:135), respectively. Either of these mutations reduces the ability of a transduced cell comprising the antigen binding receptor to release cytokines without affecting its ability to proliferate and can advantageously be used to prolong the viability and thus the therapeutic potential of the transduced cells. Or, in other words, such a non-functional mutation preferably enhances the persistence of the cells which are transduced with the herein provided antigen binding receptor in vivo. These signaling motives may, however, be present at any site within the intracellular domain of the herein provided antigen binding receptor.

Linker and Signal Peptides

Moreover, the herein provided antigen binding receptor may comprise at least one linker (or “spacer”). A linker is usually a peptide having a length of up to 20 amino acids. Accordingly, in the context of the present invention the linker may have a length of 1, 2, 3, 4, 5, 6, 7, 8, 9, 10, 11, 12, 13, 14, 15, 16, 17, 18, 19 or 20 amino acids. For example, the herein provided antigen binding receptor may comprise a linker between the extracellular domain comprising at least one antigen binding moiety, the anchoring transmembrane domain, the co-stimulatory signaling domain and/or the stimulatory signaling domain. Such linkers have the advantage that they increase the probability that the different polypeptides of the antigen binding receptor (i.e. the extracellular domain, the anchoring transmembrane domain, the co-stimulatory signaling domain and/or the stimulatory signaling domain) fold independently and behave as expected. Thus, in the context of the present invention, the extracellular domain comprising at least one antigen binding moiety capable, the anchoring transmembrane domain that does not have a cleavage site for mammalian proteases, the co-stimulatory signaling domain and the stimulatory signaling domain may be comprised in a single-chain multi-functional polypeptide chain. A fusion construct e.g. may consist of (a) polypeptide(s) comprising (an) extracellular domain(s) comprising at least one antigen binding moiety, (an) anchoring transmembrane domain(s), (a) co-stimulatory signaling domain(s) and/or (a) stimulatory signaling domain(s). In preferred embodiments, the antigen binding receptor comprises an antigen binding moiety which is not a single chain construct, i.e. the antigen binding moiety is a Fab or a crossFab fragment. Preferably such constructs will comprise a single chain heavy or light chain fusion polypeptide combined with an immunoglobulin light or heavy chain as described herein, e.g., a heavy chain fusion polypeptide comprises (an) immunoglobulin heavy chain(s), (an) anchoring transmembrane domain(s), (a) co-stimulatory signaling domain(s) and/or (a) stimulatory signaling domain(s) and is combined with (an) immunoglobulin light chain(s), or a light chain fusion polypeptide comprises (an) immunoglobulin light chain(s), (an) anchoring transmembrane domain(s), (a) co-stimulatory signaling domain(s) and/or (a) stimulatory signaling domain(s) and is combined with (an) immunoglobulin heavy chain(s). Accordingly, the antigen binding moiety, the anchoring transmembrane domain, the co-stimulatory signaling domain and the stimulatory signaling domain may be connected by one or more identical or different peptide linker as described herein. For example, in the herein provided antigen binding receptor the linker between the extracellular domain comprising at least one antigen binding moiety and the anchoring transmembrane domain may comprise or consist of the amino and amino acid sequence as shown in SEQ ID NO:20. Accordingly, the anchoring transmembrane domain, the co-stimulatory signaling domain and/or the stimulatory domain may be connected to each other by peptide linkers or alternatively, by direct fusion of the domains.

In some embodiments the antigen binding moiety comprised in the extracellular domain is a single-chain variable fragment (scFv) which is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. For example, the linker may have the amino and amino acid sequence as shown in SEQ ID NO:19.

In some embodiments according to the invention the antigen binding moiety comprised in the extracellular domain is a single chain Fab fragment or scFab which is a polypeptide consisting of an heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain.

In some embodiments according to the invention the antigen binding moiety comprised in the extracellular domain is a crossover single chain Fab fragment which is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids.

The herein provided antigen binding receptor or parts thereof may comprise a signal peptide. Such a signal peptide will bring the protein to the surface of the T cell membrane. For example, in the herein provided antigen binding receptor the signal peptide may have the amino and amino acid sequence as shown in SEQ ID NO:136 (as encoded by the DNA sequence shown in SEQ ID NO:137).

T Cell Activating Antigen Binding Receptors

The components of the antigen binding receptors as described herein can be fused to each other in a variety of configurations to generate T cell activating antigen binding receptors.

In some embodiments, the antigen binding receptor comprises an extracellular domain composed of a heavy chain variable domain (VH) and a light chain variable domain (VL) connected to an anchoring transmembrane domain. In some embodiments, the VH domain is fused at the C-terminus to the N-terminus of the VL domain, optionally through a peptide linker. In other embodiments, the antigen binding receptor further comprises a stimulatory signaling domain and/or a co-stimulatory signaling domain. In a specific such embodiment, the antigen binding receptor essentially consists of a VH domain and a VL domain, an anchoring transmembrane domain, and optionally a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain. Optionally, the antigen binding receptor further comprises a co-stimulatory signaling domain. In one such specific embodiment, the antigen binding receptor essentially consists of a VH domain and a VL domain, an anchoring transmembrane domain, a stimulatory signaling domain and a co-stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain, wherein the stimulatory signaling domain is fused at the C-terminus to the N-terminus of the co-stimulatory signaling domain. In an alternative embodiment, the co-stimulatory signaling domain is connected to the anchoring transmembrane domain instead of the stimulatory signaling domain. In a preferred embodiment, the antigen binding receptor essentially consists of a VH domain and a VL domain, an anchoring transmembrane domain, a co-stimulatory signaling domain and a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the co-stimulatory signaling domain, wherein the co-stimulatory signaling domain is fused at the C-terminus to the N-terminus of the stimulatory signaling domain.

In alternative embodiments, one of the binding moieties is a scFab fragment. In one preferred embodiment, the antigen binding moiety is fused at the C-terminus of the scFab to the N-terminus of the anchoring transmembrane domain, optionally through a peptide linker. In other embodiments, the antigen binding receptor further comprises a stimulatory signaling domain and/or a co-stimulatory signaling domain. In a specific such embodiment, the antigen binding receptor essentially consists of a scFab fragment, an anchoring transmembrane domain, and optionally a stimulatory signaling domain connected by one or more peptide linkers, wherein the scFab is fused at the C-terminus to the N-terminus of the anchoring transmembrane domain, wherein the anchoring transmembrane domain is fused at the C-terminus to the N-terminus of the stimulatory signaling domain. Preferably, the antigen binding receptor further comprises a co-stimulatory signaling domain. In one such embodiment, the antigen binding receptor essentially consists of a scFab fragment, an anchoring transmembrane domain, a stimulatory signaling domain and a co-stimulatory signaling domain connected by one or more peptide linkers, wherein the scFab is fused at the C-terminus to the N-terminus of the anchoring transmembrane domain, wherein the stimulatory signaling domain is fused at the C-terminus to the N-terminus of the co-stimulatory signaling domain. In a preferred embodiment, the co-stimulatory signaling domain is connected to the anchoring transmembrane domain instead of the stimulatory signaling domain. In a most preferred embodiment, the antigen binding receptor essentially consists of a scFab fragment, an anchoring transmembrane domain, a co-stimulatory signaling domain and a stimulatory signaling domain, wherein the scFab is fused at the C-terminus to the N-terminus of the anchoring transmembrane domain through a peptide linker, wherein the anchoring transmembrane domain is fused at the C-terminus to the N-terminus of the co-stimulatory signaling domain, wherein the co-stimulatory signaling domain is fused at the C-terminus to N-terminus of the stimulatory signaling domain.

In preferred embodiments, one of the binding moieties is a Fab fragment or a crossFab fragment. In one preferred embodiment, the antigen binding moiety is fused at the C-terminus of the Fab or crossFab heavy chain to the N-terminus of the anchoring transmembrane domain, optionally through a peptide linker. In an alternative embodiment, the antigen binding moiety is fused at the C-terminus of the Fab or crossFab light chain to the N-terminus of the anchoring transmembrane domain, optionally through a peptide linker. In other embodiments, the antigen binding receptor further comprises a stimulatory signaling domain and/or a co-stimulatory signaling domain. In a specific such embodiment, the antigen binding receptor essentially consists of a Fab or crossFab fragment, an anchoring transmembrane domain, and optionally a stimulatory signaling domain connected by one or more peptide linkers, wherein the Fab or crossFab fragment is fused at the C-terminus of the heavy or light chain to the N-terminus of the anchoring transmembrane domain, wherein the anchoring transmembrane domain is fused at the C-terminus to the N-terminus of the stimulatory signaling domain. Preferably, the antigen binding receptor further comprises a co-stimulatory signaling domain. In one such embodiment, the antigen binding receptor essentially consists of a Fab or crossFab fragment, an anchoring transmembrane domain, a stimulatory signaling domain and a co-stimulatory signaling domain connected by one or more peptide linkers, wherein the Fab or crossFab fragment is fused at the C-terminus of the heavy or light chain to the N-terminus of the anchoring transmembrane domain, wherein the stimulatory signaling domain is fused at the C-terminus to the N-terminus of the co-stimulatory signaling domain. In a preferred embodiment, the co-stimulatory signaling domain is connected to the anchoring transmembrane domain instead of the stimulatory signaling domain. In a most preferred embodiment, the antigen binding receptor essentially consists of a Fab or crossFab fragment, an anchoring transmembrane domain, a co-stimulatory signaling domain and a stimulatory signaling domain, wherein the Fab or crossFab fragment is fused at the C-terminus of the heavy chain to the N-terminus of the anchoring transmembrane domain through a peptide linker, wherein the anchoring transmembrane domain is fused at the C-terminus to the N-terminus of the co-stimulatory signaling domain, wherein the co-stimulatory signaling domain is fused at the C-terminus to N-terminus of the stimulatory signaling domain.

The antigen binding moiety, the anchoring transmembrane domain and the stimulatory signaling and/or co-stimulatory signaling domains may be fused to each other directly or through one or more peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers, wherein “n” is generally a number between 1 and 10, typically between 2 and 4. A preferred peptide linker for connecting the antigen binding moiety and the anchoring transmembrane moiety is GGGGS (G₄S) according to SEQ ID NO:20. An exemplary peptide linker suitable for connecting variable heavy chain (VH) and the variable light chain (VL) is GGGSGGGSGGGSGGGS (G₄S)₄ according to SEQ ID NO:19.

Additionally, linkers may comprise (a portion of) an immunoglobulin hinge region. Particularly where an antigen binding moiety is fused to the N-terminus of an anchoring transmembrane domain, it may be fused via an immunoglobulin hinge region or a portion thereof, with or without an additional peptide linker.

As described herein, the antigen binding receptors of the present invention comprise an extracellular domain comprising at least one antigen binding moiety. An antigen binding receptor with a single antigen binding moiety capable of specific binding to a target cell antigen is useful and preferred, particularly in cases where high expression of the antigen binding receptor is needed. In such cases, the presence of more than one antigen binding moiety specific for the target cell antigen may limit the expression efficiency of the antigen binding receptor. In other cases, however, it will be advantageous to have an antigen binding receptor comprising two or more antigen binding moieties specific for a target cell antigen, for example to optimize targeting to the target site or to allow crosslinking of target cell antigens.

In a preferred embodiment, the antigen binding moiety is a Fab fragment. In one embodiment, the antigen binding moiety is fused at the C-terminus of the Fab heavy chain to the N-terminus of an anchoring transmembrane domain. In one embodiment, the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof. In a preferred embodiment, the anchoring transmembrane domain is the CD28 transmembrane domain or a fragment thereof. In a particular embodiment, the anchoring transmembrane domain is FWVLVVVGGVLACYSLLVTVAFIIFWV (SEQ ID NO:14). In one embodiment, the antigen binding receptor further comprises a co-stimulatory signaling domain (CSD). In one embodiment, the anchoring transmembrane domain of the antigen binding receptor is fused at the C-terminus to the N-terminus of a co-stimulatory signaling domain. In one embodiment, the co-stimulatory signaling domain is individually selected from the group consisting of the intracellular domain of CD27, CD28, CD137, OX40, ICOS, DAP10 and DAP12, or fragments thereof as described herein before. In a preferred embodiment, the co-stimulatory signaling domain is the intracellular domain of CD28 or a fragment thereof. In a particular embodiment the co-stimulatory signaling domain comprises or consists of the sequence: RSKRSRLLHSDYMNMTPRRPGPTRKHYQPYAPPRDFAAYRS (SEQ ID NO:15). In one embodiment, the antigen binding receptor further comprises a stimulatory signaling domain. In one embodiment, the co-stimulatory signaling domain of the antigen binding receptor is fused at the C-terminus to the N-terminus of the stimulatory signaling domain. In one embodiment, the at least one stimulatory signaling domain is individually selected from the group consisting of the intracellular domain of CD3z, FCGR3A and NKG2D, or fragments thereof. In a preferred embodiment, the stimulatory signaling domain is the intracellular domain of CD3z or a fragment thereof. In a particular embodiment the stimulatory signaling domain comprises or consists of the sequence:

(SEQ ID NO: 16)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In one embodiment, the antigen binding receptor comprising the Fab fragment is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) of SEQ ID NO:21.

In a particular embodiment, the antigen binding receptor comprises an anchoring transmembrane domain and an extracellular domain comprising at least one antigen binding moiety, wherein the at least one antigen binding moiety is a Fab fragment capable of specific binding to CD20. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration Fab-ATD-CSD-SSD. In a preferred embodiment, the antigen binding receptor has the configuration Fab-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker.

In a particular embodiment, the antigen binding moiety is capable of specific binding to a CD20, wherein the antigen binding moiety is a Fab fragment comprising at least one heavy chain complementarity determining region (CDR) selected from the group consisting of SEQ ID NO:1, SEQ ID NO:2 and SEQ ID NO:3 and at least one light chain CDR selected from the group of SEQ ID NO:4, SEQ ID NO:5, SEQ ID NO:6.

In a preferred embodiment, the antigen binding moiety is a Fab fragment capable of specific binding to CD20, wherein the antigen binding moiety comprises the complementarity determining region (CDR H) 1 amino acid sequence YSWIN (SEQ ID NO:1), the CDR H2 amino acid sequence RIFPGDGDTDYNGKFKG (SEQ ID NO:2), the CDR H3 amino acid sequence NVFDGYWLVY (SEQ ID NO:3), the light chain complementary-determining region (CDR L) 1 amino acid sequence RSSKSLLHSNGITYLY (SEQ ID NO:4), the CDR L2 amino acid sequence QMSNLVS (SEQ ID NO:5) and the CDR L3 amino acid sequence AQNLELPYT (SEQ ID NO:6).

In one embodiment the present invention provides an antigen binding receptor comprising in order from the N-terminus to the C-terminus

(i) an antigen binding moiety which is a Fab molecule capable of specific binding to CD20, comprising the heavy chain complementarity determining region (CDR) 1 of SEQ ID NO:1, the heavy chain CDR 2 of SEQ ID NO:2, the heavy chain CDR 3 of SEQ ID NO:3, the light chain CDR 1 of SEQ ID NO:4, the light chain CDR 2 of SEQ ID NO:5 and the light chain CDR 3 of SEQ ID NO:6;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment the present invention provides an antigen binding receptor capable of specific binding to CD20 comprising:
a) a heavy chain fusion polypeptide comprising in order from the N-terminus to the C-terminus;
   (i) a heavy chain comprising the heavy chain complementarity determining region (CDR) 1 of SEQ ID NO:1, the heavy chain CDR 2 of SEQ ID NO:2, the heavy chain CDR 3 of SEQ ID NO:3;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a light chain comprising the light chain CDR 1 of SEQ ID NO:4, the light chain CDR 2 of SEQ ID NO:5 and the light chain CDR 3 of SEQ ID NO:6.

In an alternative embodiment the present invention provides an antigen binding receptor capable of specific binding to CD20 comprising:
a) a light chain fusion polypeptide comprising in order from the N-terminus to the C-terminus;
   (i) a light chain comprising the light chain complementarity determining region (CDR) 1 of SEQ ID NO:4, the light chain CDR 2 of SEQ ID NO:5, the light chain CDR 3 of SEQ ID NO:6;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a heavy chain comprising the heavy chain CDR 1 of SEQ ID NO:1, the heavy chain CDR 2 of SEQ ID NO:2 and the heavy chain CDR 3 of SEQ ID NO:3.

In one embodiment the antigen binding moiety capable of specific binding to CD20 is a Fab fragment comprising a heavy chain comprising or consisting of an amino acid sequence of SEQ ID NO:8 and a light chain comprising or consisting of the amino acid sequence of SEQ ID NO:9.

In one embodiment the present invention provides an antigen binding receptor capable of specific binding to CD20 comprising:
a) a heavy chain fusion polypeptide comprising in order from the N-terminus to the C-terminus;
   (i) the heavy chain of SEQ ID NO:8;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the light chain of SEQ ID NO:9.

In an alternative embodiment the present invention provides an antigen binding receptor capable of specific binding to CD20 comprising:
a) a light chain fusion polypeptide comprising in order from the N-terminus to the C-terminus;
   (i) the light chain of SEQ ID NO:9;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the heavy chain of SEQ ID NO:8.

In a particular embodiment, the antigen binding moiety is a Fab fragment capable of specific binding to CD20, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:7 and a light chain polypeptide comprising an amino acid sequence that is at least about 95%, 96%, 97%, 98%, 99% or 100% identical to the amino acid sequence of SEQ ID NO:9.

In a particular embodiment, the antigen binding moiety is a Fab fragment capable of specific binding to CD20, wherein the antigen binding receptor comprises a light chain fusion polypeptide comprising an amino acid sequence that is at least about 95%, 96%, 97%, 98%, 99% or 100% identical to the amino acid sequence of SEQ ID NO:50 and a heavy chain polypeptide comprising an amino acid sequence that is at least about 95%, 96%, 97%, 98%, 99% or 100% identical to the amino acid sequence of SEQ ID NO:8.

In a preferred embodiment, the antigen binding moiety is a Fab fragment capable of specific binding to CD20, wherein the antigen binding receptor comprises a light chain fusion polypeptide comprising the amino acid sequence of SEQ ID NO:7 and a heavy chain polypeptide comprising the amino acid sequence of SEQ ID NO:9.

In another particular embodiment, the antigen binding moiety is capable of specific binding to a PDL1, wherein the antigen binding moiety is a Fab fragment comprising at least one heavy chain complementarity determining region (CDR) selected from the group consisting of SEQ ID NO:68, SEQ ID NO:69 and SEQ ID NO:70 and at least one light chain CDR selected from the group of SEQ ID NO:71, SEQ ID NO:72, SEQ ID NO:73.

In a preferred embodiment, the antigen binding moiety is a Fab fragment capable of specific binding to PDL1, wherein the antigen binding moiety comprises the complementarity determining region (CDR H) 1 amino acid sequence DSWIH (SEQ ID NO:68), the CDR H2 amino acid sequence WISPYGGSTYYADSVKG (SEQ ID NO:69), the CDR H3 amino acid sequence RHWPGGFDY (SEQ ID NO:70), the light chain complementary-determining region (CDR L) 1 amino acid sequence RASQDVSTAVA (SEQ ID NO:71), the CDR L2 amino acid sequence SASFLYS (SEQ ID NO:72) and the CDR L3 amino acid sequence QQYLYHPAT (SEQ ID NO:73).

In one embodiment the present invention provides an antigen binding receptor comprising in order from the N-terminus to the C-terminus

(i) an antigen binding moiety which is a Fab molecule capable of specific binding to PDL1, comprising the heavy chain complementarity determining region (CDR) 1 of SEQ ID NO:68, the heavy chain CDR 2 of SEQ ID NO:69, the heavy chain CDR 3 of SEQ ID NO:70, the light chain CDR 1 of SEQ ID NO:71, the light chain CDR 2 of SEQ ID NO:72 and the light chain CDR 3 of SEQ ID NO:73;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment the present invention provides an antigen binding receptor capable of specific binding to PDL1 comprising:
a) a heavy chain fusion polypeptide comprising in order from the N-terminus to the C-terminus;
   (i) a heavy chain comprising the heavy chain complementarity determining region (CDR) 1 of SEQ ID NO:68, the heavy chain CDR 2 of SEQ ID NO:69, the heavy chain CDR 3 of SEQ ID NO:70;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a light chain comprising the light chain CDR 1 of SEQ ID NO:71, the light chain CDR 2 of SEQ ID NO:72 and the light chain CDR 3 of SEQ ID NO:73.

In an alternative embodiment the present invention provides an antigen binding receptor capable of specific binding to PDL1 comprising:
a) a light chain fusion polypeptide comprising in order from the N-terminus to the C-terminus;
   (i) a light chain comprising the light chain complementarity determining region (CDR) 1 of SEQ ID NO:71, the light chain CDR 2 of SEQ ID NO:72, the light chain CDR 3 of SEQ ID NO:73;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and

b) a heavy chain comprising the heavy chain CDR 1 of SEQ ID NO:68, the heavy chain CDR 2 of SEQ ID NO:69 and the heavy chain CDR 3 of SEQ ID NO:70.

In one embodiment the antigen binding moiety is a Fab fragment capable of specific binding to PDL1 comprising a heavy chain comprising or consisting of an amino acid sequence of SEQ ID NO:75 and a light chain comprising or consisting of the amino acid sequence of SEQ ID NO:76.

In one embodiment the present invention provides an antigen binding receptor capable of specific binding to PDL1 comprising:
a) a heavy chain fusion polypeptide comprising in order from the N-terminus to the C-terminus;
   (i) the heavy chain of SEQ ID NO:75;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the light chain of SEQ ID NO:76.

In an alternative embodiment the present invention provides an antigen binding receptor capable of specific binding to PDL1 comprising:
a) a light chain fusion polypeptide comprising in order from the N-terminus to the C-terminus;
   (i) the light chain of SEQ ID NO:76;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the heavy chain of SEQ ID NO:75.

In a particular embodiment, the antigen binding moiety is a Fab fragment capable of specific binding to PDL1, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:74 and a light chain polypeptide comprising an amino acid sequence that is at least about 95%, 96%, 97%, 98%, 99% or 100% identical to the amino acid sequence of SEQ ID NO:76.

In another particular embodiment, the antigen binding moiety is a Fab fragment capable of specific binding to PDL1, wherein the antigen binding receptor comprises a light chain fusion polypeptide comprising an amino acid sequence that is at least about 95%, 96%, 97%, 98%, 99% or 100% identical to the amino acid sequence of SEQ ID NO:85 and a heavy chain polypeptide comprising an amino acid sequence that is at least about 95%, 96%, 97%, 98%, 99% or 100% identical to the amino acid sequence of SEQ ID NO:75.

In a preferred embodiment, the antigen binding moiety is a Fab fragment capable of specific binding to PDL1, wherein the antigen binding receptor comprises a light chain fusion polypeptide comprising the amino acid sequence of SEQ ID NO:74 and a heavy chain polypeptide comprising the amino acid sequence of SEQ ID NO:76.

In another preferred embodiment, the antigen binding moiety is a crossFab fragment. In certain embodiments as described herein below the antigen binding receptor comprises a polypeptide wherein the Fab light chain variable region of the antigen binding moiety shares a carboxy-terminal peptide bond with the Fab heavy chain constant region of the antigen binding moiety (i.e. a the antigen binding moiety comprises a crossFab heavy chain, wherein the heavy chain variable region is replaced by a light chain variable region), which in turn shares a carboxy-terminal peptide bond with the anchoring transmembrane domain (VL-CH1-ATD). In some embodiments the antigen binding receptor further comprises a polypeptide wherein the Fab heavy chain variable region of the first antigen binding moiety shares a carboxy-terminal peptide bond with the Fab light chain constant region of the first antigen binding moiety (VH-CL). In certain embodiments the polypeptides are covalently linked, e.g., by a disulfide bond. In alternative embodiments the antigen binding receptor comprises a polypeptide wherein the Fab heavy chain variable region of the antigen binding moiety shares a carboxy-terminal peptide bond with the Fab light chain constant region of the antigen binding moiety (i.e. the antigen binding moiety comprises a crossFab heavy chain, wherein the heavy chain constant region is replaced by a light chain constant region), which in turn shares a carboxy-terminal peptide bond with an anchoring transmembrane domain (VH-CL-ATD). In some embodiments the antigen binding receptor further comprises a polypeptide wherein the Fab light chain variable region of the antigen binding moiety shares a carboxy-terminal peptide bond with the Fab heavy chain constant region of the antigen binding moiety (VL-CH1) In certain embodiments the polypeptides are covalently linked, e.g., by a disulfide bond.

In one embodiment, the antigen binding moiety is fused at the C-terminus of the heavy chain constant domain to the N-terminus of an anchoring transmembrane domain. In an alternative embodiment, the antigen binding moiety is fused at the C-terminus of the light chain constant domain to the N-terminus of an anchoring transmembrane domain. In one embodiment, the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof. In a preferred embodiment, the anchoring transmembrane domain is the CD28 transmembrane domain or a fragment thereof. In a particular embodiment, the anchoring transmembrane domain is FWVLVVVGGVLACYSLLVTVAFIIFWV (SEQ ID NO:14). In one embodiment, the antigen binding receptor further comprises a co-stimulatory signaling domain (CSD). In one embodiment, the anchoring transmembrane domain of the antigen binding receptor is fused at the C-terminus to the N-terminus of a co-stimulatory signaling domain. In one embodiment, the co-stimulatory signaling domain is individually selected from the group consisting of the intracellular domain of CD27, CD28, CD137, OX40, ICOS, DAP10 and DAP12, or fragments thereof as described herein before. In a preferred embodiment, the co-stimulatory signaling domain is the intracellular domain of CD28 or a fragment thereof. In a particular embodiment the co-stimulatory signaling domain comprises or consists of the sequence:

RSKRSRLLHSDYMNMTPRRPGPTRKHYQPYAPPRDFAAYRS (SEQ ID NO:15). In one embodiment, the antigen binding receptor further comprises a stimulatory signaling domain. In one embodiment, the co-stimulatory signaling domain of the antigen binding receptor is fused at the C-terminus to the N-terminus of the stimulatory signaling domain. In one embodiment, the at least one stimulatory signaling domain is individually selected from the group consisting of the intracellular domain of CD3z, FCGR3A and NKG2D, or fragments thereof. In a preferred embodiment, the stimulatory signaling domain is the intracellular domain of CD3z or a fragment thereof. In a particular embodiment the stimulatory signaling domain comprises or consists of the sequence:

(SEQ ID NO: 16)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In one embodiment, the antigen binding receptor comprising the crossFab fragment is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) of SEQ ID NO:21.

In a particular embodiment, the antigen binding receptor comprises an anchoring transmembrane domain and an extracellular domain comprising at least one antigen binding moiety, wherein the at least one antigen binding moiety is a crossFab fragment capable of specific binding to CD20. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration crossFab-ATD-CSD-SSD. In a preferred embodiment, the antigen binding receptor has the configuration crossFab-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker. In a particular embodiment, the antigen binding moiety is capable of specific binding to a CD20, wherein the antigen binding moiety is a crossFab fragment comprising at least one heavy chain complementarity determining region (CDR) selected from the group consisting of SEQ ID NO:1, SEQ ID NO:2 and SEQ ID NO:3 and at least one light chain CDR selected from the group of SEQ ID NO:4, SEQ ID NO:5, SEQ ID NO:6.

In a preferred embodiment, the antigen binding moiety is a crossFab fragment capable of specific binding to CD20, wherein the antigen binding moiety comprises the complementarity determining region (CDR H) 1 amino acid sequence YSWIN (SEQ ID NO:1), the CDR H2 amino acid sequence RIFPGDGDTDYNGKFKG (SEQ ID NO:2), the CDR H3 amino acid sequence NVFDGYWLVY (SEQ ID NO:3), the light chain complementary-determining region (CDR L) 1 amino acid sequence RSSKSLLHSNGITYLY (SEQ ID NO:4), the CDR L2 amino acid sequence QMSNLVS (SEQ ID NO:5) and the CDR L3 amino acid sequence AQNLELPYT (SEQ ID NO:6).

In one embodiment the present invention provides an antigen binding receptor comprising in order from the N-terminus to the C-terminus

(i) an antigen binding moiety which is a crossFab molecule capable of specific binding to CD20, comprising the heavy chain complementarity determining region (CDR) 1 of SEQ ID NO:1, the heavy chain CDR 2 of SEQ ID NO:2, the heavy chain CDR 3 of SEQ ID NO:3, the light chain CDR 1 of SEQ ID NO:4, the light chain CDR 2 of SEQ ID NO:5 and the light chain CDR 3 of SEQ ID NO:6;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment the present invention provides an antigen binding receptor comprising:
a) a heavy chain fusion polypeptide comprising in order from the N-terminus to the C-terminus;
   (i) a heavy chain comprising the heavy chain complementarity determining region (CDR) 1 of SEQ ID NO:1, the heavy chain CDR 2 of SEQ ID NO:2, the heavy chain CDR 3 of SEQ ID NO:3;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a light chain comprising the light chain CDR 1 of SEQ ID NO:4, the light chain CDR 2 of SEQ ID NO:5 and the light chain CDR 3 of SEQ ID NO:6.

In an alternative embodiment the present invention provides an antigen binding receptor comprising:
a) a light chain fusion polypeptide comprising in order from the N-terminus to the C-terminus;
   (i) a light chain comprising the light chain complementarity determining region (CDR) 1 of SEQ ID NO:4, the light chain CDR 2 of SEQ ID NO:5, the light chain CDR 3 of SEQ ID NO:6;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a heavy chain comprising the heavy chain CDR 1 of SEQ ID NO:1, the heavy chain CDR 2 of SEQ ID NO:2 and the heavy chain CDR 3 of SEQ ID NO:3.

In one embodiment the antigen binding moiety is a crossFab fragment comprising a heavy chain comprising or consisting of an amino acid sequence of SEQ ID NO:38 and a light chain comprising or consisting of the amino acid sequence of SEQ ID NO:37.

In an alternative embodiment the antigen binding moiety is a crossFab fragment comprising a heavy chain comprising or consisting of an amino acid sequence of SEQ ID NO:42 and a light chain comprising or consisting of the amino acid sequence of SEQ ID NO:43.

In one embodiment the present invention provides an antigen binding receptor comprising:
a) a heavy chain fusion polypeptide comprising in order from the N-terminus to the C-terminus;
   (i) the heavy chain of SEQ ID NO:42;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the light chain of SEQ ID NO:43.

In an alternative embodiment the present invention provides an antigen binding receptor comprising:
a) a light chain fusion polypeptide comprising in order from the N-terminus to the C-terminus;
   (i) the light chain of SEQ ID NO:37;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the heavy chain of SEQ ID NO:38.

In a particular embodiment, the antigen binding moiety is a crossFab fragment capable of specific binding to CD20, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:41 and a light chain polypeptide comprising an amino acid sequence that is at least about 95%, 96%, 97%, 98%, 99% or 100% identical to the amino acid sequence of SEQ ID NO:43.

In a particular embodiment, the antigen binding moiety is a Fab fragment capable of specific binding to CD20, wherein the antigen binding receptor comprises a light chain fusion polypeptide comprising an amino acid sequence that is at least about 95%, 96%, 97%, 98%, 99% or 100% identical to the amino acid sequence of SEQ ID NO:36 and a heavy chain polypeptide comprising an amino acid sequence that is at least about 95%, 96%, 97%, 98%, 99% or 100% identical to the amino acid sequence of SEQ ID NO:38.

In a preferred embodiment, the antigen binding moiety is a Fab fragment capable of specific binding to CD20, wherein the antigen binding receptor comprises a light chain fusion polypeptide comprising the amino acid sequence of SEQ ID NO:36 and a heavy chain polypeptide comprising the amino acid sequence of SEQ ID NO:38.

In another particular embodiment, the antigen binding receptor comprises an anchoring transmembrane domain and an extracellular domain comprising at least one antigen binding moiety, wherein the at least one antigen binding moiety is a crossFab fragment capable of specific binding to PDL1. In one embodiment, the antigen binding receptor of the invention comprises an anchoring transmembrane domain (ATD), a co-stimulatory signaling domain (C SD) and a stimulatory signaling domain (SSD). In one such embodiment, the antigen binding receptor has the configuration crossFab-ATD-CSD-SSD. In a preferred embodiment, the antigen binding receptor has the configuration crossFab-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker. In a particular embodiment, the antigen binding moiety is capable of specific binding to a PDL1, wherein the antigen binding moiety is a crossFab fragment comprising at least one heavy chain complementarity determining region (CDR) selected from the group consisting of SEQ ID NO:68, SEQ ID NO:69 and SEQ ID NO:70 and at least one light chain CDR selected from the group of SEQ ID NO:71, SEQ ID NO:72, SEQ ID NO:73.

In a preferred embodiment, the antigen binding moiety is a crossFab fragment capable of specific binding to PDL1, wherein the antigen binding moiety comprises the complementarity determining region (CDR H) 1 amino acid sequence DSWIH (SEQ ID NO:68), the CDR H2 amino acid sequence WISPYGGSTYYADSVKG (SEQ ID NO:69), the CDR H3 amino acid sequence RHWPGGFDY (SEQ ID NO:70), the light chain complementary-determining region (CDR L) 1 amino acid sequence RASQDVSTAVA (SEQ ID NO:71), the CDR L2 amino acid sequence SASFLYS (SEQ ID NO:72) and the CDR L3 amino acid sequence QQYLYHPAT (SEQ ID NO:73).

In one embodiment the present invention provides an antigen binding receptor comprising in order from the N-terminus to the C-terminus:

(i) an antigen binding moiety which is a crossFab molecule capable of specific binding to PDL1, comprising the heavy chain complementarity determining region (CDR) 1 of SEQ ID NO:68, the heavy chain CDR 2 of SEQ ID NO:69, the heavy chain CDR 3 of SEQ ID NO:70, the light chain CDR 1 of SEQ ID NO:71, the light chain CDR 2 of SEQ ID NO:72 and the light chain CDR 3 of SEQ ID NO:73;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment the present invention provides an antigen binding receptor capable of specific binding to PDL1 comprising:
a) a heavy chain fusion polypeptide comprising in order from the N-terminus to the C-terminus;
   (i) a heavy chain comprising the heavy chain complementarity determining region (CDR) 1 of SEQ ID NO:68, the heavy chain CDR 2 of SEQ ID NO:69, the heavy chain CDR 3 of SEQ ID NO:70;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a light chain comprising the light chain CDR 1 of SEQ ID NO:71, the light chain CDR 2 of SEQ ID NO:72 and the light chain CDR 3 of SEQ ID NO:73.

In an alternative embodiment the present invention provides an antigen binding receptor capable of specific binding to PDL1 comprising:
a) a light chain fusion polypeptide comprising in order from the N-terminus to the C-terminus;
   (i) a light chain comprising the light chain complementarity determining region (CDR) 1 of SEQ ID NO:71, the light chain CDR 2 of SEQ ID NO:72, the light chain CDR 3 of SEQ ID NO:73;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) a heavy chain comprising the heavy chain CDR 1 of SEQ ID NO:68, the heavy chain CDR 2 of SEQ ID NO:69 and the heavy chain CDR 3 of SEQ ID NO:70.

In one embodiment the antigen binding moiety is a crossFab fragment comprising a heavy chain comprising or consisting of an amino acid sequence of SEQ ID NO:81 and a light chain comprising or consisting of the amino acid sequence of SEQ ID NO:80.

In an alternative embodiment the antigen binding moiety is a crossFab fragment comprising a heavy chain comprising or consisting of an amino acid sequence of SEQ ID NO:83 and a light chain comprising or consisting of the amino acid sequence of SEQ ID NO:84.

In one embodiment the present invention provides an antigen binding receptor capable of specific binding to PDL1 comprising:
a) a heavy chain fusion polypeptide comprising in order from the N-terminus to the C-terminus;
   (i) the heavy chain of SEQ ID NO:83;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and
b) the light chain of SEQ ID NO:84.

In an alternative embodiment the present invention provides an antigen binding receptor capable of specific binding to PDL1 comprising:
a) a light chain fusion polypeptide comprising in order from the N-terminus to the C-terminus;
   (i) the light chain of SEQ ID NO:80;
   (ii) a peptide linker, in particular the peptide linker of SEQ ID NO:20;
   (iii) an anchoring transmembrane domain, in particular the anchoring transmembrane domain of SEQ ID NO:14;
   (iii) a co-stimulatory signaling domain, in particular the co-stimulatory signaling domain of SEQ ID NO:15; and
   (iv) a stimulatory signaling domain, in particular the stimulatory signaling domain of SEQ ID NO:16 and

b) the heavy chain of SEQ ID NO:81.

In a particular embodiment, the antigen binding moiety is a crossFab fragment capable of specific binding to PDL1, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:82 and a light chain polypeptide comprising an amino acid sequence that is at least about 95%, 96%, 97%, 98%, 99% or 100% identical to the amino acid sequence of SEQ ID NO:84.

In another preferred embodiment, the antigen binding moiety is a Fab fragment capable of specific binding to PDL1, wherein the antigen binding receptor comprises a heavy chain fusion polypeptide comprising the amino acid sequence of SEQ ID NO:82 and a light chain polypeptide comprising the amino acid sequence of SEQ ID NO:84.

In a particular embodiment, the antigen binding moiety is a Fab fragment capable of specific binding to PDL1, wherein the antigen binding receptor comprises a light chain fusion polypeptide comprising an amino acid sequence that is at least about 95%, 96%, 97%, 98%, 99% or 100% identical to the amino acid sequence of SEQ ID NO:79 and a heavy chain polypeptide comprising an amino acid sequence that is at least about 95%, 96%, 97%, 98%, 99% or 100% identical to the amino acid sequence of SEQ ID NO:81.

In one preferred embodiment, the antigen binding moiety is a Fab fragment capable of specific binding to PDL1, wherein the antigen binding receptor comprises a light chain fusion polypeptide comprising the amino acid sequence of SEQ ID NO:79 and a heavy chain polypeptide comprising the amino acid sequence of SEQ ID NO:81.

In certain alternative embodiments, the antigen binding receptor of the invention, the Fab light chain polypeptide and the Fab heavy chain fusion polypeptide are fused to each other, optionally via a linker peptide. Accordingly, in one embodiment, the antigen binding moiety is a single chain Fab (scFab) fragment. In one embodiment, the Fab light chain polypeptide and the Fab heavy chain fusion polypeptide are fused to each other via a peptide linker. In one embodiment the peptide linker comprises the amino acid sequence GGGGSGGGGSGGGGS GGGGSGGGGSGGGGSGG (SEQ ID NO:54). In one embodiment, the antigen binding moiety is fused at the C-terminus of the scFab to the N-terminus of an anchoring transmembrane domain, optionally through a peptide linker. In one embodiment the peptide linker comprises the amino acid sequence GGGGS (SEQ ID NO:20). In one embodiment, the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof. In a preferred embodiment, the anchoring transmembrane domain is the CD28 transmembrane domain or a fragment thereof. In a particular embodiment, the anchoring transmembrane domain comprises or consist of the amino acid sequence of FWVLVVVGGVLACYSLLVTVAFIIFWV (SEQ ID NO:14). In one embodiment, the antigen binding receptor further comprises a co-stimulatory signaling domain (C SD). In one embodiment, the anchoring transmembrane domain of the antigen binding receptor is fused at the C-terminus to the N-terminus of a co-stimulatory signaling domain. In one embodiment, the co-stimulatory signaling domain is individually selected from the group consisting of the intracellular domain of CD27, of CD28, of CD137, of OX40, of ICOS, of DAP10 and of DAP12, or fragments thereof as described herein before. In a preferred embodiment, the co-stimulatory signaling domain is the intracellular domain of CD28 or a fragment thereof. In a particular embodiment the co-stimulatory signaling domain comprises or consists of the sequence RSKRSRLLHSDYMNMTPRRPGPTRKHYQPYAPPRDFAAYRS (SEQ ID NO:15). In one embodiment, the antigen binding receptor further comprises a stimulatory signaling domain. In one embodiment, the co-stimulatory signaling domain of the antigen binding receptor is fused at the C-terminus to the N-terminus of the stimulatory signaling domain. In one embodiment, the at least one stimulatory signaling domain is individually selected from the group consisting of the intracellular domain of CD3z, FCGR3A and NKG2D, or fragments thereof. In a preferred embodiment, the stimulatory signaling domain is the intracellular domain of CD3z or a fragment thereof. In a particular embodiment the stimulatory signaling domain comprises or consists of the sequence:

(SEQ ID NO: 16)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In one embodiment, the antigen binding receptor comprising the scFab is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) according to SEQ ID NO:21.

In a particular embodiment, the antigen binding receptor comprises an anchoring transmembrane domain and an extracellular domain comprising at least one antigen binding moiety, wherein the at least one antigen binding moiety is a scFab fragment capable of specific binding to CD20. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration scFab-ATD-CSD-SSD. In a preferred embodiment, the antigen binding receptor has the configuration scFab-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker.

In a particular embodiment, the antigen binding moiety is a scFab fragment capable of specific binding to CD20, wherein the antigen binding moiety comprises at least one heavy chain complementarity determining region (CDR) selected from the group consisting of SEQ ID NO:1, SEQ ID NO:2 and SEQ ID NO:3 and at least one light chain CDR selected from the group of SEQ ID NO:4, SEQ ID NO:5, SEQ ID NO:6.

In a preferred embodiment, the antigen binding moiety is a scFab capable of specific binding to CD20, wherein the antigen binding moiety comprises the complementarity determining region (CDR H) 1 amino acid sequence YSWIN (SEQ ID NO:1), the CDR H2 amino acid sequence RIFPGDGDTDYNGKFKG (SEQ ID NO:2), the CDR H3 amino acid sequence NVFDGYWLVY (SEQ ID NO:3), the light chain complementary-determining region (CDR L) 1 amino acid sequence RSSKSLLHSNGITYLY (SEQ ID NO:4), the CDR L2 amino acid sequence QMSNLVS (SEQ ID NO:5) and the CDR L3 amino acid sequence AQNLELPYT (SEQ ID NO:6).

In one embodiment the present invention provides an antigen binding receptor comprising in order from the N-terminus to the C-terminus:

(i) an antigen binding moiety which is a scFab fragment capable of specific binding to CD20, wherein the scFab fragment comprises a heavy chain variable region (VH) comprising the heavy chain complementarity determining region (CDR) 1 of SEQ ID NO:1, the heavy chain CDR 2 of SEQ ID NO:2, the heavy chain CDR 3 of SEQ ID NO:3, and a light chain variable region (VH) comprising the light chain CDR 1 of SEQ ID NO:4, the light chain CDR 2 of SEQ ID NO:5 and the light chain CDR 3 of SEQ ID NO:6;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment, the present invention provides an antigen binding receptor comprising in order from the N-terminus to the C-terminus:

(i) an antigen binding moiety which is a scFab molecule capable of specific binding to CD20, wherein the scFab comprises a heavy chain variable domain (VH) of SEQ ID NO:12 and the light chain variable domain (VL) of SEQ ID NO:10;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFab molecule capable of specific binding to CD20, wherein the scFab comprises the heavy chain variable domain (VH) SEQ ID NO:12 and the light chain variable domain (VL) SEQ ID NO:10;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFab molecule capable of specific binding to CD20, wherein the scFab comprises an amino acid sequence of SEQ ID NO:52;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a particular embodiment, the antigen binding moiety is capable of specific binding to CD20, wherein the antigen binding receptor comprises an amino acid sequence that is at least about 95%, 96%, 97%, 98%, 99% or 100% identical to the amino acid sequence of: SEQ ID NO:51. In a preferred embodiment, the antigen binding moiety is capable of specific binding to CD20, wherein the antigen binding receptor comprises the amino acid sequence of SEQ ID NO:51.

In a particular embodiment, the antigen binding receptor comprises an anchoring transmembrane domain and an extracellular domain comprising at least one antigen binding moiety, wherein the at least one antigen binding moiety is a scFab fragment capable of specific binding to PDL1. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration scFab-ATD-CSD-SSD. In a preferred embodiment, the antigen binding receptor has the configuration scFab-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker.

In a particular embodiment, the antigen binding moiety is a scFab fragment capable of specific binding to PDL1, wherein the antigen binding moiety comprises at least one heavy chain complementarity determining region (CDR) selected from the group consisting of SEQ ID NO:68, SEQ ID NO:69 and SEQ ID NO:70 and at least one light chain CDR selected from the group of SEQ ID NO:71, SEQ ID NO:72, SEQ ID NO:73.

In a preferred embodiment, the antigen binding moiety is a scFab capable of specific binding to PDL1, wherein the antigen binding moiety comprises the complementarity determining region (CDR H) 1 amino acid sequence DSWIH (SEQ ID NO:68), the CDR H2 amino acid sequence WISPYGGSTYYADSVKG (SEQ ID NO:69), the CDR H3 amino acid sequence RHWPGGFDY (SEQ ID NO:70), the light chain complementary-determining region (CDR L) 1 amino acid sequence RASQDVSTAVA (SEQ ID NO:71), the CDR L2 amino acid sequence SASFLYS (SEQ ID NO:72) and the CDR L3 amino acid sequence QQYLYHPAT (SEQ ID NO:73).

In one embodiment the present invention provides an antigen binding receptor comprising in order from the N-terminus to the C-terminus:

(i) an antigen binding moiety which is a scFab fragment capable of specific binding to PDL1, wherein the scFab fragment comprises a heavy chain variable region (VH) comprising the heavy chain complementarity determining region (CDR) 1 of SEQ ID NO:68, the heavy chain CDR 2 of SEQ ID NO:69, the heavy chain CDR 3 of SEQ ID NO:70, and a light chain variable region (VH) comprising the light chain CDR 1 of SEQ ID NO:71, the light chain CDR 2 of SEQ ID NO:72 and the light chain CDR 3 of SEQ ID NO:73;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment, the present invention provides an antigen binding receptor comprising in order from the N-terminus to the C-terminus:

(i) an antigen binding moiety which is a scFab molecule capable of specific binding to PDL1, wherein the scFab comprises a heavy chain variable domain (VH) of SEQ ID NO:78 and the light chain variable domain (VL) of SEQ ID NO:77;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFab molecule capable of specific binding to PDL1, wherein the scFab comprises the heavy chain variable domain (VH) SEQ ID NO:78 and the light chain variable domain (VL) SEQ ID NO:77;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFab molecule capable of specific binding to PDL1, wherein the scFab comprises an amino acid sequence of SEQ ID NO:87;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a particular embodiment, the antigen binding moiety is capable of specific binding to PDL1, wherein the antigen binding receptor comprises an amino acid sequence that is at least about 95%, 96%, 97%, 98%, 99% or 100% identical to the amino acid sequence of: SEQ ID NO:86. In a preferred embodiment, the antigen binding moiety is capable of specific binding to CD20, wherein the antigen binding receptor comprises the amino acid sequence of SEQ ID NO:86.

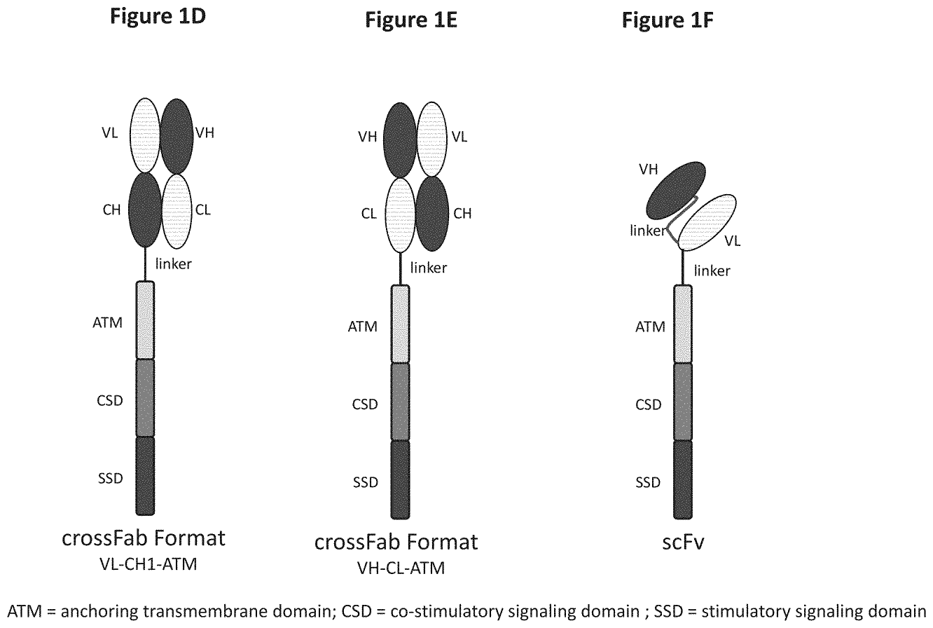

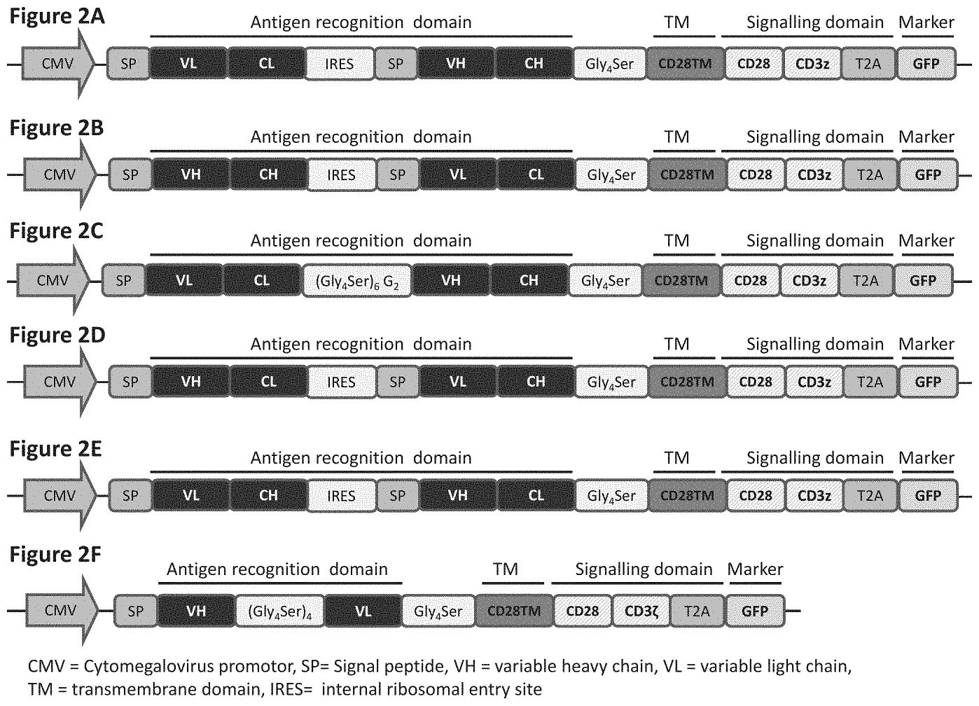

Fusion of the Fab heavy and light chains as described can improve pairing of Fab heavy and light chains, and also reduces the number of plasmids needed for expression of some of the antigen binding receptor of the invention. An alternative strategy to reduce the number of plasmids needed for expression of the antigen binding receptor is the use of an internal ribosomal entry side to enable expression of both heavy and light chain constructs from the same plasmid as illustrated e.g. in FIG. 2.

In one embodiment, the antigen binding moiety is a scFv fragment. In one embodiment, the antigen binding moiety is fused at the C-terminus of the scFv fragment to the N-terminus of an anchoring transmembrane domain, optionally through a peptide linker. In one embodiment the peptide linker comprises the amino acid sequence GGGGS (SEQ ID NO:20). In one embodiment, the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof. In a preferred embodiment, the anchoring transmembrane domain is the CD28 transmembrane domain or a fragment thereof. In a particular embodiment, the anchoring transmembrane domain comprises or consist of the amino acid sequence of FWVLVVVGGVLACYSLLVTVAFIIFWV (SEQ ID NO:14). In one embodiment, the antigen binding receptor further comprises a co-stimulatory signaling domain (CSD). In one embodiment, the anchoring transmembrane domain of the antigen binding receptor is fused at the C-terminus to the N-terminus of a co-stimulatory signaling domain. In one embodiment, the co-stimulatory signaling domain is individually selected from the group consisting of the intracellular domain of CD27, of CD28, of CD137, of OX40, of ICOS, of DAP10 and of DAP12, or fragments thereof as described herein before. In a preferred embodiment, the co-stimulatory signaling domain is the intracellular domain of CD28 or a fragment thereof. In a particular embodiment the co-stimulatory signaling domain comprises or consists of the sequence RSKRSRLLHSDYMNMTPRRPGPTRKHYQPYAPPRDFAAYRS (SEQ ID NO:15). In one embodiment, the antigen binding receptor further comprises a stimulatory signaling domain. In one embodiment, the co-stimulatory signaling domain of the antigen binding receptor is fused at the C-terminus to the N-terminus of the stimulatory signaling domain. In one embodiment, the at least one stimulatory signaling domain is individually selected from the group consisting of the intracellular domain of CD3z, FCGR3A and NKG2D, or fragments thereof. In a preferred embodiment, the stimulatory signaling domain is the intracellular domain of CD3z or a fragment thereof. In a particular embodiment the stimulatory signaling domain comprises or consists of the sequence:

(SEQ ID NO: 16)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In one embodiment, the antigen binding receptor comprising the scFv fragment is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) according to SEQ ID NO:21.

In a particular embodiment, the antigen binding receptor comprises an anchoring transmembrane domain and an extracellular domain comprising at least one antigen binding moiety, wherein the at least one antigen binding moiety is a scFv fragment capable of specific binding to CD20. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration scFv-ATD-CSD-SSD. In a preferred embodiment, the antigen binding receptor has the configuration scFv-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker.

In a particular embodiment, the antigen binding moiety is a scFv fragment capable of specific binding to CD20, wherein the antigen binding moiety comprises at least one heavy chain complementarity determining region (CDR) selected from the group consisting of SEQ ID NO:1, SEQ ID NO:2 and SEQ ID NO:3 and at least one light chain CDR selected from the group of SEQ ID NO:4, SEQ ID NO:5, SEQ ID NO:6.

In a preferred embodiment, the antigen binding moiety is a scFv capable of specific binding to CD20, wherein the antigen binding moiety comprises the complementarity determining region (CDR H) 1 amino acid sequence YSWIN (SEQ ID NO:1), the CDR H2 amino acid sequence RIFPGDGDTDYNGKFKG (SEQ ID NO:2), the CDR H3 amino acid sequence NVFDGYWLVY (SEQ ID NO:3), the light chain complementary-determining region (CDR L) 1 amino acid sequence RSSKSLLHSNGITYLY (SEQ ID NO:4), the CDR L2 amino acid sequence QMSNLVS (SEQ ID NO:5) and the CDR L3 amino acid sequence AQNLELPYT (SEQ ID NO:6).

In one embodiment the present invention provides an antigen binding receptor comprising in order from the N-terminus to the C-terminus:

(i) an antigen binding moiety which is a scFv fragment capable of specific binding to CD20, wherein the scFv fragment comprises a heavy chain variable region (VH) comprising the heavy chain complementarity determining region (CDR) 1 of SEQ ID NO:1, the heavy chain CDR 2 of SEQ ID NO:2, the heavy chain CDR 3 of SEQ ID NO:3, and a light chain variable region (VH) comprising the light chain CDR 1 of SEQ ID NO:4, the light chain CDR 2 of SEQ ID NO:5 and the light chain CDR 3 of SEQ ID NO:6;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment, the present invention provides an antigen binding receptor comprising in order from the N-terminus to the C-terminus:

(i) an antigen binding moiety which is a scFv molecule capable of specific binding to CD20, wherein the scFv comprises a heavy chain variable domain (VH) selected from SEQ ID NO:12 and SEQ ID NO:65 and the light chain variable domain (VL) selected from SEQ ID NO:10 and SEQ ID NO:66;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFv molecule capable of specific binding to CD20, wherein the scFv comprises the heavy chain variable domain (VH) SEQ ID NO:65 and the light chain variable domain (VL) SEQ ID NO:66;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFv molecule capable of specific binding to CD20, wherein the scFv comprises an amino acid sequence of SEQ ID NO:61;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a particular embodiment, the antigen binding moiety is capable of specific binding to CD20, wherein the antigen binding receptor comprises an amino acid sequence that is at least about 95%, 96%, 97%, 98%, 99% or 100% identical to the amino acid sequence of: SEQ ID NO:60. In a preferred embodiment, the antigen binding moiety is capable of specific binding to CD20, wherein the antigen binding receptor comprises the amino acid sequence of SEQ ID NO:60.

In a particular embodiment, the antigen binding receptor comprises an anchoring transmembrane domain and an extracellular domain comprising at least one antigen binding moiety, wherein the at least one antigen binding moiety is a scFv fragment capable of specific binding to PDL1. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration scFv-ATD-CSD-SSD. In a preferred embodiment, the antigen binding receptor has the configuration scFv-G₄S-ATD-CSD-SSD, wherein G₄S is a linker comprising the sequence GGGGS of SEQ ID NO:20. Optionally, a reporter protein can be added to the C-terminus of the antigen binding receptor, optionally through a peptide linker.

In one embodiment, the antigen binding moiety is a scFv fragment capable of specific binding to PDL1, wherein the antigen binding moiety comprises at least one heavy chain complementarity determining region (CDR) selected from the group consisting of SEQ ID NO:68, SEQ ID NO:69 and SEQ ID NO:70 and at least one light chain CDR selected from the group of SEQ ID NO:71, SEQ ID NO:72, SEQ ID NO:73.

In a preferred embodiment, the antigen binding moiety is a scFv capable of specific binding to PDL1, wherein the antigen binding moiety comprises the complementarity determining region (CDR H) 1 amino acid sequence DSWIH (SEQ ID NO:68), the CDR H2 amino acid sequence WISPYGGSTYYADSVKG (SEQ ID NO:69), the CDR H3 amino acid sequence RHWPGGFDY (SEQ ID NO:70), the light chain complementary-determining region (CDR L) 1 amino acid sequence RASQDVSTAVA (SEQ ID NO:71), the CDR L2 amino acid sequence SASFLYS (SEQ ID NO:72) and the CDR L3 amino acid sequence QQYLYHPAT (SEQ ID NO:73).

In one embodiment the present invention provides an antigen binding receptor comprising in order from the N-terminus to the C-terminus:

(i) an antigen binding moiety which is a scFv fragment capable of specific binding to PDL1, wherein the scFv fragment comprises a heavy chain variable region (VH) comprising the heavy chain complementarity determining region (CDR) 1 of SEQ ID NO:68, the heavy chain CDR 2 of SEQ ID NO:69, the heavy chain CDR 3 of SEQ ID NO:70, and a light chain variable region (VH) comprising the light chain CDR 1 of SEQ ID NO:71, the light chain CDR 2 of SEQ ID NO:72 and the light chain CDR 3 of SEQ ID NO:73;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In one embodiment, the present invention provides an antigen binding receptor comprising in order from the N-terminus to the C-terminus:

(i) an antigen binding moiety which is a scFv molecule capable of specific binding to PDL1, wherein the scFv comprises a heavy chain variable domain (VH) selected from SEQ ID NO:78 and SEQ ID NO:90 and the light chain variable domain (VL) selected from SEQ ID NO:77 and SEQ ID NO:91;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFv molecule capable of specific binding to PDL1, wherein the scFv comprises the heavy chain variable domain (VH) SEQ ID NO:90 and the light chain variable domain (VL) SEQ ID NO:91;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a preferred embodiment, the present invention provides an antigen binding receptor comprising in order from the N-terminus to the C-terminus

(i) an antigen binding moiety which is a scFv molecule capable of specific binding to PDL1, wherein the scFv comprises an amino acid sequence of SEQ ID NO:89;

(ii) a peptide linker, in particular the peptide linker of SEQ ID NO:20;

(iii) an anchoring transmembrane domain, in particular the anchoring transmembrane domain of SEQ ID NO:14;

(iii) a co-stimulatory signaling domain, in particular the co-stimulatory signaling domain of SEQ ID NO:15; and

(iv) a stimulatory signaling domain, in particular the stimulatory signaling domain of SEQ ID NO:16.

In a particular embodiment, the antigen binding moiety is capable of specific binding to PDL1, wherein the antigen binding receptor comprises an amino acid sequence that is at least about 95%, 96%, 97%, 98%, 99% or 100% identical to the amino acid sequence of: SEQ ID NO:88. In a preferred embodiment, the antigen binding moiety is capable of specific binding to PDL1, wherein the antigen binding receptor comprises the amino acid sequence of SEQ ID NO:88.

According to any of the above embodiments, components of the antigen binding receptor (e.g., VH and VL, antigen binding moiety, anchoring transmembrane domain, co-stimulatory signaling domain, stimulatory signaling domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (G₄S)ₙ, or G₄(SG₄)ₙ peptide linkers, wherein n is generally a number between 1 and 10, preferably between 1 and 4.

Modifications in the Fab and CrossFab Domains

In another aspect, and to improve correct pairing if more than one antigen binding receptors are comprised in the same cell as described herein, i.e. the same T cell, a first antigen binding receptor comprising a first Fab or crossFab fragment that specifically binds to a first target antigen and a second antigen binding receptor comprising a second Fab or crossFab fragment that specifically binds to a second target antigen, can contain different charged amino acid substitutions (so-called “charged residues”). These modifications are introduced in the crossed or non-crossed CH1 and CL domains. Such modifications are described e.g. in WO2015/150447, WO2016/020309 and PCT/EP2016/073408.

In a particular aspect, the invention is concerned with an antigen binding receptor comprising a Fab, wherein in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In a particular aspect, the invention relates to an antigen binding receptor comprising a Fab fragment that specifically binds to a target antigen, wherein in the CL domain the amino acid at position 123 (EU numbering) has been replaced by arginine (R) and the amino acid at position 124 (EU numbering) has been substituted by lysine (K) and wherein in the CH1 domain the amino acids at position 147 (EU numbering) and at position 213 (EU numbering) have been substituted by glutamic acid (E).

In a further aspect the invention relates to two antigen binding receptors which can be co-transduced into a cell, i.e. a T cell, wherein correct pairing of the heavy and light chains is improved. In one such aspect, (i) in the CL domain of the Fab or crossFab fragment of the first antigen binding receptor the amino acid at position 124 (numbering according to Kabat) is substituted by a positively charged amino acid, and wherein in the CH1 domain of the Fab or crossFab fragment of the first antigen binding receptor the amino acid at position 147 or the amino acid at position 213 (numbering according to Kabat EU index) is substituted by a negatively charged amino acid, and/or (ii) in the CL domain of the Fab or crossFab fragment of the second antigen binding receptor the amino acid at position 124 (numbering according to Kabat) is substituted by a positively charged amino acid, and wherein in the CH1 domain of the Fab or crossFab fragment of the second antigen binding receptor the amino acid at position 147 or the amino acid at position 213 (numbering according to Kabat EU index) is substituted by a negatively charged amino acid.

In another aspect, (i) in the CL domain of the Fab or crossFab fragment of the first antigen binding receptor the amino acid at position 124 is substituted independently by lysine (K), arginine (R) or histidine (H) (numbering according to Kabat) (in one preferred embodiment independently by lysine (K) or arginine (R)), and wherein in the CH domain of the Fab or crossFab fragment of the first antigen binding receptor the amino acid at position 147 or the amino acid at position 213 is substituted independently by glutamic acid (E) or aspartic acid (D) (numbering according to Kabat EU index), and/or (ii) in the CL domain of the Fab or crossFab fragment of the second antigen binding receptor the amino acid at position 124 is substituted independently by lysine (K), arginine (R) or histidine (H) (numbering according to Kabat) (in one preferred embodiment independently by lysine (K) or arginine (R)), and wherein in the CH domain of the Fab or crossFab fragment of the second antigen binding receptor the amino acid at position 147 or the amino acid at position 213 is substituted independently by glutamic acid (E) or aspartic acid (D) (numbering according to Kabat EU index).

In one aspect, in the CL domain of the Fab or crossFab fragment of the first antigen binding receptor the amino acids at position 124 and 123 are substituted by K (numbering according to Kabat EU index).

In one aspect, in the CL domain of the Fab or crossFab fragment of the second antigen binding receptor the amino acid at position 123 is substituted by R and the amino acid as position 124 is substituted by K (numbering according to Kabat EU index).

In one aspect, in the CH domain of the Fab or crossFab fragment of the second antigen binding receptor the amino acids at position 147 and 213 are substituted by E (numbering according to EU index of Kabat).

In one aspect, in the CL domain of the Fab or crossFab fragment of the first antigen binding receptor the amino acids at position 124 and 123 are substituted by K, and in the CH domain of the Fab or crossFab fragment of the first antigen binding receptor the amino acids at position 147 and 213 are substituted by E (numbering according to Kabat EU index).

In one aspect, in the CL domain of the Fab or crossFab fragment of the first antigen binding receptor the amino acid at position 123 is substituted by R and the amino acid at position 124 is substituted by K, and in the CH domain of the Fab or crossFab fragment of the first antigen binding receptor the amino acids at position 147 and 213 are both substituted by E (numbering according to Kabat EU index).

In one aspect, in the CL domain of the Fab or crossFab fragment of the second antigen binding receptor the amino acids at position 124 and 123 are substituted by K, and wherein in the CH domain of the Fab or crossFab fragment of the second antigen binding receptor the amino acids at position 147 and 213 are substituted by E, and in the VL domain of the Fab or crossFab fragment of the first antigen binding receptor the amino acid at position 38 is substituted by K, in the VH domain of the Fab or crossFab fragment of the first antigen binding receptor the amino acid at position 39 is substituted by E, in the VL domain of the Fab or crossFab fragment of the second antigen binding receptor the amino acid at position 38 is substituted by K, and in the VH domain of the Fab or crossFab fragment of the second antigen binding receptor the amino acid at position 39 is substituted by E (numbering according to Kabat EU index).

Exemplary T Cell Activating Antigen Binding Receptors

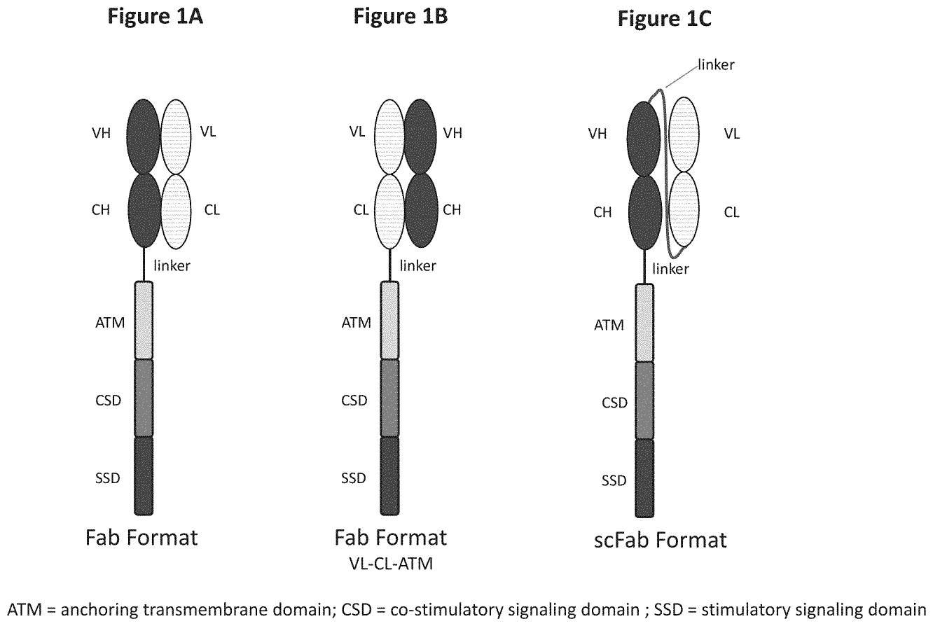

As illustratively shown in the appended Examples and in FIG. 1A, as a proof of concept of the present invention, the antigen binding receptor “Anti-CD20-Fab-CD28ATD-CD28CSD-CD3zSSD pETR17097” (SEQ ID NOs: 7, 9) was constructed which comprises one Fab antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid and DNA) of the antigen binding receptor “Anti-CD20-Fab-CD28ATD-CD28CSD-CD3zSSD pETR17097” are shown in Tables 2 and 3.

As a further proof of concept of the present invention, the antigen binding receptor “Anti-CD20-crossFab(VH-CL)-CD28ATD-CD28CSD-CD3zSSD pETR17098” (SEQ ID NOs: 36, 38) was constructed which comprises one crossFab antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-CD20-crossFab(VH-CL)-CD28ATD-CD28CSD-CD3zSSD pETR17098” are shown in Table 4.

As a further proof of concept of the present invention, the antigen binding receptor “Anti-CD20-crossFab(VL-CH)-CD28ATD-CD28CSD-CD3zSSD” (SEQ ID NOs: 41, 43) was constructed which comprises one crossFab antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid and DNA) of the antigen binding receptor “Anti-CD20-crossFab(VL-CH)-CD28ATD-CD28CSD-CD3zSSD” are shown in Tables 5 and 6.

As a further proof of concept of the present invention, the antigen binding receptor “Anti-CD20-Fab(VL-CL)-CD28ATD-CD28CSD-CD3zSSD” (SEQ ID NOs: 50, 8) was constructed which comprises one crossFab antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-CD20-Fab(VL-CL)-CD28ATD-CD28CSD-CD3zSSD” are shown in Table 7.

As a further proof of concept of the present invention, the antigen binding receptor “Anti-CD20-scFab-CD28ATD-CD28CSD-CD3zSSD” (SEQ ID NOs: 51) was constructed which comprises one scFab antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid and DNA) of the antigen binding receptor “Anti-CD20-scFab-CD28ATD-CD28CSD-CD3zSSD” are shown in Tables 8 and 9.

As a further proof and reference, the antigen binding receptor “Anti-CD20-scFv-CD28ATD-CD28CSD-CD3zSSD pETR17162” (SEQ ID NO:60) was constructed which comprises one stabilized scFv antigen binding moiety binding to/directed against/interacting with or on CD20. The construct further comprises the CD28 transmembrane domain, a fragment of CD28 as co-stimulatory signaling domain and a fragment of CD3z as stimulatory signaling domain. The sequences (amino acid and cDNA) of the antibody binding molecule “Anti-CD20-scFv-CD28ATD-CD28CSD-CD3zSSD pETR17162” are shown in Tables 10 and 11.

As a further proof of concept of the present invention, the antigen binding receptor “Anti-PDL1-Fab-CD28ATD-CD28CSD-CD3zSSD” (SEQ ID NOs: 74, 76) was constructed which comprises one Fab antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-PDL1-Fab-CD28ATD-CD28CSD-CD3zSSD” are shown in Table 12.

As a further proof of concept of the present invention, the antigen binding receptor “Anti-PDL1-crossFab(VH-CL)-CD28ATD-CD28CSD-CD3zSSD” (SEQ ID NOs: 79, 81) was constructed which comprises one crossFab antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-PDL1-crossFab(VH-CL)-CD28ATD-CD28CSD-CD3zSSD” are shown in Table 13.

As a further proof of concept of the present invention, the antigen binding receptor “Anti-PDL1-crossFab(VL-CH)-CD28ATD-CD28CSD-CD3zSSD” (SEQ ID NOs: 82, 84) was constructed which comprises one crossFab antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-PDL1-crossFab(VL-CH)-CD28ATD-CD28CSD-CD3zSSD” are shown in Table 14.

As a further proof of concept of the present invention, the antigen binding receptor “Anti-PDL1-Fab (VL-CL)-CD28ATD-CD28CSD-CD3zSSD” (SEQ ID NOs: 85, 75) was constructed which comprises one crossFab antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-PDL1-Fab(VL-CL)-CD28ATD-CD28CSD-CD3zSSD” are shown in Table 15.

As a further proof of concept of the present invention, the antigen binding receptor “Anti-PDL1-scFab-CD28ATD-CD28CSD-CD3zSSD” (SEQ ID NOs: 86) was constructed which comprises one scFab antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antigen binding receptor “Anti-PDL1-scFab-CD28ATD-CD28CSD-CD3zSSD” are shown in Table 16.

As a further proof and reference, the antigen binding receptor “Anti-PDL1-scFv-CD28ATD-CD28CSD-CD3zSSD pETR17162” (SEQ ID NO:88) was constructed which comprises one stabilized scFv antigen binding moiety binding to/directed against/interacting with or on PDL1. The construct further comprises the CD28 transmembrane domain, a fragment of CD28 as co-stimulatory signaling domain and a fragment of CD3z as stimulatory signaling domain. The sequences (amino acid) of the antibody binding molecule “Anti-PDL1-scFv-CD28ATD-CD28CSD-CD3zSSD pETR17162” are shown in Table 17.

Kits

A further aspect of the present invention are kits comprising or consisting of a nucleic acid encoding an antigen binding receptor of the invention and/or cells, preferably T cells transduced with antigen binding receptors of the invention. Parts of the kit of the invention can be packaged individually in vials or bottles or in combination in containers or multicontainer units. Additionally, the kit of the present invention may comprise a (closed) bag cell incubation system where patient cells, preferably T cells as described herein, can be transduced with (an) antigen binding receptor(s) of the invention and incubated under GMP (good manufacturing practice, as described in the guidelines for good manufacturating practice published by the European Commission under http://ec.europa.eu/health/documents/eudralex/index_en.htm) conditions. In one embodiment, the kit of the present invention comprises a (closed) bag cell incubation system where isolated/obtained patients T cells can be transduced with (an) antigen binding receptor(s) of the invention and incubated under GMP. Furthermore, in the context of the present invention, the kit may also comprise a vector encoding (the) antigen binding receptor(s) as described herein. The kit of the present invention may be advantageously used, inter alia, for carrying out the method of the invention and could be employed in a variety of applications referred herein, e.g., as research tools or medical tools. The manufacture of the kits preferably follows standard procedures which are known to the person skilled in the art.

In this context, patient derived cells, preferably T cells, can be transduced with an antigen binding receptor of the invention as described herein using the kit as described above. The patient derived cells transduced with the kits of the invention will acquire the capability of specific binding the target of the antigen binding moiety, e.g. a tumor associated antigen and will become capable of inducing elimination/lysis of target cells via. Binding of the extracellular domain of the antigen binding receptor as described herein activates that T cell and brings it into physical contact with the tumor cell. Accordingly, T cells expressing the inventive antigen binding receptor molecule have the ability to lyse target cells as described herein in vivo and/or in vitro. Corresponding target cells comprise cells expressing a surface molecule, i.e. a tumor-specific antigen naturally occurring on the surface of a tumor cell, which is recognized by at least one antigen binding moiety as described herein. Such surface molecules are characterized herein below.

Lysis of the target cell can be detected by methods known in the art. Accordingly, such methods comprise, inter alia, physiological in vitro assays. Such physiological assays may monitor cell death, for example by loss of cell membrane integrity (e.g. FACS based propidium Iodide assay, trypan blue influx assay, photometric enzyme release assays (LDH), radiometric 51Cr release assay, fluorometric Europium release and CalceinAM release assays). Further assays comprise monitoring of cell viability, for example by photometric MTT, XTT, WST-1 and alamarBlue assays, radiometric 3H-Thd incorporation assay, clonogenic assay measuring cell division activity, and fluorometric Rhodamine123 assay measuring mitochondrial transmembrane gradient. In addition, apoptosis may be monitored for example by FACS-based phosphatidylserin exposure assay, ELISA-based TUNEL test, caspase activity assay (photometric, fluorometric or ELISA-based) or analyzing changed cell morphology (shrinking, membrane blebbing).

Transduced T Cells Capable of Expressing Antigen Binding Receptors of the Invention

A further aspect of the present invention is a transduced T cell capable of expressing (an) antigen binding receptor(s) of the present invention. The antigen binding receptors as described herein relate to molecules which are naturally not comprised in and/or on the surface of T cells and which are not (endogenously) expressed in or on normal (non-transduced) T cells. Thus, the antigen binding receptor of the invention in and/or on T cells is artificially introduced into T cells. In the context of the present invention said T cells, preferably CD8+ T cells, may be isolated/obtained from a subject to be treated as defined herein. Accordingly, the antigen binding receptors as described herein which are artificially introduced and subsequently presented in and/or on the surface of said T cells comprise domains comprising one or more antigen binding moiety accessible (in vitro or in vivo) to tumor associated antigens. In the context of the present invention, these artificially introduced molecules are presented in and/or on the surface of said T cells after (retroviral or lentiviral) transduction as described herein below. Accordingly, after transduction, T cells according to the invention can be tumor associated antigens, preferably antigens presented/accessible on the surface of tumor cells.

The invention also relates to transduced T cells comprising (a) nucleic acid molecule(s) encoding the antigen binding receptor of the present invention. Accordingly, in the context of the present invention, the transduced cell may comprise a nucleic acid molecule encoding the antigen binding receptor of the present invention or a vector of the present invention which is capable to induce expression of an antigen binding receptor of the present invention.

In the context of the present invention, the term “transduced T cell” relates to a genetically modified T cell (i.e. a T cell wherein a nucleic acid molecule has been introduced deliberately). The herein provided transduced T cell may comprise the vector of the present invention. Preferably, the herein provided transduced T cell comprises the nucleic acid molecule encoding the antigen binding receptor of the present invention and/or the vector of the present invention. The transduced T cell of the invention may be a T cell which transiently or stably expresses the foreign DNA (i.e. the nucleic acid molecule which has been introduced into the T cell). In particular, the nucleic acid molecule encoding the antigen binding receptor of the present invention can be stably integrated into the genome of the T cell by using a retroviral or lentiviral transduction. By using mRNA transfection, the nucleic acid molecule encoding the antigen binding receptor of the present invention may be expressed transiently. Preferably, the herein provided transduced T cell has been genetically modified by introducing a nucleic acid molecule in the T cell via a viral vector (e.g. a retroviral vector or a lentiviral vector). Accordingly, the expression of the antigen binding receptors may be constitutive and the extracellular domain of the antigen binding receptor may be detectable on the cell surface. This extracellular domain of the antigen binding receptor may comprise the complete extracellular domain of an antigen binding receptor as defined herein but also parts thereof. The minimal size required being the antigen binding site of the antigen binding moiety in the antigen binding receptor.

The expression may also be conditional or inducible in the case that the antigen binding receptor is introduced into T cells under the control of an inducible or repressible promoter. Examples for such inducible or repressible promoters can be a transcriptional system containing the alcohol dehydrogenase I (alcA) gene promoter and the transactivator protein AlcR. Different agricultural alcohol-based formulations are used to control the expression of a gene of interest linked to the alcA promoter. Furthermore, tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. Further, steroid-responsive promoters, metal-regulated or pathogenesis-related (PR) protein related promoters can be used.

The expression can be constitutive or constitutional, depending on the system used. The antigen binding receptors of the present invention can be expressed on the surface of the herein provided transduced T cell. The extracellular portion of the antigen binding receptor (i.e. the extracellular domain of the antigen binding receptor can be detected on the cell surface, while the intracellular portion (i.e. the co-stimulatory signaling domain(s) and the stimulatory signaling domain) are not detectable on the cell surface. The detection of the extracellular domain of the antigen binding receptor can be carried out by using an antibody which specifically binds to this extracellular domain or by the antigen the extracellular domain is capable to bind. The extracellular domain can be detected using these antibodies or antigens by flow cytometry or microscopy.

The transduced cells of the present invention may be any immune cell. These include but are not limited to B-cells, T cells, Natural Killer (NK) cells, Natural Killer (NK) T cells, γδ T cells, innate lymphoid cells, macrophages, monocytes, dendritic cells, or neutrophils. Preferentially, said immune cell would be a lymphocyte, preferentially a NK or T cells. The said T cells include CD4 T cells and CD8 T cells. Triggering of the antigen binding receptor of the present invention on the surface of the leukocyte will render the cell cytotoxic against a target cell irrespective of the lineage the cell originated from. Cytotoxicity will happen irrespective of the stimulatory signaling domain or co-stimulatory signaling domain chosen for the antigen binding receptor and is not dependent on the exogenous supply of additional cytokines. Accordingly, the transduced cell of the present invention may be, e.g., a CD4+ T cell, a CD8+-T cell, a γδ T cell, a Natural Killer (NK) T cell, a Natural Killer (NK) cell, a tumor-infiltrating lymphocyte (TIL) cell, a myeloid cell, or a mesenchymal stem cell. Preferably, the herein provided transduced cell is a T cell (e.g. an autologous T cell), more preferably, the transduced cell is a CD8+ T cell. Accordingly, in the context of the present invention, the transduced cell is a CD8+ T cell. Further, in the context of the present invention, the transduced cell is an autologous T cell. Accordingly, in the context of the present invention, the transduced cell is preferably an autologous CD8+ T cell. In addition to the use of autologous cells (e.g. T cells) isolated from the subject, the present invention also comprehends the use of allogeneic cells. Accordingly, in the context of the present invention the transduced cell may also be an allogeneic cell, such as an allogeneic CD8+ T cell. The use of allogeneic cells is based on the fact that cells, preferably T cells can recognize a specific antigen epitope presented by foreign antigen-presenting cells (APC), provided that the APC express the MHC molecule, class I or class II, to which the specific responding cell population, i.e. T cell population is restricted, along with the antigen epitope recognized by the T cells. Thus, the term allogeneic refers to cells from an unrelated coming from an unrelated donor individual/subject which is human leukocyte antigen (HLA) compatible to the individual/subject which will be treated by e.g. the herein described antigen binding receptor expressing transduced cell. Autologous cells refer to cells which are isolated/obtained as described herein above from the subject to be treated with the transduced cell described herein.

The transduced cell of the invention may be co-transduced with further nucleic acid molecules, e.g. with a nucleic acid molecule encoding a T cell receptor.

The present invention also relates to a method for the production of a transduced T cell expressing an antigen binding receptor of the invention, comprising the steps of transducing a T cell with a vector of the present invention, culturing the transduced T cell under conditions allowing the expressing of the antigen binding receptor in or on said transduced cell and recovering said transduced T cell.

In the context of the present invention, the transduced cell of the present invention is preferably produced by the following process: cells (e.g., T cells, preferably CD8+ T cells) are isolated/obtained from a subject (preferably a human patient). Methods for isolating/obtaining cells (e.g. T cells, preferably CD8+ T cells) from patients or from donors are well known in the art and in the context of the present the cells (e.g. T cells, preferably CD8+ T cells) from patients or from donors may be isolated by blood draw or removal of bone marrow. After isolating/obtaining cells as a sample of the patient, the cells (e.g. T cells) are separated from the other ingredients of the sample. Several methods for separating cells (e.g. T cells) from the sample are known and include, without being limiting, e.g. leukapheresis for obtaining cells from the peripheral blood sample from a patient or from a donor, isolating/obtaining cells by using a FACSort apparatus, picking living of dead cells from fresh biopsy specimens harboring living cells by hand or by using a micromanipulator (see, e.g., Dudley, Immunother. 26 (2003), 332-342; Robbins, Clin. Oncol. 29 (201 1), 917-924 or Leisegang, J. Mol. Med. 86 (2008), 573-58). The isolated/obtained cells T cells, preferably CD8+ T cells, are subsequently cultivated and expanded, e.g., by using an anti-CD3 antibody, by using anti-CD3 and anti-CD28 monoclonal antibodies and/or by using an anti-CD3 antibody, an anti-CD28 antibody and interleukin-2 (IL-2) (see, e.g., Dudley, Immunother. 26 (2003), 332-342 or Dudley, Clin. Oncol. 26 (2008), 5233-5239).

In a subsequent step the cells (e.g. T cells) are artificially/genetically modified/transduced by methods known in the art (see, e.g., Lemoine, J Gene Med 6 (2004), 374-386). Methods for transducing cells (e.g. T cells) are known in the art and include, without being limited, in a case where nucleic acid or a recombinant nucleic acid is transduced, for example, an electroporation method, calcium phosphate method, cationic lipid method or liposome method. The nucleic acid to be transduced can be conventionally and highly efficiently transduced by using a commercially available transfection reagent, for example, Lipofectamine (manufactured by Invitrogen, catalogue no.: 11668027). In a case where a vector is used, the vector can be transduced in the same manner as the above-mentioned nucleic acid as long as the vector is a plasmid vector (i.e. a vector which is not a viral vector In the context of the present invention, the methods for transducing cells (e.g. T cells) include retroviral or lentiviral T cell transduction, non-viral vectors (e.g., sleeping beauty minicircle vector) as well as mRNA transfection. “mRNA transfection” refers to a method well known to those skilled in the art to transiently express a protein of interest, like in the present case the antigen binding receptor of the present invention, in a cell to be transduced. In brief cells may be electroporated with the mRNA coding for the antigen binding receptor of the present by using an electroporation system (such as e.g. Gene Pulser, Bio-Rad) and thereafter cultured by standard cell (e.g. T cell) culture protocol as described above (see Zhao et al., Mol Ther. 13(1) (2006), 151-159.) The transduced cell of the invention is a T cell, most preferably a CD8+ T cell, and is generated by lentiviral, or most preferably retroviral T cell transduction.

In this context, suitable retroviral vectors for transducing T cells are known in the art such as SAMEN CMV/SRa (Clay et al., J. Immunol. 163 (1999), 507-513), LZRS-id3-IHRES (Heemskerk et al., J. Exp. Med. 186 (1997), 1597-1602), FeLV (Neil et al., Nature 308 (1984), 814-820), SAX (Kantoff et al., Proc. Natl. Acad. Sci. USA 83 (1986), 6563-6567), pDOL (Desiderio, J. Exp. Med. 167 (1988), 372-388), N2 (Kasid et al., Proc. Natl. Acad. Sci. USA 87 (1990), 473-477), LNL6 (Tiberghien et al., Blood 84 (1994), 1333-1341), pZipNEO (Chen et al., J. Immunol. 153 (1994), 3630-3638), LASN (Mullen et al., Hum. Gene Ther. 7 (1996), 1123-1129), pG1XsNa (Taylor et al., J. Exp. Med. 184 (1996), 2031-2036), LCNX (Sun et al., Hum. Gene Ther. 8 (1997), 1041-1048), SFG (Gallardo et al., Blood 90 (1997), and LXSN (Sun et al., Hum. Gene Ther. 8 (1997), 1041-1048), SFG (Gallardo et al., Blood 90 (1997), 952-957), HMB-Hb-Hu (Vieillard et al., Proc. Natl. Acad. Sci. USA 94 (1997), 11595-11600), pMV7 (Cochlovius et al., Cancer Immunol. Immunother. 46 (1998), 61-66), pSTITCH (Weitjens et al., Gene Ther 5 (1998), 1195-1203), pLZR (Yang et al., Hum. Gene Ther. 10 (1999), 123-132), pBAG (Wu et al., Hum. Gene Ther. 10 (1999), 977-982), rKat.43.267bn (Gilham et al., J. Immunother. 25 (2002), 139-151), pLGSN (Engels et al., Hum. Gene Ther. 14 (2003), 1155-1168), pMP71 (Engels et al., Hum. Gene Ther. 14 (2003), 1155-1168), pGCSAM (Morgan et al., J. Immunol. 171 (2003), 3287-3295), pMSGV (Zhao et al., J. Immunol. 174 (2005), 4415-4423), or pMX (de Witte et al., J. Immunol. 181 (2008), 5128-5136). In the context of the present invention, suitable lentiviral vector for transducing cells (e.g. T cells) are, e.g. PL-SIN lentiviral vector (Hotta et al., Nat Methods. 6(5) (2009), 370-376), p156RRL-sinPPT-CMV-GFP-PRE/NheI (Campeau et al., PLoS One 4(8) (2009), e6529), pCMVR8.74 (Addgene Catalogoue No.:22036), FUGW (Lois et al., Science 295(5556) (2002), 868-872, pLVX-EF1 (Addgene Catalogue No.: 64368), pLVE (Brunger et al., Proc Natl Acad Sci U S A 111(9) (2014), E798-806), pCDH1-MCS1-EF1 (Hu et al., Mol Cancer Res. 7(11) (2009), 1756-1770), pSLIK (Wang et al., Nat Cell Biol. 16(4) (2014), 345-356), pLJM1 (Solomon et al., Nat Genet. 45(12) (2013), 1428-30), pLX302 (Kang et al., Sci Signal. 6(287) (2013), rs13), pHR-IG (Xie et al., J Cereb Blood Flow Metab. 33(12) (2013), 1875-85), pRRLSIN (Addgene Catalogoue No.: 62053), pLS (Miyoshi et al., J Virol. 72(10) (1998), 8150-8157), pLL3.7 (Lazebnik et al., J Biol Chem. 283(7) (2008), 11078-82), FRIG (Raissi et al., Mol Cell Neurosci. 57 (2013), 23-32), pWPT (Ritz-Laser et al., Diabetologia. 46(6) (2003), 810-821), pBOB (Marr et al., J Mol Neurosci. 22(1-2) (2004), 5-11), or pLEX (Addgene Catalogue No.: 27976).

The transduced T cell/T cells of the present invention is/are preferably grown under controlled conditions, outside of their natural environment. In particular, the term “culturing” means that cells (e.g. the transduced cell(s) of the invention) which are derived from multi-cellular eukaryotes (preferably from a human patient) are grown in vitro. Culturing cells is a laboratory technique of keeping cells alive which are separated from their original tissue source. Herein, the transduced cell of the present invention is cultured under conditions allowing the expression of the antigen binding receptor of the present invention in or on said transduced cells. Conditions which allow the expression or a transgene (i.e. of the antigen binding receptor of the present invention) are commonly known in the art and include, e.g., agonistic anti-CD3- and anti-CD28 antibodies and the addition of cytokines such as interleukin 2 (IL-2), interleukin 7 (IL-7), interleukin 12 (IL-12) and/or interleukin 15 (IL-15). After expression of the antigen binding receptor of the present invention in the cultured transduced cell (e.g., a CD8+ T), the transduced cell is recovered (i.e. re-extracted) from the culture (i.e. from the culture medium). Accordingly, also encompassed by the invention is a transduced cell, preferably a T cell, in particular a CD8+ T expressing an antigen binding receptor encoded by a nucleic acid molecule of the invention obtainable by the method of the present invention.

Nucleic Acid Molecules

A further aspect of the present invention is nucleic acids and vectors encoding one or several antigen binding receptors of the present invention. Exemplary nucleic acid molecules encoding antigen binding receptors of the present invention are shown in SEQ ID NOs:22, 46, 55 and 64. The nucleic acid molecules of the invention may be under the control of regulatory sequences. For example, promoters, transcriptional enhancers and/or sequences which allow for induced expression of the antigen binding receptor of the invention may be employed. In the context of the present invention, the nucleic acid molecules are expressed under the control of constitutive or inducible promoter. Suitable promoters are e.g. the CMV promoter (Qin et al., PLoS One 5(5) (2010), e10611), the UBC promoter (Qin et al., PLoS One 5(5) (2010), e10611), PGK (Qin et al., PLoS One 5(5) (2010), e10611), the EF1A promoter (Qin et al., PLoS One 5(5) (2010), e10611), the CAGG promoter (Qin et al., PLoS One 5(5) (2010), e10611), the SV40 promoter (Qin et al., PLoS One 5(5) (2010), e10611), the COPIA promoter (Qin et al., PLoS One 5(5) (2010), e10611), the ACTSC promoter (Qin et al., PLoS One 5(5) (2010), e10611), the TRE promoter (Qin et al., PLoS One. 5(5) (2010), e10611), the Oct3/4 promoter (Chang et al., Molecular Therapy 9 (2004), S367-S367 (doi: 10.1016/j.ymthe.2004.06.904)), or the Nanog promoter (Wu et al., Cell Res. 15(5) (2005), 317-24). The present invention therefore also relates to (a) vector(s) comprising the nucleic acid molecule(s) described in the present invention. Herein the term vector relates to a circular or linear nucleic acid molecule which can autonomously replicate in a host cell (i.e. in a transduced cell) into which it has been introduced. Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook et al. (loc cit.) and Ausubel, Current Protocols in Molecular Biology, Green Publishing Associates and Wiley Interscience, N.Y. (1989), (1994). Alternatively, the polynucleotides and vectors of the invention can be reconstituted into liposomes for delivery to target cells. As discussed in further details below, a cloning vector was used to isolate individual sequences of DNA. Relevant sequences can be transferred into expression vectors where expression of a particular polypeptide is required. Typical cloning vectors include pBluescript SK, pGEM, pUC9, pBR322, pGA18 and pGBT9. Typical expression vectors include pTRE, pCAL-n-EK, pESP-1, pOP13CAT.

The invention also relates to (a) vector(s) comprising (a) nucleic acid molecule(s) which is (are) a regulatory sequence operably linked to said nucleic acid molecule(s) encoding an antigen binding receptor as defined herein. In the context of the present invention the vector can be polycistronic. Such regulatory sequences (control elements) are known to the skilled person and may include a promoter, a splice cassette, translation initiation codon, translation and insertion site for introducing an insert into the vector(s). In the context of the present invention, said nucleic acid molecule(s) is (are) operatively linked to said expression control sequences allowing expression in eukaryotic or prokaryotic cells. It is envisaged that said vector(s) is (are) an expression vector(s) comprising the nucleic acid molecule(s) encoding the antigen binding receptor as defined herein. Operably linked refers to a juxtaposition wherein the components so described are in a relationship permitting them to function in their intended manner. A control sequence operably linked to a coding sequence is ligated in such a way that expression of the coding sequence is achieved under conditions compatible with the control sequences. In case the control sequence is a promoter, it is obvious for a skilled person that double-stranded nucleic acid is preferably used.

In the context of the present invention the recited vector(s) is (are) an expression vector(s). An expression vector is a construct that can be used to transform a selected cell and provides for expression of a coding sequence in the selected cell. An expression vector(s) can for instance be cloning (a) vector(s), (a) binary vector(s) or (a) integrating vector(s). Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40 , RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences encoding signal peptides capable of directing the polypeptide to a cellular compartment or secreting it into the medium may be added to the coding sequence of the recited nucleic acid sequence and are well known in the art; see also, e.g., appended Examples.

The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode an antigen binding receptor including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pCDM8, pRc/CMV, pcDNA1, pcDNA3 (In-vitrogene), pEF-DHFR, pEF-ADA or pEF-neo (Raum et al. Cancer Immunol Immunother 50 (2001), 141-150) or pSPORT1 (GIBCO BRL).

In the context of the present invention, the expression control sequences will be eukaryotic promoter systems in vectors capable of transforming or transfecting eukaryotic cells, but control sequences for prokaryotic cells may also be used. Once the vector has been incorporated into the appropriate cell, the cell is maintained under conditions suitable for high level expression of the nucleotide sequences, and as desired. Additional regulatory elements may include transcriptional as well as translational enhancers. Advantageously, the above-described vectors of the invention comprise a selectable and/or scorable marker. Selectable marker genes useful for the selection of transformed cells and, e.g., plant tissue and plants are well known to those skilled in the art and comprise, for example, antimetabolite resistance as the basis of selection for dhfr, which confers resistance to methotrexate (Reiss, Plant Physiol. (Life Sci. Adv.) 13 (1994), 143-149), npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or B-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule(s) can be used alone or as part of (a) vector(s) to express the antigen binding receptors of the invention in cells, for, e.g., adoptive T cell therapy but also for gene therapy purposes. The nucleic acid molecules or vector(s) containing the DNA sequence(s) encoding any one of the herein described antigen binding receptors is introduced into the cells which in turn produce the polypeptide of interest. Gene therapy, which is based on introducing therapeutic genes into cells by ex-vivo or in-vivo techniques is one of the most important applications of gene transfer. Suitable vectors, methods or gene-delivery systems for in methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91 (1998), 30-36; Verma, Gene Ther. 5 (1998), 692-699; Nabel, Ann. N.Y. Acad. Sci. 811 (1997), 289-292; Verzeletti, Hum. Gene Ther. 9 (1998), 2243-51; Wang, Nature Medicine 2 (1996), 714-716; WO 94/29469; WO 97/00957; US 5,580,859; US 5,589,466; or Schaper, Current Opinion in Biotechnology 7 (1996), 635-640. The recited nucleic acid molecule(s) and vector(s) may be designed for direct introduction or for introduction via liposomes, or viral vectors (e.g., adenoviral, retroviral) into the cell. In the context of the present invention, said cell is a T cells, such as CD8+ T cells, CD4+ T cells, CD3+ T cells, γδ T cells or natural killer (NK) T cells, preferably CD8+ T cells.

In accordance with the above, the present invention relates to methods to derive vectors, particularly plasmids, cosmids and bacteriophages used conventionally in genetic engineering that comprise a nucleic acid molecule encoding the polypeptide sequence of an antigen binding receptor defined herein. In the context of the present invention, said vector is an expression vector and/or a gene transfer or targeting vector. Expression vectors derived from viruses such as retroviruses, vaccinia virus, adeno-associated virus, herpes virus, or bovine papilloma virus, may be used for delivery of the recited polynucleotides or vector into targeted cell populations. Methods which are well known to those skilled in the art can be used to construct (a) recombinant vector(s); see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra. The recited vector may, inter alia, be the pEF-DHFR, pEF-ADA or pEF-neo. The vectors pEF-DHFR, pEF-ADA and pEF-neo have been described in the art, e.g. in Mack et al. Proc. Natl. Acad. Sci. USA 92 (1995), 7021-7025 and Raum et al. Cancer Immunol Immunother 50 (2001) , 141-150.

The invention also provides for a T cell transformed or transfected with a vector as described herein. Said T cell may be produced by introducing at least one of the above described vector or at least one of the above described nucleic acid molecules into the T cell or its precursor cell. The presence of said at least one vector or at least one nucleic acid molecule in the T cell may mediate the expression of a gene encoding the above described antigen binding receptor comprising an extracellular domain comprising an antigen binding moiety. The vector of the present invention can be polycistronic.

The described nucleic acid molecule(s) or vector(s) which is (are) introduced in the T cell or its precursor cell may either integrate into the genome of the cell or it may be maintained extrachromosomally.

Tumor Specific Antigens

As mentioned above, the antigen binding receptors according to the invention comprise an antigen-interaction-site/antigen binding moiety with specificity for a cell surface molecule, i.e. a tumor-specific antigen that naturally occurs on the surface of a tumor cell. In the context of the present invention, such antigen-interaction-sites will bring transduced T cells as described herein comprising the antigen binding receptor of the invention in physical contact with a tumor cell, wherein the transduced T cell becomes activated. Activation of transduced T cells of the present invention can result with lysis of the tumor cell as described herein.

Examples of tumor markers/tumor associated antigens that naturally occur on the surface of tumor cells are given herein below and comprise, but are not limited to FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen—antigen D related (HLA-DR), MUC-1 (Mucin-1), A33-antigen, PSMA (prostate-specific membrane antigen), FMS-like tyrosine kinase 3 (FLT-3), PDL1 (programmed death-ligand 1), PSMA (prostate specific membrane antigen), PSCA (prostate stem cell antigen), transferrin-receptor, TNC (tenascin), carbon anhydrase IX (CA-IX), and/or peptides bound to a molecule of the human major histocompatibility complex (MHC).

Accordingly, in the context of the present invention, the antigen binding receptor as described herein an antigen/marker that naturally occurs on the surface of tumor cells selected from the group consisting of FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen—antigen D related (HLA-DR), MUC-1 (Mucin-1), A33-antigen, PSMA (prostate-specific membrane antigen), FMS-like tyrosine kinase 3 (FLT-3), PDL1 (programmed death-ligand 1), PSMA (prostate specific membrane antigen), PSCA (prostate stem cell antigen), transferrin-receptor, TNC (tenascin), carbon anhydrase IX (CA-IX), and/or peptides bound to a molecule of the human major histocompatibility complex (MHC).

The sequence(s) of the (human) members of the A33-antigen, BCMA (B-cell maturation antigen), cancer antigen 12-5 (CA-12-5), carbon anhydrase IX (CA-IX), CD19, CD20, CD22, CD33, CD38, CEA (carcinoembryonic antigen), EpCAM (epithelial cell adhesion molecule), FAP (fibroblast activation protein), FMS-like tyrosine kinase 3 (FLT-3), folate receptor 1 (FOLR1), HER-1 (human epidermal growth factor 1), HER-2 (human epidermal growth factor 2), HER-3 (human epidermal growth factor 3), human leukocyte antigen—antigen D related (HLA-DR), MSLN (mesothelin), MCSP (melanoma chondroitin sulfate proteoglycan), MUC-1 (Mucin-1), PDL1 (programmed death-ligand 1), PSMA (prostate specific membrane antigen), PSMA (prostate-specific membrane antigen), PSCA (prostate stem cell antigen), p95 (p95HER2), transferrin-receptor, TNC (tenascin), human trophoblast cell-surface antigen 2 (Trop-2) are available in the UniProtKB/Swiss-Prot database and can be retrieved from http://www.uniprot.org/uniprot/? query=reviewed%3Ayes. These (protein) sequences also relate to annotated modified sequences. The present invention also provides techniques and methods wherein homologous sequences, and also genetic allelic variants and the like of the concise sequences provided herein are used. Preferably such variants and the like of the concise sequences herein are used. Preferably, such variants are genetic variants. The skilled person may easily deduce the relevant coding region of these (protein) sequences in these databank entries, which may also comprise the entry of genomic DNA as well as mRNA/cDNA. The sequence(s) of the (human) FAP (fibroblast activation protein) can be obtained from the Swiss-Prot database entry Q12884 (entry version 168, sequence version 5); The sequence(s) of the (human) CEA (carcinoembryonic antigen) can be obtained from the Swiss-Prot database entry P06731 (entry version 171, sequence version 3); the sequence(s) of the (human) EpCAM (Epithelial cell adhesion molecule) can be obtained from the Swiss-Prot database entry P16422 (entry version 117, sequence version 2); the sequence(s) of the (human) MSLN (mesothelin) can be obtained from the UniProt Entry number Q13421 (version number 132; sequence version 2); the sequence(s) of the (human) FMS-like tyrosine kinase 3 (FLT-3) can be obtained from the Swiss-Prot database entry P36888 (primary citable accession number) or Q13414 (secondary accession number) with the version number 165 and the sequence version 2; the sequences of (human) MCSP (melanoma chondroitin sulfate proteoglycan) can be obtained from the UniProt Entry number Q6UVK1 (version number 118; sequence version 2); the sequence(s) of the (human) folate receptor 1 (FOLR1) can be obtained from the UniProt Entry number P15328 (primary citable accession number) or Q53EW2 (secondary accession number) with the version number 153 and the sequence version 3; the sequence(s) of the (human) trophoblast cell-surface antigen 2 (Trop-2) can be obtained from the UniProt Entry number P09758 (primary citable accession number) or Q15658 (secondary accession number) with the version number 172 and the sequence version 3; the sequence(s) of the (human) PSCA (prostate stem cell antigen) can be obtained from the UniProt Entry number 043653 (primary citable accession number) or Q6UW92 (secondary accession number) with the version number 134 and the sequence version 1; the sequence(s) of the (human) HER-1 (Epidermal growth factor receptor) can be obtained from the Swiss-Prot database entry P00533 (entry version 177, sequence version 2); the sequence(s) of the (human) HER-2 (Receptor tyrosine-protein kinase erbB-2) can be obtained from the Swiss-Prot database entry P04626 (entry version 161, sequence version 1); the sequence(s) of the (human) HER-3 (Receptor tyrosine-protein kinase erbB-3) can be obtained from the Swiss-Prot database entry P21860 (entry version 140, sequence version 1); the sequence(s) of the (human) CD20 (B-lymphocyte antigen CD20) can be obtained from the Swiss-Prot database entry P11836 (entry version 117, sequence version 1); the sequence(s) of the (human) CD22 (B-lymphocyte antigen CD22) can be obtained from the Swiss-Prot database entry P20273 (entry version 135, sequence version 2); the sequence(s) of the (human) CD33 (B-lymphocyte antigen CD33) can be obtained from the Swiss-Prot database entry P20138 (entry version 129, sequence version 2); the sequence(s) of the (human) CA-12-5 (Mucin 16) can be obtained from the Swiss-Prot database entry Q8WXI7 (entry version 66, sequence version 2); the sequence(s) of the (human) HLA-DR can be obtained from the Swiss-Prot database entry Q29900 (entry version 59, sequence version 1); the sequence(s) of the (human) MUC-1 (Mucin-1) can be obtained from the Swiss-Prot database entry P15941 (entry version 135, sequence version 3); the sequence(s) of the (human) A33 (cell surface A33 antigen) can be obtained from the Swiss-Prot database entry Q99795 (entry version 104, sequence version 1); the sequence(s) of the (human) PDL1 (programmed death-ligand 1) can be obtained from the Swiss-Prot database entry Q9NZQ7 (entry version 148, sequence version 1); the sequence(s) of the (human) PSMA (Glutamate carboxypeptidase 2) can be obtained from the Swiss-Prot database entry Q04609 (entry version 133, sequence version 1); the sequence(s) of the (human) transferrin receptor can be obtained from the Swiss-Prot database entries Q9UP52 (entry version 99, sequence version 1) and P02786 (entry version 152, sequence version 2); the sequence of the (human) TNC (tenascin) can be obtained from the Swiss-Prot database entry P24821 (entry version 141, sequence version 3); or the sequence(s) of the (human) CA-IX (carbonic anhydrase IX) can be obtained from the Swiss-Prot database entry Q16790 (entry version 115, sequence version 2).

Therapeutic Use and Methods of Treatment

The molecules or constructs (i.e., antigen binding receptors, transduced T cells and kits) provided herein are particularly useful in medical settings, in particular for treatment of a malignant disease. For examples a tumor may be treated with a transduced T cell expressing an antigen binding receptor of the present invention. Accordingly, in certain embodiments, the antigen binding receptor, the transduced T cell or the kit are used in the treatment of a malignant disease, in particular wherein the malignant disease is selected from cancer of epithelial, endothelial or mesothelial origin and cancer of the blood.

The tumor specificity of the treatment is provided by the antigen binding moiety/moieties of the antigen binding receptor(s) of the invention.

In this context the malignant disease may be a cancer/carcinoma of epithelial, endothelial or mesothelial origin or a cancer of the blood. In the context of the present invention the cancer/carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitourinary tract, e.g., testis cancer, ovarial cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

For example, tumorous diseases and/or lymphomas may be treated with a specific construct directed against these medical indication(s). The indication for a transduced T cell of the present invention is given by specificity of the antigen binding receptor to a tumor antigen. For example, gastrointestinal cancer, pancreatic cancer, cholangiocellular cancer, lung cancer, breast cancer, ovarian cancer, skin cancer and/or oral cancer may be treated with an antigen binding receptor directed against (human) EpCAM (as the tumor-specific antigen naturally occurring on the surface of a tumor cell).

Gastrointestinal cancer, pancreatic cancer, cholangiocellular cancer, lung cancer, breast cancer, ovarian cancer, skin cancer and/or oral cancer may be treated with a transduced T cell of the present invention directed against HER1, preferably human HER1. Furthermore, gastrointestinal cancer, pancreatic cancer, cholangiocellular cancer, lung cancer, breast cancer, ovarian cancer, skin cancer, glioblastoma and/or oral cancer may be treated with a transduced T cell of the present invention directed against MCSP, preferably human MCSP. Gastrointestinal cancer, pancreatic cancer, cholangiocellular cancer, lung cancer, breast cancer, ovarian cancer, skin cancer, glioblastoma and/or oral cancer may be treated with a transduced T cell of the present directed against FOLR1, preferably human FOLR1. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention directed against Trop-2, preferably human Trop-2. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention directed against PSCA, preferably human PSCA. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention directed against EGFRvIII, preferably human EGFRvIII. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention directed against MSLN, preferably human MSLN. Gastric cancer, breast cancer and/or cervical cancer may be treated with a transduced T cell of the present invention directed against HER2, preferably human HER2. Gastric cancer and/or lung cancer may be treated with a transduced T cell of the present invention directed against HER3, preferably human HER3. B-cell lymphoma and/or T cell lymphoma may be treated with a transduced T cell of the present invention directed against CD20, preferably human CD20. B-cell lymphoma and/or T cell lymphoma may be treated with a transduced T cell of the present invention directed against CD22, preferably human CD22. Myeloid leukemia may be treated with a transduced T cell of the present invention directed against CD33, preferably human CD33. Ovarian cancer, lung cancer, breast cancer and/or gastrointestinal cancer may be treated with a transduced T cell of the present invention directed against CA12-5, preferably human CA12-5. Gastrointestinal cancer, leukemia and/or nasopharyngeal carcinoma may be treated with a transduced T cell of the present invention directed against HLA-DR, preferably human HLA-DR. Colon cancer, breast cancer, ovarian cancer, lung cancer and/or pancreatic cancer may be treated with a transduced T cell of the present invention directed against MUC-1, preferably human MUC-1. Colon cancer may be treated with a transduced T cell of the present invention directed against A33, preferably human A33. Prostate cancer may be treated with a transduced T cell of the present invention directed against PSMA, preferably human PSMA. Gastrointestinal cancer, pancreatic cancer, cholangiocellular cancer, lung cancer, breast cancer, ovarian cancer, skin cancer and/or oral cancer may be treated with a transduced T cell of the present invention directed against the transferrin receptor, preferably the human transferring receptor. Pancreatic cancer, lunger cancer and/or breast cancer may be treated with a transduced T cell of the present invention directed against the transferrin receptor, preferably the human transferring receptor. Renal cancer may be with a transduced T cell of the present invention directed against CA-IX, preferably human CA-IX.

More than one of the herein described T cells can be co-applied and/or, more than one antigen binding receptor according to the invention can be co-expressed and/or co-transduced in the same T cell. The invention further provided methods to combine more than one antigen binding receptor within the same cell without reducing activity of the single antigen binding receptors compared to the situation where a single antigen binding receptor is expressed and/or transduced in a T cell of the invention.

In this context, the invention also relates to a method for the treatment of a disease, a malignant disease such as cancer of epithelial, endothelial or mesothelial origin and/or cancer of blood. In the context of the present invention, said subject is a human.

In the context of the present invention a particular method for the treatment of a disease comprises the steps of
(a) isolating T cells, preferably CD8+ T cells, from a subject;
(b) transducing said isolated T cells, preferably CD8+ T cells, with at least one antigen binding receptor as described herein; and
(c) administering the transduced T cells, preferably CD8+ T cells, to said subject. In the context of the present invention, said transduced T cells, preferably CD8+ T cells, and/or therapeutic antibody/antibodies are co-administered to said subject by intravenous infusion. Moreover, in the context of the present invention the present invention, provides a method for the treatment of a disease comprising the steps of
(a) isolating T cells, preferably CD8+ T cells, from a subject;
(b) transducing said isolated T cells, preferably CD8+ T cells, with at least one antigen binding receptor as described herein;
(c) optionally co-transducing said isolated T cells, preferably CD8+ T cells, with a T cell receptor;
(d) expanding the T cells, preferably CD8+ T cells, by anti-CD3 and anti-CD28 antibodies; and
(e) administering the transduced T cells, preferably CD8+ T cells, to said subject.

The above mentioned step (d) (referring to the expanding step of the T cells such as TIL by anti-CD3 and/or anti-CD28 antibodies) may also be performed in the presence of (stimulating) cytokines such as interleukin-2 and/or interleukin-15 (IL-15). In the context of the present invention, the above mentioned step (d) (referring to the expanding step of the T cells such as TIL by anti-CD3 and/or anti-CD28 antibodies) may also be performed in the presence of interleukin-12 (IL-12), interleukin-7 (IL-7) and/or interleukin-21 (IL-21).

In the context of the present invention the administration of the transduced T cells will be performed by intravenous infusion. In the context of the present invention that transduced T cells can be isolated/obtained from the subject to be treated.

Compositions

Furthermore, the invention provides compositions (medicaments) comprising (an) transduced T cell(s) comprising one or more antigen binding receptor(s) of the invention, (a) nucleic acid molecule(s) and (a) vector(s) encoding the antigen binding receptors according to the invention, and/or and kits comprising one or more of said compositions. In the context of the present invention, the composition is a pharmaceutical composition further comprising, optionally, suitable formulations of carrier, stabilizers and/or excipients. Accordingly, in the context of the present invention a pharmaceutical composition (medicament) is provided that comprises a transduced T cell comprising an antigen binding receptor as described herein.

In accordance with this invention, the term “pharmaceutical composition” relates to a composition for administration to a patient, preferably a human patient. Furthermore, in the context of the present invention that patient suffers from a disease, wherein said disease is a malignant disease, especially cancers/carcinomas of ephithelial, endothelial or mesothelial origin or a cancer of the blood. In the context of the present invention the cancers/carcinomas is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitor-urinary tract, e.g., testis cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

In a preferred embodiment, the pharmaceutical composition/medicament comprises a transduced T cell as defined herein for parenteral, transdermal, intraluminal, intraarterial, intravenous, intrathecal administration or by direct injection into the tissue or tumor. In the context of the present invention the composition/medicament comprises transduced T cells comprising an antigen binding receptor as defined herein. In the context of the present invention the pharmaceutical composition/medicament comprises a transduced T cell comprising an antigen binding receptor as defined herein, in particular wherein said T cell was obtained from a subject to be treated.

It is particular envisaged, that said pharmaceutical composition(s)/medicament(s) is (are) to be administered to a patient via infusion or injection. In the context of the present invention the transduced T cells comprising an antigen binding receptor as described herein is to be administered to a patient via infusion or injection. Administration of the suitable compositions/medicaments may be effected by different ways, intravenous, intraperitoneal, subcutaneous, intramuscular, topical or intradermal administration.

The pharmaceutical composition/medicament of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 5 g units per day. However, a more preferred dosage for continuous infusion might be in the range of 0.01 μg to 2 mg, preferably 0.01 μg to 1 mg, more preferably 0.01 μg to 100 μg, even more preferably 0.01 μg to 50 μg and most preferably 0.01 μg to 10 μg units per kilogram of body weight per hour. Particularly preferred dosages are recited herein below. Progress can be monitored by periodic assessment. Dosages will vary but a preferred dosage for intravenous administration of DNA is from approximately 10⁶ to 10¹² copies of the DNA molecule.

The compositions of the invention may be administered locally or systematically. Administration will generally be parenterally, e.g., intravenously; transduced T cells may also be administered directed to the target site, e.g., by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present invention might comprise proteinaceous carriers, like, e.g., serum albumine or immunoglobuline, preferably of human origin. It is envisaged that the pharmaceutical composition of the invention might comprise, in addition to the cells, further biologically active agents, depending on the intended use of the pharmaceutical composition. Such agents might be drugs acting on the gastro-intestinal system, drugs acting as cytostatica, drugs preventing hyperurikemia, drugs inhibiting immunereactions (e.g. corticosteroids), drugs acting on the circulatory system and/or agents such as T cell co-stimulatory molecules or cytokines known in the art.

Possible indication for administration of the composition(s)/medicament(s) of the invention are malignant diseases such as cancer of epithelial, endothelial or mesothelial origin and cancer of the blood, especially epithelial cancers/carcinomas such as breast cancer, colon cancer, prostate cancer, head and neck cancer, skin cancer (melanoma), cancers of the genitor-urinary tract, e.g., ovarial cancer, testis cancer, endothelial cancer, cervix cancer and kidney cancer, lung cancer, gastric cancer, cancer of the bile duct, esophagus cancer, cancer of the salivatory glands and cancer of the thyroid gland or other tumorous diseases like haematological tumors, gliomas, sarcomas or osteosarcomas.

The invention further envisages the co-administration protocols with other compounds, e.g., molecules capable of providing an activation signal for immune effector cells, for cell proliferation or for cell stimulation. Said molecule may be, e.g., a further primary activation signal for T cells (e.g. a further costimulatory molecule: molecules of B7 family, Ox40L, 4.1 BBL, CD40L, anti-CTLA-4, anti-PD-1), or a further cytokine interleukin (e.g., IL-2).

The composition of the invention as described above may also be a diagnostic composition further comprising, optionally, means and methods for detection.

Accordingly, in preferred embodiments, provided are the kit, the antigen binding receptors or the transduced T cell as described herein for use as a medicament. In the context of the present invention, the antigen binding receptor according to the invention for use as a medicament is provided, wherein transduced T cells, preferably CD8+ T cells, comprising and/or expressing an antigen binding receptor as defined herein are administered to a subject and wherein said T cells, preferably CD8+ T cells, were obtained from the subject to be treated. Said medicament may be employed in a method of treatment of malignant diseases especially cancers/carcinomas of epithelial, endothelial or mesothelial origin or of the blood. In the context of the present invention the cancer/carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitor-urinary tract, e.g., testis cancer, ovarial cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

Furthermore, in the context of the present invention antigen binding receptor binds to a tumor-specific antigen naturally occurring on the surface of a tumor cell. In the context of the present invention the cancer/carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitor-urinary tract, e.g., testis cancer, ovarial cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

Furthermore, in accordance to the invention, a molecule or construct (i.e., an antigen binding receptor as described herein) comprising an extracellular domain comprising one or more, preferably one, antigen binding moieties directed to/binding to/interacting with a tumor antigen, preferably a human tumor associated antigen, (as the tumor-specific antigen naturally occurring on the surface of a tumor cell), wherein the herein defined extracellular domains of the antigen binding receptor of the present invention is directed to/binding to/interacting with the tumor associated antigen, is provided for in the treatment of gastrointestinal cancer, pancreatic cancer, cholangiocellular cancer, lung cancer, breast cancer, ovarian cancer, skin cancer and/or oral cancer. Thus, in the context of the present invention an antigen binding receptor comprising an extracellular domain directed to/binding to/interacting with a tumor associated antigen, for use in the treatment of epithelial, endothelial or mesothelial origin and cancer of the blood is provided.

In one embodiment, provided is an antigen binding receptor according to the invention directed to/binding to/interacting with a tumor antigen, for use in the treatment of gastrointestinal cancer, pancreatic cancer, cholangiocellular cancer, lung cancer, breast cancer, ovarian cancer, skin cancer and/or oral cancer.

In one embodiment, provided is an antigen binding receptor according to the invention directed to/binding to/interacting with HER1, preferably human HER1, for use in the treatment of gastrointestinal cancer, pancreatic cancer, cholangiocellular cancer, lung cancer, breast cancer, ovarian cancer, skin cancer and/or oral cancer.

In one embodiment, provided is an antigen binding receptor according to the invention directed to/binding to/interacting with HER2, preferably human HER2, for use in the treatment of gastric cancer, breast cancer and/or cervical cancer.

In one embodiment, provided is an antigen binding receptor according to the invention directed to/binding to/interacting with HER3, preferably human HER3, for use in the treatment of gastric cancer and/or lung cancer.

In one embodiment, provided is an antigen binding receptor according to the invention directed to/binding to/interacting with CEA, preferably human CEA, for use in the treatment of cancer of epithelial, endothelial or mesothelial origin and cancer of the blood.

In one embodiment, provided is an antigen binding receptor according to the invention directed to/binding to/interacting with p95, preferably human p95, for use in the treatment of cancer of epithelial, endothelial or mesothelial origin and cancer of the blood.

In one embodiment, provided is an antigen binding receptor according to the invention directed to/binding to/interacting with BCMA, preferably human BCMA, for use in the treatment of cancer of epithelial, endothelial or mesothelial origin and cancer of the blood.

In one embodiment, provided is an antigen binding receptor according to the invention directed to/binding to/interacting with MSLN, preferably human MSLN, for use in the treatment of cancer of epithelial, endothelial or mesothelial origin and cancer of the blood.

In one embodiment, provided is an antigen binding receptor according to the invention directed to/binding to/interacting with MCSP, preferably human MCSP, for use in the treatment of cancer of epithelial, endothelial or mesothelial origin and cancer of the blood.

In one embodiment, provided is an antigen binding receptor according to the invention directed to/binding to/interacting with t CD19, preferably human CD19, for use in the treatment of cancer of epithelial, endothelial or mesothelial origin and cancer of the blood.

In one embodiment, provided is an antigen binding receptor according to the invention directed to/binding to/interacting with CD20, preferably human CD20, for use in the treatment of B-cell lymphoma and/or T cell lymphoma.

In one embodiment, provided is an antigen binding receptor according to the invention directed to/binding to/interacting with CD22, preferably human CD22, for use in the treatment of B-cell lymphoma and/or T cell lymphoma.

In one embodiment, provided is an antigen binding receptor according to the invention directed to/binding to/interacting with CD38, preferably human CD38, for use in the treatment of cancer of epithelial, endothelial or mesothelial origin and cancer of the blood.

In one embodiment, provided is an antigen binding receptor according to the invention directed to/binding to/interacting with CD52Flt3, preferably human CD52Flt3, for use in the treatment of cancer of epithelial, endothelial or mesothelial origin and cancer of the blood.

In one embodiment, provided is an antigen binding receptor according to the invention directed to/binding to/interacting with Fo1R1, preferably human FolR1, for use in the treatment of cancer of epithelial, endothelial or mesothelial origin and cancer of the blood.

In one embodiment, provided is an antigen binding receptor according to the invention directed to/binding to/interacting with Trop-2, preferably human Trop-2, for use in the treatment of gastrointestinal cancer, pancreatic cancer, cholangiocellular cancer, lung cancer, breast cancer, ovarian cancer, skin cancer, glioblastoma and/or oral cancer.

In one embodiment, provided is an antigen binding receptor according to the invention directed to/binding to/interacting with CA-12-5, preferably human CA-12-5, for use in the treatment of ovarian cancer, lung cancer, breast cancer and/or gastrointestinal cancer.

In one embodiment, provided is anantigen binding receptor according to the invention directed to/binding to/interacting with DR, preferably human HLA-DR, for use in the treatment of gastrointestinal cancer, leukemia and/or nasopharyngeal carcinoma.

In one embodiment, provided an antigen binding receptor according to the invention directed to/binding to/interacting with MUC-1, preferably human MUC-1, for use in the treatment cancer of colon cancer, breast cancer, ovarian cancer, lung cancer and/or pancreatic cancer.

In one embodiment, provided is an antigen binding receptor according to the invention directed to/binding to/interacting with A33, preferably human A33, for use in the treatment of colon cancer.

In one embodiment, provided is an antigen binding receptor according to the invention directed to/binding to/interacting with PSMA, preferably human PSMA, for use in the treatment of prostate cancer.

In one embodiment, provided is an the antigen binding receptor according to the invention directed to/binding to/interacting with PSCA, preferably human PSCA, for use in the treatment cancer of epithelial, endothelial or mesothelial origin and cancer of the blood.

In one embodiment, provided is an antigen binding receptor according to the invention directed to/binding to/interacting with the transferrin-receptor, preferably the human transferring-receptor, for use in the treatment of cancer of epithelial, endothelial or mesothelial origin and cancer of the blood.

In one embodiment, provided is an antigen binding receptor according to the invention directed to/binding to/interacting with tenascin, preferably human tenascin, for use in the treatment of cancer of epithelial, endothelial or mesothelial origin and cancer of the blood.

In one embodiment, provided is an antigen binding receptor according to the invention directed to/binding to/interacting with CA-IX, preferably human XA-IX, for use in the treatment of cancer of epithelial, endothelial or mesothelial origin and cancer of the blood.

In one embodiment, provided is an antigen binding receptor according to the invention directed to/binding to/interacting with PDL1, preferably PDL1, for use in the treatment of cancer of epithelial, endothelial or mesothelial origin and cancer of the blood.

Exemplary Embodiments

1. An antigen binding receptor comprising an anchoring transmembrane domain and an extracellular domain comprising an antigen binding moiety, wherein the antigen binding moiety is a Fab, crossFab or a scFab fragment, in particular a Fab or crossFab fragment.

2. The antigen binding receptor of embodiment 1, wherein the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof.

3. The antigen binding receptor of any one of embodiments 1 or 2, wherein the anchoring transmembrane domain is the CD28 transmembrane domain or a fragment thereof, in particular wherein the anchoring transmembrane domain comprises the amino acid sequence of SEQ ID NO:14.

4. The antigen binding receptor of any one of embodiments 1 to 3 further comprising at least one stimulatory signaling domain and/or at least one co-stimulatory signaling domain.

5. The antigen binding receptor of any one of embodiments 1 to 4, wherein the at least one stimulatory signaling domain is individually selected from the group consisting of the intracellular domain of CD3z, of FCGR3A and of NKG2D, or fragments thereof.

6. The antigen binding receptor of any one of embodiments 1 to 5, wherein the at least one stimulatory signaling domain is the intracellular domain of CD3z or a fragment thereof, in particular wherein the at least one stimulatory signaling domain comprises the amino acid sequence of SEQ ID NO:16.

7. The antigen binding receptor of any one of embodiments 1 to 6, wherein the at least one co-stimulatory signaling domain is individually selected from the group consisting of the intracellular domain of CD27, of CD28, of CD137, of OX40, of ICOS, of DAP10 and of DAP12, or fragments thereof.

8. The antigen binding receptor of any one of embodiments 1 to 7, wherein the at least one co-stimulatory signaling domain is the CD28 intracellular domain or a fragment thereof, in particular, wherein the at least one co-stimulatory signaling domain comprises the amino acid sequence of SEQ ID NO:15.

9. The antigen binding receptor of any one of embodiments 1 to 8, wherein the antigen binding receptor comprises one stimulatory signaling domain comprising the intracellular domain of CD3z, or a fragment thereof, and wherein the antigen binding receptor comprises one co-stimulatory signaling domain comprising the intracellular domain of CD28, or a fragment thereof.

10. The antigen binding receptor of embodiment 9, wherein the stimulatory signaling domain comprises the amino acid sequence of SEQ ID NO:16 and the co-stimulatory signaling domain comprises the amino acid sequence of SEQ ID NO:15.

11. The antigen binding receptor of any one of embodiments 1 to 10, wherein the extracellular domain is connected to the anchoring transmembrane domain, optionally through a peptide linker.

12. The antigen binding receptor of embodiment 11, wherein the peptide linker comprises the amino acid sequence GGGGS (SEQ ID NO:20).

13. The antigen binding receptor of any one of embodiments 1 to 12, wherein the anchoring transmembrane domain is connected to a co-signaling domain or to a signaling domain, optionally through a peptide linker.

14. The antigen binding receptor of any one of embodiments 1 to 13, wherein the signaling and/or co-signaling domains are connected, optionally through at least one peptide linker.

15. The antigen binding receptor of any one of embodiments 1 to 14, wherein the antigen binding moiety comprises a heavy chain constant (CH) domain and a light chain constant domain (CL), wherein the CH domain or the CL domain is connected at the C-terminus to the N-terminus of the anchoring transmembrane domain, optionally through a peptide linker.

16. The antigen binding receptor of any one of embodiments 4 to 15, wherein the antigen binding receptor comprises one co-signaling domain, wherein the co-signaling domain is connected at the N-terminus to the C-terminus of the anchoring transmembrane domain.

17. The antigen binding receptor of embodiment 16, wherein the antigen binding receptor additionally comprises one stimulatory signaling domain, wherein the stimulatory signaling domain is connected at the N-terminus to the C-terminus of the co-stimulatory signaling domain.

18. The antigen binding receptor of any one of embodiments 1 to 17, wherein the antigen binding moiety is capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

19. The antigen binding receptor of any one of embodiments 1 to 18, wherein the antigen binding moiety is capable of specific binding to an antigen selected from the group consisting of fibroblast activation protein (FAP), carcinoembryonic antigen (CEA), mesothelin (MSLN), CD20, folate receptor 1 (FOLR1), tenascin (TNC) and programmed death-ligand 1(PDL1).

20. The antigen binding receptor of any one of embodiments 1 to 19, wherein the antigen binding moiety is a capable of specific binding to CD20, wherein the antigen binding moiety comprises:

(i) a heavy chain variable region (VH) comprising
(a) the heavy chain complementarity-determining region (CDR H) 1 amino acid sequence YSWIN (SEQ ID NO:1);
(b) the CDR H2 amino acid sequence RIFPGDGDTDYNGKFKG (SEQ ID NO:2); and
(c) the CDR H3 amino acid sequence NVFDGYWLVY (SEQ ID NO:3); and

(ii) a light chain variable region (VL) comprising
(d) the light chain complementary-determining region (CDR L) 1 amino acid sequence RSSKSLLHSNGITYLY (SEQ ID NO:4);
(e) the CDR L2 amino acid sequence QMSNLVS (SEQ ID NO:5); and
(f) the CDR L3 amino acid sequence AQNLELPYT (SEQ ID NO:6).

21. The antigen binding receptor of any one of embodiments 1 to 20, wherein the antigen binding moiety is capable of specific binding to CD20, wherein the antigen binding moiety comprises a heavy chain variable region (VH) comprising an amino acid sequence that is at least about 95%, 96%, 97%, 98%, 99% or 100% identical to the amino acid of SEQ ID NO:12, and a light chain variable region (VL) comprising an amino acid sequence that is at least about 95%, 96%, 97%, 98%, 99% or 100% identical to the amino acid sequence of SEQ ID NO:10.

22. The antigen binding receptor of any one of embodiments 1 to 21, wherein the antigen binding moiety comprises the heavy chain variable region (VH) of SEQ ID NO:12 and the light chain variable region (VL) of SEQ ID NO:10.

23. The antigen binding receptor of any one of embodiments 1 to 22, wherein the antigen binding moiety is a Fab fragment capable of specific binding to CD20, wherein the antigen binding receptor comprises

a) a first polypeptide that is at least about 95%, 96%, 97%, 98%, 99% or 100% identical to an amino acid sequence selected from the group consisting of SEQ ID NO:7 and SEQ ID NO:50; and

b) a second polypeptide that is at least about 95%, 96%, 97%, 98%, 99% or 100% identical to an amino acid sequence selected from the group consisting of SEQ ID NO:9 and SEQ ID NO:8.

24. The antigen binding receptor of embodiment 23, comprising

a) a first polypeptide of SEQ ID NO:7; and

b) a second polypeptide of SEQ ID NO:9.

25. The antigen binding receptor of embodiment 23, comprising

a) a first polypeptide of SEQ ID NO:50; and

b) a second polypeptide of SEQ ID NO:8.

26. The antigen binding receptor of any one of embodiments 1 to 22, wherein the antigen binding moiety is a crossFab fragment capable of specific binding to CD20, wherein the antigen binding receptor comprises

a) a first polypeptide that is at least about 95%, 96%, 97%, 98%, 99% or 100% identical to an amino acid sequence selected from the group consisting of SEQ ID NO:36 and SEQ ID NO:41; and

b) a second polypeptide that is at least about 95%, 96%, 97%, 98%, 99% or 100% identical to an amino acid sequence selected from the group consisting of SEQ ID NO:38 and SEQ ID NO:43.

27. The antigen binding receptor of embodiment 26, comprising

a) a first polypeptide of SEQ ID NO:36; and

b) a second polypeptide of SEQ ID NO:38.

28. The antigen binding receptor of embodiment 26, comprising

a) a first polypeptide of SEQ ID NO:41; and

b) a second polypeptide of SEQ ID NO:43.

29. The antigen binding receptor of any one of embodiments 1 to 22, wherein the antigen binding moiety is a scFab fragment capable of specific binding to CD20, wherein the antigen binding receptor comprises a polypeptide that is at least about 95%, 96%, 97%, 98%, 99% or 100% identical to the amino acid sequence of SEQ ID NO:51.

30. The antigen binding receptor of embodiment 29, comprising the polypeptide of SEQ ID NO:51.

31. The antigen binding receptor of any one of embodiments 1 to 19, wherein the antigen binding moiety is a capable of specific binding to PDL1, wherein the antigen binding moiety comprises:

(i) a heavy chain variable region (VH) comprising
(a) the heavy chain complementarity-determining region (CDR H) 1 amino acid sequence DSWIH (SEQ ID NO:68);
(b) the CDR H2 amino acid sequence WISPYGGSTYYADSVKG (SEQ ID NO:69); and
(c) the CDR H3 amino acid sequence RHWPGGFDY (SEQ ID NO:70); and

(ii) a light chain variable region (VL) comprising
(d) the light chain complementary-determining region (CDR L) 1 amino acid sequence RASQDVSTAVA (SEQ ID NO:71);
(e) the CDR L2 amino acid sequence SASFLYS (SEQ ID NO:72); and
(f) the CDR L3 amino acid sequence QQYLYHPAT (SEQ ID NO:73).

32. The antigen binding receptor of any one of embodiments 1 to 19 and 31, wherein the antigen binding moiety is capable of specific binding to PDL1, wherein the antigen binding moiety comprises a heavy chain variable region (VH) comprising an amino acid sequence that is at least about 95%, 96%, 97%, 98%, 99% or 100% identical to the amino acid of SEQ ID NO:78, and a light chain variable region (VL) comprising an amino acid sequence that is at least about 95%, 96%, 97%, 98%, 99% or 100% identical to the amino acid sequence of SEQ ID NO:77.

33. The antigen binding receptor of any one of embodiments 1 to 19 and 31 to 32, wherein the antigen binding moiety comprises the heavy chain variable region (VH) of SEQ ID NO:78 and the light chain variable region (VL) of SEQ ID NO:77.

34. The antigen binding receptor of any one of embodiments 1 to 19 and 31 to 33, wherein the antigen binding moiety is a Fab fragment capable of specific binding to PDL1, wherein the antigen binding receptor comprises

a) a first polypeptide that is at least about 95%, 96%, 97%, 98%, 99% or 100% identical to an amino acid sequence selected from the group consisting of SEQ ID NO:74 and SEQ ID NO:85; and

b) a second polypeptide that is at least about 95%, 96%, 97%, 98%, 99% or 100% identical to an amino acid sequence selected from the group consisting of SEQ ID NO:76 and SEQ ID NO:75.

35. The antigen binding receptor of embodiment 34, comprising

a) a first polypeptide of SEQ ID NO:74; and

b) a second polypeptide of SEQ ID NO:76.

36. The antigen binding receptor of embodiment 34, comprising

a) a first polypeptide of SEQ ID NO:85; and

b) a second polypeptide of SEQ ID NO:75.

37. The antigen binding receptor of any one of embodiments 1 to 19 and 31 to 33, wherein the antigen binding moiety is a crossFab fragment capable of specific binding to PDL1, wherein the antigen binding receptor comprises

a) a first polypeptide that is at least about 95%, 96%, 97%, 98%, 99% or 100% identical to an amino acid sequence selected from the group consisting of SEQ ID NO:79 and SEQ ID NO:82; and

b) a second polypeptide that is at least about 95%, 96%, 97%, 98%, 99% or 100% identical to an amino acid sequence selected from the group consisting of SEQ ID NO:81 and SEQ ID NO:84.

38. The antigen binding receptor of embodiment 37, comprising

a) a first polypeptide of SEQ ID NO:79; and

b) a second polypeptide of SEQ ID NO:81.

39. The antigen binding receptor of embodiment 37, comprising

a) a first polypeptide of SEQ ID NO:82; and

b) a second polypeptide of SEQ ID NO:84.

40. The antigen binding receptor of any one of embodiments 1 to 19 and 31 to 33, wherein the antigen binding moiety is a scFab fragment capable of specific binding to PDL1, wherein the antigen binding receptor comprises a polypeptide that is at least about 95%, 96%, 97%, 98%, 99% or 100% identical to the amino acid sequence of SEQ ID NO:85.

41. The antigen binding receptor of embodiment 40, comprising the polypeptide of SEQ ID NO:85.

42. The antigen binding receptor of any one of embodiments 1 to 19, wherein the antigen binding moiety is a capable of specific binding to CEA, wherein the antigen binding moiety comprises:

(i) a heavy chain variable region (VH) comprising
(a) the heavy chain complementarity-determining region (CDR H) 1 amino acid sequence EFGMN (SEQ ID NO:138);
(b) the CDR H2 amino acid sequence WINTKTGEATYVEEFKG (SEQ ID NO:139); and
(c) the CDR H3 amino acid sequence WDFAYYVEAMDY (SEQ ID NO:140); and

(ii) a light chain variable region (VL) comprising
(d) the light chain complementary-determining region (CDR L) 1 amino acid sequence KASAAVGTYVA (SEQ ID NO:141);
(e) the CDR L2 amino acid sequence SASYRKR (SEQ ID NO:142); and
(f) the CDR L3 amino acid sequence HQYYTYPLFT (SEQ ID NO:143).

43. The antigen binding receptor of any one of embodiments 1 to 19, wherein the antigen binding moiety is a capable of specific binding to CEA, wherein the antigen binding moiety comprises:

(i) a heavy chain variable region (VH) comprising
(a) the heavy chain complementarity-determining region (CDR H) 1 amino acid sequence DTYMH (SEQ ID NO:148);
(b) the CDR H2 amino acid sequence RIDPANGNSKYVPKFQG (SEQ ID NO:149); and
(c) the CDR H3 amino acid sequence FGYYVSDYAMAY (SEQ ID NO:150); and

(ii) a light chain variable region (VL) comprising
(d) the light chain complementary-determining region (CDR L) 1 amino acid sequence RAGESVDIFGVGFLH (SEQ ID NO:151);
(e) the CDR L2 amino acid sequence RASNRAT (SEQ ID NO:152); and
(f) the CDR L3 amino acid sequence QQTNEDPYT (SEQ ID NO:153).

44. The antigen binding receptor of any one of embodiments 1 to 19 and 42 to 43, wherein the antigen binding moiety is capable of specific binding to CEA, wherein the antigen binding moiety comprises a heavy chain variable region (VH) comprising an amino acid sequence that is at least about 95%, 96%, 97%, 98%, 99% or 100% identical to an amino acid selected from the group consisting of SEQ ID NO:146 and SEQ ID NO:156, and a light chain variable region (VL) comprising an amino acid sequence that is at least about 95%, 96%, 97%, 98%, 99% or 100% identical to an amino acid sequence selected from the group consisting of SEQ ID NO:147 and SEQ ID NO:157.

45. The antigen binding receptor of any one of embodiments 1 to 19 and 42 to 44, wherein the antigen binding moiety comprises the heavy chain variable region (VH) of SEQ ID NO:146 and the light chain variable region (VL) of SEQ ID NO:147.

46. The antigen binding receptor of any one of embodiments 1 to 19 and 42 to 44, wherein the antigen binding moiety comprises the heavy chain variable region (VH) of SEQ ID NO:156 and the light chain variable region (VL) of SEQ ID NO:157.

47. The antigen binding receptor of any one of embodiments 1 to 46, wherein the antigen binding moiety comprises a CL domain and a CH1 domain, comprising at least one amino acid substitution of a charged amino acid (charged residues) in the CH1 and CL domains.

48. The antigen binding receptor of embodiment 47, wherein in the CL domain the amino acid at position 124 is substituted independently by lysine (K), arginine (R) or histidine (H) (numbering according to Kabat EU Index), and wherein in the CH1 domain the amino acids at positions 147 and 213 are substituted independently by glutamic acid (E) or aspartic acid (D) (numbering according to Kabat EU index).

49. An isolated polynucleotide encoding the antigen binding receptor of any one of embodiments 1 to 48.

50. A composition encoding the antigen binding receptor of any one of embodiments 1 to 48, comprising a first isolated polynucleotide encoding a first polypeptide, and a second isolated polynucleotide encoding a second polypeptide.

51. A polypeptide encoded by the polynucleotide of embodiment 49 or by the composition of embodiment 50.

52. A vector, particularly an expression vector, comprising the polynucleotide of embodiment 49 or the composition of embodiment 50.

53. A transduced T cell comprising the polynucleotide of embodiment 49, the composition of embodiment 50 or the vector of embodiment 52.

54. A transduced T cell capable of expressing at least one of the antigen binding receptors of any one of embodiments 1 to 48.

55. The transduced T cell of embodiment 54, wherein the cell comprises

(i) not more than one antigen binding receptor comprising a Fab (VH-CH-ATD) antigen binding domain;

(ii) not more than one antigen binding receptor comprising a Fab (VL-CL-ATD) antigen binding domain;

(iii) not more than one antigen binding receptor comprising a crossFab (VL-CH-ATD) antigen binding domain; and

(iv) not more one antigen binding receptor comprising a crossFab (VH-CL-ATD) antigen binding domain.

56. The transduced T cell of any one of embodiments 53 to 55, wherein the cell comprises a first antigen binding receptor according to any one of embodiments 1 to 48, wherein a first antigen binding receptor comprises a Fab antigen binding moiety, and wherein the cell comprises a second antigen binding receptor according to any one of embodiments 1 to 48, wherein the second antigen binding receptor comprises a crossFab antigen binding moiety.

57. The transduced T cell of any one of embodiments 53 to 55, wherein the cell comprises a first antigen binding receptor according to any one of embodiments 1 to 48, wherein the first antigen binding receptor comprises a Fab (VH-CH-ATD) antigen binding moiety, and wherein the cell comprises a second antigen binding receptor according to any one of embodiments 1 to 48, wherein the second antigen binding receptor comprises a Fab (VL-CL-ATD) antigen binding moiety.

58. The transduced T cell of any one of embodiments 53 to 55, wherein the cell comprises a first antigen binding receptor according to any one of embodiments 1 to 48, wherein the first antigen binding receptor comprises a crossFab (VL-CH-ATD) antigen binding moiety, and wherein the cell comprises a second antigen binding receptor according to any one of embodiments 1 to 48, wherein the second antigen binding receptor comprises a crossFab (VH-CL-ATD) antigen binding moiety.

59. The transduced T cell of any one of embodiments 53 to 55, wherein the cell comprises a first antigen binding receptor according to any one of embodiments 1 to 48, wherein a first antigen binding receptor comprises a scFab antigen binding moiety, and wherein the cell comprises a second antigen binding receptor according to any one of embodiments 1 to 48, wherein the second antigen binding receptor comprises an scFv, a Fab or crossFab antigen binding moiety.

60. The transduced T cell of any one of embodiments 53 to 59, wherein the cell comprises a first antigen binding receptor capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

61. The transduced T cell of any one of embodiments 54 to 60, wherein the cell comprises a second antigen binding receptor capable of specific binding to an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1, or to a peptide bound to a molecule of the human major histocompatibility complex (MHC).

62. The transduced T cell of any one of embodiments 53 to 61, wherein the cell comprises a first antigen binding receptor capable of specific binding to a first tumor associated antigen (TAA), and wherein the cell comprises a second antigen binding receptor capable of specific binding to a TAA.

63. The transduced T cell of any one of embodiments 53 to 62, wherein the cell comprises a first antigen binding receptor capable of specific binding to programmed death-ligand 1 (PDL1), and wherein the cell comprises a second antigen binding receptor capable of specific binding to an antigen selected from the group consisting of fibroblast activation protein (FAP), carcinoembryonic antigen (CEA), mesothelin (MSLN), CD20, folate receptor 1 (FOLR1), and tenascin (TNC).

64. The transduced T cell of any one of embodiments 53 to 63, wherein the cell comprises a first antigen binding receptor capable of specific binding to PDL1, and wherein the cell comprises a second antigen binding receptor capable of specific binding to CD20.

65. The transduced T cell of any one of embodiments 53 or 64, wherein the transduced T cell is co-transduced with a T cell receptor (TCR) capable of specific binding of a target antigen.

66. The antigen binding receptor of any one of embodiments 1 to 48 or the transduced T cell of any one of embodiments 53 to 65 for use as a medicament.

67. The antigen binding receptor of any one of embodiments 1 to 48 or the transduced T cell of any one of embodiments 53 to 65 for use in the treatment of a malignant disease, wherein the treatment comprises administration of a transduced T cell expressing the antigen binding receptor.

68. The antigen binding receptor or the transduced T cell for use according to embodiment 53 or 65, wherein said malignant disease is selected from cancer of epithelial, endothelial or mesothelial origin and cancer of the blood.

69. The transduced T cell for use according to any one of embodiments 66 to 68, wherein the transduced T cell is derived from a cell isolated from the subject to be treated.

70. The transduced T cell for use according to any one of embodiments 66 to 68, wherein the transduced T cell is not derived from a cell isolated from the subject to be treated.

71. A method of treating a disease in a subject, comprising administering to the subject a transduced T cell capable of expressing the antigen binding receptor of any one of embodiments 1 to 48.

72. The method of embodiment 71, additionally comprising isolating a T cell from the subject and generating the transduced T cell by transducing the isolated T cell with the polynucleotide of embodiment 49, the composition of embodiment 50 or the vector of embodiment 52.

73. The method of embodiment 72, wherein the T cell is transduced with a retroviral or lentiviral vector construct or with a non-viral vector construct.

74. The method of embodiment 73, wherein the non-viral vector construct is a sleeping beauty minicircle vector.

75. The method of any one of embodiments 71 to 74, wherein the transduced T cell is administered to the subject by intravenous infusion.

76. The method of any one of embodiments 71 to 75, wherein the transduced T cell is contacted with anti-CD3 and/or anti-CD28 antibodies prior to administration to the subject.

77. The method of any one of embodiments 71 to 76, wherein the transduced T cell is contacted with at least one cytokine prior to administration to the subject, preferably with interleukin-2 (IL-2), interleukin-7 (IL-7), interleukin-15 (IL-15), and/or interleukin-21, or variants thereof.

78. The method of any one of embodiments 71 to 77, wherein the disease is a malignant disease.

79. The method of any one of embodiments 71 to 78, wherein the disease is selected from cancer of epithelial, endothelial or mesothelial origin and cancer of the blood.

80. A method for inducing lysis of a target cell, comprising contacting the target cell with a transduced T cell capable of expressing the antigen binding receptor of any one of embodiments 1 to 48.

81. The method of embodiment 80, wherein the target cell is a cancer cell. 82. The method of any one of embodiments 80 or 81, wherein the target cell expresses an antigen selected from the group consisting of FAP, CEA, p95, BCMA, EpCAM, MSLN, MCSP, HER-1, HER-2, HER-3, CD19, CD20, CD22, CD33, CD38, CD52Flt3, FOLR1, Trop-2, CA-12-5, HLA-DR, MUC-1 (mucin), A33-antigen, PSMA, PSCA, transferrin-receptor, TNC (tenascin), CA-IX and PDL1.

83. The method of any one of embodiments 80 to 82, wherein the target cell expresses an antigen selected from the group consisting of fibroblast activation protein (FAP), carcinoembryonic antigen (CEA), mesothelin (MSLN), CD20, folate receptor 1 (FOLR1), tenascin (TNC), and programmed death-ligand 1 (PDL1).

84. Use of the antigen binding receptor of any one of embodiments 1 to 48, the polynucleotide of embodiment 49, the composition of embodiment 50, or the transduced T cell of any one of embodiments 53 to 65 for the manufacture of a medicament.

85. The use of embodiment 84, wherein the medicament is for treatment of a malignant disease.

86. The use of embodiment 85, characterized in that said malignant disease is selected from cancer of epithelial, endothelial or mesothelial origin and cancer of the blood.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example, the public database “Medline”, available on the Internet, may be utilized, for example under http://www.ncbi.nlm.nih.gov/PubMed/medline.html. Further databases and addresses, such as http://www.ncbi.nlm.nih.gov/, http://www.infobiogen.fe, http://www.fmi.ch/biology/research tools.html, http://www.tigr.org/, are known to the person skilled in the art and can also be obtained using, e.g., http://www.lycos.com.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE and size exclusion chromatography (SEC).

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2 x PBS on a Dionex HPLC-System. The eluted protein was quantified by UV absorbance and integration of peak areas. BioRad Gel Filtration Standard 151-1901 served as a standard.

Lentiviral Transduction of Jurkat NFAT T Cells

To produce lentiviral vectors, respective DNA sequences for the correct assembly of the antigen binding receptor were cloned in frame in a lentiviral polynucleotide vector under a constitutively active human cytomegalovirus immediate early promoter (CMV). The retroviral vector contained a woodchuck hepatitis virus posttranscriptional regulatory element (WPRE), a central polypurine tract (cPPT) element, a pUC origin of replication and a gene encoding for antibiotic resistance facilitating the propagation and selection in bacteria.

To produce functional virus particles, Lipofectamine LTX™ based transfection was performed using 60-70% confluent Hek293T cells (ATCC CRL3216) and CAR containing vectors as well as pCMV-VSV-G:pRSV-REV:pCgpV transfer vectors at 3:1:1:1 ratio. After 48h supernatant was collected, centrifuge for 5 minutes at 250 g to remove cell debris and filtrated through 0.45 or 0.22 μm polyethersulfon filter. Concentrated virus particles (Lenti-x-Concentrator, Takara) were used to transduce Jurkat NFAT cells (Signosis). Positive transduced cells were sorted as pool or single clones using FACSARIA sorter (BD Bioscience). After cell expansion to appropriate density Jurkat NFAT T cells were used for experiments.

### Example 1

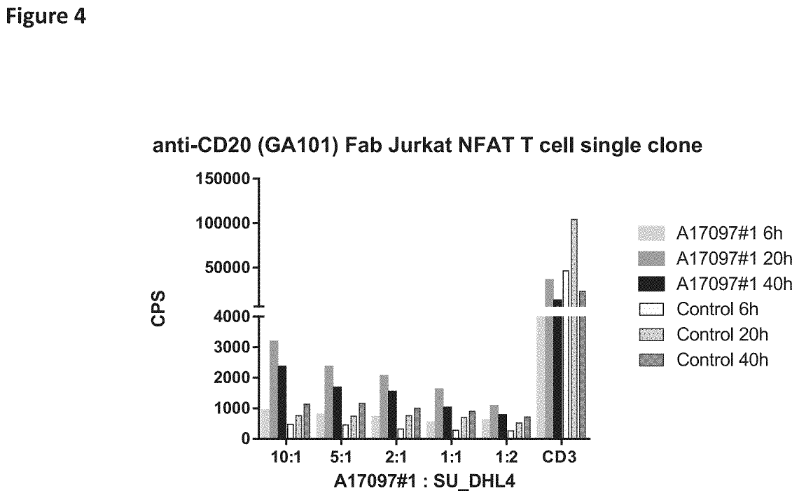

Described herein is a Jurkat NFAT T cell reporter assay using CD20 expressing SUDHDL4 tumor cells as target cells and a sorted single clone of Anti-CD20-Fab-CD28ATD-CD28CSD-CD3zSSD expressing Jurkat NFAT T cells as target cells (FIG. 4). As positive control, some wells of a 96 well plate (Cellstar Greiner-bio-one, CAT-No. 655185) were coated with 10 λg/ml CD3 antibody (from Biolegend®) in phosphate buffered saline (PBS) either for 4° C. over night or for at least 1 h at 37° C. The CD3 antibody coated wells were washed twice with PBS, after the final washing step PBS was fully removed. Jurkat NFAT wild type cells or Jurkat NFAT CAR cells engineered to express the antigen binding receptor Anti-CD20-Fab-CD28ATD-CD28CSD-CD3zS SD (further termed as effector cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and effector cells were plated in 10:1, 5:1, 2:1 or 1:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows the activation of Anti-CD20-Fab-CD28ATD-CD28CSD-CD3zSSD expressing Jurkat NFAT T cells dependent on different E:T ratios and dependent of the time of co-cultivation with target cells. It is shown that Jurkat NFAT T cell activation is dependent on the duration of the co-cultivation with target cells and dependent on the E:T ratio. For all tested conditions an incubation time of 20 hours displays the highest luminescence signal. Further, among the different E:T ratios the 10:1 E:T ratio depicts the highest detectable luminescence signal. Jurkat NFAT wild type T cells show only a time dependent increase in luminescence signal, whereby after 40 hours the highest luminescence signal can be detected. The detected luminescence signal is independent of E:T ratio and in general also clearly lower than each luminescence signal detected for Anti-CD20-Fab-CD28ATD-CD28CSD-CD3zS SD expressing Jurkat NFAT T cells at the respective time points. In general, the highest luminescence signal is detectable if cells were incubated in CD3 antibody coated wells. The Anti-CD20-Fab-CD28ATD-CD28CSD-CD3zSSD expressing Jurkat NFAT T cells depict a higher signal compared to not transduced Jurkat NFAT control T cells. Each point represents the mean of a technical duplicate.

### Example 2

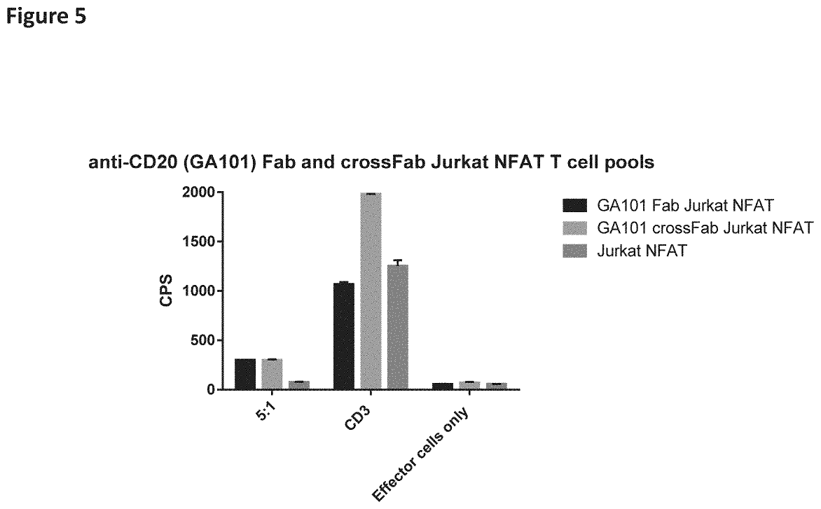

Described herein is a Jurkat NFAT T cell reporter assay using CD20 expressing SUDHDL4 tumor cells as target cells and a sorted single clone of Anti-CD20-Fab-CD28ATD-CD28CSD-CD3zSSD or Anti-CD20-crossFab-CD28ATD-CD28CSD-CD3zSSD expressing Jurkat NFAT T cells as target cells (FIG. 5). As positive control, wells of a 96 well plate (Cellstar Greiner-bio-one, CAT-No. 655185) were coated with 10 μg/ml CD3 antibody (from Biolegend®) in phosphate buffered saline (PBS) at 4° C. over night. The CD3 antibody coated wells were washed twice with PBS, after the final washing step PBS was fully removed. Jurkat NFAT wild type cells or Jurkat NFAT T cells engineered to express Anti-CD20-Fab-CD28ATD-CD28CSD-CD3zSSD or Anti-CD20-crossFab-CD28ATD-CD28CSD-CD3zSSD (further termed as effector cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells, expressing the antigen of interest were counted and checked for their viability as well. Cell number was adjusted to 1×10⁶ viable cells/ml in growth medium. Target cells and effector cells were plated in 5:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows activation of Anti-CD20-Fab-CD28ATD-CD28CSD-CD3zSSD expressing Jurkat NFAT T cells and Anti-CD20-crossFab-CD28ATD-CD28CSD-CD3zSSD expressing Jurkat NFAT T cells upon co-cultivation with target cells. If Anti-CD20-Fab-CD28ATD-CD28CSD-CD3zSSD or Anti-CD20-crossFab-CD28ATD-CD28CSD-CD3zSSD expressing Jurkat NFAT T cells or Jurkat NFAT control T cells were cultivated without target cells, no luminescence signal was detected. The highest luminescence signal was detected when either Anti-CD20-Fab-CD28ATD-CD28CSD-CD3zSSD or Anti-CD20-crossFab-CD28ATD-CD28CSD-CD3zSSD expressing Jurkat NFAT T cells or Jurkat NFAT control T cells were co-cultivated with target cells in CD3 antibody coated plates. Surprisingly, the crossFab format leads to strong activation of Jurkat NFAT T cells in conjunction with CD3 mediated signaling. Each point represents the mean value of technical triplicates. Standard deviation is indicated by error bars.

### Example 3

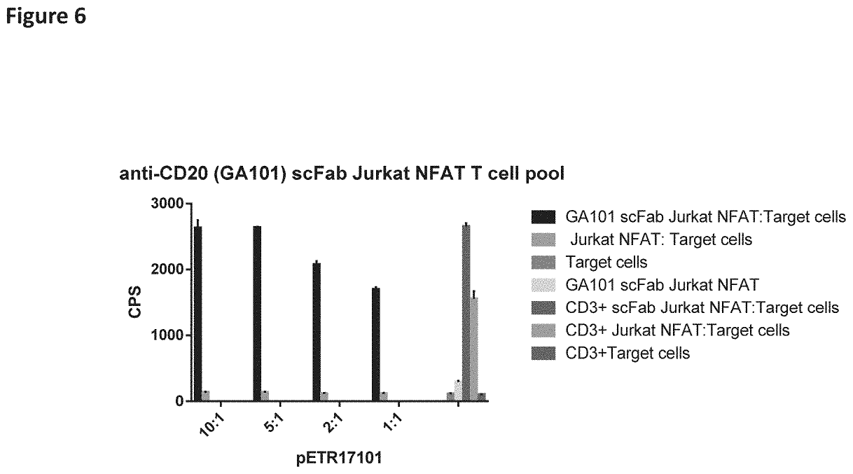

Described herein is a Jurkat NFAT T cell reporter assay performed using CD20 expressing SUDHDL4 tumor cells as target cells and a sorted pool of Anti-CD20-scFab-CD28ATD-CD28CSD-CD3zSSD expressing Jurkat NFAT T cells as target cells (FIG. 6).

As positive control, wells of a 96 well plate (Cellstar Greiner-bio-one, CAT-No. 655185) were coated with 10 μg/ml CD3 antibody (from Biolegend®) in phosphate buffered saline (PBS) either for 4° C. over night or for at least 1 h at 37° C. The CD3 antibody coated wells were washed twice with PBS, after the final washing step PBS was fully removed. Jurkat NFAT wild type T cells or Jurkat NFAT T cells engineered to express Anti-CD20-scFab-CD28ATD-CD28CSD-CD3zSSD (further termed as effector cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and effector cells were plated in 10:1, 5:1, 2:1 or 1:1 E:T ratio (110.000 cells per well in total) in triplicates in a 96-well suspension culture plate (Greiner-bio one) in a final volume of 200 After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows the activation of Anti-CD20-scFab-CD28ATD-CD28CSD-CD3zSSD expressing Jurkat NFAT T cells after 20 hours co-cultivation with SUDHL4 target cells in different E:T ratios. Among the different E:T ratios, the 10:1 and 5:1 E:T ratio show the highest luminescence signal (FIG. 6 black bars). Also Anti-CD20-scFab-CD28ATD-CD28CSD-CD3zSSD expressing JurkatNFAT T cells co-cultured at 10:1 E:T ratio in CD3 antibody coated wells, show a high luminescence signal comparable to the same condition without CD3 stimulus.

Further Jurkat NFAT wild type cells do not show any activation independent of different E:T ratios, but if co-cultivated in 10:1 E:T ratio in CD3 antibody coated wells a clear luminescence signal is delectable, that proves their functionality.

Further control experiments show that target cells or Anti-CD20-scFab-CD28ATD-CD28CSD-CD3zSSD expressing T cells alone as well as CD3 antibody coated wells with target cells do not show any activation. Each point represents the mean value of technical triplicates. Standard deviation is indicated by error bars.

### Example 4

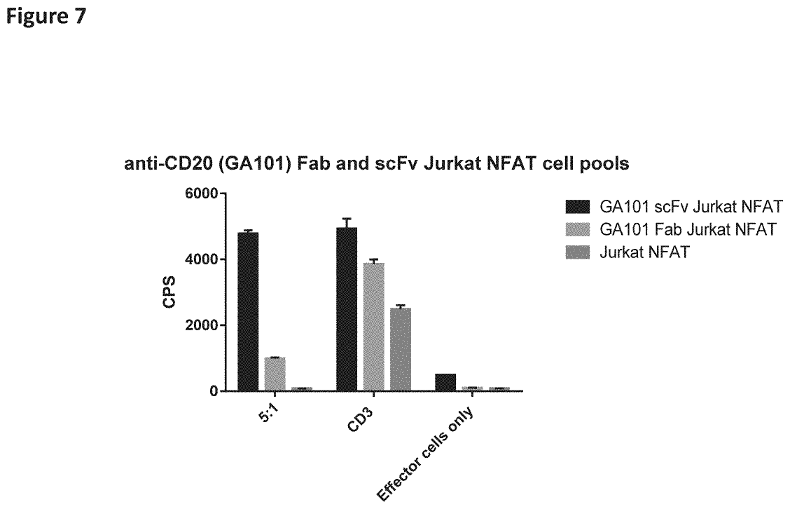

Described herein is a Jurkat NFAT T cell reporter assay performed using CD20 expressing SUDHDL4 tumor cells as target cells and a sorted pool of anti-CD20-Fab-CD28ATD-CD28CSD-CD3zSSD expressing Jurkat NFAT T cells or anti-CD20-scFv-CD28ATD-CD28CSD-CD3zSSD expressing Jurkat NFAT T cells as target cells (FIG. 7).

As positive control, wells of a 96 well plate (Cellstar Greiner-bio-one, CAT-No. 655185) were coated with 10 μg/ml CD3 antibody (from Biolegend®) in phosphate buffered saline (PBS) either for 4° C. over night or for at least 1 h at 37° C. The CD3 antibody coated wells were washed twice with PBS, after the final washing step PBS was fully removed. Jurkat NFAT wild type T cells or Jurkat NFAT T cells engineered to express anti-CD20-Fab-CD28ATD-CD28CSD-CD3zSSD or anti-CD20-scFv-CD28ATD-CD28CSD-CD3zSSD (further termed as effector cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and effector cells were plated in 10:1, 5:1, 2:1 or 1:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows the activation of Anti-CD20-scFv-CD28ATD-CD28CSD-CD3zSSD expressing Jurkat NFAT T cells after 20 hours co-cultivation with SUDHL4 target cells at 5:1 E:T ratio. Anti-CD20-scFv-CD28ATD-CD28CSD-CD3zSSD expressing Jurkat NFAT T cells co-cultured with target cells in CD3 antibody coated wells, showed the highest luminescence signal, which is comparable to the same condition without CD3 stimulus. Surprisingly, the crossFab format leads to differentiated activation of Jurkat NFAT T cells wherein strong activation is found in conjunction with CD3 mediated signaling. Further Jurkat NFAT wild type cells do not show any activation, but if co-cultivated in 10:1 E:T ratio in CD3 antibody coated wells a clear luminescence signal is delectable, that proves their functionality. Each bar represents the mean value of technical triplicates. Standard deviation is indicated by error bars.

### Example 5

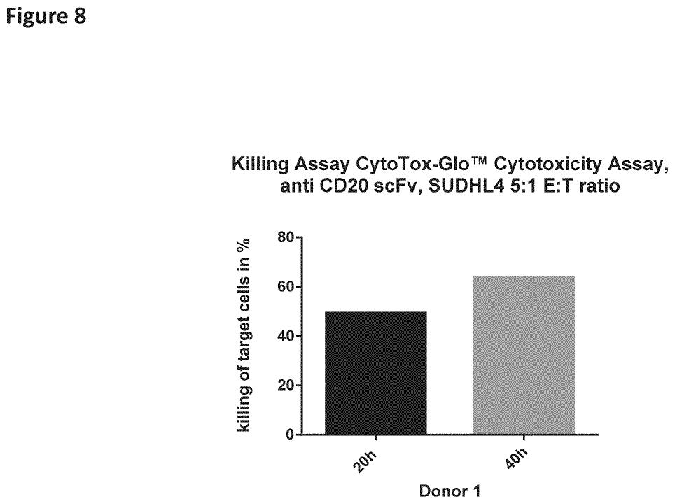

Described herein is a killing assay performed using CD20 expressing SUDHDL4 tumor cells as target cells and a pool of anti-CD20-scFv-CD28ATD-CD28CSD-CD3zSSD expressing T cells as target cells (FIG. 8).

Frozen PBMCs were thawed and seeded in T cell medium (CTS™ OpTmizer™ T Cell Expansion SFM, Cat. A1048501 plus 200 U/ml IL-2) in CD3/CD28 coated wells to activate them for two days. In parallel HEK cells were transiently transfected to produce virus particles. T cells were transduced using spinnfection at 32° C., 800 rpm for 90 min.

2 Mio SUDHL4 cells were irradiated for 1 min and 59 s at 5000 rad and seeded in a 96 well plate. Transduced T cells were seeded on top and co-cultivated for 5 days. Cells were then harvested and put on puromycine selection (1 ug/ml) for additional 3 days to get rid of the feeder cells and not transduced T cells. Remaining T cells, further termed as effector cells, were harvested, counted and checked for their viability. Appropriate cell number was spun down and resuspended in T cell medium. Effector cells were seeded in a volume of 10 μl. SUDHL4 were harvested, counted and checked for their viability. Cell number was adjusted to achieve a 5:1 E: T ratio. The final volume per well in a 384 well plate was 20 μl. As control for spontaneous and maximal release, target cells only were seeded in volume of 10 μl and topped up to a total volume of 204 Further effector cells only, were seeded in 10 μl and topped up to 20 μl with T cells medium.

After incubation time of 20 hours or 40 hours the CytoTox-Glo™ Cytotoxicity Assay from Promega (Cat. G9291) was used to detect caspase activity of dead cells. To determine the maximal release of the target cells 12 μl of Lysis Buffer was added to the appropriate wells and the plate was incubated for 15 min on a rotary shaker (Eppendorf, 300 rpm). After that 12 μl Assay buffer was added to all of the wells and the plate was incubated for another 10 min on rotary shaker. Luminescence was measured for 0.5 sec at a luminescence plate reader (Victor). To calculate the killing data, the sum of the spontaneous release of the effector cells as well as the spontaneous release of target cells was calculated and then subtracted from the measured value of the target and effector cells that were co-cultivated. The percentage of killing was further calculated by comparing it to the maximal release at 100%. The bar diagramm displays the mean of a technical triplicate representing the percentage of killing by the anti-CD20 transduced CART cells after 20 hours and 40 hours.

Exemplary Sequences

TABLE 2
Anti-CD20 Fab amino acid sequences

| | | SEQ |
|---|---|---|
| Construct | Protein Sequence | ID NO |
| Anti-CD20 (GA101) | YSWIN | 1 |
| CDR H1 Kabat | | |
| Anti-CD20 (GA101) | RIFPGDGDTDYNGKFKG | 2 |
| CDR H2 Kabat | | |
| Anti-CD20 (GA101) | NVFDGYWLVY | 3 |
| CDR H3 Kabat | | |
| Anti-CD20 (GA101) | RSSKSLLHSNGITYLY | 4 |
| CDR L1 Kabat | | |
| Anti-CD20 (GA101) | QMSNLVS | 5 |
| CDR L2 Kabat | | |
| Anti-CD20 (GA101) | AQNLELPYT | 6 |
| CDR L3 Kabat | | |
| Anti-CD20-(GA101)- | QVQLVQSGAEVKKPGSSVKVSCKASGYAFSYSWINWVR | 7 |
| Fab heavy chain- | QAPGQGLEWMGRIFPGDGDTDYNGKFKGRVTITADKST | |
| CD28ATD- | STAYMELSSLRSEDTAVYYCARNVFDGYWLVYWGQGT | |
| CD28CSD- | LVTVSSASTKGPSVFPLAPSSKSTSGGTAALGCLVKDYFP | |
| CD3zSSD fusion | EPVTVSWNSGALTSGVHTFPAVLQSSGLYSLSSVVTVPSS | |
| pETR 17097 | SLGTQTYICNVNHKPSNTKVDKKVEPKSCGGGGSFWVL | |
| | VVVGGVLACYSLLVTVAFIIFWVRSKRSRLLHSDYMNM | |
| | TPRRPGPTRKHYQPYAPPRDFAAYRSRVKFSRSADAPAY | |
| | QQGQNQLYNELNLGRREEYDVLDKRRGRDPEMGGKPR | |
| | RKNPQEGLYNELQKDKMAEAYSEIGMKGERRRGKGHD | |
| | GLYQGLSTATKDTYDALHMQALPPR | |
| Anti-CD20-(GA101)- | QVQLVQSGAEVKKPGSSVKVSCKASGYAFSYSWINWVR | 8 |
| Fab heavy chain | QAPGQGLEWMGRIFPGDGDTDYNGKFKGRVTITADKST | |
| | STAYMELSSLRSEDTAVYYCARNVFDGYWLVYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPSS | |
| | SLGTQTYICNVNHKPSNTKVDKKVEPKSC | |
| Anti-CD20-(GA101)- | DIVMTQTPLSLPVTPGEPASISCRSSKSLLHSNGITYLYWY | 9 |
| Fab light chain | LQKPGQSPQLLIYQMSNLVSGVPDRFSGSGSGTDFTLKIS | |
| | RVEAEDVGVYYCAQNLELPYTFGGGTKVEIKRTVAAPS | |
| | VFIFPPSDEQLKSGTASVVCLLNNFYPREAKVQWKVDNA | |
| | LQSGNSQESVIEQDSKDSTYSLSSTLTLSKADYEKHKVY | |
| | ACEVTHQGLSSPVTKSFNRGEC | |
| Anti-CD20-(GA101) | DIVMTQTPLSLPVTPGEPASISCRSSKSLLHSNGITYLYWY | 10 |
| VL | LQKPGQSPQLLIYQMSNLVSGVPDRFSGSGSGTDFTLKIS | |
| | RVEAEDVGVYYCAQNLELPYTFGGGTKVEIK | |
| CL | RTVAAPSVFIFPPSDEQLKSGTASVVCLLNNFYPREAKVQ | 11 |
| | WKVDNALQSGNSQESVTEQDSKDSTYSLSSTLTLSKADY | |
| | EKHKVYACEVTHQGLSSPVTKSFNRGEC | |
| Anti-CD20-(GA101) | QVQLVQSGAEVKKPGSSVKVSCKASGYAFSYSWINWVR | 12 |
| VH | QAPGQGLEWMGRIFPGDGDTDYNGKFKGRVTITADKST | |
| | STAYMELSSLRSEDTAVYYCARNVFDGYWLVYWGQGT | |
| | LVTVSS | |
| CH1 | ASTKGPSVFPLAPSSKSTSGGTAALGCLVKDYFPEPVTVS | 13 |
| | WNSGALTSGVHTFPAVLQSSGLYSLSSVVTVPSSSLGTQT | |
| | YICNVNHKPSNTKVDKKVEPKSC | |
| CD28ATD | FWVLVVVGGVLACYSLLVTVAFIIFWV | 14 |
| CD28CSD | RSKRSRLLHSDYMNMTPRRPGPTRKHYQPYAPPRDFAA | 15 |
| | YRS | |
| CD3zSSD | RVKFSRSADAPAYQQGQNQLYNELNLGRREEYDVLDKR | 16 |
| | RGRDPEMGGKPRRKNPQEGLYNELQKDKMAEAYSEIG | |
| | MKGERRRGKGHDGLYQGLSTATKDTYDALHMQALPPR | |
| CD28ATD-CD28CSD- | FWVLVVVGGVLACYSLLVTVAFIIFWVRSKRSRLLHSDY | 17 |
| CD3zSSD | MNMTPRRPGPTRKHYQPYAPPRDFAAYRSRVKFSRSAD | |
| | APAYQQGQNQLYNELNLGRREEYDVLDKRRGRDPEMG | |
| | GKPRRKNPQEGLYNELQKDKMAEAYSEIGMKGERRRGK | |
| | GHDGLYQGLSTATKDTYDALHMQALPPR | |
| eGFP | VSKGEELFTGVVPILVELDGDVNGHKFSVSGEGEGDATY | 18 |
| | GKLTLKFICTTGKLPVPWPTLVTTLTYGVQCFSRYPDHM | |
| | KQHDFFKSAMPEGYVQERTIFFKDDGNYKTRAEVKFEG | |
| | DTLVNRIELKGIDFKEDGNILGHKLEYNYNSHNVYIMAD | |
| | KQKNGIKVNFKIRHNIEDGSVQLADHYQQNTPIGDGPVL | |
| | LPDNHYLSTQSALSKDPNEKRDHMVLLEFVTAAGITLGM | |
| | DELYK | |
| (G4S)4 linker | GGGGSGGGGSGGGGSGGGGS | 19 |
| G4S linker | GGGGS | 20 |
| T2A linker | GEGRGSLLTCGDVEENPGP | 21 |

TABLE 3
Anti-CD20 Fab DNA sequences

| | | SEQ |
|---|---|---|
| Construct | DNA Sequenz | ID NO |
| Anti-CD20-(GA101)- | ATGGGATGGAGCTGTATCATCCTCTTCTTGGTAGCAAC | 22 |
| Fab- | AGCTACCGGTGTGCATTCCGATATCGTGATGACCCAG | |
| CD28ATD- | ACTCCACTCTCCCTGCCCGTCACCCCTGGAGAGCCCGC | |
| CD28CSD- | CAGCATTAGCTGCAGGTCTAGCAAGAGCCTCTTGCAC | |
| CD3zSSD | AGCAATGGCATCACTTATTTGTATTGGTACCTGCAAAA | |
| pETR17097 | GCCAGGGCAGTCTCCACAGCTCCTGATTTATCAAATGT | |
| | CCAACCTTGTCTCTGGCGTCCCTGACCGGTTCTCCGGA | |
| | TCCGGGTCAGGCACTGATTTCACACTGAAAATCAGCA | |
| | GGGTGGAGGCTGAGGATGTTGGAGTTTATTACTGCGC | |
| | TCAGAATCTAGAACTTCCTTACACCTTCGGCGGAGGG | |
| | ACCAAGGTGGAGATCAAACCGTACGGTGGCTGCACCA | |
| | TCTGTCTTCATCTTCCCGCCATCTGATGAGCAGTTGAA | |
| | ATCTGGAACTGCCTCTGTTGTGTGCCTGCTGAATAACT | |
| | TCTATCCCAGAGAGGCCAAAGTACAGTGGAAGGTGGA | |
| | TAACGCCCTCCAATCGGGTAACTCCCAGGAGAGTGTC | |
| | ACAGAGCAGGACAGCAAGGACAGCACCTACAGCCTCA | |
| | GCAGCACCCTGACGCTGAGCAAAGCAGACTACGAGAA | |
| | ACACAAAGTCTACGCCTGCGAAGTCACCCATCAGGGC | |
| | CTGAGCTCGCCCGTCACAAAGAGCTTCAACAGGGGAG | |
| | AGTGTTAGAATAGAATTCCCCGAAGTAACTTAGAAGC | |
| | TGTAAATCAACGATCAATAGCAGGTGTGGCACACCAG | |
| | TCATACCTTGATCAAGCACTTCTGTTTCCCCGGACTGA | |
| | GTATCAATAGGCTGCTCGCGCGGCTGAAGGAGAAAAC | |
| | GTTCGTTACCCGACCAACTACTTCGAGAAGCTTAGTAC | |
| | CACCATGAACGAGGCAGGGTGTTTCGCTCAGCACAAC | |
| | CCCAGTGTAGATCAGGCTGATGAGTCACTGCAACCCC | |
| | CATGGGCGACCATGGCAGTGGCTGCGTTGGCGGCCTG | |
| | CCCATGGAGAAATCCATGGGACGCTCTAATTCTGACA | |
| | TGGTGTGAAGTGCCTATTGAGCTAACTGGTAGTCCTCC | |
| | GGCCCCTGATTGCGGCTAATCCTAACTGCGGAGCACA | |
| | TGCTCACAAACCAGTGGGTGGTGTGTCGTAACGGGCA | |
| | ACTCTGCAGCGGAACCGACTACTTTGGGTGTCCGTGTT | |
| | TCCTTTTATTCCTATATTGGCTGCTTATGGTGACAATCA | |
| | AAAAGTTGTTACCATATAGCTATTGGATTGGCCATCCG | |
| | GTGTGCAACAGGGCAACTGTTTACCTATTTATTGGTTT | |
| | TGTACCATTATCACTGAAGTCTGTGATCACTCTCAAAT | |
| | TCATTTTGACCCTCAACACAATCAAACGCCACCATGGG | |
| | ATGGAGCTGTATCATCCTCTTCTTGGTAGCAACAGCTA | |
| | CCGGTGTGCACTCCCAGGTGCAATTGGTGCAGTCTGGC | |
| | GCTGAAGTTAAGAAGCCTGGGAGTTCAGTGAAGGTCT | |
| | CCTGCAAGGCTTCGGGATACGCCTTCAGCTATTCTTGG | |
| | ATCAATTGGGTGCGGCAGGCGCCTGGACAAGGGCTCG | |
| | AGTGGATGGGACGGATCTTTCCCGGCGATGGGGATAC | |
| | TGACTACAATGGGAAATTCAAGGGCAGAGTCACAATT | |
| | ACCGCCGACAAATCCACTAGCACAGCCTATATGGAGC | |
| | TGAGCAGCCTGAGATCTGAGGACACGGCCGTGTATTA | |
| | CTGTGCAAGAAATGTCTTTGATGGTTACTGGCTTGTTT | |
| | ACTGGGGCCAGGGAACCCTGGTCACCGTCTCCAGCGC | |
| | TAGCACCAAGGGCCCCTCCGTGTTCCCCCTGGCCCCCA | |
| | GCAGCAAGAGCACCAGCGGCGGCACAGCCGCTCTGGG | |
| | CTGCCTGGTCAAGGACTACTTCCCCGAGCCCGTGACCG | |
| | TGTCCTGGAACAGCGGAGCCCTGACCTCCGGCGTGCA | |
| | CACCTTCCCCGCCGTGCTGCAGAGTTCTGGCCTGTATA | |
| | GCCTGAGCAGCGTGGTCACCGTGCCTTCTAGCAGCCTG | |
| | GGCACCCAGACCTACATCTGCAACGTGAACCACAAGC | |
| | CCAGCAACACCAAGGTGGACAAGAAGGTGGAGCCCA | |
| | AGAGCTGCGGAGGGGGCGGATCCTTCTGGGTGCTGGT | |
| | GGTGGTGGGCGGCGTGCTGGCCTGCTACAGCCTGCTG | |
| | GTGACCGTGGCCTTCATCATCTTCTGGGTGAGGAGCAA | |
| | GAGGAGCAGGCTGCTGCACAGCGACTACATGAACATG | |
| | ACCCCCAGGAGGCCCGGCCCCACCAGGAAGCACTACC | |
| | AGCCCTACGCCCCCCCCAGGGACTTCGCCGCCTACAG | |
| | GAGCAGGGTGAAGTTCAGCAGGAGCGCCGACGCCCCC | |
| | GCCTACCAGCAGGGCCAGAACCAGCTGTATAACGAGC | |
| | TGAACCTGGGCAGGAGGGAGGAGTACGACGTGCTGGA | |
| | CAAGAGGAGGGGCAGGGACCCCGAGATGGGCGGCAA | |
| | GCCCAGGAGGAAGAACCCCCAGGAGGGCCTGTATAAC | |
| | GAGCTGCAGAAGGACAAGATGGCCGAGGCCTACAGC | |
| | GAGATCGGCATGAAGGGCGAGAGGAGGAGGGGCAAG | |
| | GGCCACGACGGCCTGTACCAGGGCCTGAGCACCGCCA | |
| | CCAAGGACACCTACGACGCCCTGCACATGCAGGCCCT | |
| | GCCCCCCAGG | |
| Anti-CD20-(GA101)- | GATATCGTGATGACCCAGACTCCACTCTCCCTGCCCGT | 23 |
| Fab-VL | CACCCCTGGAGAGCCCGCCAGCATTAGCTGCAGGTCT | |
| | AGCAAGAGCCTCTTGCACAGCAATGGCATCACTTATTT | |
| | GTATTGGTACCTGCAAAAGCCAGGGCAGTCTCCACAG | |
| | CTCCTGATTTATCAAATGTCCAACCTTGTCTCTGGCGT | |
| | CCCTGACCGGTTCTCCGGATCCGGGTCAGGCACTGATT | |
| | TCACACTGAAAATCAGCAGGGTGGAGGCTGAGGATGT | |
| | TGGAGTTTATTACTGCGCTCAGAATCTAGAACTTCCTT | |
| | ACACCTTCGGCGGAGGGACCAAGGTGGAGATCAAA | |
| Fab CL | CGTACGGTGGCTGCACCATCTGTCTTCATCTTCCCGCC | 24 |
| | ATCTGATGAGCAGTTGAAATCTGGAACTGCCTCTGTTG | |
| | TGTGCCTGCTGAATAACTTCTATCCCAGAGAGGCCAA | |
| | AGTACAGTGGAAGGTGGATAACGCCCTCCAATCGGGT | |
| | AACTCCCAGGAGAGTGTCACAGAGCAGGACAGCAAG | |
| | GACAGCACCTACAGCCTCAGCAGCACCCTGACGCTGA | |
| | GCAAAGCAGACTACGAGAAACACAAAGTCTACGCCTG | |
| | CGAAGTCACCCATCAGGGCCTGAGCTCGCCCGTCACA | |
| | AAGAGCTTCAACAGGGGAGAGTGTTAG | |
| Anti-CD20-(GA101)- | CAGGTGCAATTGGTGCAGTCTGGCGCTGAAGTTAAGA | 25 |
| Fab-VH | AGCCTGGGAGTTCAGTGAAGGTCTCCTGCAAGGCTTC | |
| | GGGATACGCCTTCAGCTATTCTTGGATCAATTGGGTGC | |
| | GGCAGGCGCCTGGACAAGGGCTCGAGTGGATGGGACG | |
| | GATCTTTCCCGGCGATGGGGATACTGACTACAATGGG | |
| | AAATTCAAGGGCAGAGTCACAATTACCGCCGACAAAT | |
| | CCACTAGCACAGCCTATATGGAGCTGAGCAGCCTGAG | |
| | ATCTGAGGACACGGCCGTGTATTACTGTGCAAGAAAT | |
| | GTCTTTGATGGTTACTGGCTTGTTTACTGGGGCCAGGG | |
| | AACCCTGGTCACCGTCTCCAGC | |
| Fab CH1 | GCTAGCACCAAGGGCCCCTCCGTGTTCCCCCTGGCCCC | 26 |
| | CAGCAGCAAGAGCACCAGCGGCGGCACAGCCGCTCTG | |
| | GGCTGCCTGGTCAAGGACTACTTCCCCGAGCCCGTGA | |
| | CCGTGTCCTGGAACAGCGGAGCCCTGACCTCCGGCGT | |
| | GCACACCTTCCCCGCCGTGCTGCAGAGTTCTGGCCTGT | |
| | ATAGCCTGAGCAGCGTGGTCACCGTGCCTTCTAGCAG | |
| | CCTGGGCACCCAGACCTACATCTGCAACGTGAACCAC | |
| | AAGCCCAGCAACACCAAGGTGGACAAGAAGGTGGAG | |
| | CCCAAGAGCTGC | |
| IRES EV71 internal | CCCGAAGTAACTTAGAAGCTGTAAATCAACGATCAAT | 27 |
| ribosomal entry side | AGCAGGTGTGGCACACCAGTCATACCTTGATCAAGCA | |
| | CTTCTGTTTCCCCGGACTGAGTATCAATAGGCTGCTCG | |
| | CGCGGCTGAAGGAGAAAACGTTCGTTACCCGACCAAC | |
| | TACTTCGAGAAGCTTAGTACCACCATGAACGAGGCAG | |
| | GGTGTTTCGCTCAGCACAACCCCAGTGTAGATCAGGCT | |
| | GATGAGTCACTGCAACCCCCATGGGCGACCATGGCAG | |
| | TGGCTGCGTTGGCGGCCTGCCCATGGAGAAATCCATG | |
| | GGACGCTCTAATTCTGACATGGTGTGAAGTGCCTATTG | |
| | AGCTAACTGGTAGTCCTCCGGCCCCTGATTGCGGCTAA | |
| | TCCTAACTGCGGAGCACATGCTCACAAACCAGTGGGT | |
| | GGTGTGTCGTAACGGGCAACTCTGCAGCGGAACCGAC | |
| | TACTTTGGGTGTCCGTGTTTCCTTTTATTCCTATATTGG | |
| | CTGCTTATGGTGACAATCAAAAAGTTGTTACCATATAG | |
| | CTATTGGATTGGCCATCCGGTGTGCAACAGGGCAACT | |
| | GTTTACCTATTTATTGGTTTTGTACCATTATCACTGAAG | |
| | TCTGTGATCACTCTCAAATTCATTTTGACCCTCAACAC | |
| | AATCAAAC | |
| G4S linker | GGAGGGGGCGGATCC | 28 |
| CD28ATD | TTTTGGGTGCTGGTGGTGGTTGGTGGAGTCCTGGCTTG | 29 |
| | CTATAGCTTGCTAGTAACAGTGGCCTTTATTATTTTCT | |
| | GGGTG | |
| CD28CSD | AGGAGTAAGAGGAGCAGGCTCCTGCACAGTGACTACA | 30 |
| | TGAACATGACTCCCCGCCGCCCCGGGCCCACCCGCAA | |
| | GCATTACCAGCCCTATGCCCCACCACGCGACTTCGCAG | |
| | CCTATCGCTCC | |
| CD3zSSD | AGAGTGAAGTTCAGCAGGAGCGCAGACGCCCCCGCGT | 31 |
| | ACCAGCAGGGCCAGAACCAGCTCTATAACGAGCTCAA | |
| | TCTAGGACGAAGAGAGGAGTACGATGTTTTGGACAAG | |
| | AGACGTGGCCGGGACCCTGAGATGGGGGGAAAGCCG | |
| | AGAAGGAAGAACCCTCAGGAAGGCCTGTACAATGAAC | |
| | TGCAGAAAGATAAGATGGCGGAGGCCTACAGTGAGAT | |
| | TGGGATGAAAGGCGAGCGCCGGAGGGGCAAGGGGCA | |
| | CGATGGCCTTTACCAGGGTCTCAGTACAGCCACCAAG | |
| | GACACCTACGACGCCCTTCACATGCAGGCCCTGCCCCC | |
| | TCGC | |
| CD28ATD-CD28CSD- | TTCTGGGTGCTGGTGGTGGTGGGCGGCGTGCTGGCCTG | 32 |
| CD3zSSD | CTACAGCCTGCTGGTGACCGTGGCCTTCATCATCTTCT | |
| | GGGTGAGGAGCAAGAGGAGCAGGCTGCTGCACAGCG | |
| | ACTACATGAACATGACCCCCAGGAGGCCCGGCCCCAC | |
| | CAGGAAGCACTACCAGCCCTACGCCCCCCCCAGGGAC | |
| | TTCGCCGCCTACAGGAGCAGGGTGAAGTTCAGCAGGA | |
| | GCGCCGACGCCCCCGCCTACCAGCAGGGCCAGAACCA | |
| | GCTGTATAACGAGCTGAACCTGGGCAGGAGGGAGGAG | |
| | TACGACGTGCTGGACAAGAGGAGGGGCAGGGACCCC | |
| | GAGATGGGCGGCAAGCCCAGGAGGAAGAACCCCCAG | |
| | GAGGGCCTGTATAACGAGCTGCAGAAGGACAAGATGG | |
| | CCGAGGCCTACAGCGAGATCGGCATGAAGGGCGAGA | |
| | GGAGGAGGGGCAAGGGCCACGACGGCCTGTACCAGG | |
| | GCCTGAGCACCGCCACCAAGGACACCTACGACGCCCT | |
| | GCACATGCAGGCCCTGCCCCCCAGG | |
| T2A linker | TCCGGAGAGGGCAGAGGAAGTCTTCTAACATGCGGTG | 33 |
| | ACGTGGAGGAGAATCCCGGCCCTAGG | |
| eGFP | GTGAGCAAGGGCGAGGAGCTGTTCACCGGGGTGGTGC | 34 |
| | CCATCCTGGTCGAGCTGGACGGCGACGTAAACGGCCA | |
| | CAAGTTCAGCGTGTCCGGCGAGGGCGAGGGCGATGCC | |
| | ACCTACGGCAAGCTGACCCTGAAGTTCATCTGCACCA | |
| | CCGGCAAGCTGCCCGTGCCCTGGCCCACCCTCGTGACC | |
| | ACCCTGACCTACGGCGTGCAGTGCTTCAGCCGCTACCC | |
| | CGACCACATGAAGCAGCACGACTTCTTCAAGTCCGCC | |
| | ATGCCCGAAGGCTACGTCCAGGAGCGCACCATCTTCTT | |
| | CAAGGACGACGGCAACTACAAGACCCGCGCCGAGGTG | |
| | AAGTTCGAGGGCGACACCCTGGTGAACCGCATCGAGC | |
| | TGAAGGGCATCGACTTCAAGGAGGACGGCAACATCCT | |
| | GGGGCACAAGCTGGAGTACAACTACAACAGCCACAAC | |
| | GTCTATATCATGGCCGACAAGCAGAAGAACGGCATCA | |
| | AGGTGAACTTCAAGATCCGCCACAACATCGAGGACGG | |
| | CAGCGTGCAGCTCGCCGACCACTACCAGCAGAACACC | |
| | CCCATCGGCGACGGCCCCGTGCTGCTGCCCGACAACC | |
| | ACTACCTGAGCACCCAGTCCGCCCTGAGCAAAGACCC | |
| | CAACGAGAAGCGCGATCACATGGTCCTGCTGGAGTTC | |
| | GTGACCGCCGCCGGGATCACTCTCGGCATGGACGAGC | |
| | TGTACAAGTGA | |
| Anti-CD20-(GA101)- | ATGGGATGGAGCTGTATCATCCTCTTCTTGGTAGCAAC | 35 |
| Fab- | AGCTACCGGTGTGCATTCCGATATCGTGATGACCCAG | |
| CD28ATD- | ACTCCACTCTCCCTGCCCGTCACCCCTGGAGAGCCCGC | |
| CD28CSD- | CAGCATTAGCTGCAGGTCTAGCAAGAGCCTCTTGCAC | |
| CD3zSSD- | AGCAATGGCATCACTTATTTGTATTGGTACCTGCAAAA | |
| eGFP | GCCAGGGCAGTCTCCACAGCTCCTGATTTATCAAATGT | |
| pETR17097 | CCAACCTTGTCTCTGGCGTCCCTGACCGGTTCTCCGGA | |
| | TCCGGGTCAGGCACTGATTTCACACTGAAAATCAGCA | |
| | GGGTGGAGGCTGAGGATGTTGGAGTTTATTACTGCGC | |
| | TCAGAATCTAGAACTTCCTTACACCTTCGGCGGAGGG | |
| | ACCAAGGTGGAGATCAAACGTACGGTGGCTGCACCAT | |
| | CTGTCTTCATCTTCCCGCCATCTGATGAGCAGTTGAAA | |
| | TCTGGAACTGCCTCTGTTGTGTGCCTGCTGAATAACTT | |
| | CTATCCCAGAGAGGCCAAAGTACAGTGGAAGGTGGAT | |
| | AACGCCCTCCAATCGGGTAACTCCCAGGAGAGTGTCA | |
| | CAGAGCAGGACAGCAAGGACAGCACCTACAGCCTCAG | |
| | CAGCACCCTGACGCTGAGCAAAGCAGACTACGAGAAA | |
| | CACAAAGTCTACGCCTGCGAAGTCACCCATCAGGGCC | |
| | TGAGCTCGCCCGTCACAAAGAGCTTCAACAGGGGAGA | |
| | GTGTTAGAATAGAATTCCCCGAAGTAACTTAGAAGCT | |
| | GTAAATCAACGATCAATAGCAGGTGTGGCACACCAGT | |
| | CATACCTTGATCAAGCACTTCTGTTTCCCCGGACTGAG | |
| | TATCAATAGGCTGCTCGCGCGGCTGAAGGAGAAAACG | |
| | TTCGTTACCCGACCAACTACTTCGAGAAGCTTAGTACC | |
| | ACCATGAACGAGGCAGGGTGTTTCGCTCAGCACAACC | |
| | CCAGTGTAGATCAGGCTGATGAGTCACTGCAACCCCC | |
| | ATGGGCGACCATGGCAGTGGCTGCGTTGGCGGCCTGC | |
| | CCATGGAGAAATCCATGGGACGCTCTAATTCTGACAT | |
| | GGTGTGAAGTGCCTATTGAGCTAACTGGTAGTCCTCCG | |
| | GCCCCTGATTGCGGCTAATCCTAACTGCGGAGCACAT | |
| | GCTCACAAACCAGTGGGTGGTGTGTCGTAACGGGCAA | |
| | CTCTGCAGCGGAACCGACTACTTTGGGTGTCCGTGTTT | |
| | CCTTTTATTCCTATATTGGCTGCTTATGGTGACAATCA | |
| | AAAAGTTGTTACCATATAGCTATTGGATTGGCCATCCG | |
| | GTGTGCAACAGGGCAACTGTTTACCTATTTATTGGTTT | |
| | TGTACCATTATCACTGAAGTCTGTGATCACTCTCAAAT | |
| | TCATTTTGACCCTCAACACAATCAAACGCCACCATGGG | |
| | ATGGAGCTGTATCATCCTCTTCTTGGTAGCAACAGCTA | |
| | CCGGTGTGCACTCCCAGGTGCAATTGGTGCAGTCTGGC | |
| | GCTGAAGTTAAGAAGCCTGGGAGTTCAGTGAAGGTCT | |
| | CCTGCAAGGCTTCGGGATACGCCTTCAGCTATTCTTGG | |
| | ATCAATTGGGTGCGGCAGGCGCCTGGACAAGGGCTCG | |
| | AGTGGATGGGACGGATCTTTCCCGGCGATGGGGATAC | |
| | TGACTACAATGGGAAATTCAAGGGCAGAGTCACAATT | |
| | ACCGCCGACAAATCCACTAGCACAGCCTATATGGAGC | |
| | TGAGCAGCCTGAGATCTGAGGACACGGCCGTGTATTA | |
| | CTGTGCAAGAAATGTCTTTGATGGTTACTGGCTTGTTT | |
| | ACTGGGGCCAGGGAACCCTGGTCACCGTCTCCAGCGC | |
| | TAGCACCAAGGGCCCCTCCGTGTTCCCCCTGGCCCCCA | |
| | GCAGCAAGAGCACCAGCGGCGGCACAGCCGCTCTGGG | |
| | CTGCCTGGTCAAGGACTACTTCCCCGAGCCCGTGACCG | |
| | TGTCCTGGAACAGCGGAGCCCTGACCTCCGGCGTGCA | |
| | CACCTTCCCCGCCGTGCTGCAGAGTTCTGGCCTGTATA | |
| | GCCTGAGCAGCGTGGTCACCGTGCCTTCTAGCAGCCTG | |
| | GGCACCCAGACCTACATCTGCAACGTGAACCACAAGC | |
| | CCAGCAACACCAAGGTGGACAAGAAGGTGGAGCCCA | |
| | AGAGCTGCGGAGGGGGCGGATCCTTCTGGGTGCTGGT | |
| | GGTGGTGGGCGGCGTGCTGGCCTGCTACAGCCTGCTG | |
| | GTGACCGTGGCCTTCATCATCTTCTGGGTGAGGAGCAA | |
| | GAGGAGCAGGCTGCTGCACAGCGACTACATGAACATG | |
| | ACCCCCAGGAGGCCCGGCCCCACCAGGAAGCACTACC | |
| | AGCCCTACGCCCCCCCCAGGGACTTCGCCGCCTACAG | |
| | GAGCAGGGTGAAGTTCAGCAGGAGCGCCGACGCCCCC | |
| | GCCTACCAGCAGGGCCAGAACCAGCTGTATAACGAGC | |
| | TGAACCTGGGCAGGAGGGAGGAGTACGACGTGCTGGA | |
| | CAAGAGGAGGGGCAGGGACCCCGAGATGGGCGGCAA | |
| | GCCCAGGAGGAAGAACCCCCAGGAGGGCCTGTATAAC | |
| | GAGCTGCAGAAGGACAAGATGGCCGAGGCCTACAGC | |
| | GAGATCGGCATGAAGGGCGAGAGGAGGAGGGGCAAG | |
| | GGCCACGACGGCCTGTACCAGGGCCTGAGCACCGCCA | |
| | CCAAGGACACCTACGACGCCCTGCACATGCAGGCCCT | |
| | GCCCCCCAGGTCCGGAGAGGGCAGAGGAAGTCTTCTA | |
| | ACATGCGGTGACGTGGAGGAGAATCCCGGCCCTAGGG | |
| | TGAGCAAGGGCGAGGAGCTGTTCACCGGGGTGGTGCC | |
| | CATCCTGGTCGAGCTGGACGGCGACGTAAACGGCCAC | |
| | AAGTTCAGCGTGTCCGGCGAGGGCGAGGGCGATGCCA | |
| | CCTACGGCAAGCTGACCCTGAAGTTCATCTGCACCACC | |
| | GGCAAGCTGCCCGTGCCCTGGCCCACCCTCGTGACCA | |
| | CCCTGACCTACGGCGTGCAGTGCTTCAGCCGCTACCCC | |
| | GACCACATGAAGCAGCACGACTTCTTCAAGTCCGCCA | |
| | TGCCCGAAGGCTACGTCCAGGAGCGCACCATCTTCTTC | |
| | AAGGACGACGGCAACTACAAGACCCGCGCCGAGGTG | |
| | AAGTTCGAGGGCGACACCCTGGTGAACCGCATCGAGC | |
| | TGAAGGGCATCGACTTCAAGGAGGACGGCAACATCCT | |
| | GGGGCACAAGCTGGAGTACAACTACAACAGCCACAAC | |
| | GTCTATATCATGGCCGACAAGCAGAAGAACGGCATCA | |
| | AGGTGAACTTCAAGATCCGCCACAACATCGAGGACGG | |
| | CAGCGTGCAGCTCGCCGACCACTACCAGCAGAACACC | |
| | CCCATCGGCGACGGCCCCGTGCTGCTGCCCGACAACC | |
| | ACTACCTGAGCACCCAGTCCGCCCTGAGCAAAGACCC | |
| | CAACGAGAAGCGCGATCACATGGTCCTGCTGGAGTTC | |
| | GTGACCGCCGCCGGGATCACTCTCGGCATGGACGAGC | |
| | TGTACAAGTGA | |

TABLE 4
Anti-CD20 crossFab (VH-CL-ATD) amino acid sequences

| | | SEQ |
|---|---|---|
| Construct | Protein Sequence | ID NO |
| Anti-CD20 (GA101) | see Table 2 | 1 |
| CDR H1 Kabat | | |
| Anti-CD20 (GA101) | see Table 2 | 2 |
| CDR H2 Kabat | | |
| Anti-CD20 (GA101) | see Table 2 | 3 |
| CDR H3 Kabat | | |
| Anti-CD20 (GA101) | see Table 2 | 4 |
| CDR L1 Kabat | | |
| Anti-CD20 (GA101) | see Table 2 | 5 |
| CDR L2 Kabat | | |
| Anti-CD20 (GA101) | see Table 2 | 6 |
| CDR L3 Kabat | | |
| Anti-CD20-(GA101)- | QVQLVQSGAEVKKPGSSVKVSCKASGYAFSYSWINWVR | 36 |
| crossFab VH-CL | QAPGQGLEWMGRIFPGDGDTDYNGKFKGRVTITADKST | |
| light chain- | STAYMELSSLRSEDTAVYYCARNVFDGYWLVYWGQGT | |
| ATD- | LVTVSSASVAAPSVFIFPPSDEQLKSGTASVVCLLNNFYP | |
| CD28ATD- | REAKVQWKVDNALQSGNSQESVTEQDSKDSTYSLSSTL | |
| CD28CSD- | TLSKADYEKHKVYACEVTHQGLSSPVTKSFNRGECGGG | |
| CD3zSSD fusion | GSFWVLVVVGGVLACYSLLVTVAFIIFWVRSKRSRLLHS | |
| pETR17098 | DYMNMTPRRPGPTRKHYQPYAPPRDFAAYRSRVKFSRS | |
| | ADAPAYQQGQNQLYNELNLGRREEYDVLDKRRGRDPE | |
| | MGGKPRRKNPQEGLYNELQKDKMAEAYSEIGMKGERR | |
| | RGKGHDGLYQGLSTATKDTYDALHMQALPPR | |
| Anti-CD20-(GA101)- | QVQLVQSGAEVKKPGSSVKVSCKASGYAFSYSWINWVR | 37 |
| crossFab VH-CL | QAPGQGLEWMGRIFPGDGDTDYNGKFKGRVTITADKST | |
| light chain | STAYMELSSLRSEDTAVYYCARNVFDGYWLVYWGQGT | |
| pETR17098 | LVTVSSASVAAPSVFIFPPSDEQLKSGTASVVCLLNNFYP | |
| | REAKVQWKVDNALQSGNSQESVTEQDSKDSTYSLSSTL | |
| | TLSKADYEKHKVYACEVTHQGLSSPVTKSFNRGEC | |
| Anti-CD20-(GA101)- | DIVMTQTPLSLPVTPGEPASISCRSSKSLLHSNGITYLYWY | 38 |
| crossFab VL-CH1 | LQKPGQSPQLLIYQMSNLVSGVPDRFSGSGSGTDFTLKIS | |
| heavy chain- | RVEAEDVGVYYCAQNLELPYTFGGGTKVEIKSSASTKGP | |
| pETR17098 | SVFPLAPSSKSTSGGTAALGCLVKDYFPEPVTVSWNSGA | |
| | LTSGVHTFPAVLQSSGLYSLSSVVTVPSSSLGTQTYICNV | |
| | NHKPSNTKVDKKVEPKSC | |
| Anti-CD20-(GA101) | see Table 2 | 12 |
| VH | | |
| crossFab CL | ASVAAPSVFIFPPSDEQLKSGTASVVCLLNNFYPREAKVQ | 39 |
| | WKVDNALQSGNSQESVTEQDSKDSTYSLSSTLTLSKADY | |
| | EKHKVYACEVTHQGLSSPVTKSFNRGEC | |
| Anti-CD20-(GA101)- | see Table 2 | 10 |
| VL | | |
| crossFab CH1 | SSASTKGPSVFPLAPSSKSTSGGTAALGCLVKDYFPEPVT | 40 |
| | VSWNSGALTSGVHTFPAVLQSSGLYSLSSVVTVPSSSLGT | |
| | QTYICNVNHKPSNTKVDKKVEPKSC | |
| G4S linker | see Table 2 | 20 |
| CD28ATD-CD28CSD- | see Table 2 | 17 |
| CD3zSSD | | |
| T2A linker | see Table 2 | 21 |
| eGFP | see Table 2 | 18 |

TABLE 5
Anti-CD20 crossFab (VL-CH1-ATD) amino acid sequences

| | | SEQ |
|---|---|---|
| Construct | Protein Sequence | ID NO |
| Anti-CD20 (GA101) | see Table 2 | 1 |
| CDR H1 Kabat | | |
| Anti-CD20 (GA101) | see Table 2 | 2 |
| CDR H2 Kabat | | |
| Anti-CD20 (GA101) | see Table 2 | 3 |
| CDR H3 Kabat | | |
| Anti-CD20 (GA101) | see Table 2 | 4 |
| CDR L1 Kabat | | |
| Anti-CD20 (GA101) | see Table 2 | 5 |
| CDR L2 Kabat | | |
| Anti-CD20 (GA101) | see Table 2 | 6 |
| CDR L3 Kabat | | |
| Anti-CD20-(GA101)- | DIVMTQTPLSLPVTPGEPASISCRSSKSLLHSNGITYLYWY | 41 |
| crossFab VL-CH1 | LQKPGQSPQLLIYQMSNLVSGVPDRFSGSGSGTDFTLKIS | |
| heavy-chain- | RVEAEDVGVYYCAQNLELPYTFGGGTKVEIKSSASTKGP | |
| ATD- | SVFPLAPSSKSTSGGTAALGCLVKDYFPEPVTVSWNSGA | |
| CD28ATD- | LTSGVHTFPAVLQSSGLYSLSSVVTVPSSSLGTQTYICNV | |
| CD28CSD- | NHKPSNTKVDKKVEPKSCGGGGSFWVLVVVGGVLACY | |
| CD3zSSD fusion | SLLVTVAFIIFWVRSKRSRLLHSDYMNMTPRRPGPTRKH | |
| | YQPYAPPRDFAAYRSRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATK | |
| | DTYDALHMQALPPR | |
| Anti-CD20-(GA101)- | DIVMTQTPLSLPVTPGEPASISCRSSKSLLHSNGITYLYWY | 42 |
| crossFab VL-CH1 | LQKPGQSPQLLIYQMSNLVSGVPDRFSGSGSGTDFTLKIS | |
| heavy chain | RVEAEDVGVYYCAQNLELPYTFGGGTKVEIKSSASTKGP | |
| | SVFPLAPSSKSTSGGTAALGCLVKDYFPEPVTVSWNSGA | |
| | LTSGVHTFPAVLQSSGLYSLSSVVTVPSSSLGTQTYICNV | |
| | NHKPSNTKVDKKVEPKSC | |
| Anti-CD20-(GA101)- | QVQLVQSGAEVKKPGSSVKVSCKASGYAFSYSWINWVR | 43 |
| crossFab VH-CL | QAPGQGLEWMGRIFPGDGDTDYNGKFKGRVTITADKST | |
| light chain | STAYMELSSLRSEDTAVYYCARNVFDGYWLVYWGQGT | |
| | LVTVSSASVAAPSVFIFPPSDEQLKSGTASVVCLLNNFYP | |
| | REAKVQWKVDNALQSGNSQESVTEQDSKDSTYSLSSTL | |
| | TLSKADYEKHKVYACEVTHQGLSSPVTKSFNRGEC | |
| Anti-CD20-(GA101) | see Table 2 | 12 |
| VH | | |
| crossFab CL | ASVAAPSVFIFPPSDEQLKSGTASVVCLLNNFYPREAKVQ | 44 |
| | WKVDNALQSGNSQESVTEQDSKDSTYSLSSTLTLSKADY | |
| | EKHKVYACEVTHQGLSSPVTKSFNRGEC | |
| Anti-CD20-(GA101)- | see Table 2 | 10 |
| VL | | |
| crossFab CH1 | SSASTKGPSVFPLAPSSKSTSGGTAALGCLVKDYFPEPVT | 45 |
| | VSWNSGALTSGVHTFPAVLQSSGLYSLSSVVTVPSSSLGT | |
| | QTYICNVNHKPSNTKVDKKVEPKSC | |
| G4S linker | see Table 2 | 20 |
| CD28ATD-CD28CSD- | see Table 2 | 17 |
| CD3zSSD | | |
| T2A linker | see Table 2 | 21 |
| eGFP | see Table 2 | 18 |

TABLE 6
Anti-CD20 crossFab (VH-CL-ATD) DNA sequences

| | | SEQ |
|---|---|---|
| Construct | DNA Sequenz | ID NO |
| Anti-CD20-(GA101)- | ATGGGATGGAGCTGTATCATCCTCTTCTTGGTAGCAAC | 46 |
| crossFabVH-CL | AGCTACCGGTGTGCATTCCCAGGTGCAATTGGTGCAGT | |
| CD28ATD- | CTGGCGCTGAAGTTAAGAAGCCTGGGAGTTCAGTGAA | |
| CD28CSD- | GGTCTCCTGCAAGGCTTCCGGATACGCCTTCAGCTATT | |
| CD3zSSD | CTTGGATCAATTGGGTGCGGCAGGCGCCTGGACAAGG | |
| pETR17098 | GCTCGAGTGGATGGGACGGATCTTTCCCGGCGATGGG | |
| | GATACTGACTACAATGGGAAATTCAAGGGCAGAGTCA | |
| | CAATTACCGCCGACAAATCCACTAGCACAGCCTATAT | |
| | GGAGCTGAGCAGCCTGAGATCTGAGGACACGGCCGTG | |
| | TATTACTGTGCAAGAAATGTCTTTGATGGTTACTGGCT | |
| | TGTTTACTGGGGCCAGGGAACCCTGGTCACCGTCTCCT | |
| | CAGCTAGCGTGGCCGCTCCCTCCGTGTTCATCTTCCCA | |
| | CCTTCCGACGAGCAGCTGAAGTCCGGCACCGCTTCTGT | |
| | CGTGTGCCTGCTGAACAACTTCTACCCCCGCGAGGCCA | |
| | AGGTGCAGTGGAAGGTGGACAACGCCCTGCAGTCCGG | |
| | CAACAGCCAGGAATCCGTGACCGAGCAGGACTCCAAG | |
| | GACAGCACCTACTCCCTGTCCTCCACCCTGACCCTGTC | |
| | CAAGGCCGACTACGAGAAGCACAAGGTGTACGCCTGC | |
| | GAAGTGACCCACCAGGGCCTGTCTAGCCCCGTGACCA | |
| | AGTCTTTCAACCGGGGCGAGTGCTGATAAGGAATTCC | |
| | CCGAAGTAACTTAGAAGCTGTAAATCAACGATCAATA | |
| | GCAGGTGTGGCACACCAGTCATACCTTGATCAAGCAC | |
| | TTCTGTTTCCCCGGACTGAGTATCAATAGGCTGCTCGC | |
| | GCGGCTGAAGGAGAAAACGTTCGTTACCCGACCAACT | |
| | ACTTCGAGAAGCTTAGTACCACCATGAACGAGGCAGG | |
| | GTGTTTCGCTCAGCACAACCCCAGTGTAGATCAGGCTG | |
| | ATGAGTCACTGCAACCCCCATGGGCGACCATGGCAGT | |
| | GGCTGCGTTGGCGGCCTGCCCATGGAGAAATCCATGG | |
| | GACGCTCTAATTCTGACATGGTGTGAAGTGCCTATTGA | |
| | GCTAACTGGTAGTCCTCCGGCCCCTGATTGCGGCTAAT | |
| | CCTAACTGCGGAGCACATGCTCACAAACCAGTGGGTG | |
| | GTGTGTCGTAACGGGCAACTCTGCAGCGGAACCGACT | |
| | ACTTTGGGTGTCCGTGTTTCCTTTTATTCCTATATTGGC | |
| | TGCTTATGGTGACAATCAAAAAGTTGTTACCATATAGC | |
| | TATTGGATTGGCCATCCGGTGTGCAACAGGGCAACTG | |
| | TTTACCTATTTATTGGTTTTGTACCATTATCACTGAAGT | |
| | CTGTGATCACTCTCAAATTCATTTTGACCCTCAACACA | |
| | ATCAAACGCCACCATGGGATGGAGCTGTATCATCCTCT | |
| | TCTTGGTAGCAACAGCTACCGGTGTGCACTCCGACATC | |
| | GTGATGACCCAGACTCCACTCTCCCTGCCCGTCACCCC | |
| | TGGAGAGCCCGCCAGCATTAGCTGCAGGTCTAGCAAG | |
| | AGCCTCTTGCACAGCAATGGCATCACTTATTTGTATTG | |
| | GTACCTGCAAAAGCCAGGGCAGTCTCCACAGCTCCTG | |
| | ATTTATCAAATGTCCAACCTTGTCTCTGGCGTCCCTGA | |
| | TCGGTTCTCCGGTTCCGGGTCAGGCACTGATTTCACAC | |
| | TGAAAATCAGCAGGGTGGAGGCTGAGGATGTTGGAGT | |
| | TTATTACTGCGCTCAGAATCTAGAACTTCCTTACACCT | |
| | TCGGCGGAGGGACCAAGGTGGAGATCAAATCCAGCGC | |
| | TAGCACCAAGGGCCCCTCCGTGTTCCCCCTGGCCCCCA | |
| | GCAGCAAGAGCACCAGCGGCGGCACAGCCGCTCTGGG | |
| | CTGCCTGGTCAAGGACTACTTCCCCGAGCCCGTGACCG | |
| | TGTCCTGGAACAGCGGAGCCCTGACCTCCGGCGTGCA | |
| | CACCTTCCCCGCCGTGCTGCAGAGTTCTGGCCTGTATA | |
| | GCCTGAGCAGCGTGGTCACCGTGCCTTCTAGCAGCCTG | |
| | GGCACCCAGACCTACATCTGCAACGTGAACCACAAGC | |
| | CCAGCAACACCAAGGTGGACAAGAAGGTGGAGCCCA | |
| | AGAGCTGCGGAGGGGGCGGATCCTTCTGGGTGCTGGT | |
| | GGTGGTGGGCGGCGTGCTGGCCTGCTACAGCCTGCTG | |
| | GTGACCGTGGCCTTCATCATCTTCTGGGTGAGGAGCAA | |
| | GAGGAGCAGGCTGCTGCACAGCGACTACATGAACATG | |
| | ACCCCCAGGAGGCCCGGCCCCACCAGGAAGCACTACC | |
| | AGCCCTACGCCCCCCCCAGGGACTTCGCCGCCTACAG | |
| | GAGCAGGGTGAAGTTCAGCAGGAGCGCCGACGCCCCC | |
| | GCCTACCAGCAGGGCCAGAACCAGCTGTATAACGAGC | |
| | TGAACCTGGGCAGGAGGGAGGAGTACGACGTGCTGGA | |
| | CAAGAGGAGGGGCAGGGACCCCGAGATGGGCGGCAA | |
| | GCCCAGGAGGAAGAACCCCCAGGAGGGCCTGTATAAC | |
| | GAGCTGCAGAAGGACAAGATGGCCGAGGCCTACAGC | |
| | GAGATCGGCATGAAGGGCGAGAGGAGGAGGGGCAAG | |
| | GGCCACGACGGCCTGTACCAGGGCCTGAGCACCGCCA | |
| | CCAAGGACACCTACGACGCCCTGCACATGCAGGCCCT | |
| | GCCCCCCAGG | |
| Anti-CD20-(GA101)- | see Table 3 | 25 |
| VH | | |
| crossFab CL | GCTAGCGTGGCCGCTCCCTCCGTGTTCATCTTCCCACC | 47 |
| | TTCCGACGAGCAGCTGAAGTCCGGCACCGCTTCTGTCG | |
| | TGTGCCTGCTGAACAACTTCTACCCCCGCGAGGCCAA | |
| | GGTGCAGTGGAAGGTGGACAACGCCCTGCAGTCCGGC | |
| | AACAGCCAGGAATCCGTGACCGAGCAGGACTCCAAGG | |
| | ACAGCACCTACTCCCTGTCCTCCACCCTGACCCTGTCC | |
| | AAGGCCGACTACGAGAAGCACAAGGTGTACGCCTGCG | |
| | AAGTGACCCACCAGGGCCTGTCTAGCCCCGTGACCAA | |
| | GTCTTTCAACCGGGGCGAGTGCTGA | |
| Anti-CD20-(GA101)- | see Table 3 | 23 |
| VL | | |
| crossFab CH1 | TCCAGCGCTAGCACCAAGGGCCCCTCCGTGTTCCCCCT | 48 |
| | GGCCCCCAGCAGCAAGAGCACCAGCGGCGGCACAGCC | |
| | GCTCTGGGCTGCCTGGTCAAGGACTACTTCCCCGAGCC | |
| | CGTGACCGTGTCCTGGAACAGCGGAGCCCTGACCTCC | |
| | GGCGTGCACACCTTCCCCGCCGTGCTGCAGAGTTCTGG | |
| | CCTGTATAGCCTGAGCAGCGTGGTCACCGTGCCTTCTA | |
| | GCAGCCTGGGCACCCAGACCTACATCTGCAACGTGAA | |
| | CCACAAGCCCAGCAACACCAAGGTGGACAAGAAGGT | |
| | GGAGCCCAAGAGCTGC | |
| IRES EV71, internal | see Table 3 | 27 |
| ribosomal entry site | | |
| G4S linker | see Table 3 | 28 |
| CD28ATD-CD28CSD- | see Table 3 | 32 |
| CD3zSSD | | |
| T2A linker | see Table 3 | 33 |
| eGFP | see Table 3 | 34 |
| Anti-CD20-(GA101)- | ATGGGATGGAGCTGTATCATCCTCTTCTTGGTAGCAAC | 49 |
| crossFabVH-CL | AGCTACCGGTGTGCATTCCCAGGTGCAATTGGTGCAGT | |
| CD28ATD- | CTGGCGCTGAAGTTAAGAAGCCTGGGAGTTCAGTGAA | |
| CD28CSD- | GGTCTCCTGCAAGGCTTCCGGATACGCCTTCAGCTATT | |
| CD3zSSD- | CTTGGATCAATTGGGTGCGGCAGGCGCCTGGACAAGG | |
| eGFP | GCTCGAGTGGATGGGACGGATCTTTCCCGGCGATGGG | |
| pETR17098 | GATACTGACTACAATGGGAAATTCAAGGGCAGAGTCA | |
| | CAATTACCGCCGACAAATCCACTAGCACAGCCTATAT | |
| | GGAGCTGAGCAGCCTGAGATCTGAGGACACGGCCGTG | |
| | TATTACTGTGCAAGAAATGTCTTTGATGGTTACTGGCT | |
| | TGTTTACTGGGGCCAGGGAACCCTGGTCACCGTCTCCT | |
| | CAGCTAGCGTGGCCGCTCCCTCCGTGTTCATCTTCCCA | |
| | CCTTCCGACGAGCAGCTGAAGTCCGGCACCGCTTCTGT | |
| | CGTGTGCCTGCTGAACAACTTCTACCCCCGCGAGGCCA | |
| | AGGTGCAGTGGAAGGTGGACAACGCCCTGCAGTCCGG | |
| | CAACAGCCAGGAATCCGTGACCGAGCAGGACTCCAAG | |
| | GACAGCACCTACTCCCTGTCCTCCACCCTGACCCTGTC | |
| | CAAGGCCGACTACGAGAAGCACAAGGTGTACGCCTGC | |
| | GAAGTGACCCACCAGGGCCTGTCTAGCCCCGTGACCA | |
| | AGTCTTTCAACCGGGGCGAGTGCTGATAAGGAATTCC | |
| | CCGAAGTAACTTAGAAGCTGTAAATCAACGATCAATA | |
| | GCAGGTGTGGCACACCAGTCATACCTTGATCAAGCAC | |
| | TTCTGTTTCCCCGGACTGAGTATCAATAGGCTGCTCGC | |
| | GCGGCTGAAGGAGAAAACGTTCGTTACCCGACCAACT | |
| | ACTTCGAGAAGCTTAGTACCACCATGAACGAGGCAGG | |
| | GTGTTTCGCTCAGCACAACCCCAGTGTAGATCAGGCTG | |
| | ATGAGTCACTGCAACCCCCATGGGCGACCATGGCAGT | |
| | GGCTGCGTTGGCGGCCTGCCCATGGAGAAATCCATGG | |
| | GACGCTCTAATTCTGACATGGTGTGAAGTGCCTATTGA | |
| | GCTAACTGGTAGTCCTCCGGCCCCTGATTGCGGCTAAT | |
| | CCTAACTGCGGAGCACATGCTCACAAACCAGTGGGTG | |
| | GTGTGTCGTAACGGGCAACTCTGCAGCGGAACCGACT | |
| | ACTTTGGGTGTCCGTGTTTCCTTTTATTCCTATATTGGC | |
| | TGCTTATGGTGACAATCAAAAAGTTGTTACCATATAGC | |
| | TATTGGATTGGCCATCCGGTGTGCAACAGGGCAACTG | |
| | TTTACCTATTTATTGGTTTTGTACCATTATCACTGAAGT | |
| | CTGTGATCACTCTCAAATTCATTTTGACCCTCAACACA | |
| | ATCAAACGCCACCATGGGATGGAGCTGTATCATCCTCT | |
| | TCTTGGTAGCAACAGCTACCGGTGTGCACTCCGACATC | |
| | GTGATGACCCAGACTCCACTCTCCCTGCCCGTCACCCC | |
| | TGGAGAGCCCGCCAGCATTAGCTGCAGGTCTAGCAAG | |
| | AGCCTCTTGCACAGCAATGGCATCACTTATTTGTATTG | |
| | GTACCTGCAAAAGCCAGGGCAGTCTCCACAGCTCCTG | |
| | ATTTATCAAATGTCCAACCTTGTCTCTGGCGTCCCTGA | |
| | TCGGTTCTCCGGTTCCGGGTCAGGCACTGATTTCACAC | |
| | TGAAAATCAGCAGGGTGGAGGCTGAGGATGTTGGAGT | |
| | TTATTACTGCGCTCAGAATCTAGAACTTCCTTACACCT | |
| | TCGGCGGAGGGACCAAGGTGGAGATCAAATCCAGCGC | |
| | TAGCACCAAGGGCCCCTCCGTGTTCCCCCTGGCCCCCA | |
| | GCAGCAAGAGCACCAGCGGCGGCACAGCCGCTCTGGG | |
| | CTGCCTGGTCAAGGACTACTTCCCCGAGCCCGTGACCG | |
| | TGTCCTGGAACAGCGGAGCCCTGACCTCCGGCGTGCA | |
| | CACCTTCCCCGCCGTGCTGCAGAGTTCTGGCCTGTATA | |
| | GCCTGAGCAGCGTGGTCACCGTGCCTTCTAGCAGCCTG | |
| | GGCACCCAGACCTACATCTGCAACGTGAACCACAAGC | |
| | CCAGCAACACCAAGGTGGACAAGAAGGTGGAGCCCA | |
| | AGAGCTGCGGAGGGGGCGGATCCTTCTGGGTGCTGGT | |
| | GGTGGTGGGCGGCGTGCTGGCCTGCTACAGCCTGCTG | |
| | GTGACCGTGGCCTTCATCATCTTCTGGGTGAGGAGCAA | |
| | GAGGAGCAGGCTGCTGCACAGCGACTACATGAACATG | |
| | ACCCCCAGGAGGCCCGGCCCCACCAGGAAGCACTACC | |
| | AGCCCTACGCCCCCCCCAGGGACTTCGCCGCCTACAG | |
| | GAGCAGGGTGAAGTTCAGCAGGAGCGCCGACGCCCCC | |
| | GCCTACCAGCAGGGCCAGAACCAGCTGTATAACGAGC | |
| | TGAACCTGGGCAGGAGGGAGGAGTACGACGTGCTGGA | |
| | CAAGAGGAGGGGCAGGGACCCCGAGATGGGCGGCAA | |
| | GCCCAGGAGGAAGAACCCCCAGGAGGGCCTGTATAAC | |
| | GAGCTGCAGAAGGACAAGATGGCCGAGGCCTACAGC | |
| | GAGATCGGCATGAAGGGCGAGAGGAGGAGGGGCAAG | |
| | GGCCACGACGGCCTGTACCAGGGCCTGAGCACCGCCA | |
| | CCAAGGACACCTACGACGCCCTGCACATGCAGGCCCT | |
| | GCCCCCCAGGTCCGGAGAGGGCAGAGGAAGTCTTCTA | |
| | ACATGCGGTGACGTGGAGGAGAATCCCGGCCCTAGGG | |
| | TGAGCAAGGGCGAGGAGCTGTTCACCGGGGTGGTGCC | |
| | CATCCTGGTCGAGCTGGACGGCGACGTAAACGGCCAC | |
| | AAGTTCAGCGTGTCCGGCGAGGGCGAGGGCGATGCCA | |
| | CCTACGGCAAGCTGACCCTGAAGTTCATCTGCACCACC | |
| | GGCAAGCTGCCCGTGCCCTGGCCCACCCTCGTGACCA | |
| | CCCTGACCTACGGCGTGCAGTGCTTCAGCCGCTACCCC | |
| | GACCACATGAAGCAGCACGACTTCTTCAAGTCCGCCA | |
| | TGCCCGAAGGCTACGTCCAGGAGCGCACCATCTTCTTC | |
| | AAGGACGACGGCAACTACAAGACCCGCGCCGAGGTG | |
| | AAGTTCGAGGGCGACACCCTGGTGAACCGCATCGAGC | |
| | TGAAGGGCATCGACTTCAAGGAGGACGGCAACATCCT | |
| | GGGGCACAAGCTGGAGTACAACTACAACAGCCACAAC | |
| | GTCTATATCATGGCCGACAAGCAGAAGAACGGCATCA | |
| | AGGTGAACTTCAAGATCCGCCACAACATCGAGGACGG | |
| | CAGCGTGCAGCTCGCCGACCACTACCAGCAGAACACC | |
| | CCCATCGGCGACGGCCCCGTGCTGCTGCCCGACAACC | |
| | ACTACCTGAGCACCCAGTCCGCCCTGAGCAAAGACCC | |
| | CAACGAGAAGCGCGATCACATGGTCCTGCTGGAGTTC | |
| | GTGACCGCCGCCGGGATCACTCTCGGCATGGACGAGC | |
| | TGTACAAGTGA | |

TABLE 7
Anti-CD20 Fab (VL-CL-ATD) amino acid sequences

| | | SEQ |
|---|---|---|
| Construct | Protein Sequence | ID NO |
| Anti-CD20 (GA101) | see Table 2 | 1 |
| CDR H1 Kabat | | |
| Anti-CD20 (GA101) | see Table 2 | 2 |
| CDR H2 Kabat | | |
| Anti-CD20 (GA101) | see Table 2 | 3 |
| CDR H3 Kabat | | |
| Anti-CD20 (GA101) | see Table 2 | 4 |
| CDR L1 Kabat | | |
| Anti-CD20 (GA101) | see Table 2 | 5 |
| CDR L2 Kabat | | |
| Anti-CD20 (GA101) | see Table 2 | 6 |
| CDR L3 Kabat | | |
| Anti-CD20-(GA101)- | DIVMTQTPLSLPVTPGEPASISCRSSKSLLHSNGITYLYWY | 50 |
| Fab VL-CL | LQKPGQSPQLLIYQMSNLVSGVPDRFSGSGSGTDFTLKIS | |
| light chain- | RVEAEDVGVYYCAQNLELPYTFGGGTKVEIKRTVAAPS | |
| CD28ATD- | VFIFPPSDEQLKSGTASVVCLLNNFYPREAKVQWKVDNA | |
| CD28CSD- | LQSGNSQESVTEQDSKDSTYSLSSTLTLSKADYEKHKVY | |
| CD3zSSD fusion | ACEVTHQGLSSPVTKSFNRGECGGGGSFWVLVVVGGVL | |
| | ACYSLLVTVAFIIFWVRSKRSRLLHSDYMNMTPRRPGPT | |
| | RKHYQPYAPPRDFAAYRSRVKFSRSADAPAYQQGQNQL | |
| | YNELNLGRREEYDVLDKRRGRDPEMGGKPRRKNPQEGL | |
| | YNELQKDKMAEAYSEIGMKGERRRGKGHDGLYQGLST | |
| | ATKDTYDALHMQALPPR | |
| Anti-CD20-(GA101)- | see Table 2 | 8 |
| Fab heavy chain | | |
| Anti-CD20-(GA101)- | see Table 2 | 9 |
| Fab light chain | | |
| Anti-CD20-(GA101) | see Table 2 | 12 |
| VH | | |
| CL | see Table 2 | 11 |
| Anti-CD20-(GA101)- | see Table 2 | 10 |
| VL | | |
| CH1 | see Table 2 | 13 |
| G4S linker | see Table 2 | 20 |
| CD28ATD-CD28CSD- | see Table 2 | 17 |
| CD3zSSD | | |
| T2A linker | see Table 2 | 21 |
| eGFP | see Table 2 | 18 |

TABLE 8
Anti-CD20 scFab amino acid sequences

| | | SEQ |
|---|---|---|
| Construct | Protein Sequence | ID NO |
| Anti-CD20 (GA101) | see Table 2 | 1 |
| CDR H1 Kabat | | |
| Anti-CD20 (GA101) | see Table 2 | 2 |
| CDR H2 Kabat | | |
| Anti-CD20 (GA101) | see Table 2 | 3 |
| CDR H3 Kabat | | |
| Anti-CD20 (GA101) | see Table 2 | 4 |
| CDR L1 Kabat | | |
| Anti-CD20 (GA101) | see Table 2 | 5 |
| CDR L2 Kabat | | |
| Anti-CD20 (GA101) | see Table 2 | 6 |
| CDR L3 Kabat | | |
| Anti-CD20-(GA101)- | DIVMTQTPLSLPVTPGEPASISCRSSKSLLHSNGITYLYWY | 51 |
| scFab- | LQKPGQSPQLLIYQMSNLVSGVPDRFSGSGSGTDFTLKIS | |
| CD28ATD- | RVEAEDVGVYYCAQNLELPYTFGGGTKVEIKRTVAAPS | |
| CD28CSD- | VFIFPPSDEQLKSGTASVVCLLNNFYPREAKVQWKVDNA | |
| CD3zSSD fusion | LQSGNSQESVTEQDSKDSTYSLSSTLTLSKADYEKHKVY | |
| pETR17101 | ACEVTHQGLSSPVTKSFNRGECGGGGSGGGGSGGGGSG | |
| | GGGSGGGGSGGGGSGGQVQLVQSGAEVKKPGSSVKVSC | |
| | KASGYAFSYSWINWVRQAPGQGLEWMGRIFPGDGDTD | |
| | YNGKFKGRVTITADKSTSTAYMELSSLRSEDTAVYYCAR | |
| | NVFDGYWLVYWGQGTLVTVSSASTKGPSVFPLAPSSKST | |
| | SGGTAALGCLVKDYFPEPVTVSWNSGALTSGVHTFPAVL | |
| | QSSGLYSLSSVVTVPSSSLGTQTYICNVNHKPSNTKVDKK | |
| | VEPKSCGGGGSFWVLVVVGGVLACYSLLVTVAFIIFWVR | |
| | SKRSRLLHSDYMNMTPRRPGPTRKHYQPYAPPRDFAAY | |
| | RSRVKFSRSADAPAYQQGQNQLYNELNLGRREEYDVLD | |
| | KRRGRDPEMGGKPRRKNPQEGLYNELQKDKMAEAYSEI | |
| | GMKGERRRGKGHDGLYQGLSTATKDTYDALHMQALPP | |
| | R | |
| Anti-CD20-(GA101)- | DIVMTQTPLSLPVTPGEPASISCRSSKSLLHSNGITYLYWY | 52 |
| scFab | LQKPGQSPQLLIYQMSNLVSGVPDRFSGSGSGTDFTLKIS | |
| | RVEAEDVGVYYCAQNLELPYTFGGGTKVEIKRTVAAPS | |
| | VFIFPPSDEQLKSGTASVVCLLNNFYPREAKVQWKVDNA | |
| | LQSGNSQESVTEQDSKDSTYSLSSTLTLSKADYEKHKVY | |
| | ACEVTHQGLSSPVTKSFNRGECGGGGSGGGGSGGGGSG | |
| | GGGSGGGGSGGGGSGGQVQLVQSGAEVKKPGSSVKVSC | |
| | KASGYAFSYSWINWVRQAPGQGLEWMGRIFPGDGDTD | |
| | YNGKFKGRVTITADKSTSTAYMELSSLRSEDTAVYYCAR | |
| | NVFDGYWLVYWGQGTLVTVSSASTKGPSVFPLAPSSKST | |
| | SGGTAALGCLVKDYFPEPVTVSWNSGALTSGVHTFPAVL | |
| | QSSGLYSLSSVVTVPSSSLGTQTYICNVNHKPSNTKVDKK | |
| | VEPKSC | |
| Anti-CD20-(GA101)- | see Table 2 | 10 |
| VL | | |
| scFab-CL | RTVAAPSVFIFPPSDEQLKSGTASVVCLLNNFYPREAKVQ | 53 |
| | WKVDNALQSGNSQESVTEQDSKDSTYSLSSTLTLSKADY | |
| | EKHKVYACEVTHQGLSSPVTKSFNRGE | |
| Anti-CD20-(GA101) | see Table 2 | 12 |
| VH | | |
| CH1 | see Table 2 | 13 |
| CD28TM-CD28-CD3z | see Table 2 | 17 |
| (G4S)₆G₂ linker | GGGGSGGGGSGGGGSGGGGSGGGGSGGGGSGG | 54 |

TABLE 9
Anti-CD20 scFab DNA sequences

| | | SEQ |
|---|---|---|
| Construct | DNA Sequenz | ID NO |
| Anti-CD20-(GA101)- | ATGGGATGGAGCTGTATCATCCTCTTCTTGGTAGCAAC | 55 |
| scFab- | AGCTACGGGTGTGCATTCCGACATCGTGATGACCCAG | |
| CD28ATD- | ACTCCACTCTCCCTGCCCGTCACCCCTGGAGAGCCCGC | |
| CD28CSD- | CAGCATTAGCTGCAGGTCTAGCAAGAGCCTCTTGCAC | |
| CD3zSSD fusion | AGCAATGGCATCACTTATTTGTATTGGTACCTGCAAAA | |
| pETR17101 | GCCAGGGCAGTCTCCACAGCTCCTGATTTATCAAATGT | |
| | CCAACCTTGTCTCTGGCGTCCCTGATCGGTTCTCCGGT | |
| | TCCGGGTCAGGCACTGATTTCACACTGAAAATCAGCA | |
| | GGGTGGAGGCTGAGGATGTTGGAGTTTATTACTGCGC | |
| | TCAGAATCTAGAACTTCCTTACACCTTCGGCGGAGGG | |
| | ACCAAGGTGGAGATCAAACGTACGGTGGCTGCACCAT | |
| | CTGTCTTCATCTTCCCGCCATCTGATGAGCAGTTGAAA | |
| | TCTGGAACTGCCTCTGTTGTGTGCCTGCTGAATAACTT | |
| | CTATCCCAGAGAGGCCAAAGTACAGTGGAAGGTGGAT | |
| | AACGCCCTCCAATCGGGTAACTCCCAGGAGAGTGTCA | |
| | CAGAGCAGGACAGCAAGGACAGCACCTACAGCCTCAG | |
| | CAGCACCCTGACGCTGAGCAAAGCAGACTACGAGAAA | |
| | CACAAAGTCTACGCCTGCGAAGTCACCCATCAGGGCC | |
| | TGAGCTCGCCCGTCACAAAGAGCTTCAACAGGGGAGA | |
| | GTGTGGCGGCGGAGGATCTGGTGGCGGAGGTAGTGGT | |
| | GGTGGTGGATCTGGCGGAGGCGGCTCCGGCGGAGGTG | |
| | GAAGCGGAGGTGGTGGTTCCGGAGGACAGGTGCAATT | |
| | GGTGCAGTCTGGCGCTGAAGTTAAGAAGCCTGGGAGT | |
| | TCAGTGAAGGTCTCCTGCAAGGCTTCGGGATACGCCTT | |
| | CAGCTATTCTTGGATCAATTGGGTGCGGCAGGCGCCTG | |
| | GACAAGGGCTCGAGTGGATGGGACGGATCTTTCCCGG | |
| | CGATGGGGATACTGACTACAATGGGAAATTCAAGGGC | |
| | AGAGTCACAATTACCGCCGACAAATCCACTAGCACAG | |
| | CCTATATGGAGCTGAGCAGCCTGAGATCTGAGGACAC | |
| | GGCCGTGTATTACTGTGCAAGAAATGTCTTTGATGGTT | |
| | ACTGGCTTGTTTACTGGGGCCAGGGAACCCTGGTCACC | |
| | GTCTCCTCAGCTAGCACCAAGGGCCCCTCCGTGTTCCC | |
| | CCTGGCCCCCAGCAGCAAGAGCACCAGCGGCGGCACA | |
| | GCCGCTCTGGGCTGCCTGGTCAAGGACTACTTCCCCGA | |
| | GCCCGTGACCGTGTCCTGGAACAGCGGAGCCCTGACC | |
| | TCCGGCGTGCACACCTTCCCCGCCGTGCTGCAGAGTTC | |
| | TGGCCTGTATAGCCTGAGCAGCGTGGTCACCGTGCCTT | |
| | CTAGCAGCCTGGGCACCCAGACCTACATCTGCAACGT | |
| | GAACCACAAGCCCAGCAACACCAAGGTGGACAAGAA | |
| | GGTGGAGCCCAAGAGCTGCGGAGGGGGCGGATCCTTC | |
| | TGGGTGCTGGTGGTGGTGGGCGGCGTGCTGGCCTGCT | |
| | ACAGCCTGCTGGTGACCGTGGCCTTCATCATCTTCTGG | |
| | GTGAGGAGCAAGAGGAGCAGGCTGCTGCACAGCGACT | |
| | ACATGAACATGACCCCCAGGAGGCCCGGCCCCACCAG | |
| | GAAGCACTACCAGCCCTACGCCCCCCCCAGGGACTTC | |
| | GCCGCCTACAGGAGCAGGGTGAAGTTCAGCAGGAGCG | |
| | CCGACGCCCCCGCCTACCAGCAGGGCCAGAACCAGCT | |
| | GTATAACGAGCTGAACCTGGGCAGGAGGGAGGAGTAC | |
| | GACGTGCTGGACAAGAGGAGGGGCAGGGACCCCGAG | |
| | ATGGGCGGCAAGCCCAGGAGGAAGAACCCCCAGGAG | |
| | GGCCTGTATAACGAGCTGCAGAAGGACAAGATGGCCG | |
| | AGGCCTACAGCGAGATCGGCATGAAGGGCGAGAGGA | |
| | GGAGGGGCAAGGGCCACGACGGCCTGTACCAGGGCCT | |
| | GAGCACCGCCACCAAGGACACCTACGACGCCCTGCAC | |
| | ATGCAGGCCCTGCCCCCCAGG | |
| scFab-VL | GACATCGTGATGACCCAGACTCCACTCTCCCTGCCCGT | 56 |
| | CACCCCTGGAGAGCCCGCCAGCATTAGCTGCAGGTCT | |
| | AGCAAGAGCCTCTTGCACAGCAATGGCATCACTTATTT | |
| | GTATTGGTACCTGCAAAAGCCAGGGCAGTCTCCACAG | |
| | CTCCTGATTTATCAAATGTCCAACCTTGTCTCTGGCGT | |
| | CCCTGATCGGTTCTCCGGTTCCGGGTCAGGCACTGATT | |
| | TCACACTGAAAATCAGCAGGGTGGAGGCTGAGGATGT | |
| | TGGAGTTTATTACTGCGCTCAGAATCTAGAACTTCCTT | |
| | ACACCTTCGGCGGAGGGACCAAGGTGGAGATCAAA | |
| scFab-CL | CGTACGGTGGCTGCACCATCTGTCTTCATCTTCCCGCC | 57 |
| | ATCTGATGAGCAGTTGAAATCTGGAACTGCCTCTGTTG | |
| | TGTGCCTGCTGAATAACTTCTATCCCAGAGAGGCCAA | |
| | AGTACAGTGGAAGGTGGATAACGCCCTCCAATCGGGT | |
| | AACTCCCAGGAGAGTGTCACAGAGCAGGACAGCAAG | |
| | GACAGCACCTACAGCCTCAGCAGCACCCTGACGCTGA | |
| | GCAAAGCAGACTACGAGAAACACAAAGTCTACGCCTG | |
| | CGAAGTCACCCATCAGGGCCTGAGCTCGCCCGTCACA | |
| | AAGAGCTTCAACAGGGGAGAGTGT | |
| Anti-CD20-(GA101)- | CAGGTGCAATTGGTGCAGTCTGGCGCTGAAGTTAAGA | 58 |
| scFab-VH | AGCCTGGGAGTTCAGTGAAGGTCTCCTGCAAGGCTTC | |
| | GGGATACGCCTTCAGCTATTCTTGGATCAATTGGGTGC | |
| | GGCAGGCGCCTGGACAAGGGCTCGAGTGGATGGGACG | |
| | GATCTTTCCCGGCGATGGGGATACTGACTACAATGGG | |
| | AAATTCAAGGGCAGAGTCACAATTACCGCCGACAAAT | |
| | CCACTAGCACAGCCTATATGGAGCTGAGCAGCCTGAG | |
| | ATCTGAGGACACGGCCGTGTATTACTGTGCAAGAAAT | |
| | GTCTTTGATGGTTACTGGCTTGTTTACTGGGGCCAGGG | |
| | AACCCTGGTCACCGTCTCCTCA | |
| Fab-CH1 | see Table 3 | 26 |
| CD28ATD-CD28CSD- | see Table 2 | 32 |
| CD3zSSD | | |
| Anti-CD20-(GA101)- | ATGGAGCTGTATCATCCTCTTCTTGGTAGCAACAGCTA | 59 |
| scFab- | CGGGTGTGCATTCCGACATCGTGATGACCCAGACTCC | |
| CD28ATD- | ACTCTCCCTGCCCGTCACCCCTGGAGAGCCCGCCAGCA | |
| CD28CSD- | TTAGCTGCAGGTCTAGCAAGAGCCTCTTGCACAGCAA | |
| CD3zSSD- | TGGCATCACTTATTTGTATTGGTACCTGCAAAAGCCAG | |
| eGFP fusion | GGCAGTCTCCACAGCTCCTGATTTATCAAATGTCCAAC | |
| pETR17101 | CTTGTCTCTGGCGTCCCTGATCGGTTCTCCGGTTCCGG | |
| | GTCAGGCACTGATTTCACACTGAAAATCAGCAGGGTG | |
| | GAGGCTGAGGATGTTGGAGTTTATTACTGCGCTCAGA | |
| | ATCTAGAACTTCCTTACACCTTCGGCGGAGGGACCAA | |
| | GGTGGAGATCAAACGTACGGTGGCTGCACCATCTGTC | |
| | TTCATCTTCCCGCCATCTGATGAGCAGTTGAAATCTGG | |
| | AACTGCCTCTGTTGTGTGCCTGCTGAATAACTTCTATC | |
| | CCAGAGAGGCCAAAGTACAGTGGAAGGTGGATAACG | |
| | CCCTCCAATCGGGTAACTCCCAGGAGAGTGTCACAGA | |
| | GCAGGACAGCAAGGACAGCACCTACAGCCTCAGCAGC | |
| | ACCCTGACGCTGAGCAAAGCAGACTACGAGAAACACA | |
| | AAGTCTACGCCTGCGAAGTCACCCATCAGGGCCTGAG | |
| | CTCGCCCGTCACAAAGAGCTTCAACAGGGGAGAGTGT | |
| | GGCGGCGGAGGATCTGGTGGCGGAGGTAGTGGTGGTG | |
| | GTGGATCTGGCGGAGGCGGCTCCGGCGGAGGTGGAAG | |
| | CGGAGGTGGTGGTTCCGGAGGACAGGTGCAATTGGTG | |
| | CAGTCTGGCGCTGAAGTTAAGAAGCCTGGGAGTTCAG | |
| | TGAAGGTCTCCTGCAAGGCTTCGGGATACGCCTTCAGC | |
| | TATTCTTGGATCAATTGGGTGCGGCAGGCGCCTGGAC | |
| | AAGGGCTCGAGTGGATGGGACGGATCTTTCCCGGCGA | |
| | TGGGGATACTGACTACAATGGGAAATTCAAGGGCAGA | |
| | GTCACAATTACCGCCGACAAATCCACTAGCACAGCCT | |
| | ATATGGAGCTGAGCAGCCTGAGATCTGAGGACACGGC | |
| | CGTGTATTACTGTGCAAGAAATGTCTTTGATGGTTACT | |
| | GGCTTGTTTACTGGGGCCAGGGAACCCTGGTCACCGTC | |
| | TCCTCAGCTAGCACCAAGGGCCCCTCCGTGTTCCCCCT | |
| | GGCCCCCAGCAGCAAGAGCACCAGCGGCGGCACAGCC | |
| | GCTCTGGGCTGCCTGGTCAAGGACTACTTCCCCGAGCC | |
| | CGTGACCGTGTCCTGGAACAGCGGAGCCCTGACCTCC | |
| | GGCGTGCACACCTTCCCCGCCGTGCTGCAGAGTTCTGG | |
| | CCTGTATAGCCTGAGCAGCGTGGTCACCGTGCCTTCTA | |
| | GCAGCCTGGGCACCCAGACCTACATCTGCAACGTGAA | |
| | CCACAAGCCCAGCAACACCAAGGTGGACAAGAAGGT | |
| | GGAGCCCAAGAGCTGCGGAGGGGGCGGATCCTTCTGG | |
| | GTGCTGGTGGTGGTGGGCGGCGTGCTGGCCTGCTACA | |
| | GCCTGCTGGTGACCGTGGCCTTCATCATCTTCTGGGTG | |
| | AGGAGCAAGAGGAGCAGGCTGCTGCACAGCGACTAC | |
| | ATGAACATGACCCCCAGGAGGCCCGGCCCCACCAGGA | |
| | AGCACTACCAGCCCTACGCCCCCCCCAGGGACTTCGC | |
| | CGCCTACAGGAGCAGGGTGAAGTTCAGCAGGAGCGCC | |
| | GACGCCCCCGCCTACCAGCAGGGCCAGAACCAGCTGT | |
| | ATAACGAGCTGAACCTGGGCAGGAGGGAGGAGTACG | |
| | ACGTGCTGGACAAGAGGAGGGGCAGGGACCCCGAGA | |
| | TGGGCGGCAAGCCCAGGAGGAAGAACCCCCAGGAGG | |
| | GCCTGTATAACGAGCTGCAGAAGGACAAGATGGCCGA | |
| | GGCCTACAGCGAGATCGGCATGAAGGGCGAGAGGAG | |
| | GAGGGGCAAGGGCCACGACGGCCTGTACCAGGGCCTG | |
| | AGCACCGCCACCAAGGACACCTACGACGCCCTGCACA | |
| | TGCAGGCCCTGCCCCCCAGGTCCGGAGAGGGCAGAGG | |
| | AAGTCTTCTAACATGCGGTGACGTGGAGGAGAATCCC | |
| | GGCCCTAGGGTGAGCAAGGGCGAGGAGCTGTTCACCG | |
| | GGGTGGTGCCCATCCTGGTCGAGCTGGACGGCGACGT | |
| | AAACGGCCACAAGTTCAGCGTGTCCGGCGAGGGCGAG | |
| | GGCGATGCCACCTACGGCAAGCTGACCCTGAAGTTCA | |
| | TCTGCACCACCGGCAAGCTGCCCGTGCCCTGGCCCACC | |
| | CTCGTGACCACCCTGACCTACGGCGTGCAGTGCTTCAG | |
| | CCGCTACCCCGACCACATGAAGCAGCACGACTTCTTC | |
| | AAGTCCGCCATGCCCGAAGGCTACGTCCAGGAGCGCA | |
| | CCATCTTCTTCAAGGACGACGGCAACTACAAGACCCG | |
| | CGCCGAGGTGAAGTTCGAGGGCGACACCCTGGTGAAC | |
| | CGCATCGAGCTGAAGGGCATCGACTTCAAGGAGGACG | |
| | GCAACATCCTGGGGCACAAGCTGGAGTACAACTACAA | |
| | CAGCCACAACGTCTATATCATGGCCGACAAGCAGAAG | |
| | AACGGCATCAAGGTGAACTTCAAGATCCGCCACAACA | |
| | TCGAGGACGGCAGCGTGCAGCTCGCCGACCACTACCA | |
| | GCAGAACACCCCCATCGGCGACGGCCCCGTGCTGCTG | |
| | CCCGACAACCACTACCTGAGCACCCAGTCCGCCCTGA | |
| | GCAAAGACCCCAACGAGAAGCGCGATCACATGGTCCT | |
| | GCTGGAGTTCGTGACCGCCGCCGGGATCACTCTCGGC | |
| | ATGGACGAGCTGTACAAGTGA | |

TABLE 10
Anti-CD20-ds-scFv amino acid sequences

| | | SEQ |
|---|---|---|
| | | ID |
| Construct | Protein Sequence | NO |
| Anti- | see Table 2 | 1 |
| CD20 | | |
| (GA101) | | |
| CDR H1 | | |
| Kabat | | |
| Anti- | see Table 2 | 2 |
| CD20 | | |
| (GA101) | | |
| CDR H2 | | |
| Kabat | | |
| Anti- | see Table 2 | 3 |
| CD20 | | |
| (GA101) | | |
| CDR H3 | | |
| Kabat | | |
| Anti- | see Table 2 | 4 |
| CD20 | | |
| (GA101) | | |
| CDR L1 | | |
| Kabat | | |
| Anti- | see Table 2 | 5 |
| CD20 | | |
| (GA101) | | |
| CDR L2 | | |
| Kabat | | |
| Anti- | see Table 2 | 6 |
| CD20 | | |
| (GA101) | | |
| CDR L3 | | |
| Kabat | | |
| Anti- | QVQLVQSGAEVKKPGSSVKVSCKASGYAFSYSWIN | 60 |
| CD20- | WVRQAPGQCLEWMGRIFPGDGDTDYNGKFKGRVTI | |
| (GA101)- | TADKSTSTAYMELSSLRSEDTAVYYCARNVFDGYW | |
| ds-scFv- | LVYWGQGTLVTVSSGGGGSGGGGSGGGGSGGGGSD | |
| CD28ATD- | IVMTQTPLSLPVTPGEPASISCRSSKSLLHSNGIT | |
| CD28CSD- | YLYWYLQKPGQSPQLLIYQMSNLVSGVPDRFSGSG | |
| CD3zSSD | SGTDFTLKISRVEAEDVGVYYCAQNLELPYTFGCG | |
| fusion | TKVEIKGGGGSFWVLVVVGGVLACYSLLVTVAFII | |
| pETR17162 | FWVRSKRSRLLHSDYMNMTPRRPGPTRKHYQPYAP | |
| | PRDFAAYRSRVKFSRSADAPAYQQGQNQLYNELNL | |
| | GRREEYDVLDKRRGRDPEMGGKPRRKNPQEGLYNE | |
| | LQKDKMAEAYSEIGMKGERRRGKGHDGLYQGLSTA | |
| | TKDTYDALHMQALPPR | |
| Anti- | QVQLVQSGAEVKKPGSSVKVSCKASGYAFSYSWIN | 61 |
| CD20- | WVRQAPGQCLEWMGRIFPGDGDTDYNGKFKGRVTI | |
| (GA101)- | TADKSTSTAYMELSSLRSEDTAVYYCARNVFDGYW | |
| ds-scFv | LVYWGQGTLVTVSSGGGGSGGGGSGGGGSGGGGSD | |
| | IVMTQTPLSLPVTPGEPASISCRSSKSLLHSNGIT | |
| | YLYWYLQKPGQSPQLLIYQMSNLVSGVPDRFSGSG | |
| | SGTDFTLKISRVEAEDVGVYYCAQNLELPYTFGCG | |
| | TKVEIK | |
| Anti- | QVQLVQSGAEVKKPGSSVKVSCKASGYAFSYSWIN | 62 |
| CD20- | WVRQAPGQCLEWMGRIFPGDGDTDYNGKFKGRVTI | |
| (GA101)- | TADKSTSTAYMELSSLRSEDTAVYYCARNVFDGYW | |
| ds-VH | LVYWGQGTLVTVSS | |
| Anti- | DIVMTQTPLSLPVTPGEPASISCRSSKSLLHSNGI | 63 |
| CD20- | TYLYWYLQKPGQSPQLLIYQMSNLVSGVPDRFSGS | |
| (GA101)- | GSGTDFTLKISRVEAEDVGVYYCAQNLELPYTFGC | |
| ds-VL | GTKVEIK | |
| CD28ATD- | see Table 2 | 17 |
| CD28CSD- | | |
| CD3zSSD | | |

TABLE 11
Anti-CD20 ds scFv DNA sequences

| | | SEQ |
|---|---|---|
| | | ID |
| Construct | DNA Sequenz | NO |
| Anti- | ATGGGATGGAGCTGTATCATCCTCTTCTTGGTAGCAAC | 64 |
| CD20- | AGCTACCGGTGTGCATTCCCAGGTGCAATTGGTGCAGT | |
| (GA101)- | CTGGCGCTGAAGTTAAGAAGCCTGGGAGTTCAGTGAA | |
| ds-Fab- | GGTCTCCTGCAAGGCTTCCGGTTACGCCTTCAGCTATT | |
| CD28ATD- | CTTGGATCAATTGGGTGCGGCAGGCGCCTGGACAATG | |
| CD28CSD- | TCTCGAGTGGATGGGACGGATCTTTCCCGGCGATGGG | |
| CD3zSSD | GATACTGACTACAATGGGAAATTCAAGGGCAGAGTCA | |
| fusion | CAATTACCGCCGACAAATCCACTAGCACAGCCTATAT | |
| pETR17162 | GGAGCTGAGCAGCCTGAGATCTGAGGACACGGCCGTG | |
| | TATTACTGTGCAAGAAATGTCTTTGATGGTTACTGGCT | |
| | TGTTTACTGGGGCCAGGGAACCCTGGTCACCGTCTCCT | |
| | CAGGAGGGGGCGGAAGTGGTGGCGGGGGAAGCGGCG | |
| | GGGGTGGCAGCGGAGGGGGCGGATCTGACATCGTGAT | |
| | GACCCAGACTCCACTCTCCCTGCCCGTCACCCCTGGAG | |
| | AGCCCGCCAGCATTAGCTGCAGGTCTAGCAAGAGCCT | |
| | CTTGCACAGCAATGGCATCACTTATTTGTATTGGTACC | |
| | TGCAAAAGCCAGGGCAGTCTCCACAGCTCCTGATTTAT | |
| | CAAATGTCCAACCTTGTCTCTGGCGTCCCTGACCGCTT | |
| | CTCCGGTTCCGGGTCAGGCACTGATTTCACACTGAAAA | |
| | TCAGCAGGGTGGAGGCTGAGGATGTTGGAGTTTATTA | |
| | CTGCGCTCAGAATCTAGAACTTCCTTACACCTTCGGCT | |
| | GTGGGACCAAGGTGGAGATCAAGGAGGGGGCGGATC | |
| | CTTCTGGGTGCTGGTGGTGGTGGGCGGCGTGCTGGCCT | |
| | GCTACAGCCTGCTGGTGACCGTGGCCTTCATCATCTTC | |
| | TGGGTGAGGAGCAAGAGGAGCAGGCTGCTGCACAGC | |
| | GACTACATGAACATGACCCCCAGGAGGCCCGGCCCCA | |
| | CCAGGAAGCACTACCAGCCCTACGCCCCCCCCAGGGA | |
| | CTTCGCCGCCTACAGGAGCAGGGTGAAGTTCAGCAGG | |
| | AGCGCCGACGCCCCCGCCTACCAGCAGGGCCAGAACC | |
| | AGCTGTATAACGAGCTGAACCTGGGCAGGAGGGAGGA | |
| | GTACGACGTGCTGGACAAGAGGAGGGGCAGGGACCC | |
| | CGAGATGGGCGGCAAGCCCAGGAGGAAGAACCCCCA | |
| | GGAGGGCCTGTATAACGAGCTGCAGAAGGACAAGATG | |
| | GCCGAGGCCTACAGCGAGATCGGCATGAAGGGCGAG | |
| | AGGAGGAGGGGCAAGGGCCACGACGGCCTGTACCAG | |
| | GGCCTGAGCACCGCCACCAAGGACACCTACGACGCCC | |
| | TGCACATGCAGGCCCTGCCCCCCAGG | |
| Anti- | CAGGTGCAATTGGTGCAGTCTGGCGCTGAAGTTAAGA | 65 |
| CD20- | AGCCTGGGAGTTCAGTGAAGGTCTCCTGCAAGGCTTC | |
| (GA101)- | CGGTTACGCCTTCAGCTATTCTTGGATCAATTGGGTGC | |
| ds-VH | GGCAGGCGCCTGGACAATGTCTCGAGTGGATGGGACG | |
| | GATCTTTCCCGGCGATGGGGATACTGACTACAATGGG | |
| | AAATTCAAGGGCAGAGTCACAATTACCGCCGACAAAT | |
| | CCACTAGCACAGCCTATATGGAGCTGAGCAGCCTGAG | |
| | ATCTGAGGACACGGCCGTGTATTACTGTGCAAGAAAT | |
| | GTCTTTGATGGTTACTGGCTTGTTTACTGGGGCCAGGG | |
| | AACCCTGGTCACCGTCTCCTCA | |
| Anti- | GACATCGTGATGACCCAGACTCCACTCTCCCTGCCCGT | 66 |
| CD20- | CACCCCTGGAGAGCCCGCCAGCATTAGCTGCAGGTCT | |
| (GA101)- | AGCAAGAGCCTCTTGCACAGCAATGGCATCACTTATTT | |
| ds-VL | GTATTGGTACCTGCAAAAGCCAGGGCAGTCTCCACAG | |
| | CTCCTGATTTATCAAATGTCCAACCTTGTCTCTGGCGT | |
| | CCCTGACCGCTTCTCCGGTTCCGGGTCAGGCACTGATT | |
| | TCACACTGAAAATCAGCAGGGTGGAGGCTGAGGATGT | |
| | TGGAGTTTATTACTGCGCTCAGAATCTAGAACTTCCTT | |
| | ACACCTTCGGCTGTGGGACCAAGGTGGAGATCAAA | |
| CD28TM- | see Table 3 | 32 |
| CD28- | | |
| CD3z | | |
| Anti- | GCCACCATGGGATGGAGCTGTATCATCCTCTTCTTGGT | 67 |
| CD20- | AGCAACAGCTACCGGTGTGCATTCCCAGGTGCAATTG | |
| (GA101)- | GTGCAGTCTGGCGCTGAAGTTAAGAAGCCTGGGAGTT | |
| ds-scFv- | CAGTGAAGGTCTCCTGCAAGGCTTCCGGTTACGCCTTC | |
| CD28ATD- | AGCTATTCTTGGATCAATTGGGTGCGGCAGGCGCCTG | |
| CD28CSD- | GACAATGTCTCGAGTGGATGGGACGGATCTTTCCCGG | |
| CD3zSSD- | CGATGGGGATACTGACTACAATGGGAAATTCAAGGGC | |
| eGFP | AGAGTCACAATTACCGCCGACAAATCCACTAGCACAG | |
| fusion | CCTATATGGAGCTGAGCAGCCTGAGATCTGAGGACAC | |
| pETR17162 | GGCCGTGTATTACTGTGCAAGAAATGTCTTTGATGGTT | |
| | ACTGGCTTGTTTACTGGGGCCAGGGAACCCTGGTCACC | |
| | GTCTCCTCAGGAGGGGGCGGAAGTGGTGGCGGGGGAA | |
| | GCGGCGGGGGTGGCAGCGGAGGGGGCGGATCTGACA | |
| | TCGTGATGACCCAGACTCCACTCTCCCTGCCCGTCACC | |
| | CCTGGAGAGCCCGCCAGCATTAGCTGCAGGTCTAGCA | |
| | AGAGCCTCTTGCACAGCAATGGCATCACTTATTTGTAT | |
| | TGGTACCTGCAAAAGCCAGGGCAGTCTCCACAGCTCC | |
| | TGATTTATCAAATGTCCAACCTTGTCTCTGGCGTCCCT | |
| | GACCGCTTCTCCGGTTCCGGGTCAGGCACTGATTTCAC | |
| | ACTGAAAATCAGCAGGGTGGAGGCTGAGGATGTTGGA | |
| | GTTTATTACTGCGCTCAGAATCTAGAACTTCCTTACAC | |
| | CTTCGGCTGTGGGACCAAGGTGGAGATCAAAGGAGGG | |
| | GGCGGATCCTTCTGGGTGCTGGTGGTGGTGGGCGGCG | |
| | TGCTGGCCTGCTACAGCCTGCTGGTGACCGTGGCCTTC | |
| | ATCATCTTCTGGGTGAGGAGCAAGAGGAGCAGGCTGC | |
| | TGCACAGCGACTACATGAACATGACCCCCAGGAGGCC | |
| | CGGCCCCACCAGGAAGCACTACCAGCCCTACGCCCCC | |
| | CCCAGGGACTTCGCCGCCTACAGGAGCAGGGTGAAGT | |
| | TCAGCAGGAGCGCCGACGCCCCCGCCTACCAGCAGGG | |
| | CCAGAACCAGCTGTATAACGAGCTGAACCTGGGCAGG | |
| | AGGGAGGAGTACGACGTGCTGGACAAGAGGAGGGGC | |
| | AGGGACCCCGAGATGGGCGGCAAGCCCAGGAGGAAG | |
| | AACCCCCAGGAGGGCCTGTATAACGAGCTGCAGAAGG | |
| | ACAAGATGGCCGAGGCCTACAGCGAGATCGGCATGAA | |
| | GGGCGAGAGGAGGAGGGGCAAGGGCCACGACGGCCT | |
| | GTACCAGGGCCTGAGCACCGCCACCAAGGACACCTAC | |
| | GACGCCCTGCACATGCAGGCCCTGCCCCCCAGGTCCG | |
| | GAGAGGGCAGAGGAAGTCTTCTAACATGCGGTGACGT | |
| | GGAGGAGAATCCCGGCCCTAGGGTGAGCAAGGGCGA | |
| | GGAGCTGTTCACCGGGGTGGTGCCCATCCTGGTCGAG | |
| | CTGGACGGCGACGTAAACGGCCACAAGTTCAGCGTGT | |
| | CCGGCGAGGGCGAGGGCGATGCCACCTACGGCAAGCT | |
| | GACCCTGAAGTTCATCTGCACCACCGGCAAGCTGCCC | |
| | GTGCCCTGGCCCACCCTCGTGACCACCCTGACCTACGG | |
| | CGTGCAGTGCTTCAGCCGCTACCCCGACCACATGAAG | |
| | CAGCACGACTTCTTCAAGTCCGCCATGCCCGAAGGCT | |
| | ACGTCCAGGAGCGCACCATCTTCTTCAAGGACGACGG | |
| | CAACTACAAGACCCGCGCCGAGGTGAAGTTCGAGGGC | |
| | GACACCCTGGTGAACCGCATCGAGCTGAAGGGCATCG | |
| | ACTTCAAGGAGGACGGCAACATCCTGGGGCACAAGCT | |
| | GGAGTACAACTACAACAGCCACAACGTCTATATCATG | |
| | GCCGACAAGCAGAAGAACGGCATCAAGGTGAACTTCA | |
| | AGATCCGCCACAACATCGAGGACGGCAGCGTGCAGCT | |
| | CGCCGACCACTACCAGCAGAACACCCCCATCGGCGAC | |
| | GGCCCCGTGCTGCTGCCCGACAACCACTACCTGAGCA | |
| | CCCAGTCCGCCCTGAGCAAAGACCCCAACGAGAAGCG | |
| | CGATCACATGGTCCTGCTGGAGTTCGTGACCGCCGCCG | |
| | GGATCACTCTCGGCATGGACGAGCTGTACAAGTGA | |

TABLE 12
Anti-PDL1 Fab amino acid sequences

| | | SEQ |
|---|---|---|
| Con- | | ID |
| struct | Protein Sequence | NO |
| Anti- | DSWIH | 68 |
| PDL1 | | |
| CDR H1 | | |
| Kabat | | |
| Anti- | WISPYGGSTYYADSVKG | 69 |
| PDL1 | | |
| CDR H2 | | |
| Kabat | | |
| Anti- | RHWPGGFDY | 70 |
| PDL1 | | |
| CDR H3 | | |
| Kabat | | |
| Anti- | RASQDVSTAVA | 71 |
| PDL1 | | |
| CDR 1 | | |
| L1 | | |
| Kabat | | |
| Anti- | SASFLYS | 72 |
| PDL1 | | |
| CDR L2 | | |
| Kabat | | |
| Anti- | QQYLYHPAT | 73 |
| PDL1 | | |
| CDR L3 | | |
| Kabat | | |
| Anti- | EVQLVESGGGLVQPGGSLRLSCAASGFTFSDSWIHWVRQ | 74 |
| PDL1 | APGKGLEWVAWISPYGGSTYYADSVKGRFTISADTSKNT | |
| Fab | AYLQMNSLRAEDTAVYYCARRHWPGGFDYWGQGTLVT | |
| heavy | VSSASTKGPSVFPLAPSSKSTSGGTAALGCLVKDYFPEPV | |
| chain- | TVSWNSGALTSGVHTFPAVLQSSGLYSLSSVVTVPSSSLG | |
| CD28ATD- | TQTYICNVNHKPSNTKVDKKVEPKSCGGGGSFWVLVVV | |
| CD28CSD- | GGVLACYSLLVTVAFIIFWVRSKRSRLLHSDYMNMTPRR | |
| CD3zSSD | PGPTRKHYQPYAPPRDFAAYRSRVKFSRSADAPAYQQG | |
| fusion | QNQLYNELNLGRREEYDVLDKRRGRDPEMGGKPRRKNP | |
| | QEGLYNELQKDKMAEAYSEIGMKGERRRGKGHDGLYQ | |
| | GLSTATKDTYDALHMQALPPR | |
| Anti- | EVQLVESGGGLVQPGGSLRLSCAASGFTFSDSWIHWVRQ | 75 |
| PDL1 | APGKGLEWVAWISPYGGSTYYADSVKGRFTISADTSKNT | |
| Fab | AYLQMNSLRAEDTAVYYCARRHWPGGFDYWGQGTLVT | |
| heavy | VSSASTKGPSVFPLAPSSKSTSGGTAALGCLVKDYFPEPV | |
| chain | TVSWNSGALTSGVHTFPAVLQSSGLYSLSSVVTVPSSSLG | |
| | TQTYICNVNHKPSNTKVDKKVEPKSC | |
| Anti- | DIQMTQSPSSLSASVGDRVTITCRASQDVSTAVAWYQQK | 76 |
| PDL1 | PGKAPKLLIYSASFLYSGVPSRFSGSGSGTDFTLTISSLQ | |
| Fab | PEDFATYYCQQYLYHPATFGQGTKVEIKRRTVAAPSVFIF | |
| light | PPSDEQLKSGTASVVCLLNNFYPREAKVQWKVDNALQSGN | |
| chain | SQESVTEQDSKDSTYSLSSTLTLSKADYEKHKVYACEVT | |
| | HQGLSSPVTKSFNRGEC | |
| Anti- | DIQMTQSPSSLSASVGDRVTITCRASQDVSTAVAWYQQK | 77 |
| PDL1- | PGKAPKLLIYSASFLYSGVPSRFSGSGSGTDFTLTISSLQ | |
| VL | PEDFATYYCQQYLYHPATFGQGTKVEIKR | |
| Fab CL | see Table 2 | 11 |
| Anti- | EVQLVESGGGLVQPGGSLRLSCAASGFTFSDSWIHWVRQ | 78 |
| PDL1- | APGKGLEWVAWISPYGGSTYYADSVKGRFTISADTSKNT | |
| VH | AYLQMNSLRAEDTAVYYCARRHWPGGFDYWGQGTLVT | |
| | VSS | |
| Fab CH1 | see Table 2 | 13 |
| G4S | see Table 2 | 20 |
| linker | | |
| CD28TM- | see Table 2 | 17 |
| CD28- | | |
| CD3z | | |
| T2A | see Table 12 | 21 |
| linker | | |
| eGFP | see Table 2 | 18 |

TABLE 13
PDL1 crossFab (VH-CL-ATD) amino acid sequences

| | | SEQ |
|---|---|---|
| Con- | | ID |
| struct | Protein Sequence | NO |
| Anti- | see Table 12 | 68 |
| PDL1 | | |
| CDR H1 | | |
| Kabat | | |
| Anti- | see Table 12 | 69 |
| PDL1 | | |
| CDR H2 | | |
| Kabat | | |
| Anti- | see Table 12 | 70 |
| PDL1 | | |
| CDR H3 | | |
| Kabat | | |
| Anti- | see Table 12 | 71 |
| PDL1 | | |
| CDR 1 | | |
| L1 Kabat | | |
| Anti- | see Table 12 | 72 |
| PDL1 | | |
| CDR L2 | | |
| Kabat | | |
| Anti- | see Table 12 | 73 |
| PDL1 | | |
| CDR L3 | | |
| Kabat | | |
| Anti- | EVQLVESGGGLVQPGGSLRLSCAASGFTFSDSWIHWVR | 79 |
| PDL1- | QAPGKGLEWVAWISPYGGSTYYADSVKGRFTISADTSK | |
| crossFab | NTAYLQMNSLRAEDTAVYYCARRHWPGGFDYWGQGTLV | |
| VH-CL | TVSSASVAAPSVFIFPPSDEQLKSGTASVVCLLNNFYP | |
| light | REAKVQWKVDNALQSGNSQESVTEQDSKDSTYSLSSTL | |
| chain- | TLSKADYEKHKVYACEVTHQGLSSPVTKSFNRGECGGG | |
| CD28ATD- | GSFWVLVVVGGVLACYSLLVTVAFIIFWVRSKRSRLLH | |
| CD28CSD- | SDYMNMTPRRPGPTRKHYQPYAPPRDFAAYRSRVKFSR | |
| CD3zSSD | SADAPAYQQGQNQLYNELNLGRREEYDVLDKRRGRDPE | |
| fusion | MGGKPRRKNPQEGLYNELQKDKMAEAYSEIGMKGERRR | |
| | GKGHDGLYQGLSTATKDTYDALHMQALPPR | |
| Anti- | EVQLVESGGGLVQPGGSLRLSCAASGFTFSDSWIHWVR | 80 |
| PDL1- | QAPGKGLEWVAWISPYGGSTYYADSVKGRFTISADTSK | |
| crossFab | NTAYLQMNSLRAEDTAVYYCARRHWPGGFDYWGQGTLV | |
| VH-CL | TVSSASVAAPSVFIFPPSDEQLKSGTASVVCLLNNFYP | |
| light | REAKVQWKVDNALQSGNSQESVTEQDSKDSTYSLSSTL | |
| chain | TLSKADYEKHKVYACEVTHQGLSSPVTKSFNRGEC | |
| Anti- | DIQMTQSPSSLSASVGDRVTITCRASQDVSTAVAWYQQ | 81 |
| PDL1- | KPGKAPKLLIYSASFLYSGVPSRFSGSGSGTDFTLTIS | |
| crossFab | SLQPEDFATYYCQQYLYHPATFGQGTKVEIKRSSASTK | |
| VL-CH | GPSVFPLAPSSKSTSGGTAALGCLVKDYFPEPVTVSWN | |
| heavy | SGALTSGVHTFPAVLQSSGLYSLSSVVTVPSSSLGTQT | |
| chain | YICNVNHKPSNTKVDKKVEPKSC | |
| Anti- | see Table 12 | 78 |
| PDL1-VH | | |
| crossFab | see Table 4 | 39 |
| CL | | |
| Anti- | see Table 12 | 77 |
| PDL1-VL | | |
| crossFab | see Table 4 | 40 |
| CH1 | | |
| G4S | see Table 2 | 20 |
| linker | | |
| CD28ATD- | see Table 2 | 17 |
| CD28CSD- | | |
| CD3zSSD | | |
| T2A | see Table 2 | 21 |
| linker | | |
| eGFP | see Table 2 | 18 |

TABLE 14
Anti-PDL1 crossFab (VL-CH1-ATD) amino acid
sequences

| | | SEQ |
|---|---|---|
| Con- | | ID |
| struct | Protein Sequence | NO |
| Anti- | see Table 12 | 68 |
| PDL1 | | |
| CDR H1 | | |
| Kabat | | |
| Anti- | see Table 12 | 69 |
| PDL1 | | |
| CDR H2 | | |
| Kabat | | |
| Anti- | see Table 12 | 70 |
| PDL1 | | |
| CDR H3 | | |
| Kabat | | |
| Anti- | see Table 12 | 71 |
| PDL1 | | |
| CDR 1 L1 | | |
| Kabat | | |
| Anti- | see Table 12 | 72 |
| PDL1 | | |
| CDR L2 | | |
| Kabat | | |
| Anti- | see Table 12 | 73 |
| PDL1 | | |
| CDR L3 | | |
| Kabat | | |
| Anti- | DIQMTQSPSSLSASVGDRVTITCRASQDVSTAVAWYQ | 82 |
| PDL1- | QKPGKAPKLLIYSASFLYSGVPSRFSGSGSGTDFTLT | |
| crossFab | ISSLQPEDFATYYCQQYLYHPATFGQGTKVEIKRSSA | |
| VL-CH | STKGPSVFPLAPSSKSTSGGTAALGCLVKDYFPEPVT | |
| heavy | VSWNSGALTSGVHTFPAVLQSSGLYSLSSVVTVPSSS | |
| chain- | LGTQTYICNVNHKPSNTKVDKKVEPKSCGGGGSFWVL | |
| ATD- | VVVGGVLACYSLLVTVAFIIFWVRSKRSRLLHSDYMN | |
| CD28ATD- | MTPRRPGPTRKHYQPYAPPRDFAAYRSRVKFSRSADA | |
| CD28CSD- | PAYQQGQNQLYNELNLGRREEYDVLDKRRGRDPEMGG | |
| CD3zSSD | KPRRKNPQEGLYNELQKDKMAEAYSEIGMKGERRRGK | |
| fusion | GHDGLYQGLSTATKDTYDALHMQALPPR | |
| Anti- | DIQMTQSPSSLSASVGDRVTITCRASQDVSTAVAWYQ | 83 |
| PDL1- | QKPGKAPKLLIYSASFLYSGVPSRFSGSGSGTDFTLT | |
| crossFab | ISSLQPEDFATYYCQQYLYHPATFGQGTKVEIKRSSA | |
| VL-CH | STKGPSVFPLAPSSKSTSGGTAALGCLVKDYFPEPVT | |
| heavy | VSWNSGALTSGVHTFPAVLQSSGLYSLSSVVTVPSSS | |
| chain | LGTQTYICNVNHKPSNTKVDKKVEPKSC | |
| Anti- | EVQLVESGGGLVQPGGSLRLSCAASGFTFSDSWIHWV | 84 |
| PDL1- | RQAPGKGLEWVAWISPYGGSTYYADSVKGRFTISADT | |
| crossFab | SKNTAYLQMNSLRAEDTAVYYCARRHWPGGFDYWGQG | |
| VH-CL | TLVTVSSASVAAPSVFIFPPSDEQLKSGTASVVCLLN | |
| light | NFYPREAKVQWKVDNALQSGNSQESVTEQDSKDSTYS | |
| chain | LSSTLTLSKADYEKHKVYACEVTHQGLSSPVTKSFNR | |
| | GEC | |
| Anti- | see Table 12 | 78 |
| PDL1-VH | | |
| crossFab | see Table 4 | 39 |
| CL | | |
| Anti- | see Table 12 | 77 |
| PDL1-VL | | |
| crossFab | see Table 4 | 40 |
| CH1 | | |
| (G4S)4 | GGGGS | 20 |
| linker | | |
| CD28ATD- | see Table 2 | 17 |
| CD28CSD- | | |
| CD3zSSD | | |
| T2A | see Table 2 | 21 |
| linker | | |
| eGFP | see Table 2 | 18 |

TABLE 15
Anti-PDL1 Fab (VL-CL-ATD) amino acid sequences

| | | SEQ |
|---|---|---|
| Con- | | ID |
| struct | Protein Sequence | NO |
| Anti- | see Table 12 | 68 |
| PDL1 | | |
| CDR H1 | | |
| Kabat | | |
| Anti- | see Table 12 | 69 |
| PDL1 | | |
| CDR H2 | | |
| Kabat | | |
| Anti- | see Table 12 | 70 |
| PDL1 | | |
| CDR H3 | | |
| Kabat | | |
| Anti- | see Table 12 | 71 |
| PDL1 | | |
| CDR 1 | | |
| L1 Kabat | | |
| Anti- | see Table 12 | 72 |
| PDL1 | | |
| CDR L2 | | |
| Kabat | | |
| Anti- | see Table 12 | 73 |
| PDL1 | | |
| CDR L3 | | |
| Kabat | | |
| Anti- | DIQMTQSPSSLSASVGDRVTITCRASQDVSTAVAWYQ | 85 |
| PDL1- | QKPGKAPKLLIYSASFLYSGVPSRFSGSGSGTDFTLT | |
| Fab | ISSLQPEDFATYYCQQYLYHPATFGQGTKVEIKRRTV | |
| light | AAPSVFIFPPSDEQLKSGTASVVCLLNNFYPREAKVQ | |
| chain- | WKVDNALQSGNSQESVTEQDSKDSTYSLSSTLTLSKA | |
| ATD- | DYEKHKVYACEVTHQGLSSPVTKSFNRGECGGGGSFW | |
| CD28ATD- | VLVVVGGVLACYSLLVTVAFIIFWVRSKRSRLLHSDY | |
| CD28CSD- | MNMTPRRPGPTRKHYQPYAPPRDFAAYRSRVKFSRSA | |
| CD3zSSD | DAPAYQQGQNQLYNELNLGRREEYDVLDKRRGRDPEM | |
| fusion | GGKPRRKNPQEGLYNELQKDKMAEAYSEIGMKGERRR | |
| | GKGHDGLYQGLSTATKDTYDALHMQALPPR | |
| Anti- | see Table 12 | 75 |
| PDL1 Fab | | |
| heavy | | |
| chain | | |
| Anti- | see Table 12 | 76 |
| PDL1 Fab | | |
| light | | |
| chain | | |
| Anti- | see Table 12 | 78 |
| PDL1-VH | | |
| CL | see Table 2 | 11 |
| Anti- | see Table 12 | 77 |
| PDL1-VL | | |
| CH1 | see Table 2 | 13 |
| G4S | see Table 2 | 20 |
| linker | | |
| CD28TM- | see Table 2 | 17 |
| CD28- | | |
| CD3z | | |
| T2A | see Table 12 | 21 |
| linker | | |
| eGFP | see Table 2 | 18 |

TABLE 16
Anti-PDL1 scFab amino acid sequences

| | | SEQ |
|---|---|---|
| Con- | | ID |
| struct | Protein Sequence | NO |
| Anti- | see Table 12 | 68 |
| PDL1 | | |
| CDR H1 | | |
| Kabat | | |
| Anti- | see Table 12 | 69 |
| PDL1 | | |
| CDR H2 | | |
| Kabat | | |
| Anti- | see Table 12 | 70 |
| PDL1 | | |
| CDR H3 | | |
| Kabat | | |
| Anti- | see Table 12 | 71 |
| PDL1 | | |
| CDR 1 | | |
| L1 Kabat | | |
| Anti- | see Table 12 | 72 |
| PDL1 | | |
| CDR L2 | | |
| Kabat | | |
| Anti- | see Table 12 | 73 |
| PDL1 | | |
| CDR L3 | | |
| Kabat | | |
| Anti- | DIQMTQSPSSLSASVGDRVTITCRASQDVSTAVAWYQ | 86 |
| PDL1- | QKPGKAPKLLIYSASFLYSGVPSRFSGSGSGTDFTLT | |
| scFab- | ISSLQPEDFATYYCQQYLYHPATFGQGTKVEIKRRTV | |
| CD28ATD- | AAPSVFIFPPSDEQLKSGTASVVCLLNNFYPREAKVQ | |
| CD28CSD- | WKVDNALQSGNSQESVTEQDSKDSTYSLSSTLTLSKA | |
| CD3zSSD | DYEKHKVYACEVTHQGLSSPVTKSFNRGECGGGGSGG | |
| fusion | GGSGGGGSGGGGSGGGGSGGGGSEVQLVESGGGLVQP | |
| | GGSLRLSCAASGFTFSDSWIHWVRQAPGKGLEWVAWI | |
| | SPYGGSTYYADSVKGRFTISADTSKNTAYLQMNSLRA | |
| | EDTAVYYCARRHWPGGFDYWGQGTLVTVSSASTKGPS | |
| | VFPLAPSSKSTSGGTAALGCLVKDYFPEPVTVSWNSG | |
| | ALTSGVHTFPAVLQSSGLYSLSSVVTVPSSSLGTQTY | |
| | ICNVNHKPSNTKVDKKVEPKSCGGGGSFWVLVVVGGV | |
| | LACYSLLVTVAFIIFWVRSKRSRLLHSDYMNMTPRRP | |
| | GPTRKHYQPYAPPRDFAAYRSRVKFSRSADAPAYQQG | |
| | QNQLYNELNLGRREEYDVLDKRRGRDPEMGGKPRRKN | |
| | PQEGLYNELQKDKMAEAYSEIGMKGERRRGKGHDGLY | |
| | QGLSTATKDTYDALHMQALPPR | |
| Anti- | DIQMTQSPSSLSASVGDRVTITCRASQDVSTAVAWYQ | 87 |
| PDL1- | QKPGKAPKLLIYSASFLYSGVPSRFSGSGSGTDFTLT | |
| scFab | ISSLQPEDFATYYCQQYLYHPATFGQGTKVEIKRRTV | |
| | AAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGECGGGGSGG | |
| | GGSGGGGSGGGGSGGGGSGGGGSEVQLVESGGGLVQP | |
| | GGSLRLSCAASGFTFSDSWIHWVRQAPGKGLEWVAWI | |
| | SPYGGSTYYADSVKGRFTISADTSKNTAYLQMNSLRA | |
| | EDTAVYYCARRHWPGGFDYWGQGTLVTVSSASTKGPS | |
| | VFPLAPSSKSTSGGTAALGCLVKDYFPEPVTVSWNSG | |
| | ALTSGVHTFPAVLQSSGLYSLSSVVTVPSSSLGTQTY | |
| | ICNVNHKPSNTKVDKKVEPKSC | |
| Anti- | see Table 12 | 77 |
| PDL1-VL | | |
| scFab-CL | see Table 8 | 53 |
| Anti- | see Table 12 | 78 |
| PDL1-VH | | |
| Fab CH1 | see Table 2 | 13 |
| CD28ATD- | see Table 2 | 17 |
| CD28CSD- | | |
| CD3zSSD | | |

TABLE 17
Anti-PDL1 ds scFv amino acid sequences

| | | SEQ |
|---|---|---|
| Con- | | ID |
| struct | Protein Sequence | NO |
| Anti- | see Table 12 | 68 |
| PDL1 | | |
| CDR H1 | | |
| Kabat | | |
| Anti- | see Table 12 | 69 |
| PDL1 | | |
| CDR H2 | | |
| Kabat | | |
| Anti- | see Table 12 | 70 |
| PDL1 | | |
| CDR H3 | | |
| Kabat | | |
| Anti- | see Table 12 | 71 |
| PDL1 | | |
| CDR 1 | | |
| L1 Kabat | | |
| Anti- | see Table 12 | 72 |
| PDL1 | | |
| CDR L2 | | |
| Kabat | | |
| Anti- | see Table 12 | 73 |
| PDL1 | | |
| CDR L3 | | |
| Kabat | | |
| Anti- | EVQLVESGGGLVQPGGSLRLSCAASGFTFSDSWIHWV | 88 |
| PDL1-ds- | RQAPGKCLEWVAWISPYGGSTYYADSVKGRFTISADT | |
| scFv- | SKNTAYLQMNSLRAEDTAVYYCARRHWPGGFDYWGQG | |
| CD28ATD- | TLVTVSSGGGGSGGGGSGGGGSGGGGSDIQMTQSPSS | |
| CD28CSD- | LSASVGDRVTITCRASQDVSTAVAWYQQKPGKAPKLL | |
| CD3zSSD | IYSASFLYSGVPSRFSGSGSGTDFTLTISSLQPEDFA | |
| fusion | TYYCQQYLYHPATFGQGCKVEIKGGGGSFWVLVVVGG | |
| | VLACYSLLVTVAFIIFWVRSKRSRLLHSDYMNMTPRR | |
| | PGPTRKHYQPYAPPRDFAAYRSRVKFSRSADAPAYQQ | |
| | GQNQLYNELNLGRREEYDVLDKRRGRDPEMGGKPRRK | |
| | NPQEGLYNELQKDKMAEAYSEIGMKGERRRGKGHDGL | |
| | YQGLSTATKDTYDALHMQALPPR | |
| Anti- | EVQLVESGGGLVQPGGSLRLSCAASGFTFSDSWIHWV | 89 |
| PDL1- | RQAPGKCLEWVAWISPYGGSTYYADSVKGRFTISADT | |
| ds-scFv | SKNTAYLQMNSLRAEDTAVYYCARRHWPGGFDYWGQG | |
| | TLVTVSSGGGGSGGGGSGGGGSGGGGSDIQMTQSPSS | |
| | LSASVGDRVTITCRASQDVSTAVAWYQQKPGKAPKLL | |
| | IYSASFLYSGVPSRFSGSGSGTDFTLTISSLQPEDFA | |
| | TYYCQQYLYHPATFGQGCKVEIK | |
| Anti- | EVQLVESGGGLVQPGGSLRLSCAASGFTFSDSWIHWV | 90 |
| PDL1- | RQAPGKCLEWVAWISPYGGSTYYADSVKGRFTISADT | |
| ds-VH | SKNTAYLQMNSLRAEDTAVYYCARRHWPGGFDYWGQG | |
| | TLVTVSS | |
| Anti- | DIQMTQSPSSLSASVGDRVTITCRASQDVSTAVAWYQ | 91 |
| PDL1- | QKPGKAPKLLIYSASFLYSGVPSRFSGSGSGTDFTLT | |
| ds-VL | ISSLQPEDFATYYCQQYLYHPATFGQGCKVEIK | |
| CD28ATD- | see Table 2 | 17 |
| CD28CSD- | | |
| CD3zSSD | | |

TABLE 18

| | | SEQ |
|---|---|---|
| Con- | | ID |
| struct | Amino acid sequence | NO |
| Human | ATGGCGCGCCCGCATCCGTGGTGGCTGTGCGTGCTGG | 92 |
| CD27 | GCACCCTGGTGGGCCTGAGCGCGACCCCGGCGCCGAA | |
| | AAGCTGCCCGGAACGCCATTATTGGGCGCAGGGCAAA | |
| | CTGTGCTGCCAGATGTGCGAACCGGGCACCTTTCTGGT | |
| | GAAAGATTGCGATCAGCATCGCAAAGCGGCGCAGTGC | |
| | GATCCGTGCATTCCGGGCGTGAGCTTTAGCCCGGATCA | |
| | TCATACCCGCCCGCATTGCGAAAGCTGCCGCCATTGCA | |
| | ACAGCGGCCTGCTGGTGCGCAACTGCACCATTACCGC | |
| | GAACGCGGAATGCGCGTGCCGCAACGGCTGGCAGTGC | |
| | CGCGATAAAGAATGCACCGAATGCGATCCGCTGCCGA | |
| | ACCCGAGCCTGACCGCGCGCAGCAGCCAGGCGCTGAG | |
| | CCCGCATCCGCAGCCGACCCATCTGCCGTATGTGAGC | |
| | GAAATGCTGGAAGCGCGCACCGCGGGCCATATGCAGA | |
| | CCCTGGCGGATTTTCGCCAGCTGCCGGCGCGCACCCTG | |
| | AGCACCCATTGGCCGCCGCAGCGCAGCCTGTGCAGCA | |
| | GCGATTTTATTCGCATTCTGGTGATTTTTAGCGGCATG | |
| | TTTCTGGTGTTTACCCTGGCGGGCGCGCTGTTTCTGCA | |
| | TCAGCGCCGCAAATATCGCAGCAACAAAGGCGAAAGC | |
| | CCGGTGGAACCGGCGGAACCGTGCCATTATAGCTGCC | |
| | CGCGCGAAGAAGAAGGCAGCACCATTCCGATTCAGGA | |
| | AGATTATCGCAAACCGGAACCGGCGTGCAGCCCG | |
| Human | MARPHPWWLCVLGTLVGLSATPAPKSCPERHYWAQGK | 93 |
| CD27 | LCCQMCEPGTFLVKDCDQHRKAAQCDPCIPGVSFSPDH | |
| | HTRPHCESCRHCNSGLLVRNCTITANAECACRNGWQCR | |
| | DKECTECDPLPNPSLTARSSQALSPHPQPTHLPYVSEM | |
| | LEARTAGHMQTLADFRQLPARTLSTHWPPQRSLCSSDF | |
| | IRILVIFSGMFLVFTLAGALFLHQRRKYRSNKGESPVE | |
| | PAEPCHYSCPREEEGSTIPIQEDYRKPEPACSP | |
| Murine | ATGGCGTGGCCGCCGCCGTATTGGCTGTGCATGCTGG | 94 |
| CD27 | GCACCCTGGTGGGCCTGAGCGCGACCCTGGCGCCGAA | |
| | CAGCTGCCCGGATAAACATTATTGGACCGGCGGCGGC | |
| | CTGTGCTGCCGCATGTGCGAACCGGGCACCTTTTTTGT | |
| | GAAAGATTGCGAACAGGATCGCACCGCGGCGCAGTGC | |
| | GATCCGTGCATTCCGGGCACCAGCTTTAGCCCGGATTA | |
| | TCATACCCGCCCGCATTGCGAAAGCTGCCGCCATTGCA | |
| | ACAGCGGCTTTCTGATTCGCAACTGCACCGTGACCGCG | |
| | AACGCGGAATGCAGCTGCAGCAAAAACTGGCAGTGCC | |
| | GCGATCAGGAATGCACCGAATGCGATCCGCCGCTGAA | |
| | CCCGGCGCTGACCCGCCAGCCGAGCGAAACCCCGAGC | |
| | CCGCAGCCGCCGCCGACCCATCTGCCGCATGGCACCG | |
| | AAAAACCGAGCTGGCCGCTGCATCGCCAGCTGCCGAA | |
| | CAGCACCGTGTATAGCCAGCGCAGCAGCCATCGCCCG | |
| | CTGTGCAGCAGCGATTGCATTCGCATTTTTGTGACCTT | |
| | TAGCAGCATGTTTCTGATTTTTGTGCTGGGCGCGATTC | |
| | TGTTTTTTCATCAGCGCCGCAACCATGGCCCGAACGAA | |
| | GATCGCCAGGCGGTGCCGGAAGAACCGTGCCCGTATA | |
| | GCTGCCCGCGCGAAGAAGAAGGCAGCGCGATTCCGAT | |
| | TCAGGAAGATTATCGCAAACCGGAACCGGCGTTTTAT | |
| | CCG | |
| Murine | MAWPPPYWLCMLGTLVGLSATLAPNSCPDKHYWTGGG | 95 |
| CD27 | LCCRMCEPGTFFVKDCEQDRTAAQCDPCIPGTSFSPDY | |
| | HTRPHCESCRHCNSGFLIRNCTVTANAECSCSKNWQCR | |
| | DQECTECDPPLNPALTRQPSETPSPQPPPTHLPHGTEK | |
| | PSWPLHRQLPNSTVYSQRSSHRPLCSSDCIRIFVTFSS | |
| | MFLIFVLGAILFFHQRRNHGPNEDRQAVPEEPCPYSCP | |
| | REEEGSAIPIQEDYRKPEPAFYP | |
| Human | ATGCTGCGCCTGCTGCTGGCGCTGAACCTGTTTCCGAG | 96 |
| CD28 | CATTCAGGTGACCGGCAACAAAATTCTGGTGAAACAG | |
| | AGCCCGATGCTGGTGGCGTATGATAACGCGGTGAACC | |
| | TGAGCTGCAAATATAGCTATAACCTGTTTAGCCGCGA | |
| | ATTTCGCGCGAGCCTGCATAAAGGCCTGGATAGCGCG | |
| | GTGGAAGTGTGCGTGGTGTATGGCAACTATAGCCAGC | |
| | AGCTGCAGGTGTATAGCAAAACCGGCTTTAACTGCGA | |
| | TGGCAAACTGGGCAACGAAAGCGTGACCTTTTATCTG | |
| | CAGAACCTGTATGTGAACCAGACCGATATTTATTTTTG | |
| | CAAAATTGAAGTGATGTATCCGCCGCCGTATCTGGAT | |
| | AACGAAAAAAGCAACGGCACCATTATTCATGTGAAAG | |
| | GCAAACATCTGTGCCCGAGCCCGCTGTTTCCGGGCCCG | |
| | AGCAAACCGTTTTGGGTGCTGGTGGTGGTGGGCGGCG | |
| | TGCTGGCGTGCTATAGCCTGCTGGTGACCGTGGCGTTT | |
| | ATTATTTTTTGGGTGCGCAGCAAACGCAGCCGCCTGCT | |
| | GCATAGCGATTATATGAACATGACCCCGCGCCGCCCG | |
| | GGCCCGACCCGCAAACATTATCAGCCGTATGCGCCGC | |
| | CGCGCGATTTTGCGGCGTATCGCAGC | |
| Human | MLRLLLALNLFPSIQVTGNKILVKQSPMLVAYDNAVNL | 97 |
| CD28 | SCKYSYNLFSREFRASLHKGLDSAVEVCVVYGNYSQQL | |
| | QVYSKTGFNCDGKLGNESVTFYLQNLYVNQTDIYFCKI | |
| | EVMYPPPYLDNEKSNGTIIHVKGKHLCPSPLFPGPSKP | |
| | FWVLVVVGGVLACYSLLVTVAFIIFWVRSKRSRLLHSD | |
| | YMNMTPRRPGPTRKHYQPYAPPRDFAAYRS | |
| Murine | ATGACCCTGCGCCTGCTGTTTCTGGCGCTGAACTTTTT | 98 |
| CD28 | TAGCGTGCAGGTGACCGAAAACAAAATTCTGGTGAAA | |
| | CAGAGCCCGCTGCTGGTGGTGGATAGCAACGAAGTGA | |
| | GCCTGAGCTGCCGCTATAGCTATAACCTGCTGGCGAA | |
| | AGAATTTCGCGCGAGCCTGTATAAAGGCGTGAACAGC | |
| | GATGTGGAAGTGTGCGTGGGCAACGGCAACTTTACCT | |
| | ATCAGCCGCAGTTTCGCAGCAACGCGGAATTTAACTG | |
| | CGATGGCGATTTTGATAACGAAACCGTGACCTTTCGCC | |
| | TGTGGAACCTGCATGTGAACCATACCGATATTTATTTT | |
| | TGCAAAATTGAATTTATGTATCCGCCGCCGTATCTGGA | |
| | TAACGAACGCAGCAACGGCACCATTATTCATATTAAA | |
| | GAAAAACATCTGTGCCATACCCAGAGCAGCCCGAAAC | |
| | TGTTTTGGGCGCTGGTGGTGGTGGCGGGCGTGCTGTTT | |
| | TGCTATGGCCTGCTGGTGACCGTGGCGCTGTGCGTGAT | |
| | TTGGACCAACAGCCGCCGCAACCGCCTGCTGCAGAGC | |
| | GATTATATGAACATGACCCCGCGCCGCCCGGGCCTGA | |
| | CCCGCAAACCGTATCAGCCGTATGCGCCGGCGCGCGA | |
| | TTTTGCGGCGTATCGCCCG | |
| Murine | MTLRLLFLALNFFSVQVTENKILVKQSPLLVVDSNEVS | 99 |
| CD28 | LSCRYSYNLLAKEFRASLYKGVNSDVEVCVGNGNFTYQ | |
| | PQFRSNAEFNCDGDFDNETVTFRLWNLHVNHTDIYFCK | |
| | IEFMYPPPYLDNERSNGTIIHIKEKHLCHTQSSPKLFW | |
| | ALVVVAGVLFCYGLLVTVALCVIWTNSRRNRLLQSDYM | |
| | NMTPRRPGLTRKPYQPYAPARDFAAYRP | |
| Human | ATGGGAAACAGCTGTTACAACATAGTAGCCACTCTGT | 100 |
| CD137 | TGCTGGTCCTCAACTTTGAGAGGACAAGATCATTGCA | |
| | GGATCCTTGTAGTAACTGCCCAGCTGGTACATTCTGTG | |
| | ATAATAACAGGAATCAGATTTGCAGTCCCTGTCCTCCA | |
| | AATAGTTTCTCCAGCGCAGGTGGACAAAGGACCTGTG | |
| | ACATATGCAGGCAGTGTAAAGGTGTTTTCAGGACCAG | |
| | GAAGGAGTGTTCCTCCACCAGCAATGCAGAGTGTGAC | |
| | TGCACTCCAGGGTTTCACTGCCTGGGGGCAGGATGCA | |
| | GCATGTGTGAACAGGATTGTAAACAAGGTCAAGAACT | |
| | GACAAAAAAAGGTTGTAAAGACTGTTGCTTTGGGACA | |
| | TTTAACGATCAGAAACGTGGCATCTGTCGACCCTGGA | |
| | CAAACTGTTCTTTGGATGGAAAGTCTGTGCTTGTGAAT | |
| | GGGACGAAGGAGAGGGACGTGGTCTGTGGACCATCTC | |
| | CAGCCGACCTCTCTCCGGGAGCATCCTCTGTGACCCCG | |
| | CCTGCCCCTGCGAGAGAGCCAGGACACTCTCCGCAGA | |
| | TCATCTCCTTCTTTCTTGCGCTGACGTCGACTGCGTTG | |
| | CTCTTCCTGCTGTTCTTCCTCACGCTCCGTTTCTCTGT | |
| | TGTTAAACGGGGCAGAAAGAAACTCCTGTATATATTCA | |
| | AACAACCATTTATGAGACCAGTACAAACTACTCAAGAG | |
| | GAAGATGGCTGTAGCTGCCGATTTCCAGAAGAAGAAG | |
| | AAGGAGGATGTGAACTGTGA | |
| Human | MGNSCYNIVATLLLVLNFERTRSLQDPCSNCPAGTFCD | 101 |
| CD137 | NNRNQICSPCPPNSFSSAGGQRTCDICRQCKGVFRTRK | |
| | ECSSTSNAECDCTPGFHCLGAGCSMCEQDCKQGQELTK | |
| | KGCKDCCFGTFNDQKRGICRPWTNCSLDGKSVLVNGTK | |
| | ERDVVCGPSPADLSPGASSVTPPAPAREPGHSPQIISF | |
| | FLALTSTALLFLLFFLTLRFSVVKRGRKKLLYIFKQPF | |
| | MRPVQTTQEEDGCSCRFPEEEEGGCEL | |
| Murine | ATGGGCAACAACTGCTATAACGTGGTGGTGATTGTGC | 102 |
| CD137 | TGCTGCTGGTGGGCTGCGAAAAAGTGGGCGCGGTGCA | |
| | GAACAGCTGCGATAACTGCCAGCCGGGCACCTTTTGC | |
| | CGCAAATATAACCCGGTGTGCAAAAGCTGCCCGCCGA | |
| | GCACCTTTAGCAGCATTGGCGGCCAGCCGAACTGCAA | |
| | CATTTGCCGCGTGTGCGCGGGCTATTTTCGCTTTAAAA | |
| | AATTTTGCAGCAGCACCCATAACGCGGAATGCGAATG | |
| | CATTGAAGGCTTTCATTGCCTGGGCCCGCAGTGCACCC | |
| | GCTGCGAAAAAGATTGCCGCCCGGGCCAGGAACTGAC | |
| | CAAACAGGGCTGCAAAACCTGCAGCCTGGGCACCTTT | |
| | AACGATCAGAACGGCACCGGCGTGTGCCGCCCGTGGA | |
| | CCAACTGCAGCCTGGATGGCCGCAGCGTGCTGAAAAC | |
| | CGGCACCACCGAAAAAGATGTGGTGTGCGGCCCGCCG | |
| | GTGGTGAGCTTTAGCCCGAGCACCACCATTAGCGTGA | |
| | CCCCGGAAGGCGGCCCGGGCGGCCATAGCCTGCAGGT | |
| | GCTGACCCTGTTTCTGGCGCTGACCAGCGCGCTGCTGC | |
| | TGGCGCTGATTTTTATTACCCTGCTGTTTAGCGTGCTG | |
| | AAATGGATTCGCAAAAAATTTCCGCATATTTTTAAACA | |
| | GCCGTTTAAAAAAACCACCGGCGCGGCGCAGGAAGAA | |
| | GATGCGTGCAGCTGCCGCTGCCCGCAGGAAGAAGAAG | |
| | GCGGCGGCGGCGGCTATGAACTG | |
| Murine | MGNNCYNVVVIVLLLVGCEKVGAVQNSCDNCQPGTFCR | 103 |
| CD137 | KYNPVCKSCPPSTFSSIGGQPNCNICRVCAGYFRFKKF | |
| | CSSTHNAECECIEGFHCLGPQCTRCEKDCRPGQELTKQ | |
| | GCKTCSLGTFNDQNGTGVCRPWTNCSLDGRSVLKTGTT | |
| | EKDVVCGPPVVSFSPSTTISVTPEGGPGGHSLQVLTLF | |
| | LALTSALLLALIFITLLFSVLKWIRKKFPHIFKQPFKK | |
| | TTGAAQEEDACSCRCPQEEEGGGGGYEL | |
| Human | ATGTGCGTGGGCGCGCGCCGCCTGGGCCGCGGCCCGT | 104 |
| OX40 | GCGCGGCGCTGCTGCTGCTGGGCCTGGGCCTGAGCAC | |
| | CGTGACCGGCCTGCATTGCGTGGGCGATACCTATCCG | |
| | AGCAACGATCGCTGCTGCCATGAATGCCGCCCGGGCA | |
| | ACGGCATGGTGAGCCGCTGCAGCCGCAGCCAGAACAC | |
| | CGTGTGCCGCCCGTGCGGCCCGGGCTTTTATAACGATG | |
| | TGGTGAGCAGCAAACCGTGCAAACCGTGCACCTGGTG | |
| | CAACCTGCGCAGCGGCAGCGAACGCAAACAGCTGTGC | |
| | ACCGCGACCCAGGATACCGTGTGCCGCTGCCGCGCGG | |
| | GCACCCAGCCGCTGGATAGCTATAAACCGGGCGTGGA | |
| | TTGCGCGCCGTGCCCGCCGGGCCATTTTAGCCCGGGCG | |
| | ATAACCAGGCGTGCAAACCGTGGACCAACTGCACCCT | |
| | GGCGGGCAAACATACCCTGCAGCCGGCGAGCAACAGC | |
| | AGCGATGCGATTTGCGAAGATCGCGATCCGCCGGCGA | |
| | CCCAGCCGCAGGAAACCCAGGGCCCGCCGGCGCGCCC | |
| | GATTACCGTGCAGCCGACCGAAGCGTGGCCGCGCACC | |
| | AGCCAGGGCCCGAGCACCCGCCCGGTGGAAGTGCCGG | |
| | GCGGCCGCGCGGTGGCGGCGATTCTGGGCCTGGGCCT | |
| | GGTGCTGGGCCTGCTGGGCCCGCTGGCGATTCTGCTGG | |
| | CGCTGTATCTGCTGCGCCGCGATCAGCGCCTGCCGCCG | |
| | GATGCGCATAAACCGCCGGGCGGCGGCAGCTTTCGCA | |
| | CCCCGATTCAGGAAGAACAGGCGGATGCGCATAGCAC | |
| | CCTGGCGAAAATT | |
| Human | MCVGARRLGRGPCAALLLLGLGLSTVTGLHCVGDTYPS | 105 |
| OX40 | NDRCCHECRPGNGMVSRCSRSQNTVCRPCGPGFYNDVV | |
| | SSKPCKPCTWCNLRSGSERKQLCTATQDTVCRCRAGTQ | |
| | PLDSYKPGVDCAPCPPGHFSPGDNQACKPWTNCTLAGK | |
| | HTLQPASNSSDAICEDRDPPATQPQETQGPPARPITVQ | |
| | PTEAWPRTSQGPSTRPVEVPGGRAVAAILGLGLVLGLL | |
| | GPLAILLALYLLRRDQRLPPDAHKPPGGGSFRTPIQEE | |
| | QADAHSTLAKI | |
| Murine | ATGTATGTGTGGGTGCAGCAGCCGACCGCGCTGCTGC | 106 |
| OX40 | TGCTGGCGCTGACCCTGGGCGTGACCGCGCGCCGCCT | |
| | GAACTGCGTGAAACATACCTATCCGAGCGGCCATAAA | |
| | TGCTGCCGCGAATGCCAGCCGGGCCATGGCATGGTGA | |
| | GCCGCTGCGATCATACCCGCGATACCCTGTGCCATCCG | |
| | TGCGAAACCGGCTTTTATAACGAAGCGGTGAACTATG | |
| | ATACCTGCAAACAGTGCACCCAGTGCAACCATCGCAG | |
| | CGGCAGCGAACTGAAACAGAACTGCACCCCGACCCAG | |
| | GATACCGTGTGCCGCTGCCGCCCGGGCACCCAGCCGC | |
| | GCCAGGATAGCGGCTATAAACTGGGCGTGGATTGCGT | |
| | GCCGTGCCCGCCGGGCCATTTTAGCCCGGGCAACAAC | |
| | CAGGCGTGCAAACCGTGGACCAACTGCACCCTGAGCG | |
| | GCAAACAGACCCGCCATCCGGCGAGCGATAGCCTGGA | |
| | TGCGGTGTGCGAAGATCGCAGCCTGCTGGCGACCCTG | |
| | CTGTGGGAAACCCAGCGCCCGACCTTTCGCCCGACCA | |
| | CCGTGCAGAGCACCACCGTGTGGCCGCGCACCAGCGA | |
| | ACTGCCGAGCCCGCCGACCCTGGTGACCCCGGAAGGC | |
| | CCGGCGTTTGCGGTGCTGCTGGGCCTGGGCCTGGGCCT | |
| | GCTGGCGCCGCTGACCGTGCTGCTGGCGCTGTATCTGC | |
| | TGCGCAAAGCGTGGCGCCTGCCGAACACCCCGAAACC | |
| | GTGCTGGGGCAACAGCTTTCGCACCCCGATTCAGGAA | |
| | GAACATACCGATGCGCATTTTACCCTGGCGAAAATT | |
| Murine | MYVWVQQPTALLLLALTLGVTARRLNCVKHTYPSGHK | 107 |
| OX40 | CCRECQPGHGMVSRCDHTRDTLCHPCETGFYNEAVNYD | |
| | TCKQCTQCNHRSGSELKQNCTPTQDTVCRCRPGTQPRQ | |
| | DSGYKLGVDCVPCPPGHFSPGNNQACKPWTNCTLSGKQ | |
| | TRHPASDSLDAVCEDRSLLATLLWETQRPTFRPTTVQS | |
| | TTVWPRTSELPSPPTLVTPEGPAFAVLLGLGLGLLAPL | |
| | TVLLALYLLRKAWRLPNTPKPCWGNSFRTPIQEEHTDA | |
| | HFTLAKI | |
| Human | ATGAAAAGCGGCCTGTGGTATTTTTTTCTGTTTTGCCT | 108 |
| ICOS | GCGCATTAAAGTGCTGACCGGCGAAATTAACGGCAGC | |
| | GCGAACTATGAAATGTTTATTTTTCATAACGGCGGCGT | |
| | GCAGATTCTGTGCAAATATCCGGATATTGTGCAGCAGT | |
| | TTAAAATGCAGCTGCTGAAAGGCGGCCAGATTCTGTG | |
| | CGATCTGACCAAAACCAAAGGCAGCGGCAACACCGTG | |
| | AGCATTAAAAGCCTGAAATTTTGCCATAGCCAGCTGA | |
| | GCAACAACAGCGTGAGCTTTTTTCTGTATAACCTGGAT | |
| | CATAGCCATGCGAACTATTATTTTTGCAACCTGAGCAT | |
| | TTTTGATCCGCCGCCGTTTAAAGTGACCCTGACCGGCG | |
| | GCTATCTGCATATTTATGAAAGCCAGCTGTGCTGCCAG | |
| | CTGAAATTTTGGCTGCCGATTGGCTGCGCGGCGTTTGT | |
| | GGTGGTGTGCATTCTGGGCTGCATTCTGATTTGCTGGC | |
| | TGACCAAAAAAAAATATAGCAGCAGCGTGCATGATCC | |
| | GAACGGCGAATATATGTTTATGCGCGCGGTGAACACC | |
| | GCGAAAAAAAGCCGCCTGACCGATGTGACCCTG | |
| Human | MKSGLWYFFLFCLRIKVLTGEINGSANYEMFIFHNGGV | 109 |
| ICOS | QILCKYPDIVQQFKMQLLKGGQILCDLTKTKGSGNTVS | |
| | IKSLKFCHSQLSNNSVSFFLYNLDHSHANYYFCNLSIF | |
| | DPPPFKVTLTGGYLHIYESQLCCQLKFWLPIGCAAFVV | |
| | VCILGCILICWLTKKKYSSSVHDPNGEYMFMRAVNTAK | |
| | KSRLTDVTL | |
| Murine | ATGAAACCGTATTTTTGCCGCGTGTTTGTGTTTTGCTT | 110 |
| ICOS | TCTGATTCGCCTGCTGACCGGCGAAATTAACGGCAGCG | |
| | CGGATCATCGCATGTTTAGCTTTCATAACGGCGGCGTG | |
| | CAGATTAGCTGCAAATATCCGGAAACCGTGCAGCAGC | |
| | TGAAAATGCGCCTGTTTCGCGAACGCGAAGTGCTGTG | |
| | CGAACTGACCAAAACCAAAGGCAGCGGCAACGCGGT | |
| | GAGCATTAAAAACCCGATGCTGTGCCTGTATCATCTGA | |
| | GCAACAACAGCGTGAGCTTTTTTCTGAACAACCCGGA | |
| | TAGCAGCCAGGGCAGCTATTATTTTTGCAGCCTGAGCA | |
| | TTTTTGATCCGCCGCCGTTTCAGGAACGCAACCTGAGC | |
| | GGCGGCTATCTGCATATTTATGAAAGCCAGCTGTGCTG | |
| | CCAGCTGAAACTGTGGCTGCCGGTGGGCTGCGCGGCG | |
| | TTTGTGGTGGTGCTGCTGTTTGGCTGCATTCTGATTAT | |
| | TTGGTTTAGCAAAAAAAAATATGGCAGCAGCGTGCATG | |
| | ATCCGAACAGCGAATATATGTTTATGGCGGCGGTGAA | |
| | CACCAACAAAAAAAGCCGCCTGGCGGGCGTGACCAGC | |
| Murine | MKPYFCRVFVFCFLIRLLTGEINGSADHRMFSFHNGGV | 111 |
| ICOS | QISCKYPETVQQLKMRLFREREVLCELTKTKGSGNAVS | |
| | IKNPMLCLYHLSNNSVSFFLNNPDSSQGSYYFCSLSIF | |
| | DPPPFQERNLSGGYLHIYESQLCCQLKLWLPVGCAAFV | |
| | VVLLFGCILIIWFSKKKYGSSVHDPNSEYMFMAAVNTN | |
| | KKSRLAGVTS | |
| Human | ATGATTCATCTGGGCCATATTCTGTTTCTGCTGCTGCT | 112 |
| DAP10 | GCCGGTGGCGGCGGCGCAGACCACCCCGGGCGAACGC | |
| | AGCAGCCTGCCGGCGTTTTATCCGGGCACCAGCGGCA | |
| | GCTGCAGCGGCTGCGGCAGCCTGAGCCTGCCGCTGCT | |
| | GGCGGGCCTGGTGGCGGCGGATGCGGTGGCGAGCCTG | |
| | CTGATTGTGGGCGCGGTGTTTCTGTGCGCGCGCCCGCG | |
| | CCGCAGCCCGGCGCAGGAAGATGGCAAAGTGTATATT | |
| | AACATGCCGGGCCGCGGC | |
| Human | MIHLGHILFLLLLPVAAAQTTPGERSSLPAFYPGTSGS | 113 |
| DAP10 | CSGCGSLSLPLLAGLVAADAVASLLIVGAVFLCARPRR | |
| | SPAQEDGKVYINMPGRG | |
| Murine | ATGGATCCGCCGGGCTATCTGCTGTTTCTGCTGCTGCT | 114 |
| DAP10 | GCCGGTGGCGGCGAGCCAGACCAGCGCGGGCAGCTGC | |
| | AGCGGCTGCGGCACCCTGAGCCTGCCGCTGCTGGCGG | |
| | GCCTGGTGGCGGCGGATGCGGTGATGAGCCTGCTGAT | |
| | TGTGGGCGTGGTGTTTGTGTGCATGCGCCCGCATGGCC | |
| | GCCCGGCGCAGGAAGATGGCCGCGTGTATATTAACAT | |
| | GCCGGGCCGCGGC | |
| Murine | MDPPGYLLFLLLLPVAASQTSAGSCSGCGTLSLPLLAG | 115 |
| DAP10 | LVAADAVMSLLIVGVVFVCMRPHGRPAQEDGRVYINMP | |
| | GRG | |
| Human | ATGGGGGGACTTGAACCCTGCAGCAGGCTCCTGCTCC | 116 |
| DAP12 | TGCCTCTCCTGCTGGCTGTAAGTGGTCTCCGTCCTGTC | |
| | CAGGCCCAGGCCCAGAGCGATTGCAGTTGCTCTACGG | |
| | TGAGCCCGGGCGTGCTGGCAGGGATCGTGATGGGAGA | |
| | CCTGGTGCTGACAGTGCTCATTGCCCTGGCCGTGTACT | |
| | TCCTGGGCCGGCTGGTCCCTCGGGGGCGAGGGGCTGC | |
| | GGAGGCAGCGACCCGGAAACAGCGTATCACTGAGACC | |
| | GAGTCGCCTTATCAGGAGCTCCAGGGTCAGAGGTCGG | |
| | ATGTCTACAGCGACCTCAACACACAGAGGCCGTATTA | |
| | CAAATGA | |
| Human | MGGLEPCSRLLLLPLLLAVSGLRPVQAQAQSDCSCSTV | 117 |
| DAP12 | SPGVLAGIVMGDLVLTVLIALAVYFLGRLVPRGRGAAE | |
| | AATRKQRITETESPYQELQGQRSDVYSDLNTQRPYYK | |
| Murine | ATGGGGGCTCTGGAGCCCTCCTGGTGCCTTCTGTTCCT | 118 |
| DAP12 | TCCTGTCCTCCTGACTGTGGGAGGATTAAGTCCCGTAC | |
| | AGGCCCAGAGTGACACTTTCCCAAGATGCGACTGTTCT | |
| | TCCGTGAGCCCTGGTGTACTGGCTGGGATTGTTCTGGG | |
| | TGACTTGGTGTTGACTCTGCTGATTGCCCTGGCTGTGT | |
| | ACTCTCTGGGCCGCCTGGTCTCCCGAGGTCAAGGGAC | |
| | AGCGGAAGGGACCCGGAAACAACACATTGCTGAGACT | |
| | GAGTCGCCTTATCAGGAGCTTCAGGGTCAGAGACCAG | |
| | AAGTATACAGTGACCTCAACACACAGAGGCAATATTA | |
| | CAGATGA | |
| Murine | MGALEPSWCLLFLPVLLTVGGLSPVQAQSDTFPRCDCS | 119 |
| DAP12 | SVSPGVLAGIVLGDLVLTLLIALAVYSLGRLVSRGQGT | |
| | AEGTRKQHIAETESPYQELQGQRPEVYSDLNTQRQYYR | |
| Human | MKWKALFTAAILQAQLPITEAQSFGLLDPKLCYLLDGI | 120 |
| CD3z | LFIYGVILTALFLRVKFSRSADAPAYQQGQNQLYNELN | |
| | LGRREEYDVLDKRRGRDPEMGGKPQRRKNPQEGLYNEL | |
| | QKDKMAEAYSEIGMKGERRRGKGHDGLYQGLSTATKDT | |
| | YDALHMQALPPR | |
| Human | ATGAAGTGGAAGGCGCTTTTCACCGCGGCCATCCTGC | 121 |
| CD3z | AGGCACAGTTGCCGATTACAGAGGCACAGAGCTTTGG | |
| | CCTGCTGGATCCCAAACTCTGCTACCTGCTGGATGGAA | |
| | TCCTCTTCATCTATGGTGTCATTCTCACTGCCTTGTTC | |
| | CTGAGAGTGAAGTTCAGCAGGAGCGCAGAGCCCCCCGC | |
| | GTACCAGCAGGGCCAGAACCAGCTCTATAACGAGCTC | |
| | AATCTAGGACGAAGAGAGGAGTACGATGTTTTGGACA | |
| | AGAGACGTGGCCGGGACCCTGAGATGGGGGGAAAGC | |
| | CGAGAAGGAAGAACCCTCAGGAAGGCCTGTACAATGA | |
| | ACTGCAGAAAGATAAGATGGCGGAGGCCTACAGTGAG | |
| | ATTGGGATGAAAGGCGAGCGCCGGAGGGGCAAGGGG | |
| | CACGATGGCCTTTACCAGGGTCTCAGTACAGCCACCA | |
| | AGGACACCTACGACGCCCTTCACATGCAGGCCCTGCC | |
| | CCCTCGCTAA | |
| Murine | MKWKVSVLACILHVRFPGAEAQSFGLLDPKLCYLLDGI | 122 |
| CD3z | LFIYGVIITALYLRAKFSRSAETAANLQDPNQLYNELN | |
| | LGRREEYDVLEKKRARDPEMGGKQQRRRNPQEGVYNAL | |
| | QKDKMAEAYSEIGTKGERRRGKGHDGLYQGLSTATKDT | |
| | YDALHMQTLAPR | |
| Murine | ATGAAGTGGAAAGTGTCTGTTCTCGCCTGCATCCTCCA | 123 |
| CD3z | CGTGCGGTTCCCAGGAGCAGAGGCACAGAGCTTTGGT | |
| | CTGCTGGATCCCAAACTCTGCTACTTGCTAGATGGAAT | |
| | CCTCTTCATCTACGGAGTCATCATCACAGCCCTGTACC | |
| | TGAGAGCAAAATTCAGCAGGAGTGCAGAGACTGCTGC | |
| | CAACCTGCAGGACCCCAACCAGCTCTACAATGAGCTC | |
| | AATCTAGGGCGAAGAGAGGAATATGACGTCTTGGAGA | |
| | AGAAGCGGGCTCGGGATCCAGAGATGGGAGGCAAAC | |
| | AGCAGAGGAGGAGGAACCCCCAGGAAGGCGTATACA | |
| | ATGCACTGCAGAAAGACAAGATGGCAGAAGCCTACAG | |
| | TGAGATCGGCACAAAAGGCGAGAGGCGGAGAGGCAA | |
| | GGGGCACGATGGCCTTTACCAGGGTCTCAGCACTGCC | |
| | ACCAAGGACACCTATGATGCCCTGCATATGCAGACCC | |
| | TGGCCCCTCGCTAA | |
| Human | MWQLLLPTALLLLVSAGMRTEDLPKAVVFLEPQWYRVL | 124 |
| FCGR3A | EKDSVTLKCQGAYSPEDNSTQWFHNESLISSQASSYFI | |
| | DAATVDDSGEYRCQTNLSTLSDPVQLEVHIGWLLLQAP | |
| | RWVFKEEDPIHLRCHSWKNTALHKVTYLQNGKGRKYFH | |
| | HNSDFYIPKATLKDSGSYFCRGLFGSKNVSSETVNITI | |
| | TQGLAVSTISSFFPPGYQVSFCLVMVLLFAVDTGLYFS | |
| | VKTNIRSSTRDWKDHKFKWRKDPQDK | |
| Human | ATGTGGCAGCTGCTGCTGCCGACCGCGCTGCTGCTGCT | 125 |
| FCGR3A | GGTGAGCGCGGGCATGCGCACCGAAGATCTGCCGAAA | |
| | GCGGTGGTGTTTCTGGAACCGCAGTGGTATCGCGTGCT | |
| | GGAAAAAGATAGCGTGACCCTGAAATGCCAGGGCGCG | |
| | TATAGCCCGGAAGATAACAGCACCCAGTGGTTTCATA | |
| | ACGAAAGCCTGATTAGCAGCCAGGCGAGCAGCTATTT | |
| | TATTGATGCGGCGACCGTGGATGATAGCGGCGAATAT | |
| | CGCTGCCAGACCAACCTGAGCACCCTGAGCGATCCGG | |
| | TGCAGCTGGAAGTGCATATTGGCTGGCTGCTGCTGCA | |
| | GGCGCCGCGCTGGGTGTTTAAAGAAGAAGATCCGATT | |
| | CATCTGCGCTGCCATAGCTGGAAAAACACCGCGCTGC | |
| | ATAAAGTGACCTATCTGCAGAACGGCAAAGGCCGCAA | |
| | ATATTTTCATCATAACAGCGATTTTTATATTCCGAAAG | |
| | CGACCCTGAAAGATAGCGGCAGCTATTTTTGCCGCGG | |
| | CCTGTTTGGCAGCAAAAACGTGAGCAGCGAAACCGTG | |
| | AACATTACCATTACCCAGGGCCTGGCGGTGAGCACCA | |
| | TTAGCAGCTTTTTTCCGCCGGGCTATCAGGTGAGCTTT | |
| | TGCCTGGTGATGGTGCTGCTGTTTGCGGTGGATACCGG | |
| | CCTGTATTTTAGCGTGAAAACCAACATTCGCAGCAGC | |
| | ACCCGCGATTGGAAAGATCATAAATTTAAATGGCGCA | |
| | AAGATCCGCAGGATAAA | |
| Murine | MFQNAHSGSQWLLPPLTILLLFAFADRQSAALPKAVVK | 126 |
| FCGR3A | LDPPWIQVLKEDMVTLMCEGTHNPGNSSTQWFHNGRSI | |
| | RSQVQASYTFKATVNDSGEYRCQMEQTRLSDPVDLGVI | |
| | SDWLLLQTPQRVFLEGETITLRCHSWRNKLLNRISFFH | |
| | NEKSVRYHHYKSNFSIPKANHSHSGDYYCKGSLGSTQH | |
| | QSKPVTITVQDPATTSSISLVWYHTAFSLVMCLLFAVD | |
| | TGLYFYVRRNLQTPREYWRKSLSIRKHQAPQDK | |
| Murine | ATGTTTCAGAATGCACACTCTGGAAGCCAATGGCTACT | 127 |
| FCGR3A | TCCACCACTGACAATTCTGCTGCTGTTTGCTTTTGCAG | |
| | ACAGGCAGAGTGCAGCTCTTCCGAAGGCTGTGGTGAA | |
| | ACTGGACCCCCCATGGATCCAGGTGCTCAAGGAAGAC | |
| | ATGGTGACACTGATGTGCGAAGGGACCCACAACCCTG | |
| | GGAACTCTTCTACCCAGTGGTTCCACAACGGGAGGTC | |
| | CATCCGGAGCCAGGTCCAAGCCAGTTACACGTTTAAG | |
| | GCCACAGTCAATGACAGTGGAGAATATCGGTGTCAAA | |
| | TGGAGCAGACCCGCCTCAGCGACCCTGTAGATCTGGG | |
| | AGTGATTTCTGACTGGCTGCTGCTCCAGACCCCTCAGC | |
| | GGGTGTTTCTGGAAGGGGAAACCATCACGCTAAGGTG | |
| | CCATAGCTGGAGGAACAAACTACTGAACAGGATCTCA | |
| | TTCTTCCATAATGAAAAATCCGTGAGGTATCATCACTA | |
| | CAAAAGTAATTTCTCTATCCCAAAAGCCAACCACAGT | |
| | CACAGTGGGGACTACTACTGCAAAGGAAGTCTAGGAA | |
| | GTACACAGCACCAGTCCAAGCCTGTCACCATCACTGTC | |
| | CAAGATCCAGCAACTACATCCTCCATCTCTCTAGTCTG | |
| | GTACCACACTGCTTTCTCCCTAGTGATGTGCCTCCTGT | |
| | TTGCAGTGGACACGGGCCTTTATTTCTACGTACGGAGA | |
| | AATCTTCAAACCCCGAGGGAGTACTGGAGGAAGTCCC | |
| | TGTCAATCAGAAAGCACCAGGCTCCTCAAGACAAGTG | |
| | A | |
| Human | MGWIRGRRSRHSWEMSEFHNYNLDLKKSDFSTRWQKQ | 128 |
| NKG2D | RCPVVKSKCRENASPFFFCCFIAVAMGIRFIIMVAIWS | |
| | AVFLNSLFNQEVQIPLTESYCGPCPKNWICYKNNCYQF | |
| | FDESKNWYESQASCMSQNASLLKVYSKEDQDLLKLVKS | |
| | YHWMGLVHIPTNGSWQWEDGSILSPNLLTIIEMQKGD | |
| | CALYASSFKGYIENCSTPNTYICMQRTV | |
| Human | ATGGGCTGGATTCGCGGCCGCCGCAGCCGCCATAGCT | 129 |
| NKG2D | GGGAAATGAGCGAATTTCATAACTATAACCTGGATCT | |
| | GAAAAAAAGCGATTTTAGCACCCGCTGGCAGAAACAG | |
| | CGCTGCCCGGTGGTGAAAAGCAAATGCCGCGAAAACG | |
| | CGAGCCCGTTTTTTTTTTGCTGCTTTATTGCGGTGGCG | |
| | ATGGGCATTCGCTTTATTATTATGGTGGCGATTTGGAG | |
| | CGCGGTGTTTCTGAACAGCCTGTTTAACCAGGAAGTGC | |
| | AGATTCCGCTGACCGAAAGCTATTGCGGCCCGTGCCC | |
| | GAAAAACTGGATTTGCTATAAAAACAACTGCTATCAG | |
| | TTTTTTGATGAAAGCAAAAACTGGTATGAAAGCCAGG | |
| | CGAGCTGCATGAGCCAGAACGCGAGCCTGCTGAAAGT | |
| | GTATAGCAAAGAAGATCAGGATCTGCTGAAACTGGTG | |
| | AAAAGCTATCATTGGATGGGCCTGGTGCATATTCCGA | |
| | CCAACGGCAGCTGGCAGTGGGAAGATGGCAGCATTCT | |
| | GAGCCCGAACCTGCTGACCATTATTGAAATGCAGAAA | |
| | GGCGATTGCGCGCTGTATGCGAGCAGCTTTAAAGGCT | |
| | ATATTGAAAACTGCAGCACCCCGAACACCTATATTTGC | |
| | ATGCAGCGCACCGTG | |
| Murine | MALIRDRKSHHSEMSKCHNYDLKPAKWDTSQEQQKQR | 130 |
| NKG2D | LALTTSQPGENGIIRGRYPIEKLKISPMFVVRVLAIAL | |
| | AIRFTLNTLMWLAIFKETFQPVLCNKEVPVSSREGYCG | |
| | PCPNN | |
| | WICHRNNCYQFFNEEKTWNQSQASCLSQNSSLLKIYSK | |
| | EEQDFLKLVKSYHWMGLVQIPANGSWQWEDGSSLSYNQ | |
| | LTLVEIPKGSCAVYGSSFKAYTEDCANLNTYICMKRAV | |
| Murine | ATGGCGCTGATTCGCGATCGCAAAAGCCATCATAGCG | 131 |
| NKG2D | AAATGAGCAAATGCCATAACTATGATCTGAAACCGGC | |
| | GAAATGGGATACCAGCCAGGAACAGCAGAAACAGCG | |
| | CCTGGCGCTGACCACCAGCCAGCCGGGCGAAAACGGC | |
| | ATTATTCGCGGCCGCTATCCGATTGAAAAACTGAAAA | |
| | TTAGCCCGATGTTTGTGGTGCGCGTGCTGGCGATTGCG | |
| | CTGGCGATTCGCTTTACCCTGAACACCCTGATGTGGCT | |
| | GGCGATTTTTAAAGAAACCTTTCAGCCGGTGCTGTGCA | |
| | ACAAAGAAGTGCCGGTGAGCAGCCGCGAAGGCTATTG | |
| | CGGCCCGTGCCCGAACAACTGGATTTGCCATCGCAAC | |
| | AACTGCTATCAGTTTTTTAACGAAGAAAAAACCTGGA | |
| | ACCAGAGCCAGGCGAGCTGCCTGAGCCAGAACAGCAG | |
| | CCTGCTGAAAATTTATAGCAAAGAAGAACAGGATTTT | |
| | CTGAAACTGGTGAAAAGCTATCATTGGATGGGCCTGG | |
| | TGCAGATTCCGGCGAACGGCAGCTGGCAGTGGGAAGA | |
| | TGGCAGCAGCCTGAGCTATAACCAGCTGACCCTGGTG | |
| | GAAATTCCGAAAGGCAGCTGCGCGGTGTATGGCAGCA | |
| | GCTTTAAAGCGTATACCGAAGATTGCGCGAACCTGAA | |
| | CACCTATATTTGCATGAAACGCGCGGTG | |
| CD28 | YMNM | 132 |
| YMNM | | |
| CD28 | PYAP | 133 |
| PYAP | | |
| CD28 | FMNM | 134 |
| FMNM | | |
| CD28 | AYAA | 135 |
| AYAA | | |
| Signal | ATMGWSCIILFLVATATGVHS | 136 |
| peptide | | |
| Signal | ATGGGATGGAGCTGTATCATCCTCTTCTTGGTAGCAAC | 137 |
| peptide | AGCTACCGGTGTGCACTCC | |
| DNA | | |
| sequence | | |

TABLE 20
Anti-CEA (98/99) sequences

| | | SEQ |
|---|---|---|
| Con- | | ID |
| struct | Protein Sequence | NO |
| Anti- | EFGMN | 138 |
| CEA | | |
| (98/99) | | |
| CDR H1 | | |
| Kabat | | |
| Anti- | WINTKTGEATYVEEFKG | 139 |
| CEA | | |
| (98/99) | | |
| CDR H2 | | |
| Kabat | | |
| Anti- | WDFAYYVEAMDY | 140 |
| CEA | | |
| (98/99) | | |
| CDR H3 | | |
| Kabat | | |
| Anti- | KASAAVGTYVA | 141 |
| CEA | | |
| (98/99) | | |
| CDR 1 | | |
| L1 Kabat | | |
| Anti- | SASYRKR | 142 |
| CEA | | |
| (98/99) | | |
| CDR L2 | | |
| Kabat | | |
| Anti- | HQYYTYPLFT | 143 |
| CEA | | |
| (98/99) | | |
| CDR L3 | | |
| Kabat | | |
| Anti- | QVQLVQSGAEVKKPGASVKVSCKASGYTFTEFGMNWV | 144 |
| CEA | RQAPGQGLEWMGWINTKTGEATYVEEFKGRVTFTTDTS | |
| (98/99)- | TSTAYMELRSLRSDDTAVYYCARWDFAYYVEAMDYWG | |
| scFv- | QGTTVTVSSGGGGSGGGGSGGGGSGGGGSDIQMTQSPS | |
| CD28ATD- | SLSASVGDRVTITCKASAAVGTYVAWYQQKPGKAPKLL | |
| CD28CSD- | IYSASYRKRGVPSRFSGSGSGTDFTLTISSLQPEDFAT | |
| CD3zSSD | YYCHQYYTYPLFTFGQGTKLEIKRTGGGGSFWVLVVVG | |
| fusion | GVLACYSLLVTVAFIIFWVRSKRSRLLHSDYMNMTPRR | |
| | PGPTRKHYQPYAPPRDFAAYRSRVKFSRSADAPAYQQG | |
| | QNQLYNELNLGRREEYDVLDKRRGRDPEMGGKPRRKNP | |
| | QEGLYNELQKDKMAEAYSEIGMKGERRRGKGHDGLYQG | |
| | LSTATKDTYDALHMQALPPR | |
| Anti- | QVQLVQSGAEVKKPGASVKVSCKASGYTFTEFGMNWV | 145 |
| CEA | RQAPGQGLEWMGWINTKTGEATYVEEFKGRVTFTTDTS | |
| (98/99)- | TSTAYMELRSLRSDDTAVYYCARWDFAYYVEAMDYWG | |
| scFv | QGTTVTVSSGGGGSGGGGSGGGGSGGGGSDIQMTQSPS | |
| | SLSASVGDRVTITCKASAAVGTYVAWYQQKPGKAPKLL | |
| | IYSASYRKRGVPSRFSGSGSGTDFTLTISSLQPEDFAT | |
| | YYCHQYYTYPLFTFGQGTKLEIKRT | |
| Anti- | QVQLVQSGAEVKKPGASVKVSCKASGYTFTEFGMNWV | 146 |
| CEA | RQAPGQGLEWMGWINTKTGEATYVEEFKGRVTFTTDTS | |
| (98/99)- | TSTAYMELRSLRSDDTAVYYCARWDFAYYVEAMDYWG | |
| VH | QGTTVTVSS | |
| Anti- | DIQMTQSPSSLSASVGDRVTITCKASAAVGTYVAWYQQ | 147 |
| CEA | KPGKAPKLLIYSASYRKRGVPSRFSGSGSGTDFTLTIS | |
| (98/99)- | SLQPEDFATYYCHQYYTYPLFTFGQGTKLEIKRT | |
| VL | | |
| CD28ATD- | see Table 2 | 17 |
| CD28CSD- | | |
| CD3zSSD | | |

TABLE 21
Anti-CEA (T84.66) sequences

| | | SEQ |
|---|---|---|
| Con- | | ID |
| struct | Protein Sequence | NO |
| Anti- | DTYMH | 148 |
| CEA | | |
| (T84.66) | | |
| CDR H1 | | |
| Kabat | | |
| Anti- | RIDPANGNSKYVPKFQG | 149 |
| CEA | | |
| (T84.66) | | |
| CDR H2 | | |
| Kabat | | |
| Anti- | FGYYVSDYAMAY | 150 |
| CEA | | |
| (T84.66) | | |
| CDR H3 | | |
| Kabat | | |
| Anti- | RAGESVDIFGVGFLH | 151 |
| CEA | | |
| (T84.66) | | |
| CDR 1 | | |
| L1 Kabat | | |
| Anti- | RASNRAT | 152 |
| CEA | | |
| (T84.66) | | |
| CDR L2 | | |
| Kabat | | |
| Anti- | QQTNEDPYT | 153 |
| CEA | | |
| (T84.66) | | |
| CDR L3 | | |
| Kabat | | |
| Anti- | QVQLVQSGAEVKKPGSSVKVSCKASGFNIKDTYM | 154 |
| CEA | HWVRQAPGQGLEWMGRIDPANGNSKYVPKFQGRV | |
| (T84.66)- | TITADTSTSTAYMELSSLRSEDTAVYYCAPFGYY | |
| scFv- | VSDYAMAYWGQGTLVTVSSGGGGSGGGGSGGGGS | |
| CD28ATD- | GGGGSEIVLTQSPATLSLSPGERATLSCRAGESV | |
| CD28CSD- | DIFGVGFLHWYQQKPGQAPRLLIYRASNRATGIP | |
| CD3zSSD | ARFSGSGSGTDFTLTISSLEPEDFAVYYCQQTNE | |
| fusion | DPYTFGQGTKLEIKGGGGSFWVLVVVGGVLACYS | |
| | LLVTVAFIIFWVRSKRSRLLHSDYMNMTPRRPGP | |
| | TRKHYQPYAPPRDFAAYRSRVKFSRSADAPAYQQ | |
| | GQNQLYNELNLGRREEYDVLDKRRGRDPEMGGKP | |
| | RRKNPQEGLYNELQKDKMAEAYSEIGMKGERRRG | |
| | KGHDGLYQGLSTATKDTYDALHMQALPPR | |
| Anti- | QVQLVQSGAEVKKPGSSVKVSCKASGFNIKDTYM | 155 |
| CEA | HWVRQAPGQGLEWMGRIDPANGNSKYVPKFQGRV | |
| (T84.66)- | TITADTSTSTAYMELSSLRSEDTAVYYCAPFGYY | |
| scFv | VSDYAMAYWGQGTLVTVSSGGGGSGGGGSGGGGS | |
| | GGGGSEIVLTQSPATLSLSPGERATLSCRAGESV | |
| | DIFGVGFLHWYQQKPGQAPRLLIYRASNRATGIP | |
| | ARFSGSGSGTDFTLTISSLEPEDFAVYYCQQTNE | |
| | DPYTFGQGTKLEIK | |
| Anti- | QVQLVQSGAEVKKPGSSVKVSCKASGFNIKDTYM | 156 |
| CEA | HWVRQAPGQGLEWMGRIDPANGNSKYVPKFQGRV | |
| (T84.66)- | TITADTSTSTAYMELSSLRSEDTAVYYCAPFGYY | |
| VH | VSDYAMAYWGQGTLVTVSS | |
| Anti- | EIVLTQSPATLSLSPGERATLSCRAGESVDIFGV | 157 |
| CEA | GFLHWYQQKPGQAPRLLIYRASNRATGIPARFSG | |
| (T84.66)- | SGSGTDFTLTISSLEPEDFAVYYCQQTNEDPYTF | |
| VL | GQGTKLEIK | |
| CD28ATD- | see Table 2 | 17 |
| CD28CSD- | | |
| CD3zSSD | | |

What is claimed is:

1. An antigen binding receptor comprising an anchoring transmembrane domain and an extracellular domain comprising an antigen binding moiety, wherein the antigen binding moiety is a Fab, crossFab or a scFab.

2. The antigen binding receptor of claims 1, wherein the anchoring transmembrane domain is a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof.

3. The antigen binding receptor of any one of claim 1 or 2, wherein the anchoring transmembrane domain is the CD28 transmembrane domain or a fragment thereof, in particular wherein the anchoring transmembrane domain comprises the amino acid sequence of SEQ ID NO:14.

4. The antigen binding receptor of any one of claims 1 to 3 further comprising at least one stimulatory signaling domain and/or at least one co-stimulatory signaling domain.

5. The antigen binding receptor of any one of claims 1 to 4, wherein the at least one stimulatory signaling domain is individually selected from the group consisting of the intracellular domain of CD3z, of FCGR3A and of NKG2D, or fragments thereof.

6. The antigen binding receptor of any one of claims 1 to 5, wherein the at least one stimulatory signaling domain is the intracellular domain of CD3z or a fragment thereof, in particular wherein the at least one stimulatory signaling domain comprises the amino acid sequence of SEQ ID NO:16.

7. The antigen binding receptor of any one of claims 1 to 6, wherein the at least one co-stimulatory signaling domain is individually selected from the group consisting of the intracellular domain of CD27, of CD28, of CD137, of OX40, of ICOS, of DAP10 and of DAP12, or fragments thereof.

8. The antigen binding receptor of any one of claims 1 to 7, wherein the at least one co-stimulatory signaling domain is the CD28 intracellular domain or a fragment thereof, in particular, wherein the at least one co-stimulatory signaling domain comprises the amino acid sequence of SEQ ID NO:15.

9. The antigen binding receptor of any one of claims 1 to 8, wherein the antigen binding receptor comprises one stimulatory signaling domain comprising the intracellular domain of CD3z, or a fragment thereof, and wherein the antigen binding receptor comprises one co-stimulatory signaling domain comprising the intracellular domain of CD28, or a fragment thereof.

10. The antigen binding receptor of any one of claims 1 to 9, wherein the antigen binding moiety comprises a heavy chain constant domain (CH) and a light chain constant domain (CL), wherein the CH domain or the CL domain is connected at the C-terminus to the N-terminus of the anchoring transmembrane domain, optionally through a peptide linker.

11. The antigen binding receptor of any one of claims 4 to 10, wherein the antigen binding receptor comprises one co-signaling domain, wherein the co-signaling domain is connected at the N-terminus to the C-terminus of the anchoring transmembrane domain.

12. The antigen binding receptor of claim 11, wherein the antigen binding receptor additionally comprises one stimulatory signaling domain, wherein the stimulatory signaling domain is connected at the N-terminus to the C-terminus of the co-stimulatory signaling domain.

13. The antigen binding receptor of any one of claims 1 to 12, wherein the antigen binding moiety is capable of specific binding to an antigen selected from the group consisting of fibroblast activation protein (FAP), carcinoembryonic antigen (CEA), mesothelin (MSLN), CD20, folate receptor 1 (FOLR1), tenascin (TNC) and programmed death-ligand 1(PDL1).

14. The antigen binding receptor of any one of claims 1 to 13, wherein the at least one antigen binding moiety is a capable of specific binding to CD20, wherein the antigen binding moiety comprises:
(i) a heavy chain variable region (VH) comprising
(a) the heavy chain complementarity-determining region (CDR H) 1 amino acid sequence YSWIN (SEQ ID NO:1);
(b) the CDR H2 amino acid sequence RIFPGDGDTDYNGKFKG (SEQ ID NO:2); and
(c) the CDR H3 amino acid sequence NVFDGYWLVY (SEQ ID NO:3); and
(ii) a light chain variable region (VL) comprising
(d) the light chain complementary-determining region (CDR L) 1 amino acid sequence RSSKSLLHSNGITYLY (SEQ ID NO:4);
(e) the CDR L2 amino acid sequence QMSNLVS (SEQ ID NO:5); and
(f) the CDR L3 amino acid sequence AQNLELPYT (SEQ ID NO:6).

15. The antigen binding receptor of any one of claims 1 to 13, wherein the antigen binding moiety is a capable of specific binding to PDL1, wherein the antigen binding moiety comprises:
(i) a heavy chain variable region (VH) comprising
(a) the heavy chain complementarity-determining region (CDR H) 1 amino acid sequence DSWIH (SEQ ID NO:68);
(b) the CDR H2 amino acid sequence WISPYGGSTYYADSVKG (SEQ ID NO:69); and
(c) the CDR H3 amino acid sequence RHWPGGFDY (SEQ ID NO:70); and
(ii) a light chain variable region (VL) comprising
(d) the light chain complementary-determining region (CDR L) 1 amino acid sequence RASQDVSTAVA (SEQ ID NO:71);
(e) the CDR L2 amino acid sequence SASFLYS (SEQ ID NO:72); and
(f) the CDR L3 amino acid sequence QQYLYHPAT (SEQ ID NO:73).

16. The antigen binding receptor of any one of claims 1 to 13, wherein the antigen binding moiety is a capable of specific binding to CEA, wherein the antigen binding moiety comprises:
(i) a heavy chain variable region (VH) comprising
(a) the heavy chain complementarity-determining region (CDR H) 1 amino acid sequence EFGMN (SEQ ID NO:138);
(b) the CDR H2 amino acid sequence WINTKTGEATYVEEFKG (SEQ ID NO:139); and
(c) the CDR H3 amino acid sequence WDFAYYVEAMDY (SEQ ID NO:140); and
(ii) a light chain variable region (VL) comprising
(d) the light chain complementary-determining region (CDR L) 1 amino acid sequence KASAAVGTYVA (SEQ ID NO:141);
(e) the CDR L2 amino acid sequence SASYRKR (SEQ ID NO:142); and
(f) the CDR L3 amino acid sequence HQYYTYPLFT (SEQ ID NO:143).

17. The antigen binding receptor of any one of claims 1 to 13, wherein the antigen binding moiety is a capable of specific binding to CEA, wherein the antigen binding moiety comprises:
(i) a heavy chain variable region (VH) comprising
(a) the heavy chain complementarity-determining region (CDR H) 1 amino acid sequence DTYMH (SEQ ID NO:148);
(b) the CDR H2 amino acid sequence RIDPANGNSKYVPKFQG (SEQ ID NO:149); and
(c) the CDR H3 amino acid sequence FGYYVSDYAMAY (SEQ ID NO:150); and
(ii) a light chain variable region (VL) comprising
(d) the light chain complementary-determining region (CDR L) 1 amino acid sequence RAGESVDIFGVGFLH (SEQ ID NO:151);
(e) the CDR L2 amino acid sequence RASNRAT (SEQ ID NO:152); and
(f) the CDR L3 amino acid sequence QQTNEDPYT (SEQ ID NO:153).

18. An isolated polynucleotide encoding the antigen binding receptor of any one of claims 1 to 17.

19. A vector, particularly an expression vector, comprising the isolated polynucleotide of claim 18.

20. A transduced T cell capable of expressing at least one of the antigen binding receptor of any one of claims 1 to 17.

21. The transduced T cell of claim 20, wherein the cell comprises a first antigen binding receptor according to any one of claims 1 to 17, wherein a first antigen binding receptor comprises a Fab antigen binding moiety, and wherein the cell comprises a second antigen binding receptor according to any one of claims 1 to 17, wherein the second antigen binding receptor comprises a crossFab antigen binding moiety.

22. The antigen binding receptor of any one of claims 1 to 17 or the transduced T cell of any one of claim 20 or 21 for use as a medicament.

23. The antigen binding receptor of any one of claims 1 to 17 or the transduced T cell of any one of claims 20 to 21 for use in the treatment of a malignant disease, wherein the treatment comprises administration of a transduced T cell expressing the antigen binding receptor.

24. The antigen binding receptor or the transduced T cell for use according to claim 23, wherein said malignant disease is selected from cancer of epithelial, endothelial or mesothelial origin and cancer of the blood.

25. A method of treating a disease in a subject, comprising administering to the subject a transduced T cell capable of expressing the antigen binding receptor of any one of embodiments 1 to 17.

26. A method for inducing lysis of a target cell, comprising contacting the target cell with a transduced T cell capable of expressing the antigen binding receptor of any one of embodiments 1 to 17.

27. Use of the antigen binding receptor of any one of embodiments 1 to 17, the isolated polynucleotide of claim 18, or the transduced T cell of any one of claim 20 or 21 for the manufacture of a medicament.

28. The use of claim 27, wherein the medicament is for treatment of a malignant disease.

29. An antigen binding receptor substantially as hereinbefore described with reference to any of the Examples or to any one of the accompanying drawings.
